SEQUENCE LISTING (266 sequences)

SEQ ID NO: 1 - protein, 123 aa, Artificial Sequence
  Synthetic sequence SARS-CoV-2 S300-v1 mAb VH
QVQLVQSGAEVKKPGASVKVSCKASGYTFTDYYIHWVRQAPGQGPEWLGWVNGYSGATRYAQKYQGRVTMTRDTSISTAYMQLSRLRPDDTAVYYCARDRPSHEWAMYFFDNWGQGTLVTVSS

SEQ ID NO: 2 - protein, 8 aa, Artificial Sequence
  Synthetic sequence SARS-CoV-2 S300-v1 mAb CDRH1
GYTFTDYY

SEQ ID NO: 3 - protein, 8 aa, Artificial Sequence
  Synthetic sequence SARS-CoV-2 S300-v1 mAb CDRH2
VNGYSGAT

SEQ ID NO: 4 - protein, 16 aa, Artificial Sequence
  Synthetic sequence SARS-CoV-2 S300-v1 mAb CDRH3
ARDRPSHEWAMYFFDN

SEQ ID NO: 5 - protein, 109 aa, Artificial Sequence
  Synthetic sequence SARS-CoV-2 S300-v1 mAb VL (VK)
QIVLTQSPGTLSLSPGERATLSCRASQSVPSSCLAWYQQKPGQAPRLLIYGASGRATGIPDRFSGSGSGTDFTLTIRRLEPEDFAVYYCQQYGSSPPLTFGGGTKVEIK

SEQ ID NO: 6 - protein, 7 aa, Artificial Sequence
  Synthetic sequence SARS-CoV-2 S300-v1 mAb CDRL1
QSVPSSC

SEQ ID NO: 7 - protein, 3 aa, Artificial Sequence
  Synthetic sequence SARS-CoV-2 S300-v1 mAb CDRL2
GAS

SEQ ID NO: 8 - protein, 10 aa, Artificial Sequence
  Synthetic sequence SARS-CoV-2 S300-v1 mAb CDRL3
QQYGSSPPLT

SEQ ID NO: 9 - protein, 123 aa, Artificial Sequence
  Synthetic sequence SARS-CoV-2 S300-v1.1 mAb VH
QVQLVQSGAEVKKPGASVKVSCKASGYTFTDYYIHWVRQAPGQGPEWLGWVQGYSGATRYAQKYQGRVTMTRDTSISTAYMQLSRLRPDDTAVYYCARDRPSHEWAMYFFDNWGQGTLVTVSS

SEQ ID NO: 10 - protein, 123 aa, Artificial Sequence
  Synthetic sequence SARS-CoV-2 S300-v1.2 mAb VH
QVQLVQSGAEVKKPGASVKVSCKASGYTFTDYYIHWVRQAPGQGPEWLGWVNAYSGATRYAQKYQGRVTMTRDTSISTAYMQLSRLRPDDTAVYYCARDRPSHEWAMYFFDNWGQGTLVTVSS

SEQ ID NO: 11 - protein, 123 aa, Artificial Sequence
  Synthetic sequence SARS-CoV-2 S300-v1.3 mAb VH
QVQLVQSGAEVKKPGASVKVSCKASGYTFTDYYIHWVRQAPGQGPEWLGWVNSYSGATRYAQKYQGRVTMTRDTSISTAYMQLSRLRPDDTAVYYCARDRPSHEWAMYFFDNWGQGTLVTVSS

SEQ ID NO: 12 - protein, 123 aa, Artificial Sequence
  Synthetic sequence SARS-CoV-2 S300-v1.4 mAb VH
QVQLVQSGAEVKKPGASVKVSCKASGYTFTDYYIHWVRQAPGQGPEWLGWVNPYSGATRYAQKYQGRVTMTRDTSISTAYMQLSRLRPDDTAVYYCARDRPSHEWAMYFFDNWGQGTLVTVSS

SEQ ID NO: 13 - protein, 123 aa, Artificial Sequence
  Synthetic sequence SARS-CoV-2 S300-v1.5 mAb VH
QVQLVQSGAEVKKPGASVKVSCKASGYTFTDYYIHWVRQAPGQGPEWLGWVNQYSGATRYAQKYQGRVTMTRDTSISTAYMQLSRLRPDDTAVYYCARDRPSHEWAMYFFDNWGQGTLVTVSS

SEQ ID NO: 14 - protein, 123 aa, Artificial Sequence
  Synthetic sequence SARS-CoV-2 S300-v1.6 mAb VH
QVQLVQSGAEVKKPGASVKVSCKASGYTFTDYYIHWVRQAPGQGPEWLGWVLGYSGATRYAQKYQGRVTMTRDTSISTAYMQLSRLRPDDTAVYYCARDRPSHEWAMYFFDNWGQGTLVTVSS

SEQ ID NO: 15 - protein, 123 aa, Artificial Sequence
  Synthetic sequence SARS-CoV-2 S300-v1.7 mAb VH
QVQLVQSGAEVKKPGASVKVSCKASGYTFTDYYIHWVRQAPGQGPEWLGWVTGYSGATRYAQKYQGRVTMTRDTSISTAYMQLSRLRPDDTAVYYCARDRPSHEWAMYFFDNWGQGTLVTVSS

SEQ ID NO: 16 - protein, 8 aa, Artificial Sequence
  Synthetic sequence SARS-CoV-2 S300-v1.1 mAb CDRH2
VQGYSGAT

SEQ ID NO: 17 - protein, 8 aa, Artificial Sequence
  Synthetic sequence SARS-CoV-2 S300-v1.2 mAb CDRH2
VNAYSGAT

SEQ ID NO: 18 - protein, 8 aa, Artificial Sequence
  Synthetic sequence SARS-CoV-2 S300-v1.3 mAb CDRH2
VNSYSGAT

SEQ ID NO: 19 - protein, 8 aa, Artificial Sequence
  Synthetic sequence SARS-CoV-2 S300-v1.4 mAb CDRH2
VNPYSGAT

SEQ ID NO: 20 - protein, 8 aa, Artificial Sequence
  Synthetic sequence SARS-CoV-2 S300-v1.5 mAb CDRH2
VNQYSGAT

SEQ ID NO: 21 - protein, 8 aa, Artificial Sequence
  Synthetic sequence SARS-CoV-2 S300-v1.6 mAb CDRH2
VLGYSGAT

SEQ ID NO: 22 - protein, 8 aa, Artificial Sequence
  Synthetic sequence SARS-CoV-2 S300-v1.7 mAb CDRH2
VTGYSGAT

SEQ ID NO: 23 - protein, 123 aa, Artificial Sequence
  Synthetic sequence SARS-CoV-2 S300-v1.8 mAb VH
QVQLVQSGAEVKKPGASVKVSCKASGYTFTDYYIHWVRQAPGQGPEWLGWVNGYSGATRYAQKYQGRVTMTRDTSISTAYMQLSRLRPDDTAVYYCARDRPSHEFAMYFFDNWGQGTLVTVSS

SEQ ID NO: 24 - protein, 123 aa, Artificial Sequence
  Synthetic sequence SARS-CoV-2 S300-v1.9 mAb VH
QVQLVQSGAEVKKPGASVKVSCKASGYTFTDYYIHWVRQAPGQGPEWLGWVNGYSGATRYAQKYQGRVTMTRDTSISTAYMQLSRLRPDDTAVYYCARDRPSHEYAMYFFDNWGQGTLVTVSS

SEQ ID NO: 25 - protein, 16 aa, Artificial Sequence
  Synthetic sequence SARS-CoV-2 S300-v1.8 mAb CDRH3
ARDRPSHEFAMYFFDN

SEQ ID NO: 26 - protein, 16 aa, Artificial Sequence
  Synthetic sequence SARS-CoV-2 S300-v1.9 mAb CDRH3
ARDRPSHEYAMYFFDN

SEQ ID NO: 27 - protein, 123 aa, Artificial Sequence
  Synthetic sequence SARS-CoV-2 S300-v2 mAb VH
QVQLVQSGAEVKKPGASVKVSCKASGYTFTDYYIHWVRQAPGQGPEWLGFVNGYSGATRYAQKYQGRVTMTRDTSISTAYMQLSRLRPDDTAVYYCARDRPSHEWAMYFFDNWGQGTLVTVSS

SEQ ID NO: 28 - protein, 123 aa, Artificial Sequence
  Synthetic sequence SARS-CoV-2 S300-v2.1 mAb VH
QVQLVQSGAEVKKPGASVKVSCKASGYTFTDYYIHWVRQAPGQGPEWLGFVQGYSGATRYAQKYQGRVTMTRDTSISTAYMQLSRLRPDDTAVYYCARDRPSHEWAMYFFDNWGQGTLVTVSS

SEQ ID NO: 29 - protein, 123 aa, Artificial Sequence
  Synthetic sequence SARS-CoV-2 S300-v2.2 mAb VH
QVQLVQSGAEVKKPGASVKVSCKASGYTFTDYYIHWVRQAPGQGPEWLGFVNAYSGATRYAQKYQGRVTMTRDTSISTAYMQLSRLRPDDTAVYYCARDRPSHEWAMYFFDNWGQGTLVTVSS

SEQ ID NO: 30 - protein, 123 aa, Artificial Sequence
  Synthetic sequence SARS-CoV-2 S300-v2.3 mAb VH
QVQLVQSGAEVKKPGASVKVSCKASGYTFTDYYIHWVRQAPGQGPEWLGFVNSYSGATRYAQKYQGRVTMTRDTSISTAYMQLSRLRPDDTAVYYCARDRPSHEWAMYFFDNWGQGTLVTVSS

SEQ ID NO: 31 - protein, 123 aa, Artificial Sequence
  Synthetic sequence SARS-CoV-2 S300-v2.4 mAb VH
QVQLVQSGAEVKKPGASVKVSCKASGYTFTDYYIHWVRQAPGQGPEWLGFVNPYSGATRYAQKYQGRVTMTRDTSISTAYMQLSRLRPDDTAVYYCARDRPSHEWAMYFFDNWGQGTLVTVSS

SEQ ID NO: 32 - protein, 123 aa, Artificial Sequence
  Synthetic sequence SARS-CoV-2 S300-v2.5 mAb VH
QVQLVQSGAEVKKPGASVKVSCKASGYTFTDYYIHWVRQAPGQGPEWLGFVNQYSGATRYAQKYQGRVTMTRDTSISTAYMQLSRLRPDDTAVYYCARDRPSHEWAMYFFDNWGQGTLVTVSS

SEQ ID NO: 33 - protein, 123 aa, Artificial Sequence
  Synthetic sequence SARS-CoV-2 S300-v2.6 mAb VH
QVQLVQSGAEVKKPGASVKVSCKASGYTFTDYYIHWVRQAPGQGPEWLGFVLGYSGATRYAQKYQGRVTMTRDTSISTAYMQLSRLRPDDTAVYYCARDRPSHEWAMYFFDNWGQGTLVTVSS

SEQ ID NO: 34 - protein, 123 aa, Artificial Sequence
  Synthetic sequence SARS-CoV-2 S300-v2.7 mAb VH
QVQLVQSGAEVKKPGASVKVSCKASGYTFTDYYIHWVRQAPGQGPEWLGFVTGYSGATRYAQKYQGRVTMTRDTSISTAYMQLSRLRPDDTAVYYCARDRPSHEWAMYFFDNWGQGTLVTVSS

SEQ ID NO: 35 - protein, 123 aa, Artificial Sequence
  Synthetic sequence SARS-CoV-2 S300-v2.8 mAb VH
QVQLVQSGAEVKKPGASVKVSCKASGYTFTDYYIHWVRQAPGQGPEWLGFVNGYSGATRYAQKYQGRVTMTRDTSISTAYMQLSRLRPDDTAVYYCARDRPSHEFAMYFFDNWGQGTLVTVSS

SEQ ID NO: 36 - protein, 123 aa, Artificial Sequence
  Synthetic sequence SARS-CoV-2 S300-v2.9 mAb VH
QVQLVQSGAEVKKPGASVKVSCKASGYTFTDYYIHWVRQAPGQGPEWLGFVNGYSGATRYAQKYQGRVTMTRDTSISTAYMQLSRLRPDDTAVYYCARDRPSHEYAMYFFDNWGQGTLVTVSS

SEQ ID NO: 37 - protein, 123 aa, Artificial Sequence
  Synthetic sequence SARS-CoV-2 S300-v3 mAb VH
QVQLVQSGAEVKKPGASVKVSCKASGYTFTDYYIHWVRQAPGQGPEWLGYVNGYSGATRYAQKYQGRVTMTRDTSISTAYMQLSRLRPDDTAVYYCARDRPSHEWAMYFFDNWGQGTLVTVSS

SEQ ID NO: 38 - protein, 123 aa, Artificial Sequence
  Synthetic sequence SARS-CoV-2 S300-v3.1 mAb VH
QVQLVQSGAEVKKPGASVKVSCKASGYTFTDYYIHWVRQAPGQGPEWLGYVQGYSGATRYAQKYQGRVTMTRDTSISTAYMQLSRLRPDDTAVYYCARDRPSHEWAMYFFDNWGQGTLVTVSS

SEQ ID NO: 39 - protein, 123 aa, Artificial Sequence
  Synthetic sequence SARS-CoV-2 S300-v3.2 mAb VH
QVQLVQSGAEVKKPGASVKVSCKASGYTFTDYYIHWVRQAPGQGPEWLGYVNAYSGATRYAQKYQGRVTMTRDTSISTAYMQLSRLRPDDTAVYYCARDRPSHEWAMYFFDNWGQGTLVTVSS

SEQ ID NO: 40 - protein, 123 aa, Artificial Sequence
  Synthetic sequence SARS-CoV-2 S300-v3.3 mAb VH
QVQLVQSGAEVKKPGASVKVSCKASGYTFTDYYIHWVRQAPGQGPEWLGYVNSYSGATRYAQKYQGRVTMTRDTSISTAYMQLSRLRPDDTAVYYCARDRPSHEWAMYFFDNWGQGTLVTVSS

SEQ ID NO: 41 - protein, 123 aa, Artificial Sequence
  Synthetic sequence SARS-CoV-2 S300-v3.4 mAb VH
QVQLVQSGAEVKKPGASVKVSCKASGYTFTDYYIHWVRQAPGQGPEWLGYVNPYSGATRYAQKYQGRVTMTRDTSISTAYMQLSRLRPDDTAVYYCARDRPSHEWAMYFFDNWGQGTLVTVSS

SEQ ID NO: 42 - protein, 123 aa, Artificial Sequence
  Synthetic sequence SARS-CoV-2 S300-v3.5 mAb VH
QVQLVQSGAEVKKPGASVKVSCKASGYTFTDYYIHWVRQAPGQGPEWLGYVNQYSGATRYAQKYQGRVTMTRDTSISTAYMQLSRLRPDDTAVYYCARDRPSHEWAMYFFDNWGQGTLVTVSS

SEQ ID NO: 43 - protein, 123 aa, Artificial Sequence
  Synthetic sequence SARS-CoV-2 S300-v3.6 mAb VH
QVQLVQSGAEVKKPGASVKVSCKASGYTFTDYYIHWVRQAPGQGPEWLGYVLGYSGATRYAQKYQGRVTMTRDTSISTAYMQLSRLRPDDTAVYYCARDRPSHEWAMYFFDNWGQGTLVTVSS

SEQ ID NO: 44 - protein, 123 aa, Artificial Sequence
  Synthetic sequence SARS-CoV-2 S300-v3.7 mAb VH
QVQLVQSGAEVKKPGASVKVSCKASGYTFTDYYIHWVRQAPGQGPEWLGYVTGYSGATRYAQKYQGRVTMTRDTSISTAYMQLSRLRPDDTAVYYCARDRPSHEWAMYFFDNWGQGTLVTVSS

SEQ ID NO: 45 - protein, 123 aa, Artificial Sequence
  Synthetic sequence SARS-CoV-2 S300-v3.8 mAb VH
QVQLVQSGAEVKKPGASVKVSCKASGYTFTDYYIHWVRQAPGQGPEWLGYVNGYSGATRYAQKYQGRVTMTRDTSISTAYMQLSRLRPDDTAVYYCARDRPSHEFAMYFFDNWGQGTLVTVSS

SEQ ID NO: 46 - protein, 123 aa, Artificial Sequence
  Synthetic sequence SARS-CoV-2 S300-v3.9 mAb VH
QVQLVQSGAEVKKPGASVKVSCKASGYTFTDYYIHWVRQAPGQGPEWLGYVNGYSGATRYAQKYQGRVTMTRDTSISTAYMQLSRLRPDDTAVYYCARDRPSHEYAMYFFDNWGQGTLVTVSS

SEQ ID NO: 47 - protein, 109 aa, Artificial Sequence
  Synthetic sequence SARS-CoV-2 S300-v10 mAb VL (VK)
QIVLTQSPGTLSLSPGERATLSCRASQSVPSSYLAWYQQKPGQAPRLLIYGASGRATGIPDRFSGSGSGTDFTLTIRRLEPEDFAVYYCQQYGSSPPLTFGGGTKVEIK

SEQ ID NO: 48 - protein, 109 aa, Artificial Sequence
  Synthetic sequence SARS-CoV-2 S300-v11 mAb VL (VK)
QIVLTQSPGTLSLSPGERATLSCRASQSVPSSSLAWYQQKPGQAPRLLIYGASGRATGIPDRFSGSGSGTDFTLTIRRLEPEDFAVYYCQQYGSSPPLTFGGGTKVEIK

SEQ ID NO: 49 - protein, 109 aa, Artificial Sequence
  Synthetic sequence SARS-CoV-2 S300-v12 mAb VL (VK)
QIVLTQSPGTLSLSPGERATLSCRASQSVPSSTLAWYQQKPGQAPRLLIYGASGRATGIPDRFSGSGSGTDFTLTIRRLEPEDFAVYYCQQYGSSPPLTFGGGTKVEIK

SEQ ID NO: 50 - protein, 109 aa, Artificial Sequence
  Synthetic sequence SARS-CoV-2 S300-v13 mAb VL (VK)
QIVLTQSPGTLSLSPGERATLSCRASQSVPSSALAWYQQKPGQAPRLLIYGASGRATGIPDRFSGSGSGTDFTLTIRRLEPEDFAVYYCQQYGSSPPLTFGGGTKVEIK

SEQ ID NO: 51 - protein, 7 aa, Artificial Sequence
  Synthetic sequence SARS-CoV-2 S300-v10 mAb CDRL1
QSVPSSY

SEQ ID NO: 52 - protein, 7 aa, Artificial Sequence
  Synthetic sequence SARS-CoV-2 S300-v11 mAb CDRL1
QSVPSSS

SEQ ID NO: 53 - protein, 7 aa, Artificial Sequence
  Synthetic sequence SARS-CoV-2 S300-v12 mAb CDRL1
QSVPSST

SEQ ID NO: 54 - protein, 7 aa, Artificial Sequence
  Synthetic sequence SARS-CoV-2 S300-v13 mAb CDRL1
QSVPSSA

SEQ ID NO: 55 - protein, 121 aa, Artificial Sequence
  Synthetic sequence SARS-CoV-2 S302 mAb VH
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDISSGWDRVFDYWGQGTLVTVSS

SEQ ID NO: 56 - protein, 8 aa, Artificial Sequence
  Synthetic sequence SARS-CoV-2 S302 mAb CDRH1
GFTFSSYG

SEQ ID NO: 57 - protein, 8 aa, Artificial Sequence
  Synthetic sequence SARS-CoV-2 S302 mAb CDRH2
ISYDGSNK

SEQ ID NO: 58 - protein, 14 aa, Artificial Sequence
  Synthetic sequence SARS-CoV-2 S302 mAb CDRH3
AKDISSGWDRVFDY

SEQ ID NO: 59 - protein, 108 aa, Artificial Sequence
  Synthetic sequence SARS-CoV-2 S302 mAb VL (VK)
EILLTQSPGTLSLSPGERATLSCRTSQSVGSSYLAWYQQKPGQAPRLLIYAASSRAIGIPDRFSGSGSGTDFTLTISRLEPEDFAVYYCQQYGSSPWTFGQGTKVEIK

SEQ ID NO: 60 - protein, 7 aa, Artificial Sequence
  Synthetic sequence SARS-CoV-2 S302 mAb CDRL1
QSVGSSY

SEQ ID NO: 61 - protein, 3 aa, Artificial Sequence
  Synthetic sequence SARS-CoV-2 S302 mAb CDRL2
AAS

SEQ ID NO: 62 - protein, 9 aa, Artificial Sequence
  Synthetic sequence SARS-CoV-2 S302 mAb CDRL3
QQYGSSPWT

SEQ ID NO: 63 - protein, 124 aa, Artificial Sequence
  Synthetic sequence SARS-CoV-2 S303-v1 mAb VH
EVQLVESGGGLVKPGGSLRLSCAASGFTFLTYSMNWVRQTPGKRLQWVSAISGSGGATYYADSVKGRFTISRDNSKNTLYLQMNTVTADDTAIYFCARERDDIFPMGLNAFDIWGQGAMVIVSS

SEQ ID NO: 64 - protein, 8 aa, Artificial Sequence
  Synthetic sequence SARS-CoV-2 S303-v1 mAb CDRH1
GFTFLTYS

SEQ ID NO: 65 - protein, 8 aa, Artificial Sequence
  Synthetic sequence SARS-CoV-2 S303-v1 mAb CDRH2
ISGSGGAT

SEQ ID NO: 66 - protein, 17 aa, Artificial Sequence
  Synthetic sequence SARS-CoV-2 S303-v1 mAb CDRH3
ARERDDIFPMGLNAFDI

SEQ ID NO: 67 - protein, 107 aa, Artificial Sequence
  Synthetic sequence SARS-CoV-2 S303-v1 mAb VL (VK)
DIQMTQSPSTLSASVGDRVTITCRASQSISNWLAWYQQKPGKAPKLLIYKASSLESGVPSRFSGSGSGTEFTLTISSLQPDDSATYYCQQYDTYSWTFGQGTKVEIK

SEQ ID NO: 68 - protein, 6 aa, Artificial Sequence
  Synthetic sequence SARS-CoV-2 S303-v1 mAb CDRL1
QSISNW

SEQ ID NO: 69 - protein, 3 aa, Artificial Sequence
  Synthetic sequence SARS-CoV-2 S303-v1 mAb CDRL2
KAS

SEQ ID NO: 70 - protein, 9 aa, Artificial Sequence
  Synthetic sequence SARS-CoV-2 S303-v1 mAb CDRL3
QQYDTYSWT

SEQ ID NO: 71 - protein, 107 aa, Artificial Sequence
  Synthetic sequence SARS-CoV-2 S303-v2 mAb VL (VK)
DIQMTQSPSTLSASVGDRVTITCRASQSISNFLAWYQQKPGKAPKLLIYKASSLESGVPSRFSGSGSGTEFTLTISSLQPDDSATYYCQQYDTYSWTFGQGTKVEIK

SEQ ID NO: 72 - protein, 107 aa, Artificial Sequence
  Synthetic sequence SARS-CoV-2 S303-v3 mAb VL (VK)
DIQMTQSPSTLSASVGDRVTITCRASQSISNYLAWYQQKPGKAPKLLIYKASSLESGVPSRFSGSGSGTEFTLTISSLQPDDSATYYCQQYDTYSWTFGQGTKVEIK

SEQ ID NO: 73 - protein, 6 aa, Artificial Sequence
  Synthetic sequence SARS-CoV-2 S303-v2 mAb CDRL1
QSISNF

SEQ ID NO: 74 - protein, 6 aa, Artificial Sequence
  Synthetic sequence SARS-CoV-2 S303-v3 mAb CDRL1
QSISNY

SEQ ID NO: 75 - protein, 107 aa, Artificial Sequence
  Synthetic sequence SARS-CoV-2 S303-v4 mAb VL (VK)
DIQMTQSPSTLSASVGDRVTITCRASQSISNWLAWYQQKPGKAPKLLIYKASSLESGVPSRFSGSGSGTEFTLTISSLQPDDSATYYCQQYDTYSFTFGQGTKVEIK

SEQ ID NO: 76 - protein, 107 aa, Artificial Sequence
  Synthetic sequence SARS-CoV-2 S303-v5 mAb VL (VK)
DIQMTQSPSTLSASVGDRVTITCRASQSISNWLAWYQQKPGKAPKLLIYKASSLESGVPSRFSGSGSGTEFTLTISSLQPDDSATYYCQQYDTYSYTFGQGTKVEIK

SEQ ID NO: 77 - protein, 9 aa, Artificial Sequence
  Synthetic sequence SARS-CoV-2 S303-v4 mAb CDRL3
QQYDTYSFT

SEQ ID NO: 78 - protein, 9 aa, Artificial Sequence
  Synthetic sequence SARS-CoV-2 S303-v5 mAb CDRL3
QQYDTYSYT

SEQ ID NO: 79 - protein, 120 aa, Artificial Sequence
  Synthetic sequence SARS-CoV-2 S304 mAb VH
EVQLVESGGGLVQPGGSLRLSCAASGFTFSSYDMHWVRQTTGKGLEWVSTIGTAGDTYYPDSVKGRFTISREDAKNSLYLQMNSLRAGDTAVYYCARGDSSGYYYYFDYWGQGTLLTVSS

SEQ ID NO: 80 - protein, 8 aa, Artificial Sequence
  Synthetic sequence SARS-CoV-2 S304 mAb CDRH1
GFTFSSYD

SEQ ID NO: 81 - protein, 7 aa, Artificial Sequence
  Synthetic sequence SARS-CoV-2 S304 mAb CDRH2
IGTAGDT

SEQ ID NO: 82 - protein, 14 aa, Artificial Sequence
  Synthetic sequence SARS-CoV-2 S304 mAb CDRH3
ARGDSSGYYYYFDY

SEQ ID NO: 83 - protein, 108 aa, Artificial Sequence
  Synthetic sequence SARS-CoV-2 S304 mAb VL (VK)
DIQMTQSPSSLSAAVGDRVTITCRASQSIGSYLNWYQQKPGKAPKLLIYAASSLQSGVPSRFSGSGSGTDFTLTISSLQPEDFAIYYCQQSYVSPTYTFGPGTKVDIK

SEQ ID NO: 84 - protein, 6 aa, Artificial Sequence
  Synthetic sequence SARS-CoV-2 S304 mAb CDRL1
QSIGSY

SEQ ID NO: 85 - protein, 3 aa, Artificial Sequence
  Synthetic sequence SARS-CoV-2 S304 mAb CDRL2
AAS

SEQ ID NO: 86 - protein, 10 aa, Artificial Sequence
  Synthetic sequence SARS-CoV-2 S304 mAb CDRL3
QQSYVSPTYT

SEQ ID NO: 87 - protein, 123 aa, Artificial Sequence
  Synthetic sequence SARS-CoV-2 S306 mAb VH
QVQLVQSGAEVKKPGASVKVSCKASTYTFTSFGISWVRQAPGQGLEWMGWITTYSGDTNYAQKFQGRVTMTTDTSTNTAYMELRSLRSDDTAVYYCASDYFDSSGYYHSFDYWGQGTLVTVSS

SEQ ID NO: 88 - protein, 8 aa, Artificial Sequence
  Synthetic sequence SARS-CoV-2 S306 mAb CDRH1
TYTFTSFG

SEQ ID NO: 89 - protein, 8 aa, Artificial Sequence
  Synthetic sequence SARS-CoV-2 S306 mAb CDRH2
ITTYSGDT

SEQ ID NO: 90 - protein, 16 aa, Artificial Sequence
  Synthetic sequence SARS-CoV-2 S306 mAb CDRH3
ASDYFDSSGYYHSFDY

SEQ ID NO: 91 - protein, 109 aa, Artificial Sequence
  Synthetic sequence SARS-CoV-2 S306 mAb VL (VK)
EIVLTQSPDTLSLSPGERATLSCRASQSVSSYLAWYQQRPGQAPRLLIYDASKRATGIPARFSGSGSGTDFTLTISSLEPEDFAVYYCQQRSNWPPGCSFGQGTKVEIK

SEQ ID NO: 92 - protein, 6 aa, Artificial Sequence
  Synthetic sequence SARS-CoV-2 S306 mAb CDRL1
QSVSSY

SEQ ID NO: 93 - protein, 3 aa, Artificial Sequence
  Synthetic sequence SARS-CoV-2 S306 mAb CDRL2
DAS

SEQ ID NO: 94 - protein, 11 aa, Artificial Sequence
  Synthetic sequence SARS-CoV-2 S306 mAb CDRL3
QQRSNWPPGCS

SEQ ID NO: 95 - protein, 127 aa, Artificial Sequence
  Synthetic sequence SARS-CoV-2 S308-v1 mAb VH
QVQLVESGGGVVQPGRSLRLSCAASRFTFSSYGMHWVRQAPGKGLEWVAVIWHDGNNKHYGDSVKGRVTISRDNSKNTLYLQMTSLRAEDTAVYYCARAVTTFKGSGRARMRGMDVWGQGTTVTVSS

SEQ ID NO: 96 - protein, 8 aa, Artificial Sequence
  Synthetic sequence SARS-CoV-2 S308-v1 mAb CDRH1
RFTFSSYG

SEQ ID NO: 97 - protein, 8 aa, Artificial Sequence
  Synthetic sequence SARS-CoV-2 S308-v1 mAb CDRH2
IWHDGNNK

SEQ ID NO: 98 - protein, 20 aa, Artificial Sequence
  Synthetic sequence SARS-CoV-2 S308-v1 mAb CDRH3
ARAVTTFKGSGRARMRGMDV

SEQ ID NO: 99 - protein, 107 aa, Artificial Sequence
  Synthetic sequence SARS-CoV-2 S308-v1 mAb VL (VK)
DIQLTQSPSFLSASVGDRVTITCRASQGINTYLAWYQQKPGKAPKLLIYAASTLQSGVPSRFSGSGSGTEFTLTISSLQPEDFATYYCQHLDTYPFTFGPGTKVDIK

SEQ ID NO: 100 - protein, 6 aa, Artificial Sequence
  Synthetic sequence SARS-CoV-2 S308-v1 mAb CDRL1
QGINTY

SEQ ID NO: 101 - protein, 3 aa, Artificial Sequence
  Synthetic sequence SARS-CoV-2 S308-v1 mAb CDRL2
AAS

SEQ ID NO: 102 - protein, 9 aa, Artificial Sequence
  Synthetic sequence SARS-CoV-2 S308-v1 mAb CDRL3
QHLDTYPFT

SEQ ID NO: 103 - protein, 127 aa, Artificial Sequence
  Synthetic sequence SARS-CoV-2 S308-v2 mAb VH
QVQLVESGGGVVQPGRSLRLSCAASRFTFSSYGMHWVRQAPGKGLEWVAVIWHDGNNKHYGDSVKGRVTISRDNSKNTLYLQMTSLRAEDTAVYYCARAVTTFKGSGRARLRGMDVWGQGTTVTVSS

SEQ ID NO: 104 - protein, 20 aa, Artificial Sequence
  Synthetic sequence SARS-CoV-2 S308-v2 mAb CDRH3
ARAVTTFKGSGRARLRGMDV

SEQ ID NO: 105 - protein, 127 aa, Artificial Sequence [sequence omitted]

SEQ ID NO: 106 - protein, 8 aa, Artificial Sequence
  Synthetic sequence SARS-CoV-2 S309-v1 mAb CDRH1
GYPFTSYG

SEQ ID NO: 107 - protein, 8 aa, Artificial Sequence
  Synthetic sequence SARS-CoV-2 S309-v1 mAb CDRH2
ISTYNGNT

SEQ ID NO: 108 - protein, 20 aa, Artificial Sequence
  Synthetic sequence SARS-CoV-2 S309-v1 mAb CDRH3
ARDYTRGAWFGESLIGGFDN

SEQ ID NO: 109 - protein, 107 aa, Artificial Sequence
  Synthetic sequence SARS-CoV-2 S309-v1 mAb VL (VK)
DIQMTQSPSSLSTSVGDRVTITCRASQGINNYVAWYQQKPGKVPKLLIYGASTLQSGVPSRFRGSGSGTGFTLTISSLQPEDVASYYCRKYNSAPWTFGQGTRVEIK

SEQ ID NO: 110 - protein, 6 aa, Artificial Sequence
  Synthetic sequence SARS-CoV-2 S309-v1 mAb CDRL1
QGINNY

SEQ ID NO: 111 - protein, 3 aa, Artificial Sequence
  Synthetic sequence SARS-CoV-2 S309-v1 mAb CDRL2
GAS

SEQ ID NO: 112 - protein, 9 aa, Artificial Sequence
  Synthetic sequence SARS-CoV-2 S309-v1 mAb CDRL3
RKYNSAPWT

SEQ ID NO: 113 - protein, 127 aa, Artificial Sequence
  Synthetic sequence SARS-CoV-2 S309-v1.1 mAb VH
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 114 - protein, 127 aa, Artificial Sequence
  Synthetic sequence SARS-CoV-2 S309-v1.2 mAb VH
QVQLVQSGAEVKKPGASVKVSCKASGYPFTSYGISWVRQAPGQGLEWMGWISTYNSNTNYAQKFQGRVTMTTDTSTTTGYMELRRLRSDDTAVYYCARDYTRGAWFGESLIGGFDNWGQGTLVTVSS

SEQ ID NO: 115 - protein, 127 aa, Artificial Sequence
  Synthetic sequence SARS-CoV-2 S309-v1.3 mAb VH
QVQLVQSGAEVKKPGASVKVSCKASGYPFTSYGISWVRQAPGQGLEWMGWISTYNANTNYAQKFQGRVTMTTDTSTTTGYMELRRLRSDDTAVYYCARDYTRGAWFGESLIGGFDNWGQGTLVTVSS

SEQ ID NO: 116 - protein, 127 aa, Artificial Sequence
  Synthetic sequence SARS-CoV-2 S309-v1.4 mAb VH
QVQLVQSGAEVKKPGASVKVSCKASGYPFTSYGISWVRQAPGQGLEWMGWISTYNQNTNYAQKFQGRVTMTTDTSTTTGYMELRRLRSDDTAVYYCARDYTRGAWFGESLIGGFDNWGQGTLVTVSS

SEQ ID NO: 117 - protein, 127 aa, Artificial Sequence
  Synthetic sequence SARS-CoV-2 S309-v1.5 mAb VH
QVQLVQSGAEVKKPGASVKVSCKASGYPFTSYGISWVRQAPGQGLEWMGWISTYLGNTNYAQKFQGRVTMTTDTSTTTGYMELRRLRSDDTAVYYCARDYTRGAWFGESLIGGFDNWGQGTLVTVSS

SEQ ID NO: 118 - protein, 127 aa, Artificial Sequence
  Synthetic sequence SARS-CoV-2 S309-v1.6 mAb VH
QVQLVQSGAEVKKPGASVKVSCKASGYPFTSYGISWVRQAPGQGLEWMGWISTYTGNTNYAQKFQGRVTMTTDTSTTTGYMELRRLRSDDTAVYYCARDYTRGAWFGESLIGGFDNWGQGTLVTVSS

SEQ ID NO: 119 - protein, 127 aa, Artificial Sequence
  Synthetic sequence SARS-CoV-2 S309-v1.7 mAb VH
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAFFGESLIGGFDNWGQGTLVTVSS

SEQ ID NO: 120 - protein, 127 aa, Artificial Sequence
  Synthetic sequence SARS-CoV-2 S309-v1.8 mAb VH
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAYFGESLIGGFDNWGQGTLVTVSS

SEQ ID NO: 121 - protein, 8 aa, Artificial Sequence
  Synthetic sequence SARS-CoV-2 S309-v1.1 mAb CDRH2
ISTYQGNT

SEQ ID NO: 122 - protein, 8 aa, Artificial Sequence
  Synthetic sequence SARS-CoV-2 S309-v1.2 mAb CDRH2
ISTYNSNT

SEQ ID NO: 123 - protein, 8 aa, Artificial Sequence
  Synthetic sequence SARS-CoV-2 S309-v1.3 mAb CDRH2
ISTYNANT

SEQ ID NO: 124 - protein, 8 aa, Artificial Sequence
  Synthetic sequence SARS-CoV-2 S309-v1.4 mAb CDRH2
ISTYNQNT

SEQ ID NO: 125 - protein, 8 aa, Artificial Sequence
  Synthetic sequence SARS-CoV-2 S309-v1.5 mAb CDRH2
ISTYLGNT

SEQ ID NO: 126 - protein, 8 aa, Artificial Sequence
  Synthetic sequence SARS-CoV-2 S309-v1.6 mAb CDRH2
ISTYTGNT

SEQ ID NO: 127 - protein, 20 aa, Artificial Sequence
  Synthetic sequence SARS-CoV-2 S309-v1.7 mAb CDRH3
ARDYTRGAFFGESLIGGFDN

SEQ ID NO: 128 - protein, 20 aa, Artificial Sequence
  Synthetic sequence SARS-CoV-2 S309-v1.8 mAb CDRH3
ARDYTRGAYFGESLIGGFDN

SEQ ID NO: 129 - protein, 127 aa, Artificial Sequence
  Synthetic sequence SARS-CoV-2 S309-v2 mAb VH
QVQLVQSGAEVKKPGASVKVSCKASGYPFTSYGISWVRQAPGQGLEWMGFISTYNGNTNYAQKFQGRVTMTTDTSTTTGYMELRRLRSDDTAVYYCARDYTRGAWFGESLIGGFDNWGQGTLVTVSS

SEQ ID NO: 130 - protein, 127 aa, Artificial Sequence
  Synthetic sequence SARS-CoV-2 S309-v2.1 mAb VH
QVQLVQSGAEVKKPGASVKVSCKASGYPFTSYGISWVRQAPGQGLEWMGFISTYQGNTNYAQKFQGRVTMTTDTSTTTGYMELRRLRSDDTAVYYCARDYTRGAWFGESLIGGFDNWGQGTLVTVSS

SEQ ID NO: 131 - protein, 127 aa, Artificial Sequence
  Synthetic sequence SARS-CoV-2 S309-v2.2 mAb VH
QVQLVQSGAEVKKPGASVKVSCKASGYPFTSYGISWVRQAPGQGLEWMGFISTYNSNTNYAQKFQGRVTMTTDTSTTTGYMELRRLRSDDTAVYYCARDYTRGAWFGESLIGGFDNWGQGTLVTVSS

SEQ ID NO: 132 - protein, 127 aa, Artificial Sequence
  Synthetic sequence SARS-CoV-2 S309-v2.3 mAb VH
QVQLVQSGAEVKKPGASVKVSCKASGYPFTSYGISWVRQAPGQGLEWMGFISTYNANTNYAQKFQGRVTMTTDTSTTTGYMELRRLRSDDTAVYYCARDYTRGAWFGESLIGGFDNWGQGTLVTVSS

SEQ ID NO: 133 - protein, 127 aa, Artificial Sequence
  Synthetic sequence SARS-CoV-2 S309-v2.4 mAb VH
QVQLVQSGAEVKKPGASVKVSCKASGYPFTSYGISWVRQAPGQGLEWMGFISTYNQNTNYAQKFQGRVTMTTDTSTTTGYMELRRLRSDDTAVYYCARDYTRGAWFGESLIGGFDNWGQGTLVTVSS

SEQ ID NO: 134 - protein, 127 aa, Artificial Sequence
  Synthetic sequence SARS-CoV-2 S309-v2.5 mAb VH
QVQLVQSGAEVKKPGASVKVSCKASGYPFTSYGISWVRQAPGQGLEWMGFISTYLGNTNYAQKFQGRVTMTTDTSTTTGYMELRRLRSDDTAVYYCARDYTRGAWFGESLIGGFDNWGQGTLVTVSS

SEQ ID NO: 135 - protein, 127 aa, Artificial Sequence
  Synthetic sequence SARS-CoV-2 S309-v2.6 mAb VH
QVQLVQSGAEVKKPGASVKVSCKASGYPFTSYGISWVRQAPGQGLEWMGFISTYTGNTNYAQKFQGRVTMTTDTSTTTGYMELRRLRSDDTAVYYCARDYTRGAWFGESLIGGFDNWGQGTLVTVSS

SEQ ID NO: 136 - protein, 127 aa, Artificial Sequence
  Synthetic sequence SARS-CoV-2 S309-v2.7 mAb VH
QVQLVQSGAEVKKPGASVKVSCKASGYPFTSYGISWVRQAPGQGLEWMGFISTYNGNTNYAQKFQGRVTMTTDTSTTTGYMELRRLRSDDTAVYYCARDYTRGAFFGESLIGGFDNWGQGTLVTVSS

SEQ ID NO: 137 - protein, 127 aa, Artificial Sequence
  Synthetic sequence SARS-CoV-2 S309-v2.8 mAb VH
QVQLVQSGAEVKKPGASVKVSCKASGYPFTSYGISWVRQAPGQGLEWMGFISTYNGNTNYAQKFQGRVTMTTDTSTTTGYMELRRLRSDDTAVYYCARDYTRGAYFGESLIGGFDNWGQGTLVTVSS

SEQ ID NO: 138 - protein, 127 aa, Artificial Sequence
  Synthetic sequence SARS-CoV-2 S309-v3 mAb VH
QVQLVQSGAEVKKPGASVKVSCKASGYPFTSYGISWVRQAPGQGLEWMGYISTYNGNTNYAQKFQGRVTMTTDTSTTTGYMELRRLRSDDTAVYYCARDYTRGAWFGESLIGGFDNWGQGTLVTVSS

SEQ ID NO: 139 - protein, 127 aa, Artificial Sequence
  Synthetic sequence SARS-CoV-2 S309-v3.1 mAb VH
QVQLVQSGAEVKKPGASVKVSCKASGYPFTSYGISWVRQAPGQGLEWMGYISTYQGNTNYAQKFQGRVTMTTDTSTTTGYMELRRLRSDDTAVYYCARDYTRGAWFGESLIGGFDNWGQGTLVTVSS

SEQ ID NO: 140 - protein, 127 aa, Artificial Sequence
  Synthetic sequence SARS-CoV-2 S309-v3.2 mAb VH
QVQLVQSGAEVKKPGASVKVSCKASGYPFTSYGISWVRQAPGQGLEWMGYISTYNSNTNYAQKFQGRVTMTTDTSTTTGYMELRRLRSDDTAVYYCARDYTRGAWFGESLIGGFDNWGQGTLVTVSS

SEQ ID NO: 141 - protein, 127 aa, Artificial Sequence
  Synthetic sequence SARS-CoV-2 S309-v3.3 mAb VH
QVQLVQSGAEVKKPGASVKVSCKASGYPFTSYGISWVRQAPGQGLEWMGYISTYNANTNYAQKFQGRVTMTTDTSTTTGYMELRRLRSDDTAVYYCARDYTRGAWFGESLIGGFDNWGQGTLVTVSS

SEQ ID NO: 142 - protein, 127 aa, Artificial Sequence
  Synthetic sequence SARS-CoV-2 S309-v3.4 mAb VH
QVQLVQSGAEVKKPGASVKVSCKASGYPFTSYGISWVRQAPGQGLEWMGYISTYNQNTNYAQKFQGRVTMTTDTSTTTGYMELRRLRSDDTAVYYCARDYTRGAWFGESLIGGFDNWGQGTLVTVSS

SEQ ID NO: 143 - protein, 127 aa, Artificial Sequence
  Synthetic sequence SARS-CoV-2 S309-v3.5 mAb VH
QVQLVQSGAEVKKPGASVKVSCKASGYPFTSYGISWVRQAPGQGLEWMGYISTYLGNTNYAQKFQGRVTMTTDTSTTTGYMELRRLRSDDTAVYYCARDYTRGAWFGESLIGGFDNWGQGTLVTVSS

SEQ ID NO: 144 - protein, 127 aa, Artificial Sequence
  Synthetic sequence SARS-CoV-2 S309-v3.6 mAb VH
QVQLVQSGAEVKKPGASVKVSCKASGYPFTSYGISWVRQAPGQGLEWMGYISTYTGNTNYAQKFQGRVTMTTDTSTTTGYMELRRLRSDDTAVYYCARDYTRGAWFGESLIGGFDNWGQGTLVTVSS

SEQ ID NO: 145 - protein, 127 aa, Artificial Sequence
  Synthetic sequence SARS-CoV-2 S309-v3.7 mAb VH
QVQLVQSGAEVKKPGASVKVSCKASGYPFTSYGISWVRQAPGQGLEWMGYISTYNGNTNYAQKFQGRVTMTTDTSTTTGYMELRRLRSDDTAVYYCARDYTRGAFFGESLIGGFDNWGQGTLVTVSS

SEQ ID NO: 146 - protein, 127 aa, Artificial Sequence
  Synthetic sequence SARS-CoV-2 S309-v3.8 mAb VH
QVQLVQSGAEVKKPGASVKVSCKASGYPFTSYGISWVRQAPGQGLEWMGYISTYNGNTNYAQKFQGRVTMTTDTSTTTGYMELRRLRSDDTAVYYCARDYTRGAYFGESLIGGFDNWGQGTLVTVSS

SEQ ID NO: 147 - protein, 107 aa, Artificial Sequence
  Synthetic sequence SARS-CoV-2 S309-v9 mAb VL (VK)
DIQMTQSPSSLSTSVGDRVTITCRASQGINNYVAWYQQKPGKVPKLLIYGASTLQSGVPSRFRGSGSGTGFTLTISSLQPEDVASYYCRKYNSAPGTFGQGTRVEIK

SEQ ID NO: 148 - protein, 107 aa, Artificial Sequence
  Synthetic sequence SARS-CoV-2 S309-v10 mAb VL (VK)
DIQMTQSPSSLSTSVGDRVTITCRASQGINNYVAWYQQKPGKVPKLLIYGASTLQSGVPSRFRGSGSGTGFTLTISSLQPEDVASYYCRKYNSAPRTFGQGTRVEIK

SEQ ID NO: 149 - protein, 107 aa, Artificial Sequence
  Synthetic sequence SARS-CoV-2 S309-v11 mAb VL (VK)
DIQMTQSPSSLSTSVGDRVTITCRASQGINNYVAWYQQKPGKVPKLLIYGASTLQSGVPSRFRGSGSGTGFTLTISSLQPEDVASYYCRKYNSAPFTFGQGTRVEIK

SEQ ID NO: 150 - protein, 107 aa, Artificial Sequence
  Synthetic sequence SARS-CoV-2 S309-v12 mAb VL (VK)
DIQMTQSPSSLSTSVGDRVTITCRASQGINNYVAWYQQKPGKVPKLLIYGASTLQSGVPSRFRGSGSGTGFTLTISSLQPEDVASYYCRKYNSAPYTFGQGTRVEIK

SEQ ID NO: 151 - protein, 9 aa, Artificial Sequence
  Synthetic sequence SARS-CoV-2 S309-v9 mAb CDRL3
RKYNSAPGT

SEQ ID NO: 152 - protein, 9 aa, Artificial Sequence
  Synthetic sequence SARS-CoV-2 S309-v10 mAb CDRL3
RKYNSAPRT

SEQ ID NO: 153 - protein, 9 aa, Artificial Sequence
  Synthetic sequence SARS-CoV-2 S309-v11 mAb CDRL3
RKYNSAPFT

SEQ ID NO: 154 - protein, 9 aa, Artificial Sequence
  Synthetic sequence SARS-CoV-2 S309-v12 mAb CDRL3
RKYNSAPYT

SEQ ID NO: 155 - protein, 126 aa, Artificial Sequence
  Synthetic sequence SARS-CoV-2 S310 mAb VH
QVQLVQSGAELKKPGSSVKVSCKASGGTFNSYSFNWVRQAPGQGLEWLGGIIPVLGTSNYAQKFQGRVAVTADEFTTTAYMELSSLRSEDTAVYYCATRTYDSSGYRPYYYGLDVWGQGTPVTVSS

SEQ ID NO: 156 - protein, 8 aa, Artificial Sequence
  Synthetic sequence SARS-CoV-2 S310 mAb CDRH1
GGTFNSYS

SEQ ID NO: 157 - protein, 8 aa, Artificial Sequence
  Synthetic sequence SARS-CoV-2 S310 mAb CDRH2
IIPVLGTS

SEQ ID NO: 158 - protein, 19 aa, Artificial Sequence
  Synthetic sequence SARS-CoV-2 S310 mAb CDRH3
ATRTYDSSGYRPYYYGLDV

SEQ ID NO: 159 - protein, 110 aa, Artificial Sequence
  Synthetic sequence SARS-CoV-2 S310 mAb VL (VK)
QSALTQPASVSGSPGQSITISCTGTSSDVGSYNLVSWYQQRPGKAPELMIYEVTKRPSGLSNRFSGSKSGNTASLTISGLQAEDEADYYCCSYAGSDTVIFGGGTKVTVL

SEQ ID NO: 160 - protein, 9 aa, Artificial Sequence
  Synthetic sequence SARS-CoV-2 S310 mAb CDRL1
SSDVGSYNL

SEQ ID NO: 161 - protein, 3 aa, Artificial Sequence
  Synthetic sequence SARS-CoV-2 S310 mAb CDRL2
EVT

SEQ ID NO: 162 - protein, 10 aa, Artificial Sequence
  Synthetic sequence SARS-CoV-2 S310 mAb CDRL3
CSYAGSDTVI

SEQ ID NO: 163 - DNA, 29902 bp, SARS-CoV-2 betacoronavirus
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggccgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 164 - protein, 7096 aa, SARS-CoV-2 betacoronavirus [sequence omitted]

SEQ ID NO: 165 - protein, 1273 aa, SARS-CoV-2 betacoronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 166 - protein, 220 aa, Artificial Sequence
  Synthetic sequence surface glycoprotein RBD [Wuhan seafood market pneumonia virus]; GenBank QHD43416.1; January 23, 2020
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTG

SEQ ID NO: 167 - protein, 72 aa, Artificial Sequence
  Synthetic sequence Receptor Binding Motif (RBM) in surface glycoprotein RBD [Wuhan seafood market pneumonia virus]; GenBank QHD43416.1; January 23, 2020
NSNNLDSKVGGNYNYLYRLFRKSNLKPFERDISTEIYQAGSTPCNGVEGFNCYFPLQSYGFQPTNGVGYQPY

SEQ ID NO: 168 - protein, 107 aa, Artificial Sequence [sequence omitted]

SEQ ID NO: 169 - protein, 7 aa, Artificial Sequence
  Synthetic sequence SARS-CoV-2 S309-v13 mAb CDRL1
QTVSSTS

SEQ ID NO: 170 - protein, 3 aa, Artificial Sequence
  Synthetic sequence SARS-CoV-2 S309-v13 mAb CDRL2
GAS

SEQ ID NO: 171 - protein, 8 aa, Artificial Sequence
  Synthetic sequence SARS-CoV-2 S309-v13 mAb CDRL3
QQHDTSLT

SEQ ID NO: 172 - protein, 127 aa, Artificial Sequence
  Synthetic sequence SARS-CoV-2 S309-v2.9 mAb VH
QVQLVQSGAEVKKPGASVKVSCKASGYPFTSYGISWVRQAPGQGLEWMGFISTYNANTNYAQKFQGRVTMTTDTSTTTGYMELRRLRSDDTAVYYCARDYTRGAFFGESLIGGFDNWGQGTLVTVSS

SEQ ID NO: 173 - protein, 330 aa, Artificial Sequence
  Synthetic sequence SARS-CoV-2 CH1-CH3 G1m17; IgG1*01 LS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 174 - protein, 107 aa, Artificial Sequence [sequence omitted]

SEQ ID NO: 175 - protein, 330 aa, Artificial Sequence
  Synthetic sequence SARS-CoV-2 CH1-CH3 G1m17; IgG1*01 LS GAALIE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 176 - protein, 123 aa, Artificial Sequence
  Synthetic sequence SARS-CoV-2 S300-v2.10 mAb VH
QVQLVQSGAEVKKPGASVKVSCKASGYTFTDYYIHWVRQAPGQGPEWLGFVNAYSGATRYAQKYQGRVTMTRDTSISTAYMQLSRLRPDDTAVYYCARDRPSHEFAMYFFDNWGQGTLVTVSS

SEQ ID NO: 177 - protein, 123 aa, Artificial Sequence
  Synthetic sequence SARS-CoV-2 S300-v2.11 mAb VH
QVQLVQSGAEVKKPGASVKVSCKASGYTFTDYYIHWVRQAPGQGPEWLGFVQGYSGATRYAQKYQGRVTMTRDTSISTAYMQLSRLRPDDTAVYYCARDRPSHEFAMYFFDNWGQGTLVTVSS

SEQ ID NO: 178 - protein, 124 aa, Artificial Sequence
  Synthetic sequence SARS-CoV-2 S315-v1 mAb VH
EVQLVESGGGLVQPGGSLRLSCAASGFTFSNYWMTWVRQAPGKGLEWVANIKQDGSEKYYVDSVKGRFTISRDNAKNSLYLQMNSLRAEDTAVYYCARDLWWNDQAHYYGMDVWGQGTTVTVSS

SEQ ID NO: 179 - protein, 8 aa, Artificial Sequence
  Synthetic sequence SARS-CoV-2 S315-v1 mAb CDRH1
GFTFSNYW

SEQ ID NO: 180 - protein, 8 aa, Artificial Sequence
  Synthetic sequence SARS-CoV-2 S315-v1 mAb CDRH2
IKQDGSEK

SEQ ID NO: 181 - protein, 17 aa, Artificial Sequence
  Synthetic sequence SARS-CoV-2 S315-v1 mAb CDRH3
ARDLWWNDQAHYYGMDV

SEQ ID NO: 182 - protein, 106 aa, Artificial Sequence
  Synthetic sequence SARS-CoV-2 S315-v1 mAb VL
SYELTQPPSVSVSPGQTARITCSGDAFPNQYAYWYQQKPGQAPVMLIYKDSERPSGIPERFFGSSSGTTVTLTIRGVQAEDEADYYCQSADSSGTVFGGGTKLTVL

SEQ ID NO: 183 - protein, 6 aa, Artificial Sequence
  Synthetic sequence SARS-CoV-2 S315-v1 mAb CDRL1
AFPNQY

SEQ ID NO: 184 - protein, 3 aa, Artificial Sequence
  Synthetic sequence SARS-CoV-2 S315-v1 mAb CDRL2
KDS

SEQ ID NO: 185 - protein, 9 aa, Artificial Sequence
  Synthetic sequence SARS-CoV-2 S315-v1 mAb CDRL3
QSADSSGTV

SEQ ID NO: 186 - DNA, 373 bp, Artificial Sequence
  Synthetic sequence SARS-CoV-2 S315-v1 mAb VH
gaggtgcagctggtggagtctgggggaggcttggtccagcctggggggtccctgagactctcctgtgcagcctctggattcacctttagtaattattggatgacctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagatctttggtggaacgaccaggctcactactacggtatggacgtctggggccaagggaccacggtcaccgtctcctcag

SEQ ID NO: 187 - DNA, 319 bp, Artificial Sequence
  Synthetic sequence SARS-CoV-2 S315-v1 mAb VL
tcctatgagctgacacagccaccctcggtgtcagtgtccccaggacagacggccaggatcacctgctctggagatgcattcccaaaccaatatgcttattggtaccagcagaagccaggccaggcccctgtgatgctgatctataaagacagtgagaggccctcagggatccctgagcgattctttggctccagctcagggacaacagtcacgttgaccatcagaggagtccaggcagaagacgaggctgactattactgtcaatcagcagacagcagtggtaccgtgttcggcggagggaccaagctgaccgtcctag

SEQ ID NO: 188 - DNA, 372 bp, Artificial Sequence
  Synthetic sequence SARS-CoV-2 S315-v1 mAb VH (nt - codon optimized)
gaagtgcagcttgtcgagagcggcggaggcctcgttcagccaggtgggagtctccgtctttcatgcgccgcttcaggatttacgttctccaactactggatgacatgggtgaggcaggcacctgggaaggggctggagtgggtggctaacatcaagcaggacggatctgaaaaatattatgtagattctgtgaaggggcggtttaccatctcaagggataatgccaaaaactctttgtatttacagatgaactctcttcgagccgaggacaccgccgtttactactgtgcccgagatctatggtggaatgaccaggctcactattatggaatggacgtgtggggccagggtactaccgttaccgtctcctca

SEQ ID NO: 189 - DNA, 318 bp, Artificial Sequence
  Synthetic sequence SARS-CoV-2 S315-v1 mAb VL (nt- codon optimized)
tcttacgagctcacccagccaccctcagtgtcagtgagccctggccaaacagctcgcatcacctgttcaggtgacgcctttccaaatcagtacgcctactggtatcagcagaaacccggccaggcacccgttatgctcatctacaaagattctgagcggccatccggtatccccgaacgctttttcggaagctccagtgggactacagttacacttactatccggggagtgcaagctgaagatgaggccgactattattgccagagcgcagactcctcaggcacagtgtttgggggcgggactaaactaactgtgctg

SEQ ID NO: 190 - protein, 106 aa, Artificial Sequence
  Synthetic sequence SARS-CoV-2 S315-v2 mAb VL
SYELTQPPSVSVSPGQTARITCSGDAFPNQYAYWYQQKPGQAPVMLIYKDSERPSGIPERFFGSSSGTTVTLTISGVQAEDEADYYCQSADSSGTVFGGGTKLTVL

SEQ ID NO: 191 - DNA, 319 bp, Artificial Sequence
  Synthetic sequence SARS-CoV-2 S315-v2 mAb VL
tcctatgagctgacacagccaccctcggtgtcagtgtccccaggacagacggccaggatcacctgctctggagatgcattcccaaaccaatatgcttattggtaccagcagaagccaggccaggcccctgtgatgctgatctataaagacagtgagaggccctcagggatccctgagcgattctttggctccagctcagggacaacagtcacgttgaccatcagtggagtccaggcagaagacgaggctgactattactgtcaatcagcagacagcagtggtaccgtgttcggcggagggaccaagctgaccgtcctag

SEQ ID NO: 192 - DNA, 318 bp, Artificial Sequence
  Synthetic sequence SARS-CoV-2 S315-v2 mAb VL (nt - codon optimized)
tcctacgagctcacccagcccccctcagtctctgtgtctcctggacagacagccagaatcacctgctcgggagatgcttttcccaaccaatacgcctactggtaccaacagaaaccaggtcaggcgcctgtcatgctgatttataaagactcagagcggccttcaggaattcccgaaagattcttcgggagttcaagcggaactaccgtgaccttaaccataagcggggtgcaggccgaagatgaagcagactattattgccagagtgccgatagtagtggcacagtctttgggggggggacaaagctgacagtactc

SEQ ID NO: 193 - protein, 106 aa, Artificial Sequence
  Synthetic sequence SARS-CoV-2 mAb CL IgLC*01
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 194 - protein, 124 aa, Artificial Sequence
  Synthetic sequence SARS-CoV-2 S315-v3 mAb VH
EVQLVESGGGLVQPGGSLRLSCAASGFTFSNYFMTWVRQAPGKGLEWVANIKQDGSEKYYVDSVKGRFTISRDNAKNSLYLQMNSLRAEDTAVYYCARDLWWNDQAHYYGMDVWGQGTTVTVSS

SEQ ID NO: 195 - protein, 8 aa, Artificial Sequence
  Synthetic sequence SARS-CoV-2 S315-v3 mAb CDRH1
GFTFSNYF

SEQ ID NO: 196 - protein, 124 aa, Artificial Sequence
  Synthetic sequence SARS-CoV-2 S315-v4 mAb VH
EVQLVESGGGLVQPGGSLRLSCAASGFTFSNYWMTWVRQAPGKGLEWVANIKQDASEKYYVDSVKGRFTISRDNAKNSLYLQMNSLRAEDTAVYYCARDLWWNDQAHYYGMDVWGQGTTVTVSS

SEQ ID NO: 197 - protein, 8 aa, Artificial Sequence
  Synthetic sequence SARS-CoV-2 S315-v4 mAb CDRH2
IKQDASEK

SEQ ID NO: 198 - protein, 124 aa, Artificial Sequence
  Synthetic sequence SARS-CoV-2 S315-v5 mAb VH
EVQLVESGGGLVQPGGSLRLSCAASGFTFSNYWMTWVRQAPGKGLEWVANIKQEGSEKYYVDSVKGRFTISRDNAKNSLYLQMNSLRAEDTAVYYCARDLWWNDQAHYYGMDVWGQGTTVTVSS

SEQ ID NO: 199 - protein, 8 aa, Artificial Sequence
  Synthetic sequence SARS-CoV-2 S315-v5 mAb CDRH2
IKQEGSEK

SEQ ID NO: 200 - protein, 124 aa, Artificial Sequence
  Synthetic sequence SARS-CoV-2 S315-v6 mAb VH
EVQLVESGGGLVQPGGSLRLSCAASGFTFSNYWMTWVRQAPGKGLEWVANIKQDGSEKYYVDSVKGRFTISRDNAKNSLYLQMNSLRAEDTAVYYCARDLFWNDQAHYYGMDVWGQGTTVTVSS

SEQ ID NO: 201 - protein, 17 aa, Artificial Sequence
  Synthetic sequence SARS-CoV-2 S315-v6 mAb CDRH3
ARDLFWNDQAHYYGMDV

SEQ ID NO: 202 - protein, 124 aa, Artificial Sequence
  Synthetic sequence SARS-CoV-2 S315-v7 mAb VH
EVQLVESGGGLVQPGGSLRLSCAASGFTFSNYWMTWVRQAPGKGLEWVANIKQDGSEKYYVDSVKGRFTISRDNAKNSLYLQMNSLRAEDTAVYYCARDLWFNDQAHYYGMDVWGQGTTVTVSS

SEQ ID NO: 203 - protein, 17 aa, Artificial Sequence
  Synthetic sequence SARS-CoV-2 S315-v7 mAb CDRH3
ARDLWFNDQAHYYGMDV

SEQ ID NO: 204 - protein, 230 aa, Artificial Sequence
  Synthetic sequence SARS-CoV-2 Heavy Chain IgHG1*01 Fd
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 205 - protein, 214 aa, Artificial Sequence
  Synthetic sequence SARS-CoV-2 Light Chain IgKC*01
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 18 aa, Artificial Sequence
  Synthetic linker sequence
GSTSGSGKPGSGEGSTKG

SEQ ID NO: 207 - protein, 10 aa, Artificial Sequence
  Synthetic linker sequence
GSGKPGSGEG

SEQ ID NO: 208 - protein, 8 aa, Artificial Sequence
  Synthetic linker sequence
GKPGSGEG

SEQ ID NO: 209 - protein, 8 aa, Artificial Sequence
  Synthetic linker sequence
SGKPGSGE

SEQ ID NO: 210 - protein, 6 aa, Artificial Sequence
  Synthetic linker sequence
  VARIANT (1)..(1): Xaa =positively charged amino acid
  VARIANT (3)..(5): Xaa = glycine (G) or serine (S)
  VARIANT (6)..(6): Xaa = glycine (G) or a negatively charged amino acid
XPXXXX

SEQ ID NO: 211 - protein, 110 aa, Artificial Sequence
  Synthetic linker sequence
  VARIANT (2)..(10): any one or all of amino acids 2-10 can either be present or absent
  VARIANT (12)..(110): any one or all of amino acids 12-110 can either be present or absent
GGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGS

SEQ ID NO: 212 - protein, 15 aa, Artificial Sequence
  Synthetic linker sequence
GGGGSGGGGSGGGGS

SEQ ID NO: 213 - protein, 50 aa, Artificial Sequence
  Synthetic linker sequence
GGGGSGGGGSGGGGSGGGGSGGGGSGGGGSGGGGSGGGGSGGGGSGGGGS

SEQ ID NO: 214 - protein, 18 aa, Artificial Sequence
  Synthetic linker sequence
GSTSGGGSGGGSGGGGSS

SEQ ID NO: 215 - protein, 14 aa, Artificial Sequence
  Synthetic linker sequence
EGKSSGSGSESKVD

SEQ ID NO: 216 - protein, 18 aa, Artificial Sequence
  Synthetic linker sequence
KESGSVSSEQLAQFRSLD

SEQ ID NO: 217 - protein, 5 aa, Artificial Sequence
  Synthetic linker sequence
GGGGS

SEQ ID NO: 218 - protein, 494 aa, Artificial Sequence
  Synthetic sequence SARS-CoV-2 S309-scFab (H-L)
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSGGGGSGGGGSGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 494 aa, Artificial Sequence
  Synthetic sequence SARS-CoV-2 S309-scFab (L-H)
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 220 - protein, 249 aa, Artificial Sequence
  Synthetic sequence SARS-CoV-2 S309-scFv (VH-VL)
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 221 - protein, 249 aa, Artificial Sequence
  Synthetic sequence SARS-CoV-2 S309-scFv (VL-VH)
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSS

SEQ ID NO: 222 - protein, 518 aa, Artificial Sequence
  Synthetic sequence SARS-CoV-2 S309-scFv (VH-VL)-(VH-VL)
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 223 - protein, 518 aa, Artificial Sequence
  Synthetic sequence SARS-CoV-2 S309-scFv-(VH-VL)-(VL-VH)
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSS

SEQ ID NO: 224 - protein, 518 aa, Artificial Sequence
  Synthetic sequence SARS-CoV-2 S309-scFv-(VL-VH)-(VH-VL)
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 225 - protein, 518 aa, Artificial Sequence
  Synthetic sequence SARS-CoV-2 S309-scFv-(VL-VH)-(VL-VH)
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSS

SEQ ID NO: 226 - protein, 494 aa, Artificial Sequence
  Synthetic sequence SARS-CoV-2 S309-scFab-(H-L) v1.1
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSGGGGSGGGGSGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 494 aa, Artificial Sequence
  Synthetic sequence SARS-CoV-2 S309-scFab-(L-H) v1.1
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 228 - protein, 249 aa, Artificial Sequence
  Synthetic sequence SARS-CoV-2 S309-scFv-(VH-VL) v1.1
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 229 - protein, 249 aa, Artificial Sequence
  Synthetic sequence SARS-CoV-2 S309-scFv-(VL-VH) v1.1
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 230 - protein, 518 aa, Artificial Sequence
  Synthetic sequence SARS-CoV-2 S309-scFv-(VH-VL)-(VH-VL) v1.1
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 231 - protein, 518 aa, Artificial Sequence
  Synthetic sequence SARS-CoV-2 S309-scFv-(VH-VL)-(VL-VH) v1.1
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 232 - protein, 518 aa, Artificial Sequence
  Synthetic sequence SARS-CoV-2 S309-scFv-(VL-VH)-(VH-VL) v1.1
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 233 - protein, 518 aa, Artificial Sequence
  Synthetic sequence SARS-CoV-2 S309-scFv-(VL-VH)-(VL-VH) v1.1
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 234 - protein, 114 aa, Artificial Sequence
  Synthetic sequence SARS-CoV-2 S300-v14 mAb VL (VK)
DIVMTQSPDSLAVSLGERATINCKSSQSVLYSSNNKNYLAWYQQKPGQPPKLLISWASTRESGVPDRFSGSGSGTDFTLTISSLQAEDVAVYYCQQYYSAPGITFGQGTRLEIK

SEQ ID NO: 235 - protein, 12 aa, Artificial Sequence
  Synthetic sequence SARS-CoV-2 S300-v14 mAb CDRL1
QSVLYSSNNKNY

SEQ ID NO: 236 - protein, 3 aa, Artificial Sequence
  Synthetic sequence SARS-CoV-2 S300-v14 mAb CDRL2
WAS

SEQ ID NO: 237 - protein, 10 aa, Artificial Sequence
  Synthetic sequence SARS-CoV-2 S300-v14 mAb CDRL3
QQYYSAPGIT

SEQ ID NO: 238 - DNA, 343 bp, Artificial Sequence
  Synthetic sequence SARS-CoV2 S300-v14 mAb VL (VK)
gacatcgtgatgacccagtctccagactcactggctgtgtctctgggcgagagggccaccatcaactgtaagtccagccagagtgttttatacagctccaacaataagaactacttagcttggtaccagcagaaaccaggacagcctcctaagctgctcatttcctgggcttctacccgggaatccggggtccctgaccgattcagtggcagcgggtctgggacagatttcactctcaccatcagcagcctgcaggctgaagatgtggcagtttattactgtcaacaatattatagtgctcccgggatcaccttcggccaggggacacgactggagattaaac

SEQ ID NO: 239 - protein, 129 aa, Artificial Sequence
  Synthetic sequence SARS-CoV2 S307 mAb VH
QVQLQESGPGLVKPSETLSLTCTVSGGSVTSGSYYWSWIRQPPGKGLEWIGYMYYSGSTNYNPSLKSRVTISVDTSKNQFSLKLSSVTAADTAVYYCARAGCTGITCLRYDYYYGLDVWGQGTTVTVSS

SEQ ID NO: 240 - protein, 10 aa, Artificial Sequence
  Synthetic sequence SARS-CoV2 S307 mAb CDRH1
GGSVTSGSYY

SEQ ID NO: 241 - protein, 7 aa, Artificial Sequence
  Synthetic sequence SARS-CoV2 S307 mAb CDRH2
MYYSGST

SEQ ID NO: 242 - protein, 21 aa, Artificial Sequence
  Synthetic sequence SARS-CoV2 S307 mAb CDRH3
ARAGCTGITCLRYDYYYGLDV

SEQ ID NO: 243 - protein, 108 aa, Artificial Sequence
  Synthetic sequence SARS-CoV2 S307 mAb VL(VK)
EIVLTQSPGTLSLSPGKRATLSCRASQSVSSSYLAWYQQRPGQAPRLLIYGASSRAAGIPDRFSGSGSGTDFTLTISRLEPEDFAVYYCQQYGSSSWTFGQGTKVEIK

SEQ ID NO: 244 - protein, 7 aa, Artificial Sequence
  Synthetic sequence SARS-CoV2 S307 mAb CDRL1
QSVSSSY

SEQ ID NO: 245 - protein, 3 aa, Artificial Sequence
  Synthetic sequence SARS-CoV2 S307 mAb CDRL2
GAS

SEQ ID NO: 246 - protein, 9 aa, Artificial Sequence
  Synthetic sequence SARS-CoV2 S307 mAb CDRL3
QQYGSSSWT

SEQ ID NO: 247 - DNA, 387 bp, Artificial Sequence
  Synthetic sequence SARS-CoV2 S307 mAb VH
caggtgcagctgcaggagtcgggcccaggactggtgaagccttcggagaccctgtccctcacctgcactgtctctggtggctccgtcaccagtggtagttactactggagctggatccggcagcccccagggaagggactggagtggattgggtatatgtattacagtgggagcaccaattacaacccctccctcaagagtcgagtcaccatatcagtagacacgtccaagaaccagttctccctgaagctgagctctgtgaccgctgcggacacggccgtgtattactgtgcgagggcaggttgtactggtatcacctgcttacgttacgactactactacggtctggacgtctggggccaagggaccacggtcaccgtctcctca

SEQ ID NO: 248 - DNA, 325 bp, Artificial Sequence
  Synthetic sequence SARS-CoV2 S307 mAb VL(VK)
gaaattgtgttgacgcagtctccaggcaccctgtctttgtctccagggaaaagagccaccctctcctgcagggccagtcagagtgttagcagcagctacttagcctggtaccagcagagacctggccaggctcccaggctcctcatctatggtgcatccagcagggccgctggcatcccagacaggttcagtggcagtgggtctgggacagacttcactctcaccatcagcagactggagcctgaagattttgcagtgtattactgtcagcagtatggtagctcatcgtggacgttcggccaagggaccaaggtggaaatcaaac

SEQ ID NO: 249 - DNA, 381 bp, Artificial Sequence
  Synthetic sequence SARS-CoV-2 S309-v1 mAb VH
caggttcagctggtgcagtctggagctgaggtgaagaagcctggggcctcagtgaaggtctcctgcaaggcttctggttacccctttaccagttatggtatcagctgggtgcgacaggcccctggacaagggcttgagtggatgggatggatcagcacttacaatggtaacacaaattatgcacagaagttccagggcagagtcaccatgaccacagacacatccacgaccacaggctacatggagctgaggaggctgagatctgacgacacggccgtgtattactgtgcgagagattatactcgtggtgcttggttcggggagtcattgatagggggctttgacaactggggccagggaaccctggtcaccgtctcctca

SEQ ID NO: 250 - DNA, 322 bp, Artificial Sequence
  Synthetic sequence SAES-CoV-2 S309-v13 mAb VL(VK)
gaaattgtgttgacgcagtctccaggcaccctgtctttgtctccaggggaaagagccaccctctcctgcagggccagtcagactgttagcagcacctccttagcctggtaccagcagaaacctggccaggctcccaggctcctcatctatggtgcatccagcagggccactggcatcccagacaggttcagcggcagtgggtctgggacagacttcactctcaccatcagcagactggagcctgaagattttgcagtgtattactgtcagcagcatgatacctcactcactttcggcggagggaccaaggtggagatcaaac

SEQ ID NO: 251 - DNA, 767 bp, Artificial Sequence
  Synthetic sequence CMV promoter
gacattgattattgactagttattaatagtaatcaattacggggtcattagttcatagcccatatatggagttccgcgttacataacttacggtaaatggcccgcctggctgaccgcccaacgacccccgcccatgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttag

SEQ ID NO: 252 - DNA, 50 bp, Artificial Sequence
  Synthetic sequence Signal peptide
atgggatggtcatgtatcatcctttttctagtagcaactgcaaccggtgt

SEQ ID NO: 253 - DNA, 135 bp, Artificial Sequence
  Synthetic sequence Poly-adenylation signal sequence
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 254 - DNA, 323 bp, Artificial Sequence
  Synthetic sequence SARS-CoV-2 Light Chain IgKC*01
gtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 255 - DNA, 993 bp, Artificial Sequence
  Synthetic sequence SARS-CoV-2 CH1-CH3 G1m17; IgG1*01
gcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 256 - protein, 16 aa, Artificial Sequence
  Synthetic sequence Signal peptide
MGWSCIILFLVATATG

SEQ ID NO: 257 - DNA, 1431 bp, Artificial Sequence
  Synthetic sequence SARS-CoV-2 S309-v1.1 mAb CH1-CH3 G1m17; IgGHG1*01 LS; GAALIE; signal peptide
atgggctggtcctgtatcatcctgttcctggtcgccacagccaccggagtgcacagccaagtgcagctggtccagagcggcgccgaggtgaagaagcccggcgctagcgtgaaggtgtcctgtaaagccagcggatatccttttaccagctacggcatctcctgggtgcggcaggcccctggccagggcctggaatggatgggctggatcagcacctaccagggaaataccaactacgcccagaagttccagggaagagtgacaatgaccacagatacatctacaaccaccggctacatggaactgaggcggctgagaagcgacgacaccgccgtgtactactgcgccagagattacaccagaggcgcttggttcggcgagagcctgatcggcggcttcgacaactggggccagggaaccctggtgacagtgtctagcgcttctaccaaaggcccttctgtctttcctctggccccttctagcaagtctacaagcggaggcaccgccgccctgggctgcctggtgaaggactacttccccgagcccgtgaccgtgagctggaatagcggcgccctgacaagcggcgtgcacaccttcccagctgtgctgcagagcagcggcctgtatagcctgagcagcgtggtcaccgtgcccagcagcagcctgggaacacagacctacatctgcaacgtgaaccacaagccttctaataccaaggtggataagaaggtggaacctaagagctgcgacaaaacacacacatgccctccatgtcctgctccagagctgctggccggccccagcgtttttctgttcccccccaaacctaaagacaccctgatgatcagcagaacccctgaggtgacctgtgtggtggtggacgtgtcccacgaagatcctgaggtgaagttcaactggtacgtggatggagtggaagtgcacaacgccaagaccaaacctagagaagagcagtacaacagcacatatagagtcgtgtccgtgcttacagtgctgcaccaggactggctgaatggaaaggaatacaagtgcaaggtgtccaacaaggccctgcctctgcctgaggagaagacaatctctaaagccaagggccaacctcgggaacctcaggtgtacacactgccccccagccgggacgagctgaccaagaaccaggtgtccctgacctgcctggtcaagggcttctacccctctgatatcgccgtggaatgggagagcaacggccaacctgagaacaactacaagaccacccctccagtgctggacagcgacggcagcttcttcctgtacagcaagctgaccgttgacaagtccagatggcagcagggcaacgtgttcagctgtagcgtcctgcacgaggccctgcattctcactacacccagaagagcctgtccctcagccctggcaagtga

SEQ ID NO: 258 - DNA, 1374 bp, Artificial Sequence
  Synthetic sequence SARS-CoV-2 S309-v1.1 mAb CH1-CH3 G1m17; IgGHG1*01 LS; GAALIE;
caagtgcagctggtccagagcggcgccgaggtgaagaagcccggcgctagcgtgaaggtgtcctgtaaagccagcggatatccttttaccagctacggcatctcctgggtgcggcaggcccctggccagggcctggaatggatgggctggatcagcacctaccagggaaataccaactacgcccagaagttccagggaagagtgacaatgaccacagatacatctacaaccaccggctacatggaactgaggcggctgagaagcgacgacaccgccgtgtactactgcgccagagattacaccagaggcgcttggttcggcgagagcctgatcggcggcttcgacaactggggccagggaaccctggtgacagtgtctagcgcttctaccaaaggcccttctgtctttcctctggccccttctagcaagtctacaagcggaggcaccgccgccctgggctgcctggtgaaggactacttccccgagcccgtgaccgtgagctggaatagcggcgccctgacaagcggcgtgcacaccttcccagctgtgctgcagagcagcggcctgtatagcctgagcagcgtggtcaccgtgcccagcagcagcctgggaacacagacctacatctgcaacgtgaaccacaagccttctaataccaaggtggataagaaggtggaacctaagagctgcgacaaaacacacacatgccctccatgtcctgctccagagctgctggccggccccagcgtttttctgttcccccccaaacctaaagacaccctgatgatcagcagaacccctgaggtgacctgtgtggtggtggacgtgtcccacgaagatcctgaggtgaagttcaactggtacgtggatggagtggaagtgcacaacgccaagaccaaacctagagaagagcagtacaacagcacatatagagtcgtgtccgtgcttacagtgctgcaccaggactggctgaatggaaaggaatacaagtgcaaggtgtccaacaaggccctgcctctgcctgaggagaagacaatctctaaagccaagggccaacctcgggaacctcaggtgtacacactgccccccagccgggacgagctgaccaagaaccaggtgtccctgacctgcctggtcaagggcttctacccctctgatatcgccgtggaatgggagagcaacggccaacctgagaacaactacaagaccacccctccagtgctggacagcgacggcagcttcttcctgtacagcaagctgaccgttgacaagtccagatggcagcagggcaacgtgttcagctgtagcgtcctgcacgaggccctgcattctcactacacccagaagagcctgtccctcagccctggcaagtga

SEQ ID NO: 259 - DNA, 1431 bp, Artificial Sequence
  Synthetic sequence SARS-CoV-2 S309-v1.1 mAb CH1-CH3 G1m17; IgGHG1*01 LS; signal peptide
atgggctggtcctgtatcatcctgttcctggtcgccacagccaccggagtgcacagccaagtgcagctggtccagagcggcgccgaggtgaagaagcccggcgctagcgtgaaggtgtcctgtaaagccagcggatatccttttaccagctacggcatctcctgggtgcggcaggcccctggccagggcctggaatggatgggctggatcagcacctaccagggaaataccaactacgcccagaagttccagggaagagtgacaatgaccacagatacatctacaaccaccggctacatggaactgaggcggctgagaagcgacgacaccgccgtgtactactgcgccagagattacaccagaggcgcttggttcggcgagagcctgatcggcggcttcgacaactggggccagggaaccctggtgacagtgtctagcgcttctaccaaaggcccttctgtctttcctctggccccttctagcaagtctacaagcggaggcaccgccgccctgggctgcctggtgaaggactacttccccgagcccgtgaccgtgagctggaatagcggcgccctgacaagcggcgtgcacaccttcccagctgtgctgcagagcagcggcctgtatagcctgagcagcgtggtcaccgtgcccagcagcagcctgggaacacagacctacatctgcaacgtgaaccacaagccttctaataccaaggtggataagaaggtggaacctaagagctgcgacaaaacacacacatgccctccatgtcctgctccagagctgctgggcggccccagcgtttttctgttcccccccaaacctaaagacaccctgatgatcagcagaacccctgaggtgacctgtgtggtggtggacgtgtcccacgaagatcctgaggtgaagttcaactggtacgtggatggagtggaagtgcacaacgccaagaccaaacctagagaagagcagtacaacagcacatatagagtcgtgtccgtgcttacagtgctgcaccaggactggctgaatggaaaggaatacaagtgcaaggtgtccaacaaggccctgcctgcccctatcgagaagacaatctctaaagccaagggccaacctcgggaacctcaggtgtacacactgccccccagccgggacgagctgaccaagaaccaggtgtccctgacctgcctggtcaagggcttctacccctctgatatcgccgtggaatgggagagcaacggccaacctgagaacaactacaagaccacccctccagtgctggacagcgacggcagcttcttcctgtacagcaagctgaccgttgacaagtccagatggcagcagggcaacgtgttcagctgtagcgtcctgcacgaggccctgcattctcactacacccagaagagcctgtccctcagccctggcaagtga

SEQ ID NO: 260 - DNA, 1374 bp, Artificial Sequence
  Synthetic sequence SARS-CoV-2 S309-v1.1 mAb CH1-CH3 G1m17; IgGHG1*01 LS
caagtgcagctggtccagagcggcgccgaggtgaagaagcccggcgctagcgtgaaggtgtcctgtaaagccagcggatatccttttaccagctacggcatctcctgggtgcggcaggcccctggccagggcctggaatggatgggctggatcagcacctaccagggaaataccaactacgcccagaagttccagggaagagtgacaatgaccacagatacatctacaaccaccggctacatggaactgaggcggctgagaagcgacgacaccgccgtgtactactgcgccagagattacaccagaggcgcttggttcggcgagagcctgatcggcggcttcgacaactggggccagggaaccctggtgacagtgtctagcgcttctaccaaaggcccttctgtctttcctctggccccttctagcaagtctacaagcggaggcaccgccgccctgggctgcctggtgaaggactacttccccgagcccgtgaccgtgagctggaatagcggcgccctgacaagcggcgtgcacaccttcccagctgtgctgcagagcagcggcctgtatagcctgagcagcgtggtcaccgtgcccagcagcagcctgggaacacagacctacatctgcaacgtgaaccacaagccttctaataccaaggtggataagaaggtggaacctaagagctgcgacaaaacacacacatgccctccatgtcctgctccagagctgctgggcggccccagcgtttttctgttcccccccaaacctaaagacaccctgatgatcagcagaacccctgaggtgacctgtgtggtggtggacgtgtcccacgaagatcctgaggtgaagttcaactggtacgtggatggagtggaagtgcacaacgccaagaccaaacctagagaagagcagtacaacagcacatatagagtcgtgtccgtgcttacagtgctgcaccaggactggctgaatggaaaggaatacaagtgcaaggtgtccaacaaggccctgcctgcccctatcgagaagacaatctctaaagccaagggccaacctcgggaacctcaggtgtacacactgccccccagccgggacgagctgaccaagaaccaggtgtccctgacctgcctggtcaagggcttctacccctctgatatcgccgtggaatgggagagcaacggccaacctgagaacaactacaagaccacccctccagtgctggacagcgacggcagcttcttcctgtacagcaagctgaccgttgacaagtccagatggcagcagggcaacgtgttcagctgtagcgtcctgcacgaggccctgcattctcactacacccagaagagcctgtccctcagccctggcaagtga

SEQ ID NO: 261 - DNA, 701 bp, Artificial Sequence
  Synthetic sequence S309-v13 Light chain k1m3; IgKC*01; signal peptide
tgggctggtcctgcatcatcctgttcctggtggccacagccaccggcgtgcacagcgagatcgtcctgacacagagccccggcacactgagcctctccccaggcgagcgggctacactgtcttgtagagcttctcagaccgtgtccagcaccagcctggcctggtatcagcagaaacctggccaggcccctagactgctgatctacggcgccagcagcagagccaccggcatccctgatagattcagcggcagcggatctggaaccgacttcaccctgaccatcagccggctggaacccgaggactttgccgtgtactactgccagcaacacgacaccagcctgaccttcggcggcggaacaaaggtggaaatcaagagaaccgtggccgcccctagcgtgttcatcttcccccccagcgacgagcagctgaagagcggtacagcttctgtggtgtgcctgctgaacaacttctacccgcgggaagccaaggtgcagtggaaggtggacaacgccctgcagagcggcaacagccaggagagcgtgacagagcaggacagcaaggacagcacctacagcctgagcagcaccctgaccctgagcaaggccgactacgagaagcacaaggtgtacgcctgtgaagtgacccaccagggcctgtctagccctgtgaccaagtcttttaacagaggcgagtgctga

SEQ ID NO: 262 - DNA, 645 bp, Artificial Sequence
  Synthetic sequence S309-v13 Light chain k1m3; IgKC*01
gagatcgtcctgacacagagccccggcacactgagcctctccccaggcgagcgggctacactgtcttgtagagcttctcagaccgtgtccagcaccagcctggcctggtatcagcagaaacctggccaggcccctagactgctgatctacggcgccagcagcagagccaccggcatccctgatagattcagcggcagcggatctggaaccgacttcaccctgaccatcagccggctggaacccgaggactttgccgtgtactactgccagcaacacgacaccagcctgaccttcggcggcggaacaaaggtggaaatcaagagaaccgtggccgcccctagcgtgttcatcttcccccccagcgacgagcagctgaagagcggtacagcttctgtggtgtgcctgctgaacaacttctacccgcgggaagccaaggtgcagtggaaggtggacaacgccctgcagagcggcaacagccaggagagcgtgacagagcaggacagcaaggacagcacctacagcctgagcagcaccctgaccctgagcaaggccgactacgagaagcacaaggtgtacgcctgtgaagtgacccaccagggcctgtctagccctgtgaccaagtcttttaacagaggcgagtgctga

SEQ ID NO: 263 - DNA, 57 bp, Artificial Sequence
  Synthetic sequence Signal peptide
atgggctggtcctgcatcatcctgttcctggtggccacagccaccggcgtgcacagc

SEQ ID NO: 264 - protein, 19 aa, Artificial Sequence
  Synthetic sequence Signal peptide
MGWSCIILFLVATATGVHS

SEQ ID NO: 265 - protein, 329 aa, Artificial Sequence
  Synthetic sequence SARS-CoV-2 CH1-CH3 G1m17; IgG1*01 LS no C-term Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 266 - protein, 329 aa, Artificial Sequence
  Synthetic sequence SARS-CoV-2 CH1-CH3 G1m17; IgG1*01 LS GAALIE no C-term Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

(19) United States
(12) Patent Application Publication
(10) Pub. No.: US 2025/0263467 A1
(43) Pub. Date: Aug. 21, 2025

(54) ANTIBODY THERAPIES FOR SARS-COV-2 INFECTION

(71) Applicant: VIR BIOTECHNOLOGY, INC., San Francisco, CA (US)

(72) Inventors: Elizabeth ALEXANDER, San Francisco, CA (US); Wendy YEH, San Francisco, CA (US); Phillip S. PANG, San Francisco, CA (US); David HONG, San Francisco, CA (US); Lynn E. CONNOLLY, San Francisco, CA (US); Erik MONGALIAN, San Francisco, CA (US); Jenny SAGAR, San Francisco, CA (US); Megan SMITHEY, San Francisco, CA (US)

(21) Appl. No.: 18/001,583

(22) PCT Filed: Jun. 11, 2021
(86) PCT No.: PCT/US2021/036979

§ 371 Date: Dec. 12, 2022

Related U.S. Application Data
(60) Provisional application No. 63/196,089, filed on Jun. 2, 2021.
(60) Provisional application No. 63/186,055, filed on May 7, 2021.
(60) Provisional application No. 63/159,404, filed on Mar. 10, 2021.
(60) Provisional application No. 63/141,430, filed on Jan. 25, 2021.
(60) Provisional application No. 63/066,966, filed on Aug. 18, 2020.
(60) Provisional application No. 63/038,738, filed on Jun. 12, 2020.

(51) Int. Cl. C07K 16/10; A61K 39/00; A61P 31/14

(52) U.S. Cl. CPC C07K 16/1003; A61P 31/14; A61K 2039/505; C07K 2317/55; C07K 2317/76

(57) ABSTRACT

The instant disclosure provides methods of treating or preventing a SARS-CoV-2 infection, e.g., in a subject having or at risk for developing COVID-19, using an antibody (or antigen-binding fragment) compositions. Disclosed methods include prophylaxis against SARS-CoV-2 infection or transmission, as well as treatment of a subject having a SARS-CoV-2 infection. A SARS-CoV-2 infection (e.g., causing COVID-19) to be treated can be at any stage of infection and/or can result in any stage of disease, for example, mild, mild-to-moderate, severe, or critical.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930585_413WO_SEQUENCE_LISTING.txt. The text file is 327 KB, was created on Jun. 10, 2021, and is being submitted electronically via EFS-Web.

### BACKGROUND

A novel betacoronavirus emerged in Wuhan, China, in late 2019. As of Jun. 9, 2021, approximately 174 million cases of infection by this virus (termed, among other names, SARS-CoV-2 and Wuhan coronavirus) were confirmed worldwide, and had resulted in approximately 3.7 million deaths. Therapies for preventing or treating SARS-CoV-2 infection and COVID-19 are needed.

### BRIEF DESCRIPTION OF THE DRAWINGS

FIG. 1 shows the design of a clinical study of recombinant monoclonal IgG1 antibody sotrovimab (also called, e.g., S309 N55Q LS herein) for treatment of mild to moderate COVID-19 disease.

FIGS. 2A-2D show a timeline of events for a clinical study of sotrovimab for treatment of mild to moderate COVID-19 disease.

FIG. 3 shows the study design for a clinical study of sotrovimab for treatment of severe to critical COVID-19 disease.

FIG. 4 shows the study design for a clinical study of sotrovimab for post-exposure prophylaxis of COVID-19 disease.

FIG. 5 shows the binding site of sotrovimab on the spike protein of SARS-CoV-2. The SARS-CoV-2 receptor-binding domain is shown, with the ACE2 receptor-binding motif in green and the sotrovimab epitope in orange. ACE2 denotes angiotensin-converting enzyme 2.

FIG. 6 shows the design of a clinical study using sotrovimab described herein. R denotes randomization. *Patients were stratified by age (≤70 vs. >70 years), symptom duration (≤3 days vs. 4-5 days), and region. †Study pharmacists reconstituted and dispensed all study medications within equal time frames to maintain blinding.

### DETAILED DESCRIPTION

Provided herein are methods of treating or preventing a SARS-CoV-2 infection, e.g., in a subject having or at risk for developing COVID-19, using an antibody, an antigen-binding fragment, or composition that comprises the same. Certain antibodies and antigen-binding fragments for use in the methods recognize a conserved epitope in SARS-CoV-2 S glycoprotein and potently neutralize SARS-CoV-2 in vitro and in vivo. Non-limiting examples of antibodies include S309 and engineered variants of S309 (e.g., sotrovimab, VIR-7832). In some embodiments, a variant of S309 comprises a N55Q substitution in the VH region. In some embodiments, an antibody or antigen-binding fragment comprises an Fc polypeptide comprising one or more amino acid mutations that, for example, can extend in vivo half-life of the antibody or antigen-binding fragment and/or can promote a vaccinal effect of the antibody or antigen-binding fragment.

Presently disclosed methods include prophylaxis against SARS-CoV-2 infection or transmission, as well as treatment of a subject having a SARS-CoV-2 infection. A SARS-CoV-2 infection (e.g., causing COVID-19) can be at any stage of infection and/or can result in any stage of disease, for example, mild, mild-to-moderate, severe, or critical. For example, as described further herein, a single dose of an antibody of the present disclosure can be sufficient to reduce hospitalization or death in subjects with mild-to-moderate COVID-19.

Administration of the antibody or antigen-binding fragment can be performed using any method, such as for example, intravenous injection and intramuscular injection. In some contexts, a single dose of the antibody or antigen-binding fragment (or composition comprising the same) is administered to a subject. Subjects may be characterized in accordance with one or more criteria, and/or possess can one or more characteristics, as provided herein. Also provided are antibodies, antigen-binding fragments, and compositions for use in methods of treating or preventing a SARS-CoV-2 infection (or COVID-19), as well as in the preparation of a medicament for the treatment or treating or prevention of a SARS-CoV-2 infection

Prior to setting forth this disclosure in more detail, it may be helpful to an understanding thereof to provide definitions of certain terms to be used herein. Additional definitions are set forth throughout this disclosure.

As used herein, “SARS-CoV-2”, also originally referred to as “Wuhan coronavirus”, “Wuhan seafood market pneumonia virus”, or “Wuhan CoV”, “novel CoV”, or “nCOV”, or “2019 nCOV”, or “Wuhan nCOV”, or a variant thereof, is a betacoronavirus of lineage B (sarbecovirus). SARS-CoV-2 was first identified in Wuhan, Hubei province, China, in late 2019 and spread within China and to other parts of the world by early 2020. SARS CoV-2 infection can result in a disease known as COVID-19; symptoms of COVID-19 include fever or chills, dry cough, dyspnea, fatigue, body aches, headache, new loss of taste or smell, sore throat, congestions or runny nose, nausea or vomiting, diarrhea, persistent pressure or pain in the chest, new confusion, inability to wake or stay awake, and bluish lips or face.

The genomic sequence of SARS-CoV-2 isolate Wuhan-Hu-1 is provided in SEQ ID NO.: 163 (see also GenBank MN908947.3, Jan. 23, 2020), and the amino acid translation of the genome is provided in SEQ ID NO.: 164 (see also GenBank QHD43416.1, Jan. 23, 2020). Like other coronaviruses (e.g., SARS CoV), SARS-CoV-2 comprises a “spike” or surface (“S”) type I transmembrane glycoprotein containing a receptor binding domain (RBD). RBD is believed to mediate entry of the lineage B SARS coronavirus to respiratory epithelial cells by binding to the cell surface receptor angiotensin-converting enzyme 2 (ACE2). In particular, a receptor binding motif (RBM) in the virus RBD is believed to interact with ACE2.

The amino acid sequence of the SARS-CoV-2 Wuhan-Hu-1 surface glycoprotein is provided in SEQ ID NO.: 165. The amino acid sequence of SARS-CoV-2 Wuhan coronavirus RBD is provided in SEQ ID NO.: 166. Wuhan coronavirus S protein has approximately 73% amino acid sequence identity with SARS-CoV. The amino acid sequence of Wuhan coronavirus RBM is provided in SEQ ID NO.: 167. Wuhan coronavirus RBD has approximately 75% to 77% amino acid sequence similarity to SARS coronavirus RBD, and Wuhan coronavirus RBM has approximately 50% amino acid sequence similarity to SARS coronavirus RBM.

Unless otherwise indicated herein, SARS-CoV-2 Wu-Hu-1 refers to a virus comprising the amino acid sequence set forth in any one or more of SEQ ID NOs.: 164, 165, and 166, optionally with the genomic sequence set forth in SEQ ID NO.: 163.

There have been a number of emerging SARS-CoV-2 variants. Some SARS-CoV-2 variants contain an N439K mutation, which has enhanced binding affinity to the human ACE2 receptor (Thomson, E. C., et al., The circulating SARS-CoV-2 spike variant N439K maintains fitness while evading antibody-mediated immunity. bioRxiv, 2020). Some SARS-CoV-2 variants contain an N501Y mutation, which is associated with increased transmissibility, including the lineages B.1.1.7 (also known as 201/501Y.V1 and VOC 202012/01) and B.1.351 (also known as 20H/501Y.V2), which were discovered in the United Kingdom and South Africa, respectively (Tegally, H., et al., Emergence and rapid spread of a new severe acute respiratory syndrome-related coronavirus 2 (SARS-CoV-2) lineage with multiple spike mutations in South Africa. medRxiv, 2020: p. 2020.12.21.20248640; Leung, K., et al., Early empirical assessment of the N501Y mutant strains of SARS-CoV-2 in the United Kingdom, October to November 2020. medRxiv, 2020: p. 2020.12.20.20248581). B.1.351 also include two other mutations in the RBD domain of SARS-CoV2 spike protein, K417N and E484K (Tegally, H., et al., Emergence and rapid spread of a new severe acute respiratory syndrome-related coronavirus 2 (SARS-CoV-2) lineage with multiple spike mutations in South Africa. medRxiv, 2020: p. 2020.12.21.20248640). Other SARS-CoV-2 variants include the Lineage B.1.1.28, which was first reported in Brazil; the Variant P.1, lineage B.1.1.28 (also known as 20J/501Y.V3), which was first reported in Japan; Variant L452R, which was first reported in California in the United States (Pan American Health Organization, Epidemiological update: Occurrence of variants of SARS-CoV-2 in the Americas, Jan. 20, 2021, available at https://reliefweb.int/sites/reliefweb.int/files/resources/2021-jan-20-phe-epi-update-SARS-CoV-2.pdf). Other SARS-CoV-2 variants include a SARS CoV-2 of clade 19A; SARS CoV-2 of clade 19B; a SARS CoV-2 of clade 20A; a SARS CoV-2 of clade 20B; a SARS CoV-2 of clade 20C; a SARS CoV-2 of clade 20D; a SARS CoV-2 of clade 20E (EU1); a SARS CoV-2 of clade 20F; a SARS CoV-2 of clade 20G; and SARS CoV-2 B1.1.207; and other SARS CoV-2 lineages described in Rambaut, A., et al., A dynamic nomenclature proposal for SARS-CoV-2 lineages to assist genomic epidemiology. Nat Microbiol 5, 1403-1407 (2020). A SARS CoV-2 infection in accordance with the present disclosure includes infection by any one or more of the aforementioned SARS-CoV-2 viruses and variants thereof.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

“Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody, antigen-binding fragment, or composition of the present disclosure is administered in an amount sufficient to elicit a therapeutic or prophylactic benefit. Therapeutic or prophylactic/preventive benefit includes, for example, improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of a SARS-CoV-2 infection or COVID-19 (i.e., in a statistically significant manner). In certain embodiments, therapeutic or prophylactic/preventive benefit includes a reduced duration of hospitalization for treatment of a SARS-CoV-2 infection or COVID-19 (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality. In certain embodiments, therapeutic and/or prophylactic benefit comprises a reduction in viral load and/or viral shedding in, e.g., a respiratory sample (lung tissue, nasal swab, sputum, or the like) from the subject. In certain embodiments, therapeutic and/or prophylactic benefit comprises preventing progression of COVID-19, e.g., from mild-to-moderate to severe, or from severe to critical, as described herein. In certain embodiments, therapeutic and/or prophylactic comprises preventing contraction and/or transmission of a SARS-CoV-2 infection, e.g., which can be symptomatic or asymptomatic.

A “therapeutically effective amount” or “effective amount” of an antibody, antigen-binding fragment, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously. A combination may comprise, for example, two different antibodies that specifically bind a SARS-CoV-2 antigen, which in certain embodiments, may be the same or different SARS-CoV-2 antigen, and/or can comprise the same or different epitopes.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátés et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In the context of a SARS-CoV-2 infection, a “host” refers to a cell or a subject infected with the SARS-CoV-2.

“Antigen” or “Ag”, as used herein, refers to an immunogenic molecule that provokes an immune response. This immune response may involve antibody production, activation of specific immunologically-competent cells, activation of complement, antibody dependent cytotoxicicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in a SARS-CoV-2 (e.g., a surface glycoprotein or portion thereof), such as present in a virion, or expressed or presented on the surface of a cell infected by the SARS-CoV-2.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

### Antibodies, Antigen-Binding Fragments, and Compositions

Certain presently disclosed methods and uses comprise administering to a subject antibody, or an antigen-binding fragment thereof, that comprises a heavy chain variable domain (VH) comprising a CDRH1, a CDRH2, and a CDRH3, and a light chain variable domain (VL) comprising a CDRL1, a CDRL2, and a CDRL3, and is capable of binding to a surface glycoprotein of a SARS-CoV-2 (e.g. as expressed on a cell surface of a host cell and/or on a SARS-CoV-2 virion).

In certain preferred embodiments, described further herein, an antibody or antigen-binding fragment thereof used in a method comprises the CDRH1, CDRH2, CDRH3, CDRL1, CDRL2, and CDRL3 amino acid sequences set forth in SEQ ID NOs.: 106, 121, 108, 169, 170, and 171, respectively, or set forth in SEQ ID NOs.: 106, 107, 108, 169, 170, and 171, respectively. In certain embodiments, an antibody or antigen-binding fragment thereof used in a method comprises the VH amino acid sequence set forth in SEQ ID NO.: 113 or 105 and the VL amino acid sequence set forth in SEQ ID NO.: 168.

In certain embodiments, an antibody or antigen-binding fragment of used in a method associates with or unites with a SARS-CoV-2 surface glycoprotein epitope or antigen comprising the epitope, while not significantly associating or uniting with any other molecules or components in a sample.

In certain embodiments, an antibody or antigen-binding fragment of the present disclosure associates with or unites (e.g., binds) to a SARS-CoV-2 surface glycoprotein epitope, and can also associate with or unite with an epitope from another coronavirus (e.g., SARS CoV) present in the sample, but not significantly associating or uniting with any other molecules or components in the sample. In other words, in certain embodiments, an antibody or antigen binding fragment of the present disclosure is cross-reactive for SARS-CoV-2 and one or more additional coronavirus.

In certain embodiments, an antibody or antigen-binding fragment of the present disclosure specifically binds to a SARS-CoV-2 surface glycoprotein. As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K_{d}) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” antibodies refer to those antibodies having a Kₐ of up to 10⁷M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

A variety of assays are known for identifying antibodies of the present disclosure that bind a particular target, as well as determining binding domain or binding protein affinities, such as Western blot, ELISA (e.g., direct, indirect, or sandwich), analytical ultracentrifugation, spectroscopy, and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known.

Binding can be determined by, for example, recombinantly expressing a SARS-CoV-2 antigen in a host cell (e.g., by transfection) and immunostaining the (e.g., fixed, or fixed and permeabilized) host cell with antibody and analyzing binding by flow cytometery (e.g., using a ZE5 Cell Analyzer (BioRad®) and FlowJo software (TreeStar). In some embodiments, positive binding can be defined by differential staining by antibody of SARS-CoV-2-expressing cells versus control (e.g., mock) cells.

In some embodiments an antibody or antigen-binding fragment of the present disclosure binds to SARS-CoV-2 S protein, as measured using biolayer interferometry. In certain embodiments, an antibody or antigen-binding fragment of the present disclosure binds to SARS-CoV-2 S protein with a K_{D} of less than about 4.5×10⁻⁹ M, less than about 5×10⁻⁹ M, less than about 1×10⁻¹⁰ M, less than about 5×10⁻¹⁰ M, less than about 1×10⁻¹¹ M, less than about 5×10⁻¹¹ M, less than about 1×10⁻¹² M, or less than about 5×10⁻¹² M. In some embodiments, an antibody or antigen-binding fragment of the present disclosure binds to SARS-CoV-2 S protein RBD with a K_{D} of less than about 4.5×10⁻⁹ M, less than about 5×10⁻⁹ M, less than about 1×10⁻¹⁰ M, less than about 5×10⁻¹⁰ M, less than about 1×10⁻¹¹ M, less than about 5×10⁻¹¹ M, less than about 1×10⁻¹² M, or less than about 5×10⁻¹² M. In certain embodiments, an antibody or antigen-binding fragment of the present disclosure binds to SARS-CoV-2 S protein (e.g., a glycosylated or a deglycosylated S protein RBD) with a K_{D}, a kₐ, and/or a k_{d} as shown herein. In particular embodiments, an antibody or antigen-binding fragment is capable of binding to a glycosylated S protein RBD with a K_{D} of about 0.35, about 0.36, about 0.37, about 0.38, about 0.39, about 0.40, about 0.41, about 0.42, about 0.43, about 0.44, or about 0.45 nM, a kₐ of about 8.5e4 l/Ms, and/or a k_{d} of about 3.3e-5 l/S. In certain embodiments, an antibody or antigen-binding fragment is capable of binding to a deglycosylated S protein RBD with a K_{D} of about 0.95, about 0.96 nM, about 0.97 nM, about 0.98 nM, about 0.99 nM, about 1.0 nM, about 1.1 nM, about 1.2 nM, about 1.3 nM, about 1.4 nM, about 1.5 nM, or about 1.6 nM, a kₐ of about 3.1e5 l/Ms, and/or a k_{d} of about 3.2e-4 l/S.

In certain embodiments, an antibody or antigen-binding fragment is capable of neutralizing infection by SARS-CoV-2. As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody or antigen-binding fragment is capable of preventing and/or neutralizing a SARS-CoV-2 infection in an in vitro model of infection and/or in an in vivo animal model of infection and/or in a human. In some embodiments, an antibody or antigen-binding fragment of the present disclosure is capable of neutralizing a SARS-CoV-2 infection with an IC50 of about 16 to about 20 μg/ml. In some embodiments, an antibody or antigen-binding fragment is capable of neutralizing a SARS-CoV-2 infection, or a virus pseudotyped with SARS-CoV-2, with an IC50 of about 0.3 to about 0.4 μg/ml. In some embodiments, an antibody or antigen-binding fragment, or a composition comprising two or more antibodies or antigen-binding fragments, of the present disclosure is capable of neutralizing a SARS-CoV-2 infection, or a virus pseudotyped with SARS-CoV-2, with an IC50 of about 0.07 to about 0.08 μg/ml.

In certain embodiments, the antibody or antigen-binding fragment (i) recognizes an epitope in the ACE2 receptor binding motif (RBM, SEQ ID NO.: 167) of SARS-CoV-2; (ii) is capable of blocking an interaction between SARS-CoV-2 and ACE2; (ii) is capable of binding to SARS-CoV-2 S protein with greater avidity than to SARS coronavirus S protein; (iv) is capable of staining about 30%, about 35%, about 40%, about 50%, about 55%, about 56%, about 57%, about 58%, about 59%, about 60%, or more of target cells expressing SARS-CoV-2 surface glycoprotein in a sample comprising about 50,000 of the target cells (e.g., ExpiCHO cells) in approximately 100 μL when the antibody or antigen-binding fragment is present at 10 μg/ml (e.g., staining as determined by a flow cytometry ELISA); (v) recognizes an epitope that is conserved in the ACE2 RBM of SARS-CoV-2 and in an ACE2 RBM of SARS coronavirus; (vi) is cross-reactive against SARS-CoV-2 and SARS coronavirus; (vii) recognizes an epitope in the SARS-CoV-2 surface glycoprotein that is not in the ACE2 RBM; or (viii) any combination of (i)-(vii).

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

The terms “V_{L}” or “VL” and “V_{H}” or “VH” refer to the variable binding region from an antibody light chain and an antibody heavy chain, respectively. In certain embodiments, a VL is a kappa (κ) class (also “VK” herein). In certain embodiments, a VL is a lambda (λ) class. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

As used herein, a “variant” of a CDR refers to a functional variant of a CDR sequence having up to 1-3 amino acid substitutions (e.g., conservative or non-conservative substitutions), deletions, or combinations thereof.

Numbering of CDR and framework regions may be according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Plückthun, J. Mol. Bio. 309:657-670 (2001)). Equivalent residue positions can be annotated and for different molecules to be compared using Antigen receptor Numbering And Receptor Classification (ANARCI) software tool (2016, Bioinformatics 15:298-300).

Accordingly, identification of CDRs of an exemplary variable domain (VH or VL) sequence as provided herein according to one numbering scheme is not exclusive of an antibody comprising CDRs of the same variable domain as determined using a different numbering scheme. In certain embodiments, an antibody or antigen-binding fragment is provided that comprises CDRs identified in a VH sequence according to any one of SEQ ID NOs.: 1, 9-15, 23, 24, 27, 28-46, 55, 63, 79, 87, 95, 103, 105, 113-120, 129-146, 155, 172, 176-178, 194, 196, 198, 200, 202, and 239, and in a VL sequence according to any one of SEQ ID NOs.: 5, 47-50, 59, 67, 71-72, 75, 76, 83, 91, 99, 109, 147-150, 159, 168, 182, 190, 234, and 243, as determined using any known CDR numbering method, including the Kabat, Chothia, EU, IMGT, Martin (Enhanced Chothia), Contact, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software.

In certain embodiments, the antibody or antigen-binding fragment comprises CDRH1, CDRH2, CDRH3, CDRL1, CDRL2, and CDRL3 amino acid sequences according to SEQ ID NOs.: 106, 121 or 107, 108, 169, 170, and 171, respectively.

In some embodiments, an antibody or an antigen-binding fragment is provided that comprises a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 comprises or consists of the amino acid sequence according to any one of SEQ ID NOs.: 2, 56, 64, 80, 88, 96, 106, 156, 179, 195, or 240, or a sequence variant thereof comprising one, two, or three acid substitutions, one or more of which substitutions is optionally a conservative substitution and/or is a substitution to a germline-encoded amino acid; (ii) the CDRH2 comprises or consists of the amino acid sequence according to any one of SEQ ID NOs.: 3, 16-22, 57, 65, 81, 89, 97, 107, 121-126, 157, 180, 197, 199, or 241, or a sequence variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence according to any one of SEQ ID NOs.: 4, 25, 26, 58, 66, 82, 90, 98, 104, 108, 127, 128, 158, 181, 201, 203, or 242, or a sequence variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence according to any one of SEQ ID NOs.: 6, 51-54, 60, 68, 73, 74, 84, 92, 100, 110, 160, 169, 183, 235, or 244, or a sequence variant thereof comprising one, two, or three amino acid substitutions, one or more of which substitutions is optionally a conservative substitution and/or is a substitution to a germline-encoded amino acid; (v) the CDRL2 comprises or consists of the amino acid sequence according to any one of SEQ ID NOs.: 7, 61, 69, 85, 93, 101, 111, 161, 170, 184, 236, or 245, or a sequence variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence according to any one of SEQ ID NOs.: 8, 62, 70, 77, 78, 86, 94, 102, 112, 151, 152, 153, 154, 162, 171, 185, 237, or 246, or a sequence variant thereof comprising having one, two, or three amino acid substitutions, one or more of which substitutions is optionally a conservative substitution and/or is a substitution to a germline-encoded amino acid, wherein the antibody or antigen binding fragment is capable of binding to a surface glycoprotein of a SARS-CoV-2 expressed on a cell surface of a host cell.

In any of the presently disclosed embodiments, the antibody or antigen-binding fragment is capable of preventing and/or neutralizing a SARS-CoV-2 infection in an in vitro model of infection and/or in an in vivo animal model of infection and/or in a human.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CH1, a CH2, and a CH3. In certain embodiments, a CL comprises an amino acid sequence having 90%, 91%, 92%, 93%, 94%, 95%, 96%, 97%, 98%, 99%, or 100% identity to the amino acid sequence of SEQ ID NO.: 174 or SEQ ID NO.: 193. In certain embodiments, a CH1-CH2-CH3 comprises an amino acid sequence having 90%, 91%, 92%, 93%, 94%, 95%, 96%, 97%, 98%, 99%, or 100% identity to the amino acid sequence of SEQ ID NO.: 173 or SEQ ID NO.: 175.

It will be understood that, for example, production in a mammalian cell line can remove one or more C-terminal lysine of an antibody heavy chain (see, e.g., Liu et al. mAbs 6 (5): 1145-1154 (2014)). Accordingly, an antibody or antigen-binding fragment of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine, and embodiments where a lysine is the C-terminal residue. Examples of CH1-CH3 amino acid sequences that lack a C-terminal lysine are provided in SEQ ID NOs.: 265 and 266.

In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy chain-derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment). Peptide linkers and exemplary linker sequences for use in scFabs are discussed in further detail herein.

A scFab can be comprise any combination of VH and VL sequences or any combination of the CDRH1, CDRH2, CDRH3, CDRL1, CDRL2, and CDRL3 sequences disclosed herein. In certain embodiments, a scFab comprises the VH sequence as provided in SEQ ID NO: 105 or SEQ ID NO: 113 and the VL sequence as provided in SEQ ID NO: 168. In certain embodiments, a scFab comprises a CDRH1 sequence as provided in SEQ ID NO: 106, a CDRH2 sequence as provided in SEQ ID NO: 107 or 121, a CDRH3 sequence as provided in SEQ ID NO: 108, a CDRL1 sequence as provided in SEQ ID NO: 169, a CDRL2 sequence as provided in SEQ ID NO: 170, and a CDRL3 sequence as provided in SEQ ID NO: 171. In certain embodiments, a scFab comprises an amino acid sequence having at least 90%, 91%, 92%, 93%, 94%, 95%, 96%, 97%, 98%, 99%, or 100% identity to the amino acid sequence provided in any one of SEQ ID NOs.: 218-219 or 226-227.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. In some embodiments, the scFv polypeptide comprises a polypeptide linker disposed between and linking the VH and VL domains that enables the scFv to retain or form the desired structure for antigen binding. Such a peptide linker can be incorporated into a fusion polypeptide using standard techniques well known in the art. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both.

Peptide linker sequences may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. No. 4,935,233, and U.S. Pat. No. 4,751,180. Other illustrative and non-limiting examples of linkers may include, for example, Glu-Gly-Lys-Ser-Ser-Gly-Ser-Gly-Ser-Glu-Ser-Lys-Val-Asp (SEQ ID NO: 215) (Chaudhary et al., Proc. Natl. Acad. Sci. USA 87:1066-1070 (1990)) and Lys-Glu-Ser-Gly-Ser-Val-Ser-Ser-Glu-Gln-Leu-Ala-Gln-Phe-Arg-Ser-Leu-Asp (SEQ ID NO: 216) (Bird et al., Science 242:423-426 (1988)) and the pentamer Gly-Gly-Gly-Gly-Ser (SEQ ID NO: 217) when present in a single iteration or repeated 1 to 5 or more times, or more; see, e.g., SEQ ID NO: 213. Any suitable linker may be used, and in general can be about 3, 4, 5, 6, 7, 8, 9, 10, 11, 12, 13, 14, 15, 16, 17, 18, 19, 20, 21, 22, 15 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human. Exemplary linkers include those comprising or consisting of the amino acid sequence set forth in any one or more of SEQ ID NOs: 206-217. In certain embodiments, the linker comprises or consists of an amino acid sequence having at least 75% (i.e., at least about 75%, 80%, 85%, 90%, 91%, 92%, 93%, 94%, 95%, 96%, 97%, 98%, 99%, or more) identity to the amino acid sequence set forth in any one of SEQ ID NOs: 206-217.

scFv can be constructed using any combination of the VH and VL sequences or any combination of the CDRH1, CDRH2, CDRH3, CDRL1, CDRL2, and CDRL3 sequences disclosed herein. In certain embodiments, a scFv comprises the VH sequence provided in SEQ ID NO: 105 or SEQ ID NO: 113 and the VL sequence provided in SEQ ID NO: 168. In certain embodiments, a scFab comprises a CDRH1 sequence as provided in SEQ ID NO: 106, a CDRH2 sequence as provided in SEQ ID NO: 107 or 121, a CDRH3 sequence as provided in SEQ ID NO: 108, a CDRL1 sequence as provided in SEQ ID NO: 169, a CDRL2 sequence as provided in SEQ ID NO: 170, and a CDRL3 sequence as provided in SEQ ID NO: 171. In certain embodiments, a scFv can comprise the amino acid sequence as provided in SEQ ID NO: 220-221 or SEQ ID NO: 228-229.

In some embodiments, linker sequences are not required; for example, when the first and second polypeptides have non-essential N-terminal amino acid regions that can be used to separate the functional domains and prevent steric interference.

Also provided herein are variant antibodies that comprise one or more amino acid alterations in a variable region (e.g., VH, VL, framework or CDR) as compared to a presently disclosed (“parent”) antibody, wherein the variant antibody is capable of binding to a SARS-CoV-2 antigen.

In certain embodiments, the VH comprises or consists of an amino acid sequence having at least 85% (i.e., 85%, 86, 87, 88, 89, 90, 91, 92, 93, 94, 95, 96, 97, 98, 99, or 100%) identity to the amino acid sequence according to any one of SEQ ID NOs.: 1, 9-15, 23, 24, 27, 28-46, 55, 63, 79, 87, 95, 103, 105, 113-120, 129-146, 155, 172, 176-178, 194, 196, 198, 200, 202, and 239, wherein the variation is optionally limited to one or more framework regions and/or the variation comprises one or more substitution to a germline-encoded amino acid; and/or (ii) the VL comprises or consists of an amino acid sequence having at least 85% (i.e., 85%, 86, 87, 88, 89, 90, 91, 92, 93, 94, 95, 96, 97, 98, 99, or 100%) identity to the amino acid sequence according to any one of SEQ ID NOs.: 5, 47-50, 59, 67, 71-72, 75, 76, 83, 91, 99, 109, 147-150, 159, 168, 182, 190, 234, and 243, wherein the variation is optionally limited to one or more framework regions and/or the variation comprises one or more substitution to a germline-encoded amino acid.

In certain embodiments, an antibody or an antigen-binding fragment of the present disclosure comprises a VH comprising or consisting of the amino acid sequence according to SEQ ID NO: 105 and a VL comprising or consisting of the amino acid sequence according to SEQ ID NO: 168. In further embodiments, an antibody or an antigen-binding fragment of the present disclosure comprises a VH comprising or consisting of the amino acid sequence according to SEQ ID NO: 105 and a VL comprising or consisting of the amino acid sequence according to SEQ ID NO: 168 and binds to SARS-CoV-2 S protein with a K_{D} of less than about 4.5×10−9 M, less than about 5×10−9 M, less than about 1×10−10 M, less than about 5×10−10 M, less than about 1×10−11 M, less than about 5×10−11 M, less than about 1×10−12 M, or less than about 5×10−12 M. In still further embodiments, an antibody or an antigen-binding fragment of the present disclosure comprises a VH comprising or consisting of the amino acid sequence according to SEQ ID NO: 105 and a VL comprising or consisting of the amino acid sequence according to SEQ ID NO: 168 and is capable of neutralizing a SARS-CoV-2 infection, or a virus pseudotyped with SARS-CoV-2, with an IC50 of about 0.3 to about 0.4 μg/ml.

In certain embodiments, an antibody or an antigen-binding fragment of the present disclosure comprises a VH comprising or consisting of the amino acid sequence according to SEQ ID NO: 105 and a VL comprising or consisting of the amino acid sequence according to SEQ ID NO: 168 and binds to SARS-CoV-2 protein RBD with an EC50 of about 11 to about 25 ng/ml. In certain embodiments, an antibody or an antigen-binding fragment of the present disclosure comprises a VH comprising or consisting of the amino acid sequence according to SEQ ID NO: 113 and a VL comprising or consisting of the amino acid sequence according to SEQ ID NO: 168 and binds to SARS-CoV-2 S protein RBD with an EC50 of about 9 to about 23 ng/ml. In certain embodiments, an antibody or an antigen-binding fragment of the present disclosure comprises a VH comprising or consisting of the amino acid sequence according to SEQ ID NO: 129 and a VL comprising or consisting of the amino acid sequence according to SEQ ID NO: 168 and binds to SARS-CoV-2 S protein RBD with an EC50 of about 8 to about 22 ng/ml. In certain embodiments, an antibody or an antigen-binding fragment of the present disclosure comprises a VH comprising or consisting of the sequence according to SEQ ID NO: 119 and a VL comprising or consisting of the sequence according to SEQ ID NO: 168 and binds to SARS-CoV-2 S protein RBD with an EC50 of about 8 to about 22 ng/ml. In certain embodiments, an antibody or an antigen-binding fragment of the present disclosure comprises a VH comprising or consisting of the amino acid sequence according to SEQ ID NO: 172 and a VL comprising or consisting of the amino acid sequence according to SEQ ID NO: 168 and binds to SARS-CoV-2 S protein RBD with an EC50 of about 7 to about 19 ng/ml.

In certain embodiments, an antibody or antigen-binding fragment of the present disclosure is monospecific (e.g., binds to a single epitope) or is multispecific (e.g., binds to multiple epitopes and/or target molecules). Antibodies and antigen binding fragments may be constructed in various formats. Exemplary antibody formats disclosed in Spiess et al., Mol. Immunol. 67 (2): 95 (2015), and in Brinkmann and Kontermann, mAbs 9 (2): 182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L, H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

In certain embodiments, the antibody or antigen-binding fragment comprises two or more of VH domains, two or more VL domains, or both (i.e., two or more VH domains and two or more VL domains). In particular embodiments, an antigen-binding fragment comprises the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different epitopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

Monospecific or multispecific antibodies or antigen-binding fragments of the present disclosure constructed comprise any combination of the VH and VL sequences and/or any combination of the CDRH1, CDRH2, CDRH3, CDRL1, CDRL2, and CDRL3 sequences disclosed herein. In certain embodiments, an antibody or antigen-binding fragment comprises the VH sequence provided in SEQ ID NO: 105 or SEQ ID NO: 113 and the VL sequence provided in SEQ ID NO: 168. In certain embodiments, an antibody or antigen-binding fragment comprises a CDRH1 sequence as provided in SEQ ID NO: 106, a CDRH2 sequence as provided in SEQ ID NO: 107 or 121, a CDRH3 sequence as provided in SEQ ID NO: 108, a CDRL1 sequence as provided in SEQ ID NO: 169, a CDRL2 sequence as provided in SEQ ID NO: 170, and a CDRL3 sequence as provided in SEQ ID NO: 171. In certain embodiments, an antibody or antigen-binding fragment comprises the amino acid sequence as provided in SEQ ID NO: 222-225 or SEQ ID NO: 230-233.A bispecific or multispecific antibody or antigen-binding fragment may, in some embodiments, comprise one, two, or more antigen-binding domains (e.g., a VH and a VL) of the instant disclosure. Two or more binding domains may be present that bind to the same or a different SARS-CoV-2 epitope, and a bispecific or multispecific antibody or antigen-binding fragment as provided herein can, in some embodiments, comprise a further SARS-CoV-2 binding domain, and/or can comprise a binding domain that binds to a different antigen or pathogen altogether.

In any of the presently disclosed embodiments, the antibody or antigen-binding fragment can be multispecific; e.g., bispecific, trispecific, or the like.

In certain embodiments, the antibody or antigen-binding fragment comprises: (i) a first VH and a first VL; and (ii) a second VH and a second VL, wherein the first VH and the second VH are different and each independently comprise an amino acid sequence having at least 85% (i.e., 85%, 86%, 87%, 88%, 89%, 90%, 91%, 92%, 93%, 94%, 95%, 96%, 97%, 98%, 99%, or 100%) identity to the amino acid sequence set forth in any one of SEQ ID NOs.: 1, 9-15, 23, 24, 27-46, 55, 63, 79, 87, 95, 103, 105, 113-120, 129-146, 155, 172, 176-178, 194, 196, 198, 200, 202, and 239, and wherein the first VL and the second VL are different and each independently comprise an amino acid sequence having at least 85% (i.e., 85%, 86%, 87%, 88%, 89%, 90%, 91%, 92%, 93%, 94%, 95%, 96%, 97%, 98%, 99%, or 100%) identity to the amino acid sequence set forth in any one of SEQ ID NOs.: 5, 47-50, 59, 67, 71, 72, 75, 76, 83, 91, 99, 109, 147-150, 159, 168, 182, 190, 234, and 243, and wherein the first VH and the first VL together form a first antigen-binding site, and wherein the second VH and the second VL together form a second antigen-binding site.

In certain embodiments, the antibody or antigen-binding fragment comprises a Fc polypeptide, or a fragment thereof. The “Fc” fragment or Fc polypeptide comprises the carboxy-terminal portions (i.e., the CH2 and CH3 domains of IgG) of both antibody H chains held together by disulfides. Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, the T250Q/M428L, M252Y/S254T/T256E, H433K/N434F, M428L/N434S, E233P/L234V/L235A/G236+A327G/A330S/P331S, E333A, S239D/A330L/I332E, P257I/Q311, K326W/E333S, S239D/I332E/G236A, N297Q, K322A, S228P, L235E+E318A/K320A/K322A, L234A/L235A (also referred to herein as “LALA”), and L234A/L235A/P329G mutations, which mutations are summarized and annotated in “Engineered Fc Regions”, published by InvivoGen (2011) and available online at invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

For example, to activate the complement cascade, the C1q protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcεR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologuous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189:4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189:4981-4988).

In some embodiments, the antibodies disclosed herein and the antigen-binding fragments thereof comprise an Fc polypeptide or fragment thereof for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB may function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into corresponding positions IgG1 and IgG4, reduces binding of IgG1 and IgG4 to FcγRI by 10³-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of at least one of E233-G236, P238, D265, N297, A327, P329, D270, Q295, A327, R292 and K414.

Two allelic forms of human FcγRIIA are the “H131” variant, which binds to IgG1 Fc with high affinity, and the “R131” variant, which binds to IgG1 Fc with low affinity. See, e.g., Bruhns et al., Blood 113:3716-3725 (2009).

Regarding FcγRIII binding, reduced binding to FcγRIIIA is found, e.g., for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgG1 Fc with low affinity, and the “V158” variant, which binds to IgG1 Fc with high affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164:5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164:5313-5318).

Also contemplated are mutations that increase binding affinity of an Fc polypeptide or fragment thereof of the present disclosure to a (i.e., one or more) Fcγ receptor (e.g., as compared to a reference Fc polypeptide or fragment thereof or containing the same that does not comprise the mutation(s)). See, e.g., Delillo and Ravetch, Cell 161 (5): 1035-1045 (2015) and Ahmed et al., J. Struc. Biol. 194 (1): 78 (2016), the Fc mutations and techniques of which are incorporated herein by reference.

In any of the herein disclosed embodiments, an antibody or antigen-binding fragment can comprise a Fc polypeptide or fragment thereof comprising a mutation selected from G236A; S239D; A330L; and I332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. In some embodiments, the Fc polypeptide or fragment thereof does not comprise S239D.

In certain embodiments, the Fc polypeptide or fragment thereof may comprise or consist of at least a portion of an Fc polypeptide or fragment thereof that is involved in binding to FcRn binding. In certain embodiments, the Fc polypeptide or fragment thereof comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide or fragment thereof (e.g., as compared to a reference Fc polypeptide or fragment thereof or antibody that is otherwise the same but does not comprise the modification(s)). In certain embodiments, the Fc polypeptide or fragment thereof comprises or is derived from a IgG Fc and a half-life-extending mutation comprises any one or more of: M428L; N434S; N434H; N434A; N434S; M252Y; S254T; T256E; T250Q; P257I Q311I; D376V; T307A; E380A (EU numbering). In certain embodiments, a half-life-extending mutation comprises M428L/N434S (also referred to herein as “MLNS”). In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A.

In some embodiments, an antibody or antigen-binding fragment includes a Fc moiety that comprises the substitution mutations M428L/N434S. In some embodiments, an antibody or antigen-binding fragment includes a Fc polypeptide or fragment thereof that comprises the substitution mutations G236A/A330L/I332E. In certain embodiments, an antibody or antigen-binding fragment includes a (e.g., IgG) Fc moiety that comprises a G236A mutation, an A330L mutation, and a I332E mutation (GAALIE), and does not comprise a S239D mutation (e.g., comprises a native S at position 239). In particular embodiments, an antibody or antigen-binding fragment includes an Fc polypeptide or fragment thereof that comprises the substitution mutations: M428L/N434S and G236A/A330L/I332E, and optionally does not comprise S239D. In certain embodiments, an antibody or antigen-binding fragment includes a Fc polypeptide or fragment thereof that comprises the substitution mutations: M428L/N434S and G236A/S239D/A330L/I332E.

In certain embodiments, the antibody or antigen-binding fragment comprises a mutation that alters glycosylation, wherein the mutation that alters glycosylation comprises N297A, N297Q, or N297G, and/or the antibody or antigen-binding fragment is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin. Cancer Res. 13 (6): 1875-82 (2007); Huang et al. MAbs 6:1-12 (2018)).

In certain embodiments, the antibody or antigen-binding fragment is capable of eliciting continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect. Without wishing to be bound by theory, it is believed that dendritic cells can internalize complexes of antibody and antigen and thereafter induce or contribute to an endogenous immune response against antigen. In certain embodiments, an antibody or antigen-binding fragment comprises one or more modifications, such as, for example, mutations in the Fc comprising G236A, A330L, and I332E, that are capable of activating dendritic cells that may induce, e.g., T cell immunity to the antigen.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc polypeptide of the present disclosure comprises two CH2-CH3 polypeptides that associate to form a dimer.

In any of the presently disclosed embodiments, the antibody or antigen-binding fragment can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example. Monoclonal antibodies may also be obtained using methods disclosed in PCT Publication No. WO 2004/076677A2.

Antibodies and antigen-binding fragments of the present disclosure include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., Hc domains.

A “human antibody” is an antibody containing only sequences that are present in an antibody that is produced by a human. However, as used herein, human antibodies may comprise residues or modifications not found in a naturally occurring human antibody (e.g., an antibody that is isolated from a human), including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426. In certain embodiments, an antibody or antigen-binding fragment of the present disclosure is chimeric, humanized, or human.

Exemplary antibodies of the present disclosure include S309, sotrovimab, and VIR-7832. S309 is a human monoclonal antibody obtained from a B cell of a SARS-CoV survivor. S309 comprises the VH amino acid sequence of SEQ ID NO.: 105 and the VL amino acid sequence of SEQ ID NO.: 168. Sotrovimab (IgG1*01 G1m17; VH of SEQ ID NO.: 113, M428L and N434S Fc mutations; VL of SEQ ID NO.: 168 (kappa light chain IgKC*01 k1m3)) and VIR-7832 ((IgG1*01 G1m17; VH of SEQ ID NO.: 113, G236A, A330L, I332E, M428L, and N434S Fc mutations; VL of SEQ ID NO.: 168 (kappa light chain IgKC*01 k1m3)) are engineered human monoclonal antibodies derived from S309.

### Polynucleotides, Vectors, and Host Cells

Presently disclosed antibodies and antigen-binding fragments (and portions thereof; e.g., a CDR, a VH, a VL, a heavy chain, or a light chain) can be encoded by a polynucleotide. The polynucleotide can comprise deoxyribonucleic acid (DNA) or ribonucleic acid (RNA). RNA can comprise messenger RNA (mRNA). Polynucleotides can be codon-optimized for expression in a host cell. Once a coding sequence is known or identified, codon optimization can be performed using known techniques and tools, e.g., using the GenScript® OptimiumGene™ tool; see also Scholten et al., Clin. Immunol. 119:135, 2006). Codon-optimized sequences include sequences that are partially codon-optimized (i.e., one or more codon is optimized for expression in the host cell) and those that are fully codon-optimized. It will also be appreciated that polynucleotides encoding antibodies and antigen-binding fragments of the present disclosure may possess different nucleotide sequences while still encoding a same antibody or antigen-binding fragment due to, for example, the degeneracy of the genetic code, splicing, and the like. It will be appreciated that a polynucleotide encoding an antibody or antigen-binding fragment can be comprised in a polynucleotide that includes other sequences and/or features for, e.g., expression of the antibody or antigen-binding fragment in a host cell. Exemplary features include a promoter sequence, a polyadenylation sequence, a sequence that encodes a signal peptide (e.g., located at the N-terminus of a expressed antibody heavy chain or light chain), or the like.

Polynucleotides can be comprised or contained in a vector. A vector can comprise any one or more of the vectors disclosed herein. A vectors can comprise, for example, a DNA plasmid construct encoding the antibody or antigen-binding fragment, or a portion thereof (e.g., so-called “DMAb”; see, e.g., Muthumani et al., J Infect Dis. 214 (3): 369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). A DNA plasmid construct can comprise a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. Substituent components of the antibody or antigen-binding fragment can be encoded by a polynucleotide comprised in a single plasmid. Alternatively, the substituent components of the antibody or antigen-binding fragment can be encoded by a polynucleotide comprised in two or more plasmids (e.g., a first plasmid comprises a polynucleotide encoding a heavy chain, VH, or VH+CH, and a second plasmid comprises a polynucleotide encoding the cognate light chain, VL, or VL+CL). A single plasmid can comprise a polynucleotide encoding a heavy chain and/or a light chain from two or more antibodies or antigen-binding fragments of the present disclosure. An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase). A vector can comprise a nucleotide sequence encoding a signal peptide. The signal peptide may or may not be present (e.g., can be enzymatically cleaved from) on the mature antibody or antigen-binding fragment. Nucleic acid sequence encoding a signal peptide include the nucleotide sequence set forth in SEQ ID NO.: 252 or SEQ ID NO.: 263. A signal peptide can comprise or consist of the amino acid sequence set forth in SEQ ID NO.: 256 or SEQ ID NO.: 264. A vector can comprise a polyadenylation signal sequence. An example of a polyadenylation signal sequence comprises or consists of the nucleotide sequence as set forth in SEQ ID NO.: 253.

A vector can comprise a CMV promoter (e.g., comprising or consisting of the nucleotide sequence as set forth in SEQ ID NO.: 251).

Examples of host cells that can be used to express a presently disclosed antigen or antigen-binding fragment cells include but are not limited to, eukaryotic cells, e.g., yeast cells, animal cells, insect cells, plant cells; and prokaryotic cells, including E. coli. In some embodiments, the cells are mammalian cells. Cells include a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

Host cells also include a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523. A cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells may be transfected stably or transiently with a vector, e.g. for expressing an antibody, or an antigen-binding fragment thereof. Cells may be stably transfected with a vector. Alternatively, cells may be transiently transfected with a vector encoding an antibody or antigen-binding fragment.

An antibody or antigen-binding fragment (or polynucleotide encoding the same) can be heterologous to the host cell. For example, the cell may be of a species that is different to the species from which the antibody was fully or partially obtained (e.g., CHO cells expressing a human antibody or an engineered human antibody). The cell type of the host cell may not express the antibody or antigen-binding fragment in nature. Moreover, the host cell may impart a post-translational modification (PTM; e.g., glysocylation or fucosylation) on the antibody or antigen-binding fragment that is not present in a native state of the antibody or antigen-binding fragment (or in a native state of a parent antibody from which the antibody or antigen binding fragment was engineered or derived). Such a PTM may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a CHO cell can comprise one or more post-translational modification that is distinct from the antibody when isolated from the human and/or produced by the native human B cell or plasma cell).

Insect cells useful expressing a binding protein of the present disclosure are known in the art and include, for example, Spodoptera frugiperda Sf9 cells, Trichoplusia ni BTI-TN5B1-4 cells, and Spodoptera frugiperda SfSWT01 “Mimic™” cells. See, e.g., Palmberger et al., J. Biotechnol. 153 (3-4): 160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing a binding protein of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

Mammalian host cells include, for example, a CHO cell, a HEK293 cell, a PER.C6 cell, a Y0 cell, a Sp2/0 cell, a NS0 cell, a human liver cell, a myeloma cell, or a hybridoma cell.

Methods for producing an antibody, or antigen-binding fragment can comprise culturing a host cell under conditions and for a time sufficient to produce the antibody, or the antigen-binding fragment. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

### Pharmaceutical Compositions

Also provided herein are compositions that comprise any one or more of the presently disclosed antibodies or antigen-binding fragments and can further comprise a pharmaceutically acceptable carrier, excipient, or diluent. Carriers, excipients, and diluents are discussed in further detail herein.

In certain embodiments, a composition comprises two or more different antibodies or antigen-binding fragments according to the present disclosure. In certain embodiments, antibodies or antigen-binding fragments to be used in a combination each independently have one or more of the following characteristics: neutralize naturally occurring SARS-CoV-2 variants; do not compete with one another for Spike protein binding; bind distinct Spike protein epitopes; have a reduced formation of resistance to SARS-CoV-2; when in a combination, have a reduced formation of resistance to SARS-CoV-2; potently neutralize live SARS-CoV-2 virus; exhibit additive or synergistic effects on neutralization of live SARS-CoV-2 virus when used in combination; exhibit effector functions; are protective in relevant animal model(s) of infection; are capable of being produced in sufficient quantities for large-scale production.

In some embodiments, a composition comprises a polynucleotide or vector that encodes an antibody or antigen-binding fragment. In certain embodiments, a composition comprises a first vector comprising a first plasmid, and a second vector comprising a second plasmid, wherein the first plasmid comprises a polynucleotide encoding a heavy chain, VH, or VH+CH, and a second plasmid comprises a polynucleotide encoding the cognate light chain, VL, or VL+CL of the antibody or antigen-binding fragment thereof. In certain embodiments, a composition comprises a polynucleotide (e.g., mRNA) coupled to a suitable delivery vehicle or carrier. Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanomed Nanobiotechnol. 11(2): e1530 (2019)). Principles, reagents, and techniques for designing appropriate mRNA and formulating mRNA-LNP and delivering the same are described in, for example, Pardi et al. (J Control Release 217345-351 (2015)); Thess et al. (Mol Ther 23:1456-1464 (2015)); Thran et al. (EMBO Mol Med 9 (10): 1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol:polyethylene glycol lipid), and subcutaneous, intramuscular, intradermal, intravenous, intraperitoneal, and intratracheal administration of the same, are incorporated herein by reference.

### Uses

Also provided herein are methods of treating a subject using an antibody or antigen-binding fragment of the present disclosure, or a composition comprising the same, wherein the subject has, is believed to have, or is at risk for having an infection by a SARS-CoV-2, optionally having, believed to have, or at risk for COVID-19.

Accordingly, in certain embodiments, methods are provided for treating a SARS-CoV-2 infection in a subject (e.g. having or at risk of contracting a SARS-CoV-2 infection or COVID-19), wherein the methods comprise administering to the subject an effective amount of an antibody, antigen-binding fragment, or composition as disclosed herein.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

Briefly, pharmaceutical compositions according to certain embodiments of the present disclosure are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described an antibody or antigen-binding in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between 0.1 and about 70% of the weight of the composition. Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises an antibody, antigen-binding fragment thereof, or antibody conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment thereof in the aqueous delivery system.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat a SARS-CoV-2 infection), which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective daily dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polynucleotides, vectors, host cells, and related compositions of the present disclosure, a therapeutically effective dose may be different than for an antibody or antigen-binding fragment.

In certain embodiments, a method according to the present disclosure comprises administering to a subject a presently disclosed antibody or antigen-binding fragment at a dose of up to 100 mg, up to 150 mg, up to 200 mg, up to 250 mg, up to 300 mg, up to 350 mg, up to 400 mg, up to 450 mg, or up to 500 mg. In certain embodiments, a method comprises administering to a subject a presently disclosed antibody or antigen-binding fragment at a dose in a range from about 50 mg to about 500 mg, or in a range from about 50 mg to about 250 mg, or in a range from about 50 mg to 100 mg, or in a range from about 100 mg to about 500 mg, or in a range from about 250 mg to about 500 mg. In some embodiments, a method comprises administering to a subject 50, 75, 100, 125, 150, 175, 200, 225, 250, 275, 300, 325, 350, 375, 400, 425, 450, 475, or 500 mg of the antibody or antigen-binding fragment. In some embodiments, a method comprises administering to a subject 50, 150, 250, or 500 mg of the antibody or antigen-binding fragment. In some embodiments, a method comprises administering to a subject 500 mg of the antibody or antigen-binding fragment.

In particular embodiments, the antibody or antigen-binding fragment comprises CDRH1, CDRH2, CDRH3, CDRL1, CDRL2, and CDRL3 amino acid sequences of SEQ ID NOs.: 106, 121, 108, 169, 170, and 171, respectively. In further embodiments, the antibody or antigen-binding fragment comprises a VH comprising the amino acid sequence of SEQ ID NO.: 113 and a VL comprising the amino acid sequence of SEQ ID NO.: 168. In certain further embodiments, the antibody or antigen-binding fragment comprises M428L and N434S Fc mutations and/or G236A, A330L, and I332E Fc mutations.

A number of criteria are believed to contribute to high risk for contraction, transmission, progression of disease, and/or severe symptoms or death associated with a SARS CoV-2 infection. These include, but are not limited to, age, occupation, general health, pre-existing health conditions, close contacts with subjects who have or are suspected to or are at risk of having a SARS-CoV-2 infection, and lifestyle habits. In some embodiments, a subject treated according to the present disclosure comprises one or more risk factors.

In certain embodiments, a human subject treated according to the present disclosure is an infant, a child, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 20-44 years old, 45-54 years old, 55-64 years old, 65-74 years old, 75-84 years old, or 85 years old, or older. Persons of middle, and especially of elderly age are believed to be at particular risk. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, a human subject (e.g., such as an at-risk subject or a high-risk subject) treated according to the present disclosure is a resident of a nursing home or a long-term care facility, is a hospice care worker, is a healthcare provider or healthcare worker, is a first responder, is a family member or other close contact of a subject diagnosed with or suspected of having a SARS-CoV-2 infection, is overweight or clinically obese, is or has been a smoker, has or had chronic obstructive pulmonary disease (COPD), is asthmatic (e.g., having moderate to severe asthma), has an autoimmune disease or condition (e.g., diabetes), and/or has a compromised or depleted immune system (e.g., due to AIDS/HIV infection, a cancer such as a blood cancer, a lymphodepleting therapy such as a chemotherapy, a bone marrow or organ transplantation, or a genetic immune condition), has chronic liver disease, has cardiovascular disease, has a pulmonary or heart defect, works or otherwise spends time in close proximity with others, such as in a factory, shipping center, hospital setting, or the like.

In some embodiments, a close contact comprises a subject that: (a) has resided with an index case in the 7 days prior to index diagnosis, and can include residence or staff in a congregate setting such as long-term care facility or nursing home; (b) is medical staff, first responder, or other care person engaging with the index case; and/or (c) is less than 3 days since last exposure (close contact with a person with SARS-CoV-2 infection) to the index case.

In certain embodiments, a subject treated according to the present disclosure has received a vaccine for SARS-CoV-2. In some embodiments, the vaccine is determined to be ineffective, e.g., by post-vaccine infection or symptoms in the subject, by clinical diagnosis or scientific or regulatory criteria. In certain embodiments, a subject treated according to the present disclosure has not received a vaccine for SARS-CoV-2. In certain embodiments, a subject treated according to the present disclosure has received convalescent plasma therapy, remdesivir, or both, for SARS-CoV-2.

In certain embodiments, treatment is administered as pre-exposure or peri-exposure prophylaxis. In certain embodiments, treatment is administered to a subject with mild-to-moderate disease, which may be in an outpatient setting. In certain embodiments, treatment is administered to a subject with moderate-to-severe disease, such as requiring hospitalization. Sequelae of severe disease can include: respiratory failure; thromboembolic disease leading to pulmonary embolism and stroke; arrhythmia; shock; or any combination thereof. In certain embodiments, severe COVID-19 comprises (i) hypoxemia (O2 saturation ≤93% on room air or PaO2/FiO2<300) requiring oxygen supplementation for more than 1 day or (ii) the subject requiring ≥4 L/min oxygen supplementation or equivalent.

In some embodiments, wherein the subject has, or is at risk for progressing to, critical COVID-19. Critical disease generally includes an increased risk of mortality as compared to severe disease. In some embodiments, critical COVID-19 comprises respiratory failure requiring at least one of the following: invasive mechanical ventilation and ECMO; shock; and multi-organ dysfunction/failure.

In certain embodiments, a subject is hospitalized with COVID-19, which can include, for example, admission or transfer to an intensive care unit (ICU).

In any of the presently disclosed embodiments, the subject having a SARS-CoV-2 infection: has mild-to-moderate COVID-19; is experiencing any one or more of: fever; cough; fatigue; shortness of breath or difficulty breathing; muscle aches; chills; sore throat; runny nose; headache; chest pain; loss of taste and/or smell; and pink eye (conjunctivitis); malaise; and abnormal imaging; has evidence of lower respiratory disease by clinical assessment or imaging and a saturation of oxygen (SaO2) greater than (>) 93 percent (%) on room air at sea level, has a positive SARS-CoV-2 viral testing result, and/or is at high risk for progressing to severe COVID-19 and/or hospitalization, e.g., the human subject (1) is 65 years of age or older (≥65); has a body mass index (BMI) of 35 or greater (≥35); has chronic kidney disease; has diabetes; (5) has immunosuppressive disease, is receiving immunosuppressive treatment; is 55 years of age or older (≥55) and has cardiovascular disease, hypertension, chronic obstructive pulmonary disease, or other chronic respiratory disease; and/or is 12-17 years of age and has a BMI ≥85% for their age and gender, or sickle cell disease, congenital or acquired heart disease, neurodevelopmental disorders (e.g., cerebral palsy), a medical-related technological dependence (e.g., tracheostomy, gastrostomy, or positive pressure ventilation not related to COVID-19), or asthma, reactive airway or other chronic respiratory disease that requires daily medication for control; has recently been diagnosed with COVID-19 (e.g., within 1 day, 2 days, 3 days, 4 days, 5 days, 6 days, 7 days, 8 days, 9 days, 10 days, 11 days, 12 days, 13 days, or 14 days) and/or is within 10 days of symptom onset; or has or is experiencing any combination of the foregoing. In some embodiments, a subject receiving therapy is 18 years old or older. In some embodiments, a subject receiving therapy is 55 years old or younger, provided that the subject is 18 years or older. In some embodiments, the subject has a laboratory confirmed COVID-19 infection by positive polymerase chain reaction (PCR; e.g., RT-PCR) test; e.g., on any type of respiratory tract sample). In some embodiments, a subject has peripheral capillary oxygen saturation (SpO₂) >94% room air (RA), who have experienced one or more symptoms of COVID-19 for ≤120 h (5 days). In some embodiments, a subject receiving therapy according to the present disclosure is receiving or has received remdesivir, dexamethasone, tocilizumab, or any combination thereof. In any of the presently disclosed embodiments, the method can comprise administering a single dose of the antibody, antigen-binding fragment, or composition to the subject.

Typical routes of administering the presently disclosed compositions thus include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, host cell, or composition to the subject.

In preferred embodiments, a method comprises administering the antibody, antigen-binding fragment, or composition to the subject intravenously or intramuscularly.

In some embodiments, the subject: (i) is aged 18 to 49 years; (ii) is 18 years old or older; (iii) has mild to moderate COVID-19; (iv) has severe COVID-19; (v) has severe to critical COVID-19; (vi) has had fewer than seven days or 5 or fewer days since onset of symptoms; (vii) has had seven days or more since onset of symptoms; (viii) has had a positive reverse-transcriptase-polymerase-chain-reaction or antigen SARS-CoV-2 test result; (ix) is 55 years of age or older; (x) has one or more of: diabetes requiring medication, obesity (body-mass index >30 kg/m²), chronic kidney disease (estimated glomerular filtration rate <60 mL/min/1.73 m²), congestive heart failure (New York Heart Association class II or higher), chronic obstructive pulmonary disease (history of chronic bronchitis, chronic obstructive lung disease, or emphysema with dyspnea on physical exertion), and moderate to severe asthma (subject requires an inhaled steroid to control symptoms or has been prescribed a course of oral steroids in the past year); or (xi) any combination of (i)-(x).

As discussed further herein, administration can include, for example, intravenous administration or intramuscular administration. In some embodiments, a single dose of the antibody or antigen-binding fragment is administered to a subject intravenously over the course of 30 minutes, 60 minutes, or 90 minutes.

In certain embodiments, a method comprises administering the antibody, antigen-binding fragment, polynucleotide, vector, host cell, or composition to the subject at 2, 3, 4, 5, 6, 7, 8, 9, 10 times, or more.

In certain embodiments, a method comprises administering the antibody, antigen-binding fragment, or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, antigen-binding fragment, polynucleotide, vector, host cell, or composition at least one time prior to the subject being infected by the SARS-CoV-2.

In certain embodiments, the subject receiving treatment is 18 or more years of age with laboratory-confirmed (e.g., by PCR test) SARS-CoV-2 infection.

In some embodiments, the subject has a clinical status of Grade 4 (hospitalized, oxygen by mask or nasal prongs), 5 (hospitalized, on non-invasive ventilation, or high flow oxygen), 6 (hospitalized, intubation and mechanical ventilation) or 7 (ventilation and additional organ support-pressors, renal replacement therapy (RRT), extracorporeal membrane oxygenation (ECMO)), as defined by the WHO clinical severity score, 9-point ordinal scale.

In some embodiments, the subject has mild-to-moderate COVID-19. In some embodiments, the subject is at-risk of progression to severe COVID-19. In some embodiments, following administration of the antibody, antigen-binding fragment, or composition to the subject, the subject is at a reduced risk of hospitalization for COVID-19. In certain embodiments, following administration of the antibody, antigen-binding fragment, or composition to the subject, the risk of hospitalization for COVID-19 is reduced by 10% or more, 20% or more, 30% or more, 40% or more, 50% or more, 60% or more, 70% or more, 80% or more, or 85% or more.

In some embodiments, the subject has or is at risk for progressing to severe COVID-19, wherein, optionally, severe COVID-19 comprises (i) hypoxemia (02 saturation ≤93% on room air or PaO2/FiO2<300) requiring oxygen supplementation for more than 1 day or (ii) the subject requiring ≥4 L/min oxygen supplementation or equivalent.

In some embodiments, the subject has or is at risk for progressing to critical COVID-19, wherein, optionally, critical COVID-19 comprises respiratory failure requiring at least one of the following: invasive mechanical ventilation and ECMO; shock; and multi-organ dysfunction/failure.

In some embodiments, the subject is less than seven days since onset of symptoms. In other embodiments, the subject is seven days or more since onset of symptoms.

In some embodiments, the subject is any one or more of (i)-(iii): (i) 18 years of age or older and has a positive SARS-CoV-2 test result (by any validated test e.g. RT-PCR on any specimen type); (ii) (1) hospitalized with severe COVID-19 disease defined as requirement for supplemental oxygen or non-invasive ventilation consistent with Grade 4 or Grade 5 disease or (2) hospitalized with critical COVID-19 disease defined as those on mechanical ventilation (Grade 6 or Grade 7 disease)); (iii) is male or female, wherein, optionally, (1) the woman is non-childbearing potential (WONCBP) or (2) is a woman of child-bearing potential (WOCBP) and uses a contraceptive method.

In any of the presently disclosed embodiments, the method can comprise administering 500 mg of the antibody or antigen-binding fragment to the subject.

In any of the presently disclosed embodiments, the subject can have had or can have close contacts to a person with a confirmed SARS-CoV-2 infection.

In any of the presently disclosed embodiments, treating can comprise preventing infection by SARS-CoV-2 and/or COVID-19. In any of the presently disclosed embodiments, treating can comprise preventing progression of COVID-19 in the subject. In any of the presently disclosed embodiments, treating can comprise preventing contraction and/or transmission of symptomatic COVID-19. In any of the presently disclosed embodiments, treating can comprise preventing contraction and/or transmission of asymptomatic COVID-19. In any of the presently disclosed embodiments, the subject can be at-risk for contracting or progressing on COVID-19.

In any of the presently disclosed embodiments, treating can comprise preventing or reducing: (1) one or more acute respiratory symptom selected from: cough; sputum production; sore throat; and shortness of breath; or (2) fever of greater than 38° C.; (3) two or more of the following symptoms: fatigue; myalgias/arthralgias; chills; nausea/vomiting; diarrhea; and anosmia/dysgeusia.

In any of the presently disclosed embodiments, treating can comprise preventing or reducing one or more of the following symptoms: fever of greater than 38° C.; chills; cough; sore throat; malaise; headache; myalgia; a change in smell or taste; nasal congestion/rhinorrhea; vomiting; diarrhea; shortness of breath on exertion.

In any of the presently disclosed embodiments, the subject can be an adult. In any of the presently disclosed embodiments, the subject can be 18 or more years of age, or can be 19 or more years of age. In any of the presently disclosed embodiments, the subject can be 55 years of age or is, or is 65 years of age or is older

In any of the presently disclosed embodiments, administering the antibody, antigen-binding fragment, or composition can comprise intravenous infusion. In any of the presently disclosed embodiments, administering the antibody, antigen-binding fragment, or composition can comprise intramuscular injection.

In any of the presently disclosed embodiments, the method can comprise administering 250 mg of the antibody or antigen-binding fragment to the subject. In any of the presently disclosed embodiments, the method can comprise administering 500 mg of the antibody or antigen-binding fragment to the subject.

In any of the presently disclosed embodiments, the subject can have a mild-to-moderate SARS-2-CoV infection (e.g., has mild-to-moderate COVID-19) and, optionally, be at risk for progression to severe disease.

In any of the presently disclosed embodiments, the subject: (i) can be 12 years old or older; and (ii) have last had contact with a person with a confirmed SARS-CoV-2 infection less than three days prior to administration of the composition.

In any of the presently disclosed embodiments, the subject has mild-to-moderate COVID-19 and the method comprises administering a single dose of the antibody, antigen-binding fragment, or composition to the subject intramuscularly.

In any of the presently disclosed embodiments, the single dose comprises 250 mg of the antibody or antigen-binding fragment. In some embodiments, a single dose according to a treatment method comprises 500 mg of the antibody or antigen-binding fragment.

In some embodiments: (i) (i)(a) the subject is 12 years of age or older and is at high risk of progression of COVID-19 or (i)(b) the subject is 65 years of age or older; and/or (ii) the subject has a positive SARS-CoV-2 test result (e.g., by PCR test), has oxygen saturation ≥94% on room air, has COVID-19 symptoms, and is less than or equal to 7 days from onset of symptoms.

In any of the presently disclosed embodiments: (i) (i)(a) the subject can be 12 years of age or older and is at high risk of progression of COVID-19 or (i)(b) the subject can be 65 years of age or older; and (ii) the subject can have a positive SARS-CoV-2 test result (e.g., by PCR test), has oxygen saturation ≥94% on room air, has COVID-19 symptoms, and is less than or equal to 7 days from onset of symptoms.

In any of the presently disclosed embodiments, the subject is not hospitalized and is at high-risk for (i) hospitalization and/or (ii) progression of COVID-19.

In any of the presently disclosed embodiments, the subject can be: (1) 12 or more years of age and, optionally, at high risk of progression of COVID-19; and/or (2) 65 or more years of age. In any of the presently disclosed embodiments, the subject can have had a positive SARS-CoV-2 test result, has oxygen saturation ≥94% on room air, has COVID-19 symptoms, and is less than or equal to 7 days from onset of symptoms.

In any of the presently disclosed embodiments, the antibody or antigen-binding fragment was obtained from a non-clonal pool of cells stably transfected with a polynucleotide encoding the antibody or antigen-binding fragment. In any of the presently disclosed embodiments, the antibody or antigen-binding fragment was obtained from a clonal master cell bank. A Master Cell Bank (MCB) is produced from an original antibody/antigen-binding fragment-producing cell line. A MCB is generally cryopreserved in multiple vials to prevent genetic variation and potential contamination by eliminating the total number of times a cell line is passaged or handled during the manufacturing process. A MCB is preferably tested for contaminants such as bacteria, fungi, and mycoplasmas; these should not be present in the MCB.

In any of the presently disclosed embodiments, the subject: is a resident of a nursing home or a long-term care facility; is a hospice care worker; is a healthcare provider or healthcare worker; is a first responder; is a family member or other close contact of a subject diagnosed with or suspected of having a SARS-CoV-2 infection, is overweight or clinically obese; is or has been a smoker; has or had chronic obstructive pulmonary disease (COPD); is asthmatic (e.g., having moderate to severe asthma); has an autoimmune disease or condition (e.g., diabetes); has a compromised or depleted immune system (e.g., due to AIDS/HIV infection, a cancer such as a blood cancer, a lymphodepleting therapy such as a chemotherapy, a bone marrow or organ transplantation, or a genetic immune condition); has chronic liver disease; has cardiovascular disease; and/or has a pulmonary or heart defect; and/or works or otherwise spends time in close proximity with others, such as in a factory, shipping center, hospital setting, or the like. In any of the presently disclosed embodiments, the subject has received a vaccine for SARS-CoV-2 and the vaccine is determined to be ineffective, e.g., by post-vaccine infection or symptoms in the subject, by clinical diagnosis or scientific or regulatory criteria. In any of the presently disclosed embodiments, the subject has not received a vaccine for SARS-CoV-2.

In any of the presently disclosed embodiments, the subject has received convalescent plasma therapy, remdesivir, or both, for SARS-CoV-2. In any of the presently disclosed embodiments, treatment comprises pre-exposure or peri-exposure prophylaxis. In any of the presently disclosed embodiments, treatment is administered to the subject having mild-to-moderate disease, optionally in an outpatient setting. In any of the presently disclosed embodiments, treatment is administered to a subject with moderate-to-severe disease, such as requiring hospitalization. In any of the presently disclosed embodiments, the subject is hospitalized with COVID-19.

In any of the presently disclosed embodiments, the subject having a SARS-CoV-2 infection: has mild-to-moderate COVID-19; is experiencing any one or more of: fever; cough; fatigue; shortness of breath or difficulty breathing; muscle aches; chills; sore throat; runny nose; headache; chest pain; loss of taste and/or smell; and pink eye (conjunctivitis); malaise; and abnormal imaging; has evidence of lower respiratory disease by clinical assessment or imaging and a saturation of oxygen (SaO2) greater than (>) 93 percent (%) on room air at sea level, has a positive SARS-CoV-2 viral testing result, and/or is at high risk for progressing to severe COVID-19 and/or hospitalization, e.g., the human subject (1) is 65 years of age or older (≥65); has a body mass index (BMI) of 35 or greater (≥35); has chronic kidney disease; has diabetes; (5) has immunosuppressive disease, is receiving immunosuppressive treatment; is 55 years of age or older (≥55) and has cardiovascular disease, hypertension, chronic obstructive pulmonary disease, or other chronic respiratory disease; and/or is 12-17 years of age and has a BMI ≥85% for their age and gender, or sickle cell disease, congenital or acquired heart disease, neurodevelopmental disorders (e.g., cerebral palsy), a medical-related technological dependence (e.g., tracheostomy, gastrostomy, or positive pressure ventilation not related to COVID-19), or asthma, reactive airway or other chronic respiratory disease that requires daily medication for control; has recently been diagnosed with COVID-19 (e.g., within 1 day, 2 days, 3 days, 4 days, 5 days, 6 days, 7 days, 8 days, 9 days, 10 days, 11 days, 12 days, 13 days, or 14 days) and/or is within 10 days of symptom onset; or has or is experiencing any combination of the foregoing.

In any of the presently disclosed embodiments, the subject is (a) 18 years old or older, or (b) 55 years old or younger, provided that the subject is 18 years or older. In any of the presently disclosed embodiments, the subject has a laboratory confirmed COVID-19 infection by positive polymerase chain reaction (PCR; e.g., RT-PCR) test; e.g., on any type of respiratory tract sample). In any of the presently disclosed embodiments, the subject has peripheral capillary oxygen saturation (SpO₂) >94% room air (RA), and has experienced one or more symptoms of COVID-19 for ≤120 h (5 days). In any of the presently disclosed embodiments, the subject is further receiving or has received remdesivir, supplemental oxygen, ventilation therapy, respiration therapy, dexamethasone, tocilizumab, or any combination thereof.

In some embodiments, one or more of the following does not apply to a subject receiving therapy according to the present disclosure: any condition that would prohibit receipt of intramuscular injections such as coagulation disorder, bleeding diathesis, or thrombocytopenia; known allergy or hypersensitivity to any constituent present in an antibody composition; previous anaphylaxis or hypersensitivity to a monoclonal antibody; has previously received a COVID-19 vaccine; has previously received SARS-CoV-2 hyperimmune intravenous immunoglobulin (hIVIG) from COVID-19 survivors; has previously received convalescent plasma from a recovered COVID-19 patient or an anti-SARS-CoV-2 mAb; is a pregnant or breast-feeding female; Alanine aminotransferase (ALT) and/or aspartate aminotransferase (AST) >5 times the upper limit of normal (ULN); Stage 4 severe chronic kidney disease or requiring dialysis (i.e., estimated glomerular filtration rate <30 mL/min/1.73 m²); has symptoms consistent with severe COVID-19 as defined by shortness of breath at rest or respiratory distress or requiring supplemental oxygen; severely immunocompromised participants including but not limited to cancer patients receiving immunosuppressive chemotherapy or immunotherapy, those with a solid organ transplant or allogeneic stem cell transplant within the last 3 months, any history of heart or lung transplant or high dose long-term systemic corticosteroids (equivalent to ≥20 mg a day of prednisone or the systemic equivalent for over 2 weeks); had diabetes (requiring medication), chronic kidney disease (i.e., eGFR <60 as determined by the Modification of Diet in Renal Disease (MDRD) study), chronic liver disease (e.g., cirrhosis), congestive heart failure (New York Heart Association (NYHA) class II or more), chronic obstructive pulmonary disease (history of chronic bronchitis, chronic obstructive lung disease, or emphysema with dyspnoea on physical exertion), and moderate to severe asthma (participant requires an inhaled steroid to control symptoms or has been prescribed a course of oral steroids in the past year); previous anaphylaxis or hypersensitivity to a monoclonal antibody; end-organ dysfunction such as—a. stroke b. meningitis c. encephalitis d. myelitis e. myocardial infarction f. myocarditis g. pericarditis h. symptomatic congestive heart failure (New York Heart Association [NYHA] class III-IV) i. arterial or deep venous thrombosis or pulmonary embolism; end-organ failure category such as—a. requirement for high-flow oxygen, non-invasive ventilation, or invasive mechanical ventilation b. extracorporeal membrane oxygenation (ECMO) c. mechanical circulatory support (e.g., intra-aortic balloon pump, ventricular assist device) d. vasopressor therapy e. commencement of renal replacement therapy (RRT) during this admission (i.e. not patients on chronic RRT); stroke; meningitis; encephalitis; myelitis; myocardial ischemia; myocarditis; pericarditis; symptomatic congestive heart failure; arterial or deep venous thrombosis or pulmonary embolism; and current or imminent requirement for invasive mechanical ventilation, ECMO (extracorporeal membrane oxygenation), Mechanical circulatory support, vasopressor therapy, or commencement of renal replacement therapy at this admission (i.e. not patients on chronic renal replacement therapy).

Compositions comprising an antibody, antigen-binding fragment, polynucleotide, vector, host cell, or composition of the present disclosure may also be administered simultaneously with, prior to, or after administration of one or more other therapeutic agents. Such combination therapy may include administration of a single pharmaceutical dosage formulation which contains a compound of the invention and one or more additional active agents, as well as administration of compositions comprising an antibody or antigen-binding fragment of the disclosure and each active agent in its own separate dosage formulation. For example, an antibody or antigen-binding fragment thereof as described herein and the other active agent can be administered to the patient together in a single oral dosage composition such as a tablet or capsule, or each agent administered in separate oral dosage formulations. Similarly, an antibody or antigen-binding fragment as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising an antibody or antigen-binding fragment and one or more additional active agents can be administered at essentially the same time, i.e., concurrently, or at separately staggered times, i.e., sequentially and in any order; combination therapy is understood to include all these regimens.

In certain embodiments, a combination therapy is provided that comprises one or more anti-SARS-CoV-2 antibody (or one or more nucleic acid, host cell, vector, or composition) of the present disclosure and one or more anti-inflammatory agent and/or one or more anti-viral agent. In particular embodiments, the one or more anti-inflammatory agent comprises a corticosteroid such as, for example, dexamethasone, prednisone, or the like. In some embodiments, the one or more anti-inflammatory agents comprise a cytokine antagonist such as, for example, an antibody that binds to IL6 (such as siltuximab), or to IL-6R (such as tocilizumab), or to IL-1β, IL-7, IL-8, IL-9, IL-10, FGF, G-CSF, GM-CSF, IFN-γ, IP-10, MCP-1, MIP-1A, MIP1-B, PDGR, TNF-α, or VEGF. In some embodiments, anti-inflammatory agents such as ruxolitinib and/or anakinra are used. In some embodiments, the one or more anti-viral agents comprise nucleotide analogs or nucleotide analog prodrugs such as, for example, remdesivir, sofosbuvir, acyclovir, and zidovudine. In particular embodiments, an anti-viral agent comprises lopinavir, ritonavir, favipiravir, leronlimab or any combination thereof. Other anti-inflammatory agents for use in a combination therapy of the present disclosure include non-steroidal anti-inflammatory drugs (NSAIDS). It will be appreciated that in such a combination therapy, the one or more antibody (or one or more nucleic acid, host cell, vector, or composition) and the one or more anti-inflammatory agent and/or one or the more antiviral agent can be administered in any order and any sequence, or together.

In some embodiments, an antibody (or one or more nucleic acid, host cell, vector, or composition) is administered to a subject who has previously received one or more anti-inflammatory agent and/or one or more antiviral agent. In some embodiments, one or more anti-inflammatory agent and/or one or more antiviral agent is administered to a subject who has previously received an antibody (or one or more nucleic acid, host cell, vector, or composition).

In certain embodiments, a combination therapy is provided that comprises two or more anti-SARS-CoV-2 antibodies of the present disclosure. A method can comprise administering a first antibody to a subject who has received a second antibody, or can comprise administering two or more antibodies together. For example, in particular embodiments, a method is provided that comprises administering to the subject (a) a first antibody or antigen-binding fragment, when the subject has received a second antibody or antigen-binding fragment; (b) the second antibody or antigen-binding fragment, when the subject has received the first antibody or antigen-binding fragment; or (c) the first antibody or antigen-binding fragment, and the second antibody or antigen-binding fragment.

In a related aspect, uses of the presently disclosed antibodies, antigen-binding fragments, vectors, host cells, and compositions are provided.

In certain embodiments, any of the presently disclosed antibodies, antigen-binding fragments, polynucleotides, vectors, host cells, or compositions is provided for use in a method (e.g., any of the presently disclosed methods) of treating a SARS-CoV-2 infection and/or COVID-19 in a subject.

In certain embodiments, any of the presently disclosed antibodies, antigen-binding fragments, or compositions is provided for use in a method of manufacturing or preparing a medicament for treating a SARS-CoV-2 infection and/or COVID-19 in a subject.

The present disclosure also provides the following Embodiments.

Embodiment 1. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 comprises or consists of the amino acid sequence according to any one of SEQ ID NOs.: 2, 56, 64, 80, 88, 96, 106, 156, 179, 195, or 240, or a sequence variant thereof comprising one, two, or three acid substitutions, one or more of which substitutions is optionally a conservative substitution and/or is a substitution to a germline-encoded amino acid; (ii) the CDRH2 comprises or consists of the amino acid sequence according to any one of SEQ ID NOs.: 3, 16-22, 57, 65, 81, 89, 97, 107, 121-126, 157, 180, 197, 199, or 241, or a sequence variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence according to any one of SEQ ID NOs.: 4, 25, 26, 58, 66, 82, 90, 98, 104, 108, 127, 128, 158, 181, 201, 203, or 242, or a sequence variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence according to any one of SEQ ID NOs.: 6, 51-54, 60, 68, 73, 74, 84, 92, 100, 110, 160, 169, 183, 235, or 244, or a sequence variant thereof comprising one, two, or three amino acid substitutions, one or more of which substitutions is optionally a conservative substitution and/or is a substitution to a germline-encoded amino acid; (v) the CDRL2 comprises or consists of the amino acid sequence according to any one of SEQ ID NOs.: 7, 61, 69, 85, 93, 101, 111, 161, 170, 184, 236 or 245, or a sequence variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence according to any one of SEQ ID NOs.: 8, 62, 70, 77, 78, 86, 94, 102, 112, 151-154, 162, 171, 185, 237, or 246, or a sequence variant thereof comprising having one, two, or three amino acid substitutions, one or more of which substitutions is optionally a conservative substitution and/or is a substitution to a germline-encoded amino acid, wherein the antibody or antigen binding fragment is capable of binding to a surface glycoprotein of a SARS-CoV-2 expressed on a cell surface of a host cell and/or on a virion.

Embodiment 2. The antibody or antigen-binding fragment of Embodiment 1, which is capable of neutralizing a SARS-CoV-2 infection in an in vitro model of infection and/or in an in vivo animal model of infection and/or in a human.

Embodiment 3. The antibody or antigen-binding fragment of any one of Embodiments 1-2, comprising CDRH1, CDRH2, CDRH3, CDRL1, CDRL2, and CDRL3 amino acid sequences according to SEQ ID NOs.: (i) 2-4 and 6-8 or 235-237, respectively; (ii) 2, any one of 16-22, 4, and 6-8 or 235-237, respectively; (iii) 2, 3, any one of 25-26, and 6-8 or 235-237, respectively; (iv) 2-4, 51, 7 or 236, and 8 or 237, respectively; (v) 2-4, 52, 7 or 236, and 8 or 237, respectively; (vi) 2-4, 53, 7 or 236, and 8 or 237, respectively; (vii) 2-5, 54, 7 or 236, and 8 or 237, respectively; (viii) 56-58 and 60-62, respectively; (ix) 64-66 and 68-70, respectively; (x) 64-66, 73 or 74, 69, and 70, respectively; (xi) 64-66, 68, 69, and 77 or 78, respectively; (xii) 80-82 and 84-86, respectively; (xiii) 88-90 and 92-94, respectively; (xiv) 96-98 and 101-102, respectively; (xv) 96, 97, 104, and 100-102, respectively; (xvi) 106-108 and 110-112 or 167-171, respectively; (xvii) 106, any one of 121-126, 108, and 110-112, respectively; (xviii) 106, 107, 127 or 128, and 110-112, respectively; (xix) 106-108, 110, 111, and 151, respectively; (xx) 106-108, 110, 111, and 152, respectively; (xxi) 106-108, 110, 111, and 153, respectively; (xxii) 106-108, 110, 111, and 154, respectively; (xxiii) 106-108 and 169-171, respectively; (xxiv) 156-158 and 160-162, respectively; (xxv) 106, 123, 127, and 169-171, respectively; (xxvi) 2, 17, 25, 6 or 235 or any one of 51-54, 7 or 236, and 8 or 237, respectively; (xxvii) 2, 20, 25, 6 or 235 or any one of 51-54, 7 or 236, and 8 or 237, respectively; or (xxviii) 179-181 and 183-185, respectively (xxix) 195, 180, 181 and 183-185, respectively; (xxx) 195, 197, 181 and 183-185, respectively; (xxxi) 195, 199, 181 and 183-185, respectively; (xxxii) 195, 197, 201 and 183-185, respectively; (xxxiii) 195, 197, 203 and 183-185, respectively; (xxxiv) 195, 199, 201 and 183-185, respectively; (xxxv) 195, 199, 203 and 183-185, respectively; (xxxvi) 179, 180, 181 and 183-185, respectively; (xxxvii) 179, 197, 181 and 183-185, respectively; (xxxviii) 179, 199, 181 and 183-185, respectively; (xxxix) 179, 197, 201 and 183-185, respectively; (xxxx) 179, 197, 203 and 183-185, respectively; (xxxxi) 179, 199, 201 and 183-185, respectively; (xxxxii) 179, 199, 203 and 183-185, respectively; (xxxxiii) 179, 180, 201 and 183-185, respectively; (xxxxiv) 179, 180, 203 and 183-185, respectively; and (xxxxv) 240-242 and 244-246, respectively.

Embodiment 4. The antibody or antigen-binding fragment of any one of Embodiments 1-5, wherein: (i) the VH comprises or consists of an amino acid sequence having at least 85% identity to the amino acid sequence according to any one of SEQ ID NOs.: 1, 9-15, 23, 24, 27, 28-46, 55, 63, 79, 87, 95, 103, 105, 113-120, 129-146, 155, 172, 176-178, 194, 196, 198, 200, 202, and 239, wherein the variation is optionally limited to one or more framework regions and/or the variation comprises one or more substitution to a germline-encoded amino acid; and/or (ii) the VL comprises or consists of an amino acid sequence having at least 85% identity to the amino acid sequence according to any one of SEQ ID NOs.: 5, 47-50, 59, 67, 71-72, 75, 76, 83, 91, 99, 109, 147-150, 159, 168, 182, 190, 234, and 243, wherein the variation is optionally limited to one or more framework regions and/or the variation comprises one or more substitution to a germline-encoded amino acid.

Embodiment 5. The antibody or antigen-binding fragment of any one of Embodiments 1-6, wherein the VH comprises or consists of any VH amino acid sequence set forth in Table 1, and wherein the VL comprises or consists of any VL amino acid sequence set forth in Table 1, wherein, optionally, the VH and the VL comprise or consist of the amino acid sequences according to SEQ ID NOs.: (i) 1 and 5 or 234, respectively; (ii) any one of 9-15 and 5 or 234, respectively; (iii) 23 or 24 and 5 or 234, respectively; (iv) 27 and 5 or 234, respectively; (v) any one of 28-46 and 5 or 234, respectively; (vi) 1 and any one of 47-50, respectively; (vii) any one of 9-15 and any one of 47-50, respectively; (viii) 23 or 24 and any one of 47-50, respectively; (ix) 27 and any one of 47-50, respectively; (x) any one of 28-46 and any one of 47-50, respectively; (xi) 55 and 59, respectively; (xii) 63 and 67, respectively; (xiii) 63 and 71 or 72, respectively; (xiv) 63 and 75 or 76, respectively; (xv) 79 and 83, respectively; (xvi) 87 and 91, respectively; (xvii) 95 and 99, respectively; (xviii) 103 and 99, respectively; (xiv) 105 and 109 or 168, respectively; (xx) any one of 113-120 and 109 or 168, respectively; (xxi) 129 and 109 or 168, respectively; (xxii) any one of 130-146 and 109 or 168, respectively; (xxiii) 105 and any one of 147-150, respectively; (xxiv) any one of 113-120 and any one of 147-150, respectively; (xxv) any one of 130-146 and any one of 147-150, respectively; (xxvi) 155 and 159, respectively; (xxvii) 172 and 168, respectively; (xxviii) 176 or 177 and 5 or 234 or any one of 47-50, respectively; (xxix) 178 and 182 or 190, respectively; (xxx) 194 and 182, respectively; (xxxi) 196 and 182, respectively; (xxxii) 198 and 182, respectively; (xxxiii) 200 and 182, respectively; (xxxiv) 202 and 182, respectively; or (xxxv) 239 and 243, respectively.

Embodiment 6. An antibody, or antigen-binding fragment thereof, comprising a heavy chain variable domain (VH) and a light chain variable domain (VL), wherein the VH comprises or consists of the amino acid sequence as set forth in SEQ ID NO: 79 and the VL comprises or consists of the amino acid sequence as set forth in SEQ ID NO: 83.

Embodiment 7. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 80-82, respectively, and the CDRL1, CDRL2, and CDRL3 comprise or consist of the amino acid sequences set forth in SEQ ID NOs: 84-86, respectively.

Embodiment 8. An antibody, or antigen-binding fragment thereof, comprising a heavy chain variable domain (VH) and a light chain variable domain (VL), wherein the VH comprises or consists of the amino acid sequence as set forth in SEQ ID NO: 105 and the VL comprises or consists of the amino acid sequence as set forth in SEQ ID NO: 168.

Embodiment 9. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 106-108, respectively, and the CDRL1, CDRL2, and CDRL3 comprise or consist of the amino acid sequences set forth in SEQ ID NOs: 169-171, respectively.

Embodiment 10. An antibody, or antigen-binding fragment thereof, comprising a heavy chain variable domain (VH) and a light chain variable domain (VL), wherein the VH comprises or consists of the amino acid sequence as set forth in SEQ ID NO: 178 and the VL comprises or consists of the amino acid sequence as set forth in SEQ ID NO: 182 or SEQ ID NO: 190.

Embodiment 11. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 179-181, respectively, and the CDRL1, CDRL2, and CDRL3 comprise or consist of the amino acid sequences set forth in SEQ ID NOs: 183-185, respectively.

Embodiment 12. The antibody or antigen-binding fragment of any one of Embodiments 1-11, which: (i) recognizes an epitope in the ACE2 receptor binding motif (RBM, SEQ ID NO.: 167) of SARS-CoV-2; (ii) is capable of blocking an interaction between SARS-CoV-2 (e.g., SARS-CoV-2 RBM) and ACE2;

(ii) is capable of binding to SARS-CoV-2 S protein with greater avidity than to SARS coronavirus S protein; (iv) is capable of staining about 30%, about 35%, about 40%, about 50%, about 55%, about 56%, about 57%, about 58%, about 59%, about 60%, or more of target cells expressing SARS-CoV-2 surface glycoprotein in a sample comprising about 50,000 of the target cells in approximately 100 μL when the antibody or antigen-binding fragment is present at 10 μg/ml; (v) recognizes an epitope that is conserved in the ACE2 RBM of SARS-CoV-2 and in an ACE2 RBM of SARS coronavirus; (vi) is cross-reactive against SARS-CoV-2 and SARS coronavirus; (vii) recognizes an epitope in the SARS-CoV-2 surface glycoprotein that is not in the ACE2 RBM; or (viii) any combination of (i)-(vii).

Embodiment 13. The antibody or antigen-binding fragment of any one of Embodiments 1-12, which is a IgG, IgA, IgM, IgE, or IgD isotype.

Embodiment 14. The antibody or antigen-binding fragment of any one of Embodiments 1-13, which is an IgG isotype selected from IgG1, IgG2, IgG3, and IgG4.

Embodiment 15. The antibody or antigen-binding fragment of any one of Embodiments 1-14, which is human, humanized, or chimeric.

Embodiment 16. The antibody or antigen-binding fragment of any one of Embodiments 1-15, wherein the antibody, or the antigen-binding fragment, comprises a human antibody, a monoclonal antibody, a purified antibody, a single chain antibody, a Fab, a Fab′, a F(ab′)2, a Fv, a scFv, or a scFab.

Embodiment 17. The antibody or antigen-binding fragment of Embodiment 16, wherein the scFab comprises: (i) the amino acid sequence as set forth in any one of SEQ ID NOs: 218-219 and 226-227; (ii) a VL comprising the amino acid sequence as set forth in SEQ ID NO: 168 and a VH comprising the amino acid sequence as set forth in SEQ ID NO: 105 or SEQ ID NO: 113; or (iii) a CDRH1 comprising the amino acid sequence as set forth in SEQ ID NO: 106, a CDRH2 comprising the amino acid sequence as set forth in SEQ ID NO: 107 or 121, a CDRH3 comprising the amino acid sequence as set forth in SEQ ID NO: 108, a CDRL1 comprising the amino acid sequence as set forth in SEQ ID NO: 169, a CDRL2 comprising the amino acid sequence as set forth in SEQ ID NO: 170, and a CDRL3 comprising the amino acid sequence as set forth in SEQ ID NO: 171.

Embodiment 18. The antibody or antigen-binding fragment of Embodiment 16, wherein the scFv comprises: (i) the amino acid sequence as set forth in any one of SEQ ID NOs: 220-221 or 228-229; (ii) a VL comprising the amino acid sequence as set forth in SEQ ID NO: 168 and a VH comprising the amino acid sequence as set forth in SEQ ID NO: 105 or SEQ ID NO: 113; or (iii) a CDRH1 comprising the amino acid sequence as set forth in SEQ ID NO: 106, a CDRH2 comprising the amino acid sequence as set forth in SEQ ID NO: 107 or 121, a CDRH3 comprising the amino acid sequence as set forth in SEQ ID NO: 108, a CDRL1 comprising the amino acid sequence as set forth in SEQ ID NO: 169, a CDRL2 comprising the amino acid sequence as set forth in SEQ ID NO: 170, and a CDRL3 comprising the amino acid sequence as set forth in SEQ ID NO: 171.

Embodiment 19. The antibody or antigen-binding fragment of Embodiment 16, wherein the scFv comprises more than one VH domain and more than one VL domain.

Embodiment 20. The antibody or antigen-binding fragment of Embodiment 19, wherein the scFv comprises: (i) the amino acid sequence as set forth in any one of SEQ ID NO: 222-225 or SEQ ID NO: 230-233; (ii) two VL domains, each comprising the amino acid sequence as set forth in SEQ ID NO: 168, and two VH domains, each comprising the amino acid sequence as set forth in SEQ ID NO: 105 or SEQ ID NO: 113; or (iii) two VL domains, each comprising a CDRL1 comprising the amino acid sequence as set forth in SEQ ID NO: 169, a CDRL2 comprising the amino acid sequence as set forth in SEQ ID NO: 170, and a CDRL3 comprising the amino acid sequence as set forth in SEQ ID NO: 171, and two VH domains, each comprising a CDRH1 comprising the amino acid sequence as set forth in SEQ ID NO: 106, a CDRH2 comprising the amino acid sequence as set forth in SEQ ID NO: 107 or 121, a CDRH3 comprising the amino acid sequence as set forth in SEQ ID NO: 108.

Embodiment 21. The antibody or antigen-binding fragment of any one of Embodiments 1-19, wherein the antibody or antigen-binding fragment is a multi-specific antibody or antigen binding fragment.

Embodiment 22. The antibody or antigen-binding fragment of Embodiment 21, wherein the antibody or antigen binding fragment is a bispecific antibody or antigen-binding fragment.

Embodiment 23. The antibody or antigen-binding fragment of Embodiment 21 or 22, comprising: (i) a first VH and a first VL; and (ii) a second VH and a second VL, wherein the first VH and the second VH are different and each independently comprise an amino acid sequence having at least 85% identity to the amino acid sequence set forth in any one of SEQ ID NOs.: 1, 9-15, 23, 24, 27-46, 55, 63, 79, 87, 95, 103, 105, 113-120, 129-146, 155, 172, 176-178, 194, 196, 198, 200, 202 and 239, and wherein the first VL and the second VL are different and each independently comprise an amino acid sequence having at least 85% identity to the amino acid sequence set forth in any one of SEQ ID NOs.: 5, 47-50, 59, 67, 71, 72, 75, 76, 83, 91, 99, 109, 147-150, 159, 168, 182, 190, 234, and 243;
and wherein the first VH and the first VL together form a first antigen-binding site, and wherein the second VH and the second VL together form a second antigen-binding site.

Embodiment 24. The antibody or antigen-binding fragment of any one of Embodiments 1-23, wherein the antibody or antigen-binding fragment further comprises a Fc polypeptide or a fragment thereof.

Embodiment 25. The antibody or antigen-binding fragment of Embodiment 24, wherein the Fc polypeptide or fragment thereof comprises: (i) a mutation that enhances binding to a FcRn as compared to a reference Fc polypeptide that does not comprise the mutation; and/or (ii) a mutation that enhances binding to a FcγR as compared to a reference Fc polypeptide that does not comprise the mutation.

Embodiment 26. The antibody or antigen-binding fragment of Embodiment 25, wherein the mutation that enhances binding to a FcRn comprises: M428L; N434S; N434H; N434A; N434S; M252Y; S254T; T256E; T250Q; P257I; Q311I; D376V; T307A; E380A; or any combination thereof.

Embodiment 27. The antibody or antigen-binding fragment of Embodiment 25 or 26, wherein the mutation that enhances binding to FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; or (viii) any combination of (i)-(vii).

Embodiment 28. The antibody or antigen-binding fragment of any one of Embodiments 25-27, wherein the mutation that enhances binding to FcRn comprises M428L/N434S.

Embodiment 29. The antibody or antigen-binding fragment of any one of Embodiments 25-28, wherein the mutation that enhances binding to a FcγR comprises S239D; I332E; A330L; G236A; or any combination thereof.

Embodiment 30. The antibody or antigen-binding fragment of any one of Embodiments 25-29, wherein the mutation that enhances binding to a FcγR comprises: (i) S239D/I332E; (ii) S239D/A330L/I332E; (iii) G236A/S239D/I332E; or (iv) G236A/A330L/I332E.

Embodiment 31. The antibody or antigen-binding fragment of any one of Embodiments 1-30, which comprises a mutation that alters glycosylation, wherein the mutation that alters glycosylation comprises N297A, N297Q, or N297G, and/or which is aglycosylated and/or afucosylated.

Embodiment 32. An isolated polynucleotide encoding the antibody or antigen-binding fragment of any one of Embodiments 1-31, or encoding a VH, a heavy chain, a VL, and/or a light chain of the antibody or the antigen-binding fragment.

Embodiment 33. The polynucleotide of Embodiment 32, wherein the polynucleotide comprises deoxyribonucleic acid (DNA) or ribonucleic acid (RNA), wherein the RNA optionally comprises messenger RNA (mRNA).

Embodiment 34. The polynucleotide of Embodiment 32 or 33, which is codon-optimized for expression in a host cell.

Embodiment 35. The polynucleotide of any one of Embodiments 32-34, comprising a polynucleotide having at least 50% identity to the polynucleotide sequence according to any one or more of SEQ ID NOs.: 186-189, 191-192, 238, 247, 248-255 and 257-262.

Embodiment 36. A recombinant vector comprising the polynucleotide of any one of Embodiments 32-35.

Embodiment 37. A host cell comprising the polynucleotide of any one of Embodiments 32-35 and/or the vector of Embodiment 36, wherein the polynucleotide is heterologous to the host cell.

Embodiment 38. A human B cell comprising the polynucleotide of any one of Embodiments 32-35, wherein polynucleotide is heterologous to the human B cell and/or wherein the human B cell is immortalized.

Embodiment 39. A composition comprising: (i) the antibody or antigen-binding fragment of any one of Embodiments 1-31 or 49-52; (ii) the polynucleotide of any one of Embodiments 32-35; (iii) the recombinant vector of Embodiment 36; (iv) the host cell of Embodiment 37; and/or (v) the human B cell of Embodiment 38, and a pharmaceutically acceptable excipient, carrier, or diluent.

Embodiment 40. The composition of Embodiment 39, comprising two or more antibodies or antigen-binding fragments of any one of Embodiments 1-31 or 49-52.

Embodiment 41. The composition of Embodiment 40, comprising:
(i) a first antibody or antigen-binding fragment, comprising a VH comprising or consisting of the amino acid sequence as set forth in SEQ ID NO: 79 and a VL comprising or consisting of the amino acid sequence as set forth in SEQ ID NO: 83; and
(ii) a second antibody or antigen-binding fragment comprising, a VH comprising or consisting of the amino acid sequence as set forth in SEQ ID NO: 105 and a VL comprising of consisting of the amino acid sequence as set forth in SEQ ID NO: 168.

Embodiment 42. The composition of Embodiment 40, comprising:
(i) a first antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 80-82, respectively, and the CDRL1, CDRL2, and CDRL3 comprise or consist of the amino acid sequences set forth in SEQ ID NOs: 84-86, respectively; and (ii) a second antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 106-108, respectively, and the CDRL1, CDRL2, and CDRL3 comprise or consist of the amino acid sequences set forth in SEQ ID NOs: 169-171, respectively.

Embodiment 43. The composition of Embodiment 40, comprising:
(i) a first antibody or antigen-binding fragment, comprising a VH comprising or consisting of the amino acid sequence as set forth in SEQ ID NO: 178 and a VL comprising or consisting of the amino acid sequence as set forth in SEQ ID NO: 182 or SEQ ID NO: 190; and (ii) a second antibody or antigen-binding fragment comprising, a VH comprising or consisting of the amino acid sequence as set forth in SEQ ID NO: 105 and a VL comprising of consisting of the amino acid sequence as set forth in SEQ ID NO: 168.

Embodiment 44. The composition of Embodiment 40, comprising:
(i) a first antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 179-181, respectively, and the CDRL1, CDRL2, and CDRL3 comprise or consist of the amino acid sequences set forth in SEQ ID NOs: 183-185, respectively; and (ii) a second antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 106-108, respectively, and the CDRL1, CDRL2, and CDRL3 comprise or consist of the amino acid sequences set forth in SEQ ID NOs: 169-171, respectively.

Embodiment 45. The composition of Embodiment 40, comprising:
(i) a first antibody or antigen-binding fragment, comprising a VH comprising or consisting of the amino acid sequence as set forth in SEQ ID NO: 178 and a VL comprising or consisting of the amino acid sequence as set forth in SEQ ID NO: 182 or SEQ ID NO: 190; and (ii) a second antibody or antigen-binding fragment comprising, a VH comprising or consisting of the amino acid sequence as set forth in SEQ ID NO: 63 and a VL comprising or consisting of the amino acid sequence as set forth in SEQ ID NO: 67, any one of SEQ ID NO: 71-71, or any one of SEQ ID NO: 75-76.

Embodiment 46. The composition of Embodiment 40, comprising:
(i) a first antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 179-181, respectively, and the CDRL1, CDRL2, and CDRL3 comprise or consist of the amino acid sequences set forth in SEQ ID NOs: 183-185, respectively; and (ii) a second antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 64-66, respectively, the CDRL1 comprises or consists of the amino acid sequences set forth in any one of SEQ ID NO: 68, SEQ ID NO: 73, or SEQ ID NO: 74, the CDRL2 comprises or consists of the amino acid sequences set forth in SEQ ID NO: 69, and the CDRL3 comprises or consists of the amino acid sequences set forth in any one of SEQ ID NO: 70, SEQ ID NO:77, or SEQ ID NO: 78.

Embodiment 47. A composition comprising the polynucleotide of any one of Embodiments 32-35 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

Embodiment 48. A method of treating a SARS-CoV-2 infection in a subject, the method comprising administering to the subject an effective amount of (i) the antibody or antigen-binding fragment of any one of Embodiments 1-31 or 51-54; (ii) the polynucleotide of any one of Embodiments 32-35; (iii) the recombinant vector of Embodiment 36; (iv) the host cell of Embodiment 37; (v) the human B cell of Embodiment 38; and/or (vi) the composition of any one of Embodiments 39-47.

Embodiment 49. The antibody or antigen-binding fragment of any one of Embodiments 1-31 or 51-54, the polynucleotide of any one of Embodiments 32-35, the recombinant vector of Embodiment 36, the host cell of Embodiment 37, the human B cell of Embodiment 38, and/or the composition of any one of Embodiments 39-47 for use in a method of treating a SARS-CoV-2 infection in a subject.

Embodiment 50. The antibody or antigen-binding fragment of any one of Embodiments 1-31 or 51-54, the polynucleotide of any one of Embodiments 32-35, the recombinant vector of Embodiment 36, the host cell of Embodiment 37, the human B cell of Embodiment 38, and/or the composition of any one of Embodiments 39-47 for use in the preparation of a medicament for the treatment of a SARS-CoV-2 infection in a subject.

Embodiment 51. The antibody or antigen-binding fragment of any one of Embodiments 24-31, wherein the Fc polypeptide comprises a L234A mutation and a L235A mutation.

Embodiment 52. The antibody or antigen-binding fragment of any one of Embodiments 1-31 or 51, wherein the antibody or antigen-binding fragment binds to SARS-CoV-2 S protein, as measured using biolayer interferometry.

Embodiment 53. The antibody or antigen-binding fragment of Embodiment 52, wherein the antibody or antigen-binding fragment binds to SARS-CoV-2 S protein with a KD of less than about 4.5×10⁻⁹ M.

Embodiment 54. The antibody or antigen-binding fragment of Embodiment 52 or 53, wherein the antibody or antigen-binding fragment binds to SARS-CoV-2 S protein with a K_{D} less than 1×10⁻¹² M.

Embodiment 55. The antibody or antigen-binding fragment of any one of Embodiments 1-31 or 51-54, wherein the antibody or antigen-binding fragment is capable of neutralizing a SARS-CoV-2 infection and/or of neutralizing an infection of a target cell with an IC50 of about 16 to about 20 μg/ml.

Embodiment 56. The antibody or antigen-binding fragment of any one of Embodiments 1-31 or 51-54, wherein the antibody or antigen-binding fragment is capable of neutralizing a SARS-CoV-2 infection and/or of neutralizing an infection of a target cell with an IC50 of about 0.3 to about 0.4 μg/ml or about 3 to about 4 nM.

Embodiment 57. A composition comprising (i) the antibody or antigen-binding fragment of Embodiment 8 or 9 and (ii) the antibody or antigen-binding fragment of Embodiment 10 or 11, wherein the composition is capable of neutralizing a SARS-CoV-2 infection with an IC50 of about 0.07 to about 0.08 μg/ml.

Embodiment 58. The antibody or antigen-binding fragment of any one of Embodiments 1-31 or 51-54, wherein the antibody or antigen-binding fragment is capable of inducing antibody-dependent cell-mediated cytotoxicity (ADCC) and/or antibody dependent cellular phagocytosis (ADCP) against a target cell infected by a SARS-CoV-2.

Embodiment 59. A method for in vitro diagnosis of a SARS-CoV-2 infection, the method comprising:
(i) contacting a sample from a subject with an antibody or antigen-binding fragment of any one of Embodiments 1-31 or 51-54; and
(ii) detecting a complex comprising an antigen and the antibody, or comprising an antigen and the antigen binding fragment.

Embodiment 60. The method of Embodiment 59, wherein the sample comprises blood isolated from the subject.

Embodiment 61. The antibody or antigen-binding fragment of any one of Embodiments 52-56, wherein a Fab of the antibody or antigen-binding fragment is capable of binding to SARS-CoV-2 S protein with a KD of 2.0×10−9 or less, 1.9×10−9 or less, or 1.8×10−9 or less.

Embodiment 62. The antibody or antigen-binding fragment of any one of Embodiments 1-31, 51-54, or 61, wherein the antibody or antigen-binding fragment is capable of neutralizing infection by the SARS-CoV-2 and does not compete with a human ACE2 for binding to the SARS-CoV-2 S protein,
wherein, optionally, the neutralizing comprises neutralizing infection in an in vitro model of infection.

Embodiment 63. The antibody or antigen-binding fragment of any one of Embodiments 1-31, 51-54, 61, or 62, wherein the antibody or antigen-binding fragment is capable of neutralizing infection by the SARS-CoV-2 with an IC50 of 3.0 nM, 3.1 nM, 3.2 nM, 3.3 nM, 3.4 nM, 3.5 nM, 3.6 nM, 3.7 nM, 3.8 nM, 3.9 nM, or 4.0 nM.

Embodiment 64. The antibody or antigen-binding fragment of Embodiment 58, wherein the inducing ADCC comprises activating a Natural Killer cell that comprises a V158 FcγRIIIa variant, a Natural Killer cell that comprises a F158 FcγRIIIa variant, or both.

Embodiment 65. The antibody or antigen-binding fragment of Embodiment 58 or 64, wherein the ADCP comprises engaging a FcγRIIa expressed on the surface of a phagocytic cell, such as a monocyte, a macrophage, or a dendritic cell.

Embodiment 66. An antibody, or an antigen-binding fragment thereof, that competes for binding to a SARS-CoV-2 surface glycoprotein with the antibody or antigen-binding fragment of any one of Embodiments 1-31, 51-54, or 61-65.

Embodiment 67. An antibody, or an antigen-binding fragment thereof, that competes for binding to a SARS-CoV-2 surface glycoprotein with antibody S309 and/or antibody S303.

Embodiment 68. An antibody, or an antigen-binding fragment thereof, that competes for binding to a SARS-CoV-2 surface glycoprotein with antibody S304 and/or antibody S315.

Embodiment 69. A combination or composition comprising:
(i) an antibody or antigen-binding fragment comprising
(a) a CDRH1 amino acid sequence GYPFTSYG, a CDRH2 amino acid sequence ISTYNGNT or ISTYQGNT, a CDRH3 amino acid sequence ARDYTRGAWFGESLIGGFDN; a CDRL1 amino acid sequence QTVSSTS, a CDRL2 amino acid sequence GAS, and a CDRL3 amino acid sequence QQHDTSLT; or
(b) a VH amino acid sequence comprising or consisting of

QVQLVQSGAEVKKPGASVKVSCKASGYPFTSYGISWVRQAPGQGLEWMG
WISTYNGNTNYAQKFQGRVTMTTDTSTTTGYMELRRLRSDDTAVYYCAR
DYTRGAWFGESLIGGFDNWGQGTLVTVSS

or comprising or consisting of

QVQLVQSGAEVKKPGASVKVSCKASGYPFTSYGISWVRQAPGQGLEWMG
WISTYQGNTNYAQKFQGRVTMTTDTSTTTGYMELRRLRSDDTAVYYCAR
DYTRGAWFGESLIGGFDNWGQGTLVTVSS,

and a VL amino acid sequence comprising or consisting of EIVLTQSPGTLSLSPGERATLSCRASQTVSSTSLAWYQQKPGQAPRLLIYGASSR ATGIPDRFSGSGSGTDFTLTISRLEPEDFAVYYCQQHDTSLTFGGGTK VEIK; and
(ii) an antibody or antigen-binding fragment comprising: (a) VH and VL amino acid sequences according to SEQ ID NOs.: 79 and 83, respectively; (b) CDRH1, CDRH2, CDRH3, CDRL1, CDRL2, and CDRL3 amino acid sequences according to SEQ ID NOS.: 80-82 and 84-86, respectively; (c); VH and VL amino acid sequences according to SEQ ID NOs.: 178 or 194 or 196 or 198 or 200 or 202 and 182 or 190, respectively; or (d) CDRH1, CDRH2, CDRH3, CDRL1, CDRL2, and CDRL3 amino acid sequences according to SEQ ID NOS.: 179 or 195, 180 or 197 or 199, 181 201 or 203, and 183-185, respectively.

Embodiment 70. A method of preventing or treating or neutralizing a coronavirus infection in a subject, the method comprising administering to the subject the combination or composition of Embodiment 69, wherein, optionally, the antibody or antigen binding fragment of (i) and the antibody or antigen binding fragment of (ii) are administered concurrently, simultaneously, or consecutively.

Embodiment 71. A method of preventing or treating or neutralizing a coronavirus infection in a subject, the method comprising administering to a subject who has received a first antibody or antigen binding fragment comprising: (a) VH and VL amino acid sequences according to SEQ ID NOs.: 79 and 83, respectively; or (b) CDRH1, CDRH2, CDRH3, CDRL1, CDRL2, and CDRL3 amino acid sequences according to SEQ ID NOS.: 80-82 and 84-86, respectively; a second antibody or antigen binding fragment comprising: (a) a VH amino acid sequence according to SEQ ID NOs.: 105 or 113, and a VL amino acid sequence according to SEQ ID NO: 168; or (b) CDRH1, CDRH2, and CDRH3 amino acids according to SEQ ID NOs.: 106-108, respectively, or SEQ ID NOs.: 106, 121, and 108, respectively, and CDRL1, CDRL2, and CDRL3 amino acid sequences according to SEQ ID NOS.: 169-171, respectively.

Embodiment 72. A method of preventing or treating or neutralizing a coronavirus infection in a subject, the method comprising administering to a subject who has received a first antibody or antigen binding fragment comprising: (a) a VH amino acid sequence according to SEQ ID NOs.: 105 or 113, and a VL amino acid sequence according to SEQ ID NO: 168; or (b) CDRH1, CDRH2, and CDRH3 amino acids according to SEQ ID NOs.: 106-108, respectively, or SEQ ID NOs.: 106, 121, and 108, respectively, and CDRL1, CDRL2, and CDRL3 amino acid sequences according to SEQ ID NOS.: 169-171, respectively; a second antibody or antigen binding fragment comprising: (a) VH and VL amino acid sequences according to SEQ ID NOs.: 79 and 83, respectively; or (b) CDRH1, CDRH2, CDRH3, CDRL1, CDRL2, and CDRL3 amino acid sequences according to SEQ ID NOS.: 80-82 and 84-86, respectively.

Embodiment 73. A method of preventing or treating or neutralizing a coronavirus infection in a subject, the method comprising administering to a subject who has received a first antibody or antigen binding fragment comprising:
(a) VH and VL amino acid sequences according to SEQ ID NOs.: 178 or 194 or 196 or 198 or 200 or 202 and 182 or 190, respectively; or (b) CDRH1, CDRH2, CDRH3, CDRL1, CDRL2, and CDRL3 amino acid sequences according to SEQ ID NOS.: 179 or 195, 180 or 197 or 199, 181 201 or 203, and 183-185, respectively; a second antibody or antigen binding fragment comprising: (a) a VH amino acid sequence according to SEQ ID NOs.: 105 or 113, and a VL amino acid sequence according to SEQ ID NO: 168; or (b) CDRH1, CDRH2, and CDRH3 amino acids according to SEQ ID NOs.: 106-108, respectively, or SEQ ID NOs.: 106, 121, and 108, respectively, and CDRL1, CDRL2, and CDRL3 amino acid sequences according to SEQ ID NOS.: 169-171, respectively.

Embodiment 74. A method of preventing or treating or neutralizing a coronavirus infection in a subject, the method comprising administering to a subject who has received a first antibody or antigen binding fragment comprising: (a) a VH amino acid sequence according to SEQ ID NOs.: 105 or 113, and a VL amino acid sequence according to SEQ ID NO: 168; or (b) CDRH1, CDRH2, and CDRH3 amino acids according to SEQ ID NOs.: 106-108, respectively, or SEQ ID NOs.: 106, 121, and 108, respectively, and CDRL1, CDRL2, and CDRL3 amino acid sequences according to SEQ ID NOS.: 169-171, respectively; a second antibody or antigen binding fragment comprising: (a) VH and VL amino acid sequences according to SEQ ID NOs.: 178 or 194 or 196 or 198 or 200 or 202 and 182 or 190, respectively; or (b) CDRH1, CDRH2, CDRH3, CDRL1, CDRL2, and CDRL3 amino acid sequences according to SEQ ID NOS.: 179 or 195, 180 or 197 or 199, 181 201 or 203, and 183-185, respectively.

Embodiment 75. A method of treating a SARS-CoV-2 infection in a subject, the method comprising administering to the subject a single dose of a composition comprising the antibody or antigen-binding fragment of any one of Embodiments 1-31, 51-54, and 61-65.

Embodiment 76. The method of Embodiment 48 or 75, wherein the antibody or antigen-binding fragment comprises CDRH1, CDRH2, CDRH3, CDRL1, CDRL2, and CDRL3 amino acid sequences according to SEQ ID NOS.: 106, 121, 108, 169, 170, and 171, respectively, and optionally further comprises a (n e.g. IgG1) Fc polypeptide comprising a M428L/N434S mutation, preferably wherein the antibody or antigen-binding fragment comprises the CH1-CH3 amino acid sequence of SEQ ID NO.: 173 and the CL amino acid sequence of SEQ ID NO.: 174.

Embodiment 77. The method of any one of Embodiments 48, 75, and 76, wherein the antibody or antigen-binding fragment comprises a VH according to SEQ ID NO: 113 and a VL according to SEQ ID NO: 168, and optionally further comprises a (n e.g. IgG1) Fc polypeptide comprising a M428L/N434S mutation, preferably wherein the antibody or antigen-binding fragment comprises the CH1-CH3 amino acid sequence of SEQ ID NO.: 173 and the CL amino acid sequence of SEQ ID NO.: 174.

Embodiment 78. The method of any one of Embodiments 48 and 75-77, wherein the subject: (i) is aged 18 to 49 years; (ii) is 18 years old or older; (iii) has mild to moderate COVID-19; (iv) has severe COVID-19; (v) has severe to critical COVID-19; (vi) has had fewer than seven days or 5 or fewer days since onset of symptoms; (vii) has had seven days or more since onset of symptoms; (viii) has had a positive reverse-transcriptase-polymerase-chain-reaction or antigen SARS-CoV-2 test result; (ix) is 55 years of age or older;
(x) has one or more of: diabetes requiring medication, obesity (body-mass index >30 kg/m²), chronic kidney disease (estimated glomerular filtration rate <60 mL/min/1.73 m²),²³ congestive heart failure (New York Heart Association class II or higher), chronic obstructive pulmonary disease (history of chronic bronchitis, chronic obstructive lung disease, or emphysema with dyspnea on physical exertion), and moderate to severe asthma (subject requires an inhaled steroid to control symptoms or has been prescribed a course of oral steroids in the past year); or
(xi) any combination of (i)-(x).

Embodiment 79. The method of any one of Embodiments 48 or 75-78, wherein the administering comprises intravenous infusion.

Embodiment 80. A method of preventing or reducing the severity of SARS-CoV-2 infection in a subject with close contacts to a person with a confirmed SARS-CoV-2 infection, the method comprising administering to the subject a single dose of a composition comprising the antibody or antigen-binding fragment of any one of Embodiments 1-31, 51-54, and 61-65.

Embodiment 81. The method of Embodiment 80, wherein the antibody or antigen-binding fragment comprises CDRH1, CDRH2, CDRH3, CDRL1, CDRL2, and CDRL3 amino acid sequences according to SEQ ID NOS.: 106, 121, 108, 169, 170, and 171, respectively, and optionally further comprises a (n e.g. IgG1) Fc polypeptide comprising a M428L/N434S mutation, preferably wherein the antibody or antigen-binding fragment comprises the CH1-CH3 amino acid sequence of SEQ ID NO.: 173 and the CL amino acid sequence of SEQ ID NO.: 174.

Embodiment 82. The method of Embodiment 80 or 81, wherein the antibody or antigen-binding fragment comprises a VH according to SEQ ID NO: 113 and a VL according to SEQ ID NO: 168, and optionally further comprises a (n e.g. IgG1) Fc polypeptide comprising a M428L/N434S mutation, preferably wherein the antibody or antigen-binding fragment comprises the CH1-CH3 amino acid sequence of SEQ ID NO.: 173 and the CL amino acid sequence of SEQ ID NO.: 174.

Embodiment 83. The method of any one of Embodiments 80-82, wherein the subject: (i) is 12 years old or older; and (ii) last had contact with a person with a confirmed SARS-CoV-2 infection less than three days prior to administration of the composition.

Embodiment 84. The method of any one of Embodiments 48 or 80-83, wherein the administering comprises intravenous infusion.

Embodiment 85. The method of any one of Embodiments 48 or 75-78, wherein the administering comprises intramuscular injection.

Embodiment 86. The method of any one of Embodiments 48 or 80-83, wherein the administering comprises intramuscular injection.

Embodiment 87. The method of any one of Embodiments 75-86, wherein the single dose of the composition comprises 250 mg of the antibody or antigen-binding fragment.

Embodiment 88. The method of any one of Embodiments 75-86, wherein the single dose of the composition comprises 500 mg of the antibody or antigen-binding fragment.

Embodiment 89.1. The method of any one of Embodiments 48 or 75-88, wherein the subject has a mild-to-moderate SARS-2-CoV infection (e.g., has mild-to-moderate COVID-19) and, optionally, is at risk for progression to severe disease.

Embodiment 89.2. A method of treating COVID-19 in a subject having mild-to-moderate COVID-19, the method comprising administering intramuscularly to the subject a single dose of a composition comprising an antibody that comprises a VH according to SEQ ID NO: 113 and a VL according to SEQ ID NO: 168, and optionally further comprises a (n e.g. IgG1) Fc polypeptide comprising a M428L/N434S mutation, preferably wherein the antibody comprises the CH1-CH3 amino acid sequence of SEQ ID NO.: 173 and the CL amino acid sequence of SEQ ID NO.: 174.

Embodiment 90. The method of Embodiment 89.2, wherein the single dose of the composition comprises 250 mg of the antibody.

Embodiment 91. The method of Embodiment 89.2, wherein the single dose of the composition comprises 500 mg of the antibody.

Embodiment 92. The method of any one of Embodiments 89.2-91, wherein:
(i) (i)(a) the subject is 12 years of age or older and is at high risk of progression of COVID-19 or (i)(b) the subject is 65 years of age or older; and/or
(ii) the subject has a positive SARS-CoV-2 test result (e.g., by PCR test), has oxygen saturation ≥94% on room air, has COVID-19 symptoms, and is less than or equal to 7 days from onset of symptoms.

Embodiment 93. The method of any one of Embodiments 89.2-92, wherein:
(i) (i)(a) the subject is 12 years of age or older and is at high risk of progression of COVID-19 or (i)(b) the subject is 65 years of age or older; and
(ii) the subject has a positive SARS-CoV-2 test result (e.g., by PCR test), has oxygen saturation ≥94% on room air, has COVID-19 symptoms, and is less than or equal to 7 days from onset of symptoms.

[Embodiments 94-100—reserved]

Embodiment 101. A method of treating a SARS-CoV-2 infection in a subject, the method comprising administering to the subject a single dose of a composition comprising an antibody or antigen-binding fragment that is capable of binding to a surface glycoprotein of a SARS-CoV-2 expressed on a cell surface of a host cell and/or on a virion, wherein the antibody comprises a heavy chain variable domain (VH) comprising a CDRH1, a CDRH2, and a CDRH3, and a light chain variable domain (VL) comprising a CDRL1, a CDRL2, and a CDRL3, wherein CDRH1, CDRH2, CDRH3, CDRL1, CDRL2, and CDRL3 comprise the amino acid sequences set forth in: (1) SEQ ID NOS.: 106, 121, 108, 169, 170, and 171, respectively; or (2) SEQ ID NOS.: 106, 107, 108, 169, 170, and 171, respectively.

Embodiment 102. A method of treating a SARS-CoV-2 infection in a subject, the method comprising administering to the subject an effective amount of (i) an antibody or antigen-binding fragment that is capable of binding to a surface glycoprotein of a SARS-CoV-2 expressed on a cell surface of a host cell and/or on a virion, or (ii) a composition comprising (ii) (a) the antibody or antigen-binding fragment and (ii) (b) a pharmaceutically acceptable excipient, carrier, or diluent, wherein the antibody comprises a heavy chain variable domain (VH) comprising a CDRH1, a CDRH2, and a CDRH3, and a light chain variable domain (VL) comprising a CDRL1, a CDRL2, and a CDRL3, wherein CDRH1, CDRH2, CDRH3, CDRL1, CDRL2, and CDRL3 comprise the amino acid sequences set forth in: (1) SEQ ID NOS.: 106, 121, 108, 169, 170, and 171, respectively; or (2) SEQ ID NOS.: 106, 107, 108, 169, 170, and 171, respectively.

Embodiment 103. The method of Embodiment 102, wherein the method comprises administering to the subject a single dose of the antibody or antigen-binding fragment of (i) or the composition of (ii).

Embodiment 104. The method of any one of Embodiments 101-103, wherein the VH comprises the amino acid sequence set forth in SEQ ID NO.: 113 and the VL comprises the amino acid sequence set forth in SEQ ID NO.: 168.

Embodiment 105. The method of any one of Embodiments 101-103, wherein the VH comprises the amino acid sequence set forth in SEQ ID NO.: 105 and the VL comprises the amino acid sequence set forth in SEQ ID NO.: 168.

Embodiment 106. The method of any one of Embodiments 101-105, wherein the antibody or antigen-binding fragment further comprises an Fc polypeptide or a fragment thereof.

Embodiment 107. The method of any one of Embodiments 101-106, which is an IgG, IgA, IgM, IgE, or IgD isotype.

Embodiment 108. The method of any one of Embodiments 101-107, which is an IgG isotype selected from IgG1, IgG2, IgG3, and IgG4.

Embodiment 109. The method of any one of Embodiments 101-108, which is an IgG1 isotype.

Embodiment 110. The method of any one of Embodiments 106-109, wherein the Fc polypeptide or fragment thereof comprises: (i) a mutation that enhances binding to a FcRn as compared to a reference Fc polypeptide that does not comprise the mutation; and/or (ii) a mutation that enhances binding to a FcγR as compared to a reference Fc polypeptide that does not comprise the mutation.

Embodiment 111. The method of Embodiment 110, wherein the mutation that enhances binding to a FcRn comprises: M428L; N434S; N434H; N434A; N434S; M252Y; S254T; T256E; T250Q; P257I; Q311I; D376V; T307A; E380A; or any combination thereof.

Embodiment 112. The method of Embodiment 110 or 111, wherein the mutation that enhances binding to FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; or (viii) any combination of (i)-(vii).

Embodiment 113. The method of any one of Embodiments 110-112, wherein the mutation that enhances binding to FcRn comprises M428L/N434S.

Embodiment 114. The method of any one of Embodiments 110-113, wherein the mutation that enhances binding to a FcγR comprises S239D; I332E; A330L; G236A; or any combination thereof, and optionally does not comprise S239D.

Embodiment 115. The method of any one of Embodiments 110-114, wherein the mutation that enhances binding to a FcγR comprises:
(i) S239D/I332E;
(ii) S239D/A330L/I332E;
(iii) G236A/S239D/I332E; or
(iv) G236A/A330L/I332E.

Embodiment 116. The method of any one of Embodiments 101-115, wherein the antibody or antigen-binding fragment comprises the CH1-CH3 amino acid sequence of SEQ ID NO.: 173 or 265 and the CL amino acid sequence of SEQ ID NO.: 174.

Embodiment 117. The method of any one of Embodiments 101-115, wherein the antibody or antigen-binding fragment comprises the CH1-CH3 amino acid sequence of SEQ ID NO.: 175 or 266 and the CL amino acid sequence of SEQ ID NO.: 174.

Embodiment 118. The method of any one of Embodiments 101-117, wherein the antibody or antigen-binding fragment comprises a heavy chain polypeptide and a light chain polypeptide, wherein:
(i) the heavy chain polypeptide comprises the VH amino acid sequence set forth in SEQ ID NO.: 113 and the CH1-CH3 amino acid sequence set forth in SEQ ID NO.: 173 or 265; and
(ii) the light chain comprises the VL amino acid sequence set forth in SEQ ID NO.: 168 and the CL amino acid sequence set forth in SEQ ID NO.: 174,
and wherein, optionally, the method comprises administering a single dose of the antibody or antigen-binding fragment the subject.

Embodiment 119. The method of any one of Embodiments 101-117, wherein the antibody or antigen-binding fragment comprises a heavy chain polypeptide and a light chain polypeptide, wherein:
(i) the heavy chain polypeptide comprises the VH amino acid sequence set forth in SEQ ID NO.: 113 and the CH1-CH3 amino acid sequence set forth in SEQ ID NO.: 175 or 266; and
(ii) the light chain comprises the VL amino acid sequence set forth in SEQ ID NO.: 168 and the CL amino acid sequence set forth in SEQ ID NO.: 174, and wherein, optionally, the method comprises administering a single dose of the antibody or antigen-binding fragment the subject.

Embodiment 120. The method of any one of Embodiments 101-119, wherein the subject:
(i) is aged 18 to 49 years;
(ii) is 18 years old or older;
(iii) has mild to moderate COVID-19;
(iv) has severe COVID-19;
(v) has severe to critical COVID-19;
(vi) has had fewer than seven days or 5 or fewer days since onset of symptoms;
(vii) has had seven days or more since onset of symptoms;
(viii) has had a positive reverse-transcriptase-polymerase-chain-reaction or antigen SARS-CoV-2 test result;
(ix) is 55 years of age or older;
(x) has one or more of: diabetes requiring medication, obesity (body-mass index >30 kg/m²), chronic kidney disease (estimated glomerular filtration rate <60 mL/min/1.73 m²), congestive heart failure (New York Heart Association class II or higher), chronic obstructive pulmonary disease (history of chronic bronchitis, chronic obstructive lung disease, or emphysema with dyspnea on physical exertion), and moderate to severe asthma (subject requires an inhaled steroid to control symptoms or has been prescribed a course of oral steroids in the past year); or
(xi) any combination of (i)-(x).

Embodiment 121. The method of any one of Embodiments 101-120, comprising administering the antibody, antigen-binding fragment, or composition to the subject intravenously.

Embodiment 122. The method of Embodiment 121, comprising administering the antibody, antigen-binding fragment, or composition to the subject intravenously over the course of 30 minutes, 60 minutes, or 90 minutes.

Embodiment 123. The method of any one of Embodiments 101-122, comprising administering the antibody, antigen-binding fragment, or composition to the subject intramuscularly.

Embodiment 124. The method of any one of Embodiments 101-123, wherein the method comprises administering the antibody or antigen-binding fragment to the subject at a dose of up to 100 mg, up to 150 mg, up to 200 mg, up to 250 mg, up to 300 mg, up to 350 mg, up to 400 mg, up to 450 mg, or up to 500 mg.

Embodiment 125. The method of any one of Embodiments 101-124, wherein the method comprises administering the antibody or antigen-binding fragment to the subject at a dose in a range from about 50 mg to about 500 mg, or in a range from about 50 mg to about 250 mg, or in a range from about 50 mg to 100 mg, or in a range from about 100 mg to about 500 mg, or in a range from about 250 mg to about 500 mg.

Embodiment 126. The method of any one of Embodiments 101-125, wherein the method comprises administering 50, 75, 100, 125, 150, 175, 200, 225, 250, 275, 300, 325, 350, 375, 400, 425, 450, 475, or 500 mg of the antibody or antigen-binding fragment to the subject.

Embodiment 127. The method of any one of Embodiments 101-126, wherein the method comprises administering 50, 150, 250, or 500 mg of the antibody or antigen-binding fragment to the subject.

Embodiment 128. The method of any one of Embodiments 101-127, wherein the method comprises administering 500 mg of the antibody or antigen-binding fragment to the subject.

Embodiment 129. The method of any one of Embodiments 101-128, comprising administering the antibody, antigen-binding fragment, or composition to the subject 2, 3, 4, 5, 6, 7, 8, 9, or 10 times, or more.

Embodiment 130. The method of any one of Embodiments 101-129, comprising administering the antibody, antigen-binding fragment, or composition to the subject a plurality of times, wherein a second or successive administration is performed at about 6, about 7, about 8, about 9, about 10, about 11, about 12, about 24, about 48, about 74, about 96 hours, or more, following the first or preceding administration.

Embodiment 131. The method of any one of Embodiments 101-130, wherein the subject is 18 or more years of age with laboratory-confirmed (e.g., by PCR test) SARS-CoV-2 infection.

Embodiment 132. The method of any one of Embodiments 101-131, wherein the subject has a clinical status of Grade 4 (hospitalized, oxygen by mask or nasal prongs), 5 (hospitalized, on non-invasive ventilation, or high flow oxygen), 6 (hospitalized, intubation and mechanical ventilation) or 7 (ventilation and additional organ support-pressors, renal replacement therapy (RRT), extracorporeal membrane oxygenation (ECMO)), as defined by the WHO clinical severity score, 9-point ordinal scale.

Embodiment 133. The method of any one of Embodiments 101-131, wherein the subject has mild-to-moderate COVID-19.

Embodiment 134. The method of Embodiment 133, wherein the subject is at-risk of progression to severe COVID-19.

Embodiment 135. The method of Embodiment 134, wherein following administration of the antibody, antigen-binding fragment, or composition to the subject, the subject is at a reduced risk of hospitalization for COVID-19.

Embodiment 136. The method of Embodiment 135, wherein following administration of the antibody, antigen-binding fragment, or composition to the subject, the risk of hospitalization for COVID-19 is reduced by 10% or more.

Embodiment 137. The method of Embodiment 135, wherein following administration of the antibody, antigen-binding fragment, or composition to the subject, the risk of hospitalization for COVID-19 is reduced by 20% or more.

Embodiment 138. The method of Embodiment 135, wherein following administration of the antibody, antigen-binding fragment, or composition to the subject, the risk of hospitalization for COVID-19 is reduced by 30% or more.

Embodiment 139. The method of Embodiment 135, wherein following administration of the antibody, antigen-binding fragment, or composition to the subject, the risk of hospitalization for COVID-19 is reduced by 40% or more.

Embodiment 140. The method of Embodiment 135, wherein following administration of the antibody, antigen-binding fragment, or composition to the subject, the risk of hospitalization for COVID-19 is reduced by 50% or more.

Embodiment 141. The method of Embodiment 135, wherein following administration of the antibody, antigen-binding fragment, or composition to the subject, the risk of hospitalization for COVID-19 is reduced by 60% or more.

Embodiment 142. The method of Embodiment 135, wherein following administration of the antibody, antigen-binding fragment, or composition to the subject, the risk of hospitalization for COVID-19 is reduced by 70% or more.

Embodiment 143. The method of Embodiment 135, wherein following administration of the antibody, antigen-binding fragment, or composition to the subject, the risk of hospitalization for COVID-19 is reduced by 80% or more.

Embodiment 144. The method of Embodiment 135, wherein following administration of the antibody, antigen-binding fragment, or composition to the subject, the risk of hospitalization for COVID-19 is reduced by 85% or more.

Embodiment 145. The method of any one of Embodiments 101-144, wherein the subject has or is at risk for progressing to severe COVID-19, wherein, optionally, severe COVID-19 comprises (i) hypoxemia (O2 saturation ≤93% on room air or PaO2/FiO2<300) requiring oxygen supplementation for more than 1 day or (ii) the subject requiring ≥4 L/min oxygen supplementation or equivalent.

Embodiment 146. The method of any one of Embodiments 101-145, wherein the subject has or is at risk for progressing to critical COVID-19, wherein, optionally, critical COVID-19 comprises respiratory failure requiring at least one of the following: invasive mechanical ventilation and ECMO; shock; and multi-organ dysfunction/failure.

Embodiment 147. The method of any one of Embodiments 101-146, wherein the subject is less than seven days since onset of symptoms.

Embodiment 148. The method of any one of Embodiments 101-146, wherein the subject is seven days or more since onset of symptoms.

Embodiment 149. The method of any one of Embodiments 101-148, wherein the subject is any one or more of (i)-(iii):
(i) 18 or older and has a positive SARS-CoV-2 test result (by any validated test e.g. RT-PCR on any specimen type);
(ii) (1) hospitalized with severe COVID-19 disease defined as requirement for supplemental oxygen or non-invasive ventilation consistent with Grade 4 or Grade 5 disease or (2) hospitalized with critical COVID-19 disease defined as those on mechanical ventilation (Grade 6 or Grade 7 disease));
(iii) is male or female, wherein, optionally, (1) the woman is non-childbearing potential (WONCBP) or (2) is a woman of child-bearing potential (WOCBP) and uses a contraceptive method.

Embodiment 150. The method of any one of Embodiments 101-149, wherein the method comprises administering 500 mg of the antibody or antigen-binding fragment to the subject.

Embodiment 151. The method of any one of Embodiments 101-150, wherein the subject had or has close contacts to a person with a confirmed SARS-CoV-2 infection.

Embodiment 152. The method of any one of Embodiments 101-151, wherein treating comprises preventing infection by SARS-CoV-2 and/or preventing COVID-19.

Embodiment 153. The method of any one of Embodiments 101-152, wherein treating comprises preventing progression of COVID-19 in the subject.

Embodiment 154. The method of any one of Embodiments 101-153, wherein treating comprises preventing contraction and/or transmission of symptomatic COVID-19.

Embodiment 155. The method of any one of Embodiments 101-153, wherein treating comprises preventing contraction and/or transmission of asymptomatic COVID-19.

Embodiment 156. The method of any one of Embodiments 101-155, wherein the subject is at-risk for contracting or progressing on COVID-19.

Embodiment 157. The method of any one of Embodiments 101-156, wherein treating comprises preventing or reducing:
(1) one or more acute respiratory symptom selected from: cough; sputum production; sore throat; and shortness of breath; or
(2) fever of greater than 38° C.;
(3) two or more of the following symptoms: fatigue; myalgias/arthralgias; chills; nausea/vomiting; diarrhea; and anosmia/dysgeusia.

Embodiment 158. The method of any one of Embodiments 101-157, wherein treating comprises preventing or reducing one or more of the following symptoms: fever of greater than 38° C.; chills; cough; sore throat; malaise; headache; myalgia; a change in smell or taste; nasal congestion/rhinorrhea; vomiting; diarrhea; shortness of breath on exertion.

Embodiment 159. The method of any one of Embodiments 101-158, wherein the subject is an adult.

Embodiment 160. The method of any one of Embodiments 101-159, wherein the subject is 18 or more years of age, or is 19 or more years of age.

Embodiment 161. The method of any one of Embodiments 101-160, wherein the subject is 55 years of age or is, or is 65 years of age or is older

Embodiment 162. The method of any one of Embodiments 101-161, wherein the administering the antibody, antigen-binding fragment, or composition comprises intravenous infusion.

Embodiment 163. The method of any one of Embodiments 101-162, wherein administering the antibody, antigen-binding fragment, or composition comprises intramuscular injection.

Embodiment 164. The method of any one of Embodiments 101-163, wherein the method comprises administering 250 mg of the antibody or antigen-binding fragment to the subject.

Embodiment 165. The method of any one of Embodiments 101-163, wherein the method comprises administering 500 mg of the antibody or antigen-binding fragment to the subject.

Embodiment 166. The method of any one of Embodiments 101-165, wherein the subject has a mild-to-moderate SARS-2-CoV infection (e.g., has mild-to-moderate COVID-19) and, optionally, is at risk for progression to severe disease.

Embodiment 167. The method of any one of Embodiments 101-166, wherein the subject:
(i) is 12 years old or older; and
(ii) last had contact with a person with a confirmed SARS-CoV-2 infection less than three days prior to administration of the composition.

Embodiment 168. The method of any one of Embodiments 101-167, wherein the subject has mild-to-moderate COVID-19 and the method comprises administering a single dose of the antibody, antigen-binding fragment, or composition to the subject intramuscularly.

Embodiment 169. The method of Embodiment 168, wherein the single dose comprises 250 mg of the antibody or antigen-binding fragment.

Embodiment 170. The method of Embodiment 168 or 169, wherein the single dose of comprises 500 mg of the antibody or antigen-binding fragment.

Embodiment 171. The method of any one of Embodiments 168-170, wherein: (i) (i)(a) the subject is 12 years of age or older and is at high risk of progression of COVID-19 or (i)(b) the subject is 65 years of age or older; and/or (ii) the subject has a positive SARS-CoV-2 test result (e.g., by PCR test), has oxygen saturation ≥94% on room air, has COVID-19 symptoms, and is less than or equal to 7 days from onset of symptoms.

Embodiment 172. The method of any one of Embodiments 101-171, wherein: (i) (i)(a) the subject is 12 years of age or older and is at high risk of progression of COVID-19 or (i)(b) the subject is 65 years of age or older; and (ii) the subject has a positive SARS-CoV-2 test result (e.g., by PCR test), has oxygen saturation ≥94% on room air, has COVID-19 symptoms, and is less than or equal to 7 days from onset of symptoms.

Embodiment 173. The method of any one of Embodiments 101-172, wherein the subject is not hospitalized and is at high-risk for (i) hospitalization and/or (ii) progression of COVID-19.

Embodiment 174. The method of any one of Embodiments 101-173, wherein the subject is: (1) 12 or more years of age and, optionally, is at high risk of progression of COVID-19; and/or (2) is 65 or more years of age.

Embodiment 175. The method of any one of Embodiments 101-174, wherein the subject has had a positive SARS-CoV-2 test result, has oxygen saturation ≥94% on room air, has COVID-19 symptoms, and is less than or equal to 7 days from onset of symptoms.

Embodiment 176. The method of any one of Embodiments 101-175, wherein the antibody or antigen-binding fragment was obtained from a non-clonal pool of cells stably transfected with a polynucleotide encoding the antibody or antigen-binding fragment.

Embodiment 177. The method of any one of Embodiments 101-175, wherein the antibody or antigen-binding fragment was obtained from a clonal master cell bank.

Embodiment 178. The method of any one of Embodiments 101-177, wherein the subject: is a resident of a nursing home or a long-term care facility; is a hospice care worker; is a healthcare provider or healthcare worker; is a first responder; is a family member or other close contact of a subject diagnosed with or suspected of having a SARS-CoV-2 infection, is overweight or clinically obese; is or has been a smoker; has or had chronic obstructive pulmonary disease (COPD); is asthmatic (e.g., having moderate to severe asthma); has an autoimmune disease or condition (e.g., diabetes); has a compromised or depleted immune system (e.g., due to AIDS/HIV infection, a cancer such as a blood cancer, a lymphodepleting therapy such as a chemotherapy, a bone marrow or organ transplantation, or a genetic immune condition); has chronic liver disease; has cardiovascular disease; and/or has a pulmonary or heart defect; and/or works or otherwise spends time in close proximity with others, such as in a factory, shipping center, hospital setting, or the like.

Embodiment 179. The method of any one of Embodiments 101-178, wherein the subject has received a vaccine for SARS-CoV-2 and the vaccine is determined to be ineffective, e.g., by post-vaccine infection or symptoms in the subject, by clinical diagnosis or scientific or regulatory criteria.

Embodiment 180. The method of any one of Embodiments 101-178, wherein the subject has not received a vaccine for SARS-CoV-2.

Embodiment 181. The method of any one of Embodiments 101-180, wherein the subject has received convalescent plasma therapy (i.e., from a convalescent COVID-19 subject), remdesivir, or both, for SARS-CoV-2.

Embodiment 182. The method of any one of Embodiments 101-181, wherein treatment comprises pre-exposure or peri-exposure prophylaxis.

Embodiment 183. The method of any one of Embodiments 101-182, wherein treatment is administered to the subject having mild-to-moderate disease, optionally in an outpatient setting.

Embodiment 184. The method of any one of Embodiments 101-183, wherein treatment is administered to a subject with moderate-to-severe disease, such as requiring hospitalization.

Embodiment 185. The method of any one of Embodiments 101-184, wherein the subject is hospitalized with COVID-19.

Embodiment 186. The method of any one of Embodiments 101-185, wherein the subject having a SARS-CoV-2 infection: has mild-to-moderate COVID-19; is experiencing any one or more of: fever; cough; fatigue; shortness of breath or difficulty breathing; muscle aches; chills; sore throat; runny nose; headache; chest pain; loss of taste and/or smell; and pink eye (conjunctivitis); malaise; and abnormal imaging; has evidence of lower respiratory disease by clinical assessment or imaging and a saturation of oxygen (SaO2) greater than (>) 93 percent (%) on room air at sea level, has a positive SARS-CoV-2 viral testing result, and/or is at high risk for progressing to severe COVID-19 and/or hospitalization, e.g., the human subject (1) is 65 years of age or older (≥65); has a body mass index (BMI) of 35 or greater (≥35); has chronic kidney disease; has diabetes; (5) has immunosuppressive disease, is receiving immunosuppressive treatment; is 55 years of age or older (≥55) and has cardiovascular disease, hypertension, chronic obstructive pulmonary disease, or other chronic respiratory disease; and/or is 12-17 years of age and has a BMI ≥85% for their age and gender, or sickle cell disease, congenital or acquired heart disease, neurodevelopmental disorders (e.g., cerebral palsy), a medical-related technological dependence (e.g., tracheostomy, gastrostomy, or positive pressure ventilation not related to COVID-19), or asthma, reactive airway or other chronic respiratory disease that requires daily medication for control; has recently been diagnosed with COVID-19 (e.g., within 1 day, 2 days, 3 days, 4 days, 5 days, 6 days, 7 days, 8 days, 9 days, 10 days, 11 days, 12 days, 13 days, or 14 days) and/or is within 10 days of symptom onset; or has or is experiencing any combination of the foregoing.

Embodiment 187. The method of any one of Embodiments 101-186, wherein the subject is (a) 18 years old or older, or (b) 55 years old or younger, provided that the subject is 18 years or older.

Embodiment 188. The method of any one of Embodiments 101-187, wherein the subject has a laboratory confirmed COVID-19 infection by positive polymerase chain reaction (PCR; e.g., RT-PCR) test; e.g., on any type of respiratory tract sample).

Embodiment 189. The method of any one of Embodiments 101-187, the subject has peripheral capillary oxygen saturation (SpO₂) >94% room air (RA), and has experienced one or more symptoms of COVID-19 for ≤120 h (5 days).

Embodiment 190. The method of any one of Embodiments 101-189, wherein the subject is further receiving or has received remdesivir, supplemental oxygen, ventilation therapy, respiration therapy, dexamethasone, tocilizumab, or any combination thereof.

TABLE 1
Sequences

| | SEQ | |
|---|---|---|
| Sequence | ID | |
| Description | NO. | Sequence |
| SARS-CoV-2 | 1 | QVQLVQSGAEVKKPGASVKVSCKASGYTFT |
| S300-v1 mAb | | DYYIHWVRQAPGQGPEWLGWVNGYSGATRY |
| VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 2 | GYTFTDYY |
| S300-v1 mAb | | |
| CDRH1 (aa) | | |
| SARS-CoV-2 | 3 | VNGYSGAT |
| S300-v1 mAb | | |
| CDRH2 (aa) | | |
| SARS-CoV-2 | 4 | ARDRPSHEWAMYFFDN |
| S300-v1 mAb | | |
| CDRH3 (aa) | | |
| SARS-CoV-2 | 5 | QIVLTQSPGTLSLSPGERATLSCRASQSVP |
| S300-v1 mAb | | SSCLAWYQQKPGQAPRLLIYGASGRATGIP |
| VL (VK) (aa) | | DRESGSGSGTDFTLTIRRLEPEDFAVYYCQ |
| | | QYGSSPPLTFGGGTKVEIK |
| SARS-CoV-2 | 6 | QSVPSSC |
| S300-v1 mAb | | |
| CDRL1 (aa) | | |
| SARS-CoV-2 | 7 | GAS |
| S300-v1 mAb | | |
| CDRL2 (aa) | | |
| SARS-CoV-2 | 8 | QQYGSSPPLT |
| S300-v1 mAb | | |
| CDRL3 (aa) | | |
| SARS-CoV-2 | 9 | QVQLVQSGAEVKKPGASVKVSCKASGYTFT |
| S300-v1.1 | | DYYIHWVRQAPGQGPEWLGWVQGYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 10 | QVQLVQSGAEVKKPGASVKVSCKASGYTFT |
| S300-v1.2 | | DYYIHWVRQAPGQGPEWLGWVNAYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 11 | QVQLVQSGAEVKKPGASVKVSCKASGYTFT |
| S300-v1.3 | | DYYIHWVRQAPGQGPEWLGWVNSYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 12 | QVQLVQSGAEVKKPGASVKVSCKASGYTFT |
| S300-v1.4 | | DYYIHWVRQAPGQGPEWLGWVNPYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 13 | QVQLVQSGAEVKKPGASVKVSCKASGYTFT |
| S300-v1.5 | | DYYIHWVRQAPGQGPEWLGWVNQYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 14 | QVQLVQSGAEVKKPGASVKVSCKASGYTFT |
| S300-v1.6 | | DYYIHWVRQAPGQGPEWLGWVLGYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 15 | QVQLVQSGAEVKKPGASVKVSCKASGYTFT |
| S300-v1.7 | | DYYIHWVRQAPGQGPEWLGWVTGYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 16 | VQGYSGAT |
| S300-v1.1 | | |
| mAb CDRH2 | | |
| (aa) | | |
| SARS-CoV-2 | 17 | VNAYSGAT |
| S300-v1.2 | | |
| mAb CDRH2 | | |
| (aa) | | |
| SARS-CoV-2 | 18 | VNSYSGAT |
| S300-v1.3 | | |
| mAb CDRH2 | | |
| (aa) | | |
| SARS-CoV-2 | 19 | VNPYSGAT |
| S300-v1.4 | | |
| mAb CDRH2 | | |
| (aa) | | |
| SARS-CoV-2 | 20 | VNQYSGAT |
| S300-v1.5 | | |
| mAb CDRH2 | | |
| (aa) | | |
| SARS-CoV-2 | 21 | VLGYSGAT |
| S300-v1.6 | | |
| mAb CDRH2 | | |
| (aa) | | |
| SARS-CoV-2 | 22 | VTGYSGAT |
| S300-v1.7 | | |
| mAb CDRH2 | | |
| (aa) | | |
| SARS-CoV-2 | 23 | QVQLVQSGAEVKKPGASVKVSCKASGYTFT |
| S300-v1.8 | | DYYIHWVRQAPGQGPEWLGWVNGYSGATRY |
| mAb VH (aa) | | AQKYQGRVTMTRDTSISTAYMQLSRLRPDD |
| | | TAVYYCARDRPSHEFAMYFFDNWGQGTLVT |
| | | VSS |
| SARS-CoV-2 | 24 | QVQLVQSGAEVKKPGASVKVSCKASGYTFT |
| S300-v1.9 | | DYYIHWVRQAPGQGPEWLGWVNGYSGATRY |
| mAb VH (aa) | | AQKYQGRVTMTRDTSISTAYMQLSRLRPDD |
| | | TAVYYCARDRPSHEYAMYFFDNWGQGTLVT |
| | | VSS |
| SARS-CoV-2 | 25 | ARDRPSHEFAMYFFDN |
| S300-v1.8 | | |
| mAb CDRH3 | | |
| (aa) | | |
| SARS-CoV-2 | 26 | ARDRPSHEYAMYFFDN |
| S300-v1.9 | | |
| mAb CDRH3 | | |
| (aa) | | |
| SARS-CoV-2 | 27 | QVQLVQSGAEVKKPGASVKVSCKASGYTFT |
| S300-v2 mAb | | DYYIHWVRQAPGQGPEWLGFVNGYSGATRY |
| VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 28 | QVQLVQSGAEVKKPGASVKVSCKASGYTFT |
| S300-v2.1 | | DYYIHWVRQAPGQGPEWLGFVQGYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 29 | QVQLVQSGAEVKKPGASVKVSCKASGYTFT |
| S300-v2.2 | | DYYIHWVRQAPGQGPEWLGFVNAYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 30 | QVQLVQSGAEVKKPGASVKVSCKASGYTFT |
| S300-v2.3 | | DYYIHWVRQAPGQGPEWLGFVNSYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 31 | QVQLVQSGAEVKKPGASVKVSCKASGYTFT |
| S300-v2.4 | | DYYIHWVRQAPGQGPEWLGFVNPYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 32 | QVQLVQSGAEVKKPGASVKVSCKASGYTFT |
| S300-v2.5 | | DYYIHWVRQAPGQGPEWLGFVNQYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 33 | QVQLVQSGAEVKKPGASVKVSCKASGYTFT |
| S300-v2.6 | | DYYIHWVRQAPGQGPEWLGFVLGYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 34 | QVQLVQSGAEVKKPGASVKVSCKASGYTFT |
| S300-v2.7 | | DYYIHWVRQAPGQGPEWLGFVTGYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 35 | QVQLVQSGAEVKKPGASVKVSCKASGYTFT |
| S300-v2.8 | | DYYIHWVRQAPGQGPEWLGFVNGYSGATRY |
| mAb VH (aa) | | AQKYQGRVTMTRDTSISTAYMQLSRLRPDD |
| | | TAVYYCARDRPSHEFAMYFFDNWGQGTLVT |
| | | VSS |
| SARS-CoV-2 | 36 | QVQLVQSGAEVKKPGASVKVSCKASGYTFT |
| S300-v2.9 | | DYYIHWVRQAPGQGPEWLGFVNGYSGATRY |
| mAb VH (aa) | | AQKYQGRVTMTRDTSISTAYMQLSRLRPDD |
| | | TAVYYCARDRPSHEYAMYFFDNWGQGTLVT |
| | | VSS |
| SARS-CoV-2 | 37 | QVQLVQSGAEVKKPGASVKVSCKASGYTFT |
| S300-v3 mAb | | DYYIHWVRQAPGQGPEWLGYVNGYSGATRY |
| VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 38 | QVQLVQSGAEVKKPGASVKVSCKASGYTFT |
| S300-v3.1 | | DYYIHWVRQAPGQGPEWLGYVQGYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 39 | QVQLVQSGAEVKKPGASVKVSCKASGYTFT |
| S300-v3.2 | | DYYIHWVRQAPGQGPEWLGYVNAYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 40 | QVQLVQSGAEVKKPGASVKVSCKASGYTFT |
| S300-v3.3 | | DYYIHWVRQAPGQGPEWLGYVNSYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 41 | QVQLVQSGAEVKKPGASVKVSCKASGYTFT |
| S300-v3.4 | | DYYIHWVRQAPGQGPEWLGYVNPYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 42 | QVQLVQSGAEVKKPGASVKVSCKASGYTFT |
| S300-v3.5 | | DYYIHWVRQAPGQGPEWLGYVNQYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 43 | QVQLVQSGAEVKKPGASVKVSCKASGYTFT |
| S300-v3.6 | | DYYIHWVRQAPGQGPEWLGYVLGYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 44 | QVQLVQSGAEVKKPGASVKVSCKASGYTFT |
| S300-v3.7 | | DYYIHWVRQAPGQGPEWLGYVTGYSGATRY |
| mAb VH (aa) | | AQKYQGRVTMTRDTSISTAYMQLSRLRPDD |
| | | TAVYYCARDRPSHEWAMYFFDNWGQGTLVT |
| | | VSS |
| SARS-CoV-2 | 45 | QVQLVQSGAEVKKPGASVKVSCKASGYTFT |
| S300-v3.8 | | DYYIHWVRQAPGQGPEWLGYVNGYSGATRY |
| mAb VH (aa) | | AQKYQGRVTMTRDTSISTAYMQLSRLRPDD |
| | | TAVYYCARDRPSHEFAMYFFDNWGQGTLVT |
| | | VSS |
| SARS-CoV-2 | 46 | QVQLVQSGAEVKKPGASVKVSCKASGYTFT |
| S300-v3.9 | | DYYIHWVRQAPGQGPEWLGYVNGYSGATRY |
| mAb VH (aa) | | AQKYQGRVTMTRDTSISTAYMQLSRLRPDD |
| | | TAVYYCARDRPSHEYAMYFFDNWGQGTLVT |
| | | VSS |
| SARS-CoV-2 | 47 | QIVLTQSPGTLSLSPGERATLSCRASQSVP |
| S300-v10 | | SSYLAWYQQKPGQAPRLLIYGASGRATGIP |
| mAb VL | | DRFSGSGSGTDFTLTIRRLEPEDFAVYYCQ |
| (VK) (aa) | | QYGSSPPLTFGGGTKVEIK |
| SARS-CoV-2 | 48 | QIVLTQSPGTLSLSPGERATLSCRASQSVP |
| S300-v11 | | SSSLAWYQQKPGQAPRLLIYGASGRATGIP |
| mAb VL | | DRFSGSGSGTDFTLTIRRLEPEDFAVYYCQ |
| (VK) (aa) | | QYGSSPPLTFGGGTKVEIK |
| SARS-CoV-2 | 49 | QIVLTQSPGTLSLSPGERATLSCRASQSVP |
| S300-v12 | | SSTLAWYQQKPGQAPRLLIYGASGRATGIP |
| mAb VL | | DRESGSGSGTDFTLTIRRLEPEDFAVYYCQ |
| (VK) (aa) | | QYGSSPPLTFGGGTKVEIK |
| SARS-CoV-2 | 50 | QIVLTQSPGTLSLSPGERATLSCRASQSVP |
| S300-v13 | | SSALAWYQQKPGQAPRLLIYGASGRATGIP |
| mAb VL | | DRFSGSGSGTDFTLTIRRLEPEDFAVYYCQ |
| (VK) (aa) | | QYGSSPPLTFGGGTKVEIK |
| SARS-CoV-2 | 51 | QSVPSSY |
| S300-v10 | | |
| mAb CDRL1 | | |
| (aa) | | |
| SARS-CoV-2 | 51 | QSVPSSS |
| S300-v11 | | |
| mAb CDRL1 | | |
| (aa) | | |
| SARS-CoV-2 | 53 | QSVPSST |
| S300-v12 | | |
| mAb CDRL1 | | |
| (aa) | | |
| SARS-CoV-2 | 54 | QSVPSSA |
| S300-v13 | | |
| mAb CDRL1 | | |
| (aa) | | |
| SARS-CoV-2 | 55 | QVQLVESGGGVVQPGRSLRLSCAASGFTFS |
| S302 mAb | | SYGMHWVRQAPGKGLEWVAVISYDGSNKYY |
| VH (aa) | | ADSVKGRFTISRDNSKNTLYLQMNSLRAED |
| | | TAVYYCAKDISSGWDRVFDYWGQGTLVTVS |
| | | S |
| SARS-CoV-2 | 56 | GFTFSSYG |
| S302 mAb | | |
| CDRHI (aa) | | |
| SARS-CoV-2 | 57 | ISYDGSNK |
| S302 mAb | | |
| CDRH2 (aa) | | |
| SARS-CoV-2 | 58 | AKDISSGWDRVFDY |
| S302 mAb | | |
| CDRH3 (aa) | | |
| SARS-CoV-2 | 59 | EILLTQSPGTLSLSPGERATLSCRTSQSVG |
| S302 mAb | | SSYLAWYQQKPGQAPRLLIYAASSRAIGIP |
| VL (VK) (aa) | | DRFSGSGSGTDFTLTISRLEPEDFAVYYCQ |
| | | QYGSSPWTFGQGTKVEIK |
| SARS-CoV-2 | 60 | QSVGSSY |
| S302 mAb | | |
| CDRLI (aa) | | |
| SARS-CoV-2 | 61 | AAS |
| S302 mAb | | |
| CDRL2 (aa) | | |
| SARS-CoV-2 | 62 | QQYGSSPWT |
| S302 mAb | | |
| CDRL3 (aa) | | |
| SARS-CoV-2 | 63 | EVQLVESGGGLVKPGGSLRLSCAASGFTFL |
| S303-v1 mAb | | TYSMNWVRQTPGKRLQWVSAISGSGGATYY |
| VH (aa) | | ADSVKGRFTISRDNSKNTLYLQMNTVTADD |
| | | TAIYFCARERDDIFPMGLNAFDIWGQGAMV |
| | | IVSS |
| SARS-CoV-2 | 64 | GFTFLTYS |
| S303-v1 mAb | | |
| CDRH1 (aa) | | |
| SARS-CoV-2 | 65 | ISGSGGAT |
| S303-v1 mAb | | |
| CDRH2 (aa) | | |
| SARS-CoV-2 | 66 | ARERDDIFPMGLNAFDI |
| S303-v1 mAb | | |
| CDRH3 (aa) | | |
| SARS-CoV-2 | 67 | DIQMTQSPSTLSASVGDRVTITCRASQSIS |
| S303-v1 mAb | | NWLAWYQQKPGKAPKLLIYKASSLESGVPS |
| VL (VK) (aa) | | RFSGSGSGTEFTLTISSLQPDDSATYYCQQ |
| | | YDTYSWTFGQGTKVEIK |
| SARS-CoV-2 | 68 | QSISNW |
| S303-v1 mAb | | |
| CDRL1 (aa) | | |
| SARS-CoV-2 | 69 | KAS |
| S303-v1 mAb | | |
| CDRL2 (aa) | | |
| SARS-CoV-2 | 70 | QQYDTYSWT |
| S303-v1 mAb | | |
| CDRL3 (aa) | | |
| SARS-CoV-2 | 71 | DIQMTQSPSTLSASVGDRVTITCRASQSIS |
| S303-v2 mAb | | NFLAWYQQKPGKAPKLLIYKASSLESGVPS |
| VL (VK) (aa) | | RFSGSGSGTEFTLTISSLQPDDSATYYCQQ |
| | | YDTYSWTFGQGTKVEIK |
| SARS-CoV-2 | 72 | DIQMTQSPSTLSASVGDRVTITCRASQSIS |
| S303-v3 mAb | | NYLAWYQQKPGKAPKLLIYKASSLESGVPS |
| VL (VK) (aa) | | RFSGSGSGTEFTLTISSLQPDDSATYYCQQ |
| | | YDTYSWTFGQGTKVEIK |
| SARS-CoV-2 | 73 | QSISNF |
| S303-v2 mAb | | |
| CDRL1 (aa) | | |
| SARS-CoV-2 | 74 | QSISNY |
| S303-v3 mAb | | |
| CDRL1 (aa) | | |
| SARS-CoV-2 | 75 | DIQMTQSPSTLSASVGDRVTITCRASQSIS |
| S303-v4 mAb | | NWLAWYQQKPGKAPKLLIYKASSLESGVPS |
| VL (VK) (aa) | | RFSGSGSGTEFTLTISSLQPDDSATYYCQQ |
| | | YDTYSFTFGQGTKVEIK |
| SARS-CoV-2 | 76 | DIQMTQSPSTLSASVGDRVTITCRASQSIS |
| S303-v5 mAb | | NWLAWYQQKPGKAPKLLIYKASSLESGVPS |
| VL (VK) (aa) | | RFSGSGSGTEFTLTISSLQPDDSATYYCQQ |
| | | YDTYSYTFGQGTKVEIK |
| SARS-CoV-2 | 77 | QQYDTYSFT |
| S303-v4 mAb | | |
| CDRL3 (aa) | | |
| SARS-CoV-2 | 78 | QQYDTYSYT |
| S303-v5 mAb | | |
| CDRL3 (aa) | | |
| SARS-CoV-2 | 79 | EVQLVESGGGLVQPGGSLRLSCAASGFTFS |
| S304 mAb | | SYDMHWVRQTTGKGLEWVSTIGTAGDTYYP |
| VH (aa) | | DSVKGRFTISREDAKNSLYLQMNSLRAGDT |
| | | AVYYCARGDSSGYYYYFDYWGQGTLLTVSS |
| SARS-CoV-2 | 80 | GFTFSSYD |
| S304 mAb | | |
| CDRH1 (aa) | | |
| SARS-CoV-2 | 81 | IGTAGDT |
| S304 mAb | | |
| CDRH2 (aa) | | |
| SARS-CoV-2 | 82 | ARGDSSGYYYYFDY |
| S304 mAb | | |
| CDRH3 (aa) | | |
| SARS-CoV-2 | 83 | DIQMTQSPSSLSAAVGDRVTITCRASQSIG |
| S304 mAb | | SYLNWYQQKPGKAPKLLIYAASSLQSGVPS |
| VL (VK) (aa) | | RFSGSGSGTDFTLTISSLQPEDFAIYYCQQ |
| | | SYVSPTYTFGPGTKVDIK |
| SARS-CoV-2 | 84 | QSIGSY |
| S304 mAb | | |
| CDRL1 (aa) | | |
| SARS-CoV-2 | 85 | AAS |
| S304 mAb | | |
| CDRL2 (aa) | | |
| SARS-CoV-2 | 86 | QQSYVSPTYT |
| S304 mAb | | |
| CDRL3 (aa) | | |
| SARS-CoV-2 | 87 | QVQLVQSGAEVKKPGASVKVSCKASTYTFT |
| S306 mAb | | SFGISWVRQAPGQGLEWMGWITTYSGDTNY |
| VH (aa) | | AQKFQGRVTMTTDTSTNTAYMELRSLRSDD |
| | | TAVYYCASDYFDSSGYYHSFDYWGQGTLVT |
| | | VSS |
| SARS-CoV-2 | 88 | TYTFTSFG |
| S306 mAb | | |
| CDRH1 (aa) | | |
| SARS-CoV-2 | 89 | ITTYSGDT |
| S306 mAb | | |
| CDRH2 (aa) | | |
| SARS-CoV-2 | 90 | ASDYFDSSGYYHSFDY |
| S306 mAb | | |
| CDRH3 (aa) | | |
| SARS-CoV-2 | 91 | EIVLTQSPDTLSLSPGERATLSCRASQSVS |
| S306 mAb | | SYLAWYQQRPGQAPRLLIYDASKRATGIPA |
| VL (VK) (aa) | | RFSGSGSGTDFTLTISSLEPEDFAVYYCQQ |
| | | RSNWPPGCSFGQGTKVEIK |
| SARS-CoV-2 | 92 | QSVSSY |
| S306 mAb | | |
| CDRL1 (aa) | | |
| SARS-CoV-2 | 93 | DAS |
| S306 mAb | | |
| CDRL2 (aa) | | |
| SARS-CoV-2 | 94 | QQRSNWPPGCS |
| S306 mAb | | |
| CDRL3 (aa) | | |
| SARS-CoV-2 | 95 | QVQLVESGGGVVQPGRSLRLSCAASRFTFS |
| S308-v1 mAb | | SYGMHWVRQAPGKGLEWVAVIWHDGNNKHY |
| VH (aa) | | GDSVKGRVTISRDNSKNTLYLQMTSLRAED |
| | | TAVYYCARAVTTFKGSGRARMRGMDVWGQG |
| | | TTVTVSS |
| SARS-CoV-2 | 96 | RFTFSSYG |
| S308-v1 mAb | | |
| CDRH1 (aa) | | |
| SARS-CoV-2 | 97 | IWHDGNNK |
| S308-v1 mAb | | |
| CDRH2 (aa) | | |
| SARS-CoV-2 | 98 | ARAVTTFKGSGRARMRGMDV |
| S308-v1 mAb | | |
| CDRH3 (aa) | | |
| SARS-CoV-2 | 99 | DIQLTQSPSFLSASVGDRVTITCRASQGIN |
| S308-v1 mAb | | TYLAWYQQKPGKAPKLLIYAASTLQSGVPS |
| VL (VK) (aa) | | RFSGSGSGTEFTLTISSLQPEDFATYYCQH |
| | | LDTYPFTFGPGTKVDIK |
| SARS-CoV-2 | 100 | QGINTY |
| S308-v1 mAb | | |
| CDRL1 (aa) | | |
| SARS-CoV-2 | 101 | AAS |
| S308-v1 mAb | | |
| CDRL2 (aa) | | |
| SARS-CoV-2 | 102 | QHLDTYPFT |
| S308-v1 mAb | | |
| CDRL3 (aa) | | |
| SARS-CoV-2 | 103 | QVQLVESGGGVVQPGRSLRLSCAASRFTFS |
| S308-v2 mAb | | SYGMHWVRQAPGKGLEWVAVIWHDGNNKHY |
| VH (aa) | | GDSVKGRVTISRDNSKNTLYLQMTSLRAED |
| | | TAVYYCARAVTTFKGSGRARLRGMDVWGQG |
| | | TTVTVSS |
| SARS-CoV-2 | 104 | ARAVTTFKGSGRARLRGMDV |
| S308-v2 mAb | | |
| CDRH3 (aa) | | |
| SARS-CoV-2 | 105 | QVQLVQSGAEVKKPGASVKVSCKASGYPFT |
| S309-v1 mAb | | SYGISWVRQAPGQGLEWMGWISTYNGNTNY |
| VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 106 | GYPFTSYG |
| S309-v1 mAb | | |
| CDRH1 (aa) | | |
| SARS-CoV-2 | 107 | ISTYNGNT |
| S309-v1 mAb | | |
| CDRH2 (aa) | | |
| SARS-CoV-2 | 108 | ARDYTRGAWFGESLIGGFDN |
| S309-v1 mAb | | |
| CDRH3 (aa) | | |
| SARS-CoV-2 | 109 | DIQMTQSPSSLSTSVGDRVTITCRASQGIN |
| S309-v1 mAb | | NYVAWYQQKPGKVPKLLIYGASTLQSGVPS |
| VL (VK) (aa) | | RFRGSGSGTGFTLTISSLQPEDVASYYCRK |
| | | YNSAPWTFGQGTRVEIK |
| SARS-CoV-2 | 110 | QGINNY |
| S309-v1 mAb | | |
| CDRL1 (aa) | | |
| SARS-CoV-2 | 111 | GAS |
| S309-v1 mAb | | |
| CDRL2 (aa) | | |
| SARS-CoV-2 | 112 | RKYNSAPWT |
| S309-v1 mAb | | |
| CDRL3 (aa) | | |
| SARS-CoV-2 | 113 | QVQLVQSGAEVKKPGASVKVSCKASGYPFT |
| S309-v1.1 | | SYGISWVRQAPGQGLEWMGWISTYQGNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 114 | QVQLVQSGAEVKKPGASVKVSCKASGYPFT |
| S309-v1.2 | | SYGISWVRQAPGQGLEWMGWISTYNSNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 115 | QVQLVQSGAEVKKPGASVKVSCKASGYPFT |
| S309-v1.3 | | SYGISWVRQAPGQGLEWMGWISTYNANTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 116 | QVQLVQSGAEVKKPGASVKVSCKASGYPFT |
| S309-v1.4 | | SYGISWVRQAPGQGLEWMGWISTYNQNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 117 | QVQLVQSGAEVKKPGASVKVSCKASGYPFT |
| S309-v1.5 | | SYGISWVRQAPGQGLEWMGWISTYLGNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 118 | QVQLVQSGAEVKKPGASVKVSCKASGYPFT |
| S309-v1.6 | | SYGISWVRQAPGQGLEWMGWISTYTGNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 119 | QVQLVQSGAEVKKPGASVKVSCKASGYPFT |
| S309-v1.7 | | SYGISWVRQAPGQGLEWMGWISTYNGNTNY |
| mAb VH (aa) | | AQKFQGRVTMTTDTSTTTGYMELRRLRSDD |
| | | TAVYYCARDYTRGAFFGESLIGGFDNWGQG |
| | | TLVTVSS |
| SARS-CoV-2 | 120 | QVQLVQSGAEVKKPGASVKVSCKASGYPFT |
| S309-v1.8 | | SYGISWVRQAPGQGLEWMGWISTYNGNTNY |
| mAb VH (aa) | | AQKFQGRVTMTTDTSTTTGYMELRRLRSDD |
| | | TAVYYCARDYTRGAYFGESLIGGFDNWGQG |
| | | TLVTVSS |
| SARS-CoV-2 | 121 | ISTYQGNT |
| S309-v1.1 | | |
| mAb CDRH2 | | |
| (aa) | | |
| SARS-CoV-2 | 122 | ISTYNSNT |
| S309-v1.2 | | |
| mAb CDRH2 | | |
| (aa) | | |
| SARS-CoV-2 | 123 | ISTYNANT |
| S309-v1.3 | | |
| mAb CDRH2 | | |
| (aa) | | |
| SARS-CoV-2 | 124 | ISTYNQNT |
| S309-v1.4 | | |
| mAb CDRH2 | | |
| (aa) | | |
| SARS-CoV-2 | 125 | ISTYLGNT |
| S309-v1.5 | | |
| mAb CDRH2 | | |
| (aa) | | |
| SARS-CoV-2 | 126 | ISTYTGNT |
| S309-v1.6 | | |
| mAb CDRH2 | | |
| (aa) | | |
| SARS-CoV-2 | 127 | ARDYTRGAFFGESLIGGEDN |
| S309-v1.7 | | |
| mAb CDRH3 | | |
| (aa) | | |
| SARS-CoV-2 | 128 | ARDYTRGAYFGESLIGGFDN |
| S309-v1.8 | | |
| mAb CDRH3 | | |
| (aa) | | |
| SARS-CoV-2 | 129 | QVQLVQSGAEVKKPGASVKVSCKASGYPFT |
| S309-v2 mAb | | SYGISWVRQAPGQGLEWMGFISTYNGNTNY |
| VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 130 | QVQLVQSGAEVKKPGASVKVSCKASGYPFT |
| S309-v2.1 | | SYGISWVRQAPGQGLEWMGFISTYQGNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 131 | QVQLVQSGAEVKKPGASVKVSCKASGYPFT |
| S309-v2.2 | | SYGISWVRQAPGQGLEWMGFISTYNSNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 132 | QVQLVQSGAEVKKPGASVKVSCKASGYPFT |
| S309-v2.3 | | SYGISWVRQAPGQGLEWMGFISTYNANTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 133 | QVQLVQSGAEVKKPGASVKVSCKASGYPFT |
| S309-v2.4 | | SYGISWVRQAPGQGLEWMGFISTYNQNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 134 | QVQLVQSGAEVKKPGASVKVSCKASGYPFT |
| S309-v2.5 | | SYGISWVRQAPGQGLEWMGFISTYLGNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 135 | QVQLVQSGAEVKKPGASVKVSCKASGYPFT |
| S309-v2.6 | | SYGISWVRQAPGQGLEWMGFISTYTGNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 136 | QVQLVQSGAEVKKPGASVKVSCKASGYPFT |
| S309-v2.7 | | SYGISWVRQAPGQGLEWMGFISTYNGNTNY |
| mAb VH (aa) | | AQKFQGRVTMTTDTSTTTGYMELRRLRSDD |
| | | TAVYYCARDYTRGAFFGESLIGGFDNWGQG |
| | | TLVTVSS |
| SARS-CoV-2 | 137 | QVQLVQSGAEVKKPGASVKVSCKASGYPFT |
| S309-v2.8 | | SYGISWVRQAPGQGLEWMGFISTYNGNTNY |
| mAb VH (aa) | | AQKFQGRVTMTTDTSTTTGYMELRRLRSDD |
| | | TAVYYCARDYTRGAYFGESLIGGFDNWGQG |
| | | TLVTVSS |
| SARS-CoV-2 | 138 | QVQLVQSGAEVKKPGASVKVSCKASGYPFT |
| S309-v3 mAb | | SYGISWVRQAPGQGLEWMGYISTYNGNTNY |
| VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 139 | QVQLVQSGAEVKKPGASVKVSCKASGYPFT |
| S309-v3.1 | | SYGISWVRQAPGQGLEWMGYISTYQGNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 140 | QVQLVQSGAEVKKPGASVKVSCKASGYPFT |
| S309-v3.2 | | SYGISWVRQAPGQGLEWMGYISTYNSNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 141 | QVQLVQSGAEVKKPGASVKVSCKASGYPFT |
| S309-v3.3 | | SYGISWVRQAPGQGLEWMGYISTYNANTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 142 | QVQLVQSGAEVKKPGASVKVSCKASGYPFT |
| S309-v3.4 | | SYGISWVRQAPGQGLEWMGYISTYNQNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 143 | QVQLVQSGAEVKKPGASVKVSCKASGYPFT |
| S309-v3.5 | | SYGISWVRQAPGQGLEWMGYISTYLGNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 144 | QVQLVQSGAEVKKPGASVKVSCKASGYPFT |
| S309-v3.6 | | SYGISWVRQAPGQGLEWMGYISTYTGNTNY |
| mAb VH (aa) | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 145 | QVQLVQSGAEVKKPGASVKVSCKASGYPFT |
| S309-v3.7 | | SYGISWVRQAPGQGLEWMGYISTYNGNTNY |
| mAb VH (aa) | | AQKFQGRVTMTTDTSTTTGYMELRRLRSDD |
| | | TAVYYCARDYTRGAFFGESLIGGFDNWGQG |
| | | TLVTVSS |
| SARS-CoV-2 | 146 | QVQLVQSGAEVKKPGASVKVSCKASGYPFT |
| S309-v3.8 | | SYGISWVRQAPGQGLEWMGYISTYNGNTNY |
| mAb VH (aa) | | AQKFQGRVTMTTDTSTTTGYMELRRLRSDD |
| | | TAVYYCARDYTRGAYFGESLIGGFDNWGQG |
| | | TLVTVSS |
| SARS-CoV-2 | 147 | DIQMTQSPSSLSTSVGDRVTITCRASQGIN |
| S309-v9 mAb | | NYVAWYQQKPGKVPKLLIYGASTLQSGVPS |
| VL (VK) (aa) | | RFRGSGSGTGFTLTISSLQPEDVASYYCRK |
| | | YNSAPGTFGQGTRVEIK |
| SARS-CoV-2 | 148 | DIQMTQSPSSLSTSVGDRVTITCRASQGIN |
| S309-v10 | | NYVAWYQQKPGKVPKLLIYGASTLQSGVPS |
| mAb VL | | RFRGSGSGTGFTLTISSLQPEDVASYYCRK |
| (VK) (aa) | | YNSAPRTFGQGTRVEIK |
| SARS-CoV-2 | 149 | DIQMTQSPSSLSTSVGDRVTITCRASQGIN |
| S309-v11 | | NYVAWYQQKPGKVPKLLIYGASTLQSGVPS |
| mAb VL | | RFRGSGSGTGFTLTISSLQPEDVASYYCRK |
| (VK) (aa) | | YNSAPFTFGQGTRVEIK |
| SARS-CoV-2 | 150 | DIQMTQSPSSLSTSVGDRVTITCRASQGIN |
| S309-v12 | | NYVAWYQQKPGKVPKLLIYGASTLQSGVPS |
| mAb VL | | RFRGSGSGTGFTLTISSLQPEDVASYYCRK |
| (VK) (aa) | | YNSAPYTFGQGTRVEIK |
| SARS-CoV-2 | 151 | RKYNSAPGT |
| S309-v9 mAb | | |
| CDRL3 (aa) | | |
| SARS-CoV-2 | 152 | RKYNSAPRT |
| S309-v10 | | |
| mAb CDRL3 | | |
| (aa) | | |
| SARS-CoV-2 | 153 | RKYNSAPFT |
| S309-v11 | | |
| mAb CDRL3 | | |
| (aa) | | |
| SARS-CoV-2 | 154 | RKYNSAPYT |
| S309-v12 | | |
| mAb CDRL3 | | |
| (aa) | | |
| SARS-CoV-2 | 155 | QVQLVQSGAELKKPGSSVKVSCKASGGTFN |
| S310 mAb | | SYSFNWVRQAPGQGLEWLGGIIPVLGTSNY |
| VH (aa) | | AQKFQGRVAVTADEFTTTAYMELSSLRSED |
| | | TAVYYCATRTYDSSGYRPYYYGLDVWGQGT |
| | | PVTVSS |
| SARS-CoV-2 | 156 | GGTFNSYS |
| S310 mAb | | |
| CDRHI (aa) | | |
| SARS-CoV-2 | 157 | IIPVLGTS |
| S310 mAb | | |
| CDRH2 (aa) | | |
| SARS-CoV-2 | 158 | ATRTYDSSGYRPYYYGLDV |
| S310 mAb | | |
| CDRH3 (aa) | | |
| SARS-CoV-2 | 159 | QSALTQPASVSGSPGQSITISCTGTSSDVG |
| S310 mAb | | SYNLVSWYQQRPGKAPELMIYEVTKRPSGL |
| VL (VK) (aa) | | SNRFSGSKSGNTASLTISGLQAEDEADYYC |
| | | CSYAGSDTVIFGGGTKVTVL |
| SARS-CoV-2 | 160 | SSDVGSYNL |
| S310 mAb | | |
| CDRL1 (aa) | | |
| SARS-CoV-2 | 161 | EVT |
| S310 mAb | | |
| CDRL2 (aa) | | |
| SARS-CoV-2 | 162 | CSYAGSDTVI |
| S310 mAb | | |
| CDRL3 (aa) | | |
| Wuhan | 163 | 1 attaaaggtt tataccttcc caggtaacaa accaaccaac tttcgatctc ttgtagatct |
| seafood | | 61 gttctctaaa cgaactttaa aatctgtgtg gctgtcactc ggctgcatgc ttagtgcact |
| market | | 121 cacgcagtat aattaataac taattactgt cgttgacagg acacgagtaa ctcgtctatc |
| pneumonia | | 181 ttctgcaggc tgcttacggt ttcgtccgtg ttgcagccga tcatcagcac atctaggttt |
| virus isolate | | 241 cgtccgggtg tgaccgaaag gtaagatgga gagccttgtc cctggtttca acgagaaaac |
| Wuhan-Hu-1 | | 301 acacgtccaa ctcagtttgc ctgttttaca ggttcgcgac gtgctcgtac gtggctttgg |
| genomic | | 361 agactccgtg gaggaggtct tatcagaggc cgtcaacat cttaaagatg gcacttgtgg |
| sequence | | 421 cttagtagaa gttgaaaaag gcgttttgcc tcaacttgaa cagccctatg tgttcatcaa |
| (GenBank: | | 481 acgttcggat gctcgaactg cacctcatgg tcatgttatg gttgagctgg tagcagaact |
| MN908947.3; | | 541 cgaaggcatt cagtacggtc gtagtggtga gacacttggt gtccttgtcc ctcatgtggg |
| Jan. 23, | | 601 cgaaatacca gtggcttacc gcaaggttct tcttcgtaag aacggtaata aaggagctgg |
| 2020) | | 661 tggccatagt tacggcgccg atctaaagtc atttgactta ggcgacgagc ttggcactga |
| | | 721 tccttatgaa gattttcaag aaaactggaa cactaaacat agcagtggtg ttacccgtga |
| | | 781 actcatgcgt gagcttaacg gaggggcata cactcgctat gtogataaca acttctgtgg |
| | | 841 ccctgatggc taccctcttg agtgcattaa agaccttcta gcacgtgctg gtaaagcttc |
| | | 901 atgcactttg tccgaacaac tggactttat tgacactaag aggggtgtat actgctgccg |
| | | 961 tgaacatgag catgaaattg cttggtacac ggaacgttct gaaaagagct atgaattgca |
| | | 1021 gacacctttt gaaattaaat tggcaaagaa atttgacacc ttcaatgggg aatgtccaaa |
| | | 1081 ttttgtattt cccttaaatt ccataatcaa gactattcaa ccaagggttg aaaagaaaaa |
| | | 1141 gcttgatggc tttatgggta gaattcgatc tgtctatcca gttgcgtcac caaatgaatg |
| | | 1201 caaccaaatg tgcctttcaa ctctcatgaa gtgtgatcat tgtggtgaaa cttcatggca |
| | | 1261 gacgggcgat tttgttaaag ccacttgcga attttgtggc actgagaatt tgactaaaga |
| | | 1321 aggtgccact acttgtggtt acttacccca aaatgctgtt gttaaaattt attgtccagc |
| | | 1381 atgtcacaat tcagaagtag gacctgagca tagtcttgcc gaataccata atgaatctgg |
| | | 1441 cttgaaaacc attcttogta agggtggtcg cactattgcc tttggaggct gtgtgttctc |
| | | 1501 ttatgttggt tgccataaca agtgtgccta ttgggttcca cgtgctagcg ctaacatagg |
| | | 1561 ttgtaaccat acaggtgttg ttggagaagg ttccgaaggt cttaatgaca accttcttga |
| | | 1621 aatactccaa aaagagaaag tcaacatcaa tattgttggt gactttaaac ttaatgaaga |
| | | 1681 gatcgccatt attttggcat ctttttctgc ttccacaagt gcttttgtgg aaactgtgaa |
| | | 1741 aggtttggat tataaagcat tcaaacaaat tgttgaatcc tgtggtaatt ttaaagttac |
| | | 1801 aaaaggaaaa gctaaaaaag gtgcctggaa tattggtgaa cagaaatcaa tactgagtcc |
| | | 1861 tctttatgca tttgcatcag aggctgcteg tgttgtacga tcaattttct cccgcactct |
| | | 1921 tgaaactgct caaaattctg tgcgtgtttt acagaaggcc gctataacaa tactagatgg |
| | | 1981 aatttcacag tattcactga gactcattga tgctatgatg ttcacatctg atttggctac |
| | | 2041 taacaatcta gttgtaatgg cctacattac aggtggtgtt gttcagttga cttcgcagtg |
| | | 2101 gctaactaac atctttggca ctgtttatga aaaactcaaa cccgtccttg attggcttga |
| | | 2161 agagaagttt aaggaaggtg tagagtttct tagagacggt tgggaaattg ttaaatttat |
| | | 2221 ctcaacctgt gcttgtgaaa ttgtcggtgg acaaattgtc acctgtgcaa aggaaattaa |
| | | 2281 ggagagtgtt cagacattct ttaagcttgt aaataaattt ttggctttgt gtgctgactc |
| | | 2341 tatcattatt ggtggagcta aacttaaagc cttgaattta ggtgaaacat ttgtcacgca |
| | | 2401 ctcaaaggga ttgtacagaa agtgtgttaa atccagagaa gaaactggcc tactcatgcc |
| | | 2461 tctaaaagcc ccaaaagaaa ttatcttctt agagggagaa acacttccca cagaagtgtt |
| | | 2521 aacagaggaa gttgtcttga aaactggtga tttacaacca ttagaacaac ctactagtga |
| | | 2581 agctgttgaa gctccattgg ttggtacacc agtttgtatt aacgggctta tgttgctoga |
| | | 2641 aatcaaagac acagaaaagt actgtgccct tgcacctaat atgatggtaa caaacaatac |
| | | 2701 cttcacactc aaaggcggtg caccaacaaa ggttactttt ggtgatgaca ctgtgataga |
| | | 2761 agtgcaaggt tacaagagtg tgaatatcac ttttgaactt gatgaaagga ttgataaagt |
| | | 2821 acttaatgag aagtgctctg cctatacagt tgaactoggt acagaagtaa atgagttcgc |
| | | 2881 ctgtgttgtg gcagatgctg tcataaaaac tttgcaacca gtatctgaat tacttacacc |
| | | 2941 actgggcatt gatttagatg agtggagtat ggctacatac tacttatttg atgagtctgg |
| | | 3001 tgagtttaaa ttggcttcac atatgtattg ttctttctac cctccagatg aggatgaaga |
| | | 3061 agaaggtgat tgtgaagaag aagagtttga gccatcaact caatatgagt atggtactga |
| | | 3121 agatgattac caaggtaaac ctttggaatt tggtgccact tctgctgctc ttcaacctga |
| | | 3181 agaagagcaa gaagaagatt ggttagatga tgatagtcaa caaactgttg gtcaacaaga |
| | | 3241 cggcagtgag gacaatcaga caactactat tcaaacaatt gttgaggttc aacctcaatt |
| | | 3301 agagatggaa cttacaccag ttgttcagac tattgaagtg aatagtttta gtggttattt |
| | | 3361 aaaacttact gacaatgtat acattaaaaa tgcagacatt gtggaagaag ctaaaaaggt |
| | | 3421 aaaaccaaca gtggttgtta atgcagccaa tgtttacctt aaacatggag gaggtgttgc |
| | | 3481 aggagcctta aataaggcta ctaacaatgc catgcaagtt gaatctgatg attacatagc |
| | | 3541 tactaatgga ccacttaaag tgggtggtag ttgtgtttta agcggacaca atcttgctaa |
| | | 3601 acactgtctt catgttgtcg gcccaaatgt taacaaaggt gaagacattc aacttcttaa |
| | | 3661 gagtgcttat gaaaatttta atcagcacga agttctactt gcaccattat tatcagctgg |
| | | 3721 tatttttggt gctgacccta tacattcttt aagagtttgt gtagatactg ttogcacaaa |
| | | 3781 tgtctactta gctgtctttg ataaaaatct ctatgacaaa cttgtttcaa gctttttgga |
| | | 3841 aatgaagagt gaaaagcaag ttgaacaaaa gatcgctgag attcctaaag aggaagttaa |
| | | 3901 gccatttata actgaaagta aaccttcagt tgaacagaga aaacaagatg ataagaaaat |
| | | 3961 caaagcttgt gttgaagaag ttacaacaac tctggaagaa actaagttcc tcacagaaaa |
| | | 4021 cttgttactt tatattgaca ttaatggcaa tottcatcca gattctgcca ctcttgttag |
| | | 4081 tgacattgac atcactttct taaagaaaga tgctccatat atagtgggtg atgttgttca |
| | | 4141 agagggtgtt ttaactgctg tggttatacc tactaaaaag gctggtggca ctactgaaat |
| | | 4201 gctagcgaaa gctttgagaa aagtgccaac agacaattat ataaccactt acccgggtca |
| | | 4261 gggtttaaat ggttacactg tagaggaggc aaagacagtg cttaaaaagt gtaaaagtgc |
| | | 4321 cttttacatt ctaccatcta ttatctctaa tgagaagcaa gaaattcttg gaactgtttc |
| | | 4381 ttggaatttg cgagaaatgc ttgcacatgc agaagaaaca cgcaaattaa tgcctgtctg |
| | | 4441 tgtggaaact aaagccatag tttcaactat acagegtaaa tataagggta ttaaaataca |
| | | 4501 agagggtgtg gttgattatg gtgctagatt ttacttttac accagtaaaa caactgtagc |
| | | 4561 gtcacttato aacacactta acgatctaaa tgaaactctt gttacaatgc cacttggcta |
| | | 4621 tgtaacacat ggcttaaatt tggaagaagc tgctcggtat atgagatctc tcaaagtgcc |
| | | 4681 agctacagtt tctgtttctt cacctgatgc tgttacagcg tataatggtt atcttacttc |
| | | 4741 ttcttctaaa acacctgaag aacattttat tgaaaccatc tcacttgctg gttcctataa |
| | | 4801 agattggtcc tattctggac aatctacaca actaggtata gaatttctta agagaggtga |
| | | 4861 taaaagtgta tattacacta gtaatcctac cacattccac ctagatggtg aagttatcac |
| | | 4921 ctttgacaat cttaagacac ttctttcttt gagagaagtg aggactatta aggtgtttac |
| | | 4981 aacagtagac aacattaacc tccacacgca agttgtggac atgtcaatga catatggaca |
| | | 5041 acagtttggt ccaacttatt tggatggagc tgatgttact aaaataaaac ctcataattc |
| | | 5101 acatgaaggt aaaacatttt atgttttacc taatgatgac actctacgtg ttgaggcttt |
| | | 5161 tgagtactac cacacaactg atcctagttt tctgggtagg tacatgtcag cattaaatca |
| | | 5221 cactaaaaag tggaaatacc cacaagttaa tggtttaact tctattaaat gggcagataa |
| | | 5281 caactgttat cttgccactg cattgttaac actccaacaa atagagttga agtttaatcc |
| | | 5341 acctgctcta caagatgctt attacagagc aagggctggt gaagctgcta acttttgtgc |
| | | 5401 acttatctta gcctactgta ataagacagt aggtgagtta ggtgatgtta gagaaacaat |
| | | 5461 gagttacttg tttcaacatg ccaatttaga ttcttgcaaa agagtcttga acgtggtgtg |
| | | 5521 taaaacttgt ggacaacagc agacaaccct taagggtgta gaagctgtta tgtacatggg |
| | | 5581 cacactttct tatgaacaat ttaagaaagg tgttcagata ccttgtacgt gtggtaaaca |
| | | 5641 agctacaaaa tatctagtac aacaggagtc accttttgtt atgatgtcag caccacctgc |
| | | 5701 tcagtatgaa cttaagcatg gtacatttac ttgtgctagt gagtacactg gtaattacca |
| | | 5761 gtgtggtcac tataaacata taacttctaa agaaactttg tattgcatag acggtgcttt |
| | | 5821 acttacaaag tcctcagaat acaaaggtcc tattacggat gttttctaca aagaaaacag |
| | | 5881 ttacacaaca accataaaac cagttactta taaattggat ggtgttgttt gtacagaaat |
| | | 5941 tgaccctaag ttggacaatt attataagaa agacaattct tatttcacag agcaaccaat |
| | | 6001 tgatcttgta ccaaaccaac catatccaaa cgcaagcttc gataatttta agtttgtatg |
| | | 6061 tgataatatc aaatttgctg atgatttaaa ccagttaact ggttataaga aacctgcttc |
| | | 6121 aagagagctt aaagttacat ttttccctga cttaaatggt gatgtggtgg ctattgatta |
| | | 6181 taaacactac acaccctctt ttaagaaagg agctaaattg ttacataaac ctattgtttg |
| | | 6241 gcatgttaac aatgcaacta ataaagccac gtataaacca aatacctggt gtatacgttg |
| | | 6301 tctttggagc acaaaaccag ttgaaacatc aaattcgttt gatgtactga agtcagagga |
| | | 6361 cgcgcaggga atggataatc ttgcctgcga agatctaaaa ccagtctctg aagaagtagt |
| | | 6421 ggaaaatcct accatacaga aagacgttct tgagtgtaat gtgaaaacta ccgaagttgt |
| | | 6481 aggagacatt atacttaaac cagcaaataa tagtttaaaa attacagaag aggttggcca |
| | | 6541 cacagatcta atggctgctt atgtagacaa ttctagtctt actattaaga aacctaatga |
| | | 6601 attatctaga gtattaggtt tgaaaaccct tgctactcat ggtttagctg ctgttaatag |
| | | 6661 tgtcccttgg gatactatag ctaattatgc taagcctttt cttaacaaag ttgttagtac |
| | | 6721 aactactaac atagttacac ggtgtttaaa ccgtgtttgt actaattata tgccttattt |
| | | 6781 ctttacttta ttgctacaat tgtgtacttt tactagaagt acaaattcta gaattaaagc |
| | | 6841 atctatgccg actactatag caaagaatac tgttaagagt gtcggtaaat tttgtctaga |
| | | 6901 ggcttcattt aattatttga agtcacctaa tttttctaaa ctgataaata ttataatttg |
| | | 6961 gtttttacta ttaagtgttt gcctaggttc tttaatctac tcaaccgctg ctttaggtgt |
| | | 7021 tttaatgtct aatttaggca tgccttctta ctgtactggt tacagagaag gctatttgaa |
| | | 7081 ctctactaat gtcactattg caacctactg tactggttct ataccttgta gtgtttgtct |
| | | 7141 tagtggttta gattctttag acacctatcc ttctttagaa actatacaaa ttaccatttc |
| | | 7201 atcttttaaa tgggatttaa ctgcttttgg cttagttgca gagtggtttt tggcatatat |
| | | 7261 tcttttcact aggtttttct atgtacttgg attggctgca atcatgcaat tgtttttcag |
| | | 7321 ctattttgca gtacatttta ttagtaattc ttggcttatg tggttaataa ttaatcttgt |
| | | 7381 acaaatggcc ccgatttcag ctatggttag aatgtacatc ttctttgcat cattttatta |
| | | 7441 tgtatggaaa agttatgtgc atgttgtaga cggttgtaat tcatcaactt gtatgatgtg |
| | | 7501 ttacaaacgt aatagagcaa caagagtcga atgtacaact attgttaatg gtgttagaag |
| | | 7561 gtccttttat gtctatgcta atggaggtaa aggcttttgc aaactacaca attggaattg |
| | | 7621 tgttaattgt gatacattct gtgctggtag tacatttatt agtgatgaag ttgcgagaga |
| | | 7681 cttgtcacta cagtttaaaa gaccaataaa tcctactgac cagtcttctt acatcgttga |
| | | 7741 tagtgttaca gtgaagaatg gttccatcca tctttacttt gataaagctg gtcaaaagac |
| | | 7801 ttatgaaaga cattctctct ctcattttgt taacttagac aacctgagag ctaataacac |
| | | 7861 taaaggttca ttgcctatta atgttatagt ttttgatggt aaatcaaaat gtgaagaatc |
| | | 7921 atctgcaaaa tcagcgtctg tttactacag tcagcttatg tgtcaaccta tactgttact |
| | | 7981 agatcaggca ttagtgtctg atgttggtga tagtgcggaa gttgcagtta aaatgtttga |
| | | 8041 tgcttacgtt aatacgtttt catcaacttt taacgtacca atggaaaaac tcaaaacact |
| | | 8101 agttgcaact gcagaagctg aacttgcaaa gaatgtgtcc ttagacaatg tcttatctac |
| | | 8161 ttttatttca gcagctcggc aagggtttgt tgattcagat gtagaaacta aagatgttgt |
| | | 8221 tgaatgtctt aaattgtcac atcaatctga catagaagtt actggcgata gttgtaataa |
| | | 8281 ctatatgctc acctataaca aagttgaaaa catgacaccc cgtgaccttg gtgcttgtat |
| | | 8341 tgactgtagt gcgcgtcata ttaatgcgca ggtagcaaaa agtcacaaca ttgctttgat |
| | | 8401 atggaacgtt aaagatttca tgtcattgtc tgaacaacta cgaaaacaaa tacgtagtgc |
| | | 8461 tgctaaaaag aataacttac cttttaagtt gacatgtgca actactagac aagttgttaa |
| | | 8521 tgttgtaaca acaaagatag cacttaaggg tggtaaaatt gttaataatt ggttgaagca |
| | | 8581 gttaattaaa gttacacttg tgttcctttt tgttgctgct attttctatt taataacacc |
| | | 8641 tgttcatgtc atgtctaaac atactgactt ttcaagtgaa atcataggat acaaggctat |
| | | 8701 tgatggtggt gtcactogtg acatagcatc tacagatact tgttttgcta acaaacatgc |
| | | 8761 tgattttgac acatggttta gccagcgtgg tggtagttat actaatgaca aagcttgccc |
| | | 8821 attgattgct gcagtcataa caagagaagt gggttttgtc gtgcctggtt tgcctggcac |
| | | 8881 gatattacgc acaactaatg gtgacttttt gcatttctta cctagagttt ttagtgcagt |
| | | 8941 tggtaacatc tgttacacac catcaaaact tatagagtac actgactttg caacatcagc |
| | | 9001 ttgtgttttg gctgctgaat gtacaatttt taaagatgct tctggtaagc cagtaccata |
| | | 9061 ttgttatgat accaatgtac tagaaggttc tgttgcttat gaaagtttac gccctgacac |
| | | 9121 acgttatgtg ctcatggatg gctctattat tcaatttect aacacctacc ttgaaggttc |
| | | 9181 tgttagagtg gtaacaactt ttgattctga gtactgtagg cacggcactt gtgaaagatc |
| | | 9241 agaagctggt gtttgtgtat ctactagtgg tagatgggta cttaacaatg attattacag |
| | | 9301 atctttacca ggagttttct gtggtgtaga tgctgtaaat ttacttacta atatgtttac |
| | | 9361 accactaatt caacctattg gtgctttgga catatcagca tctatagtag ctggtggtat |
| | | 9421 tgtagctatc gtagtaacat gccttgccta ctattttatg aggtttagaa gagcttttgg |
| | | 9481 tgaatacagt catgtagttg cctttaatac tttactattc cttatgtcat tcactgtact |
| | | 9541 ctgtttaaca ccagtttact cattcttacc tggtgtttat tctgttattt acttgtactt |
| | | 9601 gacattttat cttactaatg atgtttcttt tttagcacat attcagtgga tggttatgtt |
| | | 9661 cacaccttta gtacctttct ggataacaat tgcttatatc atttgtattt ccacaaagca |
| | | 9721 tttctattgg ttctttagta attacctaaa gagacgtgta gtctttaatg gtgtttcctt |
| | | 9781 tagtactttt gaagaagctg cgctgtgcac ctttttgtta aataaagaaa tgtatctaaa |
| | | 9841 gttgcgtagt gatgtgctat tacctcttac gcaatataat agatacttag ctctttataa |
| | | 9901 taagtacaag tattttagtg gagcaatgga tacaactagc tacagagaag ctgcttgttg |
| | | 9961 tcatctcgca aaggctctca atgacttcag taactcaggt tctgatgttc tttaccaacc |
| | | 10021 accacaaacc tctatcacct cagctgtttt gcagagtggt tttagaaaaa tggcattccc |
| | | 10081 atctggtaaa gttgagggtt gtatggtaca agtaacttgt ggtacaacta cacttaacgg |
| | | 10141 tctttggctt gatgacgtag tttactgtcc aagacatgtg atctgcacct ctgaagacat |
| | | 10201 gcttaaccct aattatgaag atttactcat togtaagtct aatcataatt tcttggtaca |
| | | 10261 ggctggtaat gttcaactca gggttattgg acattctatg caaaattgtg tacttaagct |
| | | 10321 taaggttgat acagccaatc ctaagacacc taagtataag tttgttcgca ttcaaccagg |
| | | 10381 acagactttt tcagtgttag cttgttacaa tggttcacca tctggtgttt accaatgtgc |
| | | 10441 tatgaggccc aatttcacta ttaagggttc attccttaat ggttcatgtg gtagtgttgg |
| | | 10501 ttttaacata gattatgact gtgtctcttt ttgttacatg caccatatgg aattaccaac |
| | | 10561 tggagttcat gctggcacag acttagaagg taacttttat ggaccttttg ttgacaggca |
| | | 10621 aacagcacaa gcagctggta cggacacaac tattacagtt aatgttttag cttggttgta |
| | | 10681 cgctgctgtt ataaatggag acaggtggtt tctcaatcga tttaccacaa ctcttaatga |
| | | 10741 ctttaacctt gtggctatga agtacaatta tgaacctcta acacaagacc atgttgacat |
| | | 10801 actaggacct ctttctgctc aaactggaat tgccgtttta gatatgtgtg cttcattaaa |
| | | 10861 agaattactg caaaatggta tgaatggacg taccatattg ggtagtgctt tattagaaga |
| | | 10921 tgaatttaca ccttttgatg ttgttagaca atgctcaggt gttactttcc aaagtgcagt |
| | | 10981 gaaaagaaca atcaagggta cacaccactg gttgttactc acaattttga cttcactttt |
| | | 11041 agttttagtc cagagtactc aatggtcttt gttctttttt ttgtatgaaa atgccttttt |
| | | 11101 accttttgct atgggtatta ttgctatgtc tgcttttgca atgatgtttg tcaaacataa |
| | | 11161 gcatgcattt ctctgtttgt ttttgttacc ttctcttgcc actgtagctt attttaatat |
| | | 11221 ggtctatatg cctgctagtt gggtgatgcg tattatgaca tggttggata tggttgatac |
| | | 11281 tagtttgtct ggttttaagc taaaagactg tgttatgtat gcatcagctg tagtgttact |
| | | 11341 aatccttatg acagcaagaa ctgtgtatga tgatggtgct aggagagtgt ggacacttat |
| | | 11401 gaatgtcttg acactcgttt ataaagttta ttatggtaat gctttagatc aagccatttc |
| | | 11461 catgtgggct cttataatct ctgttacttc taactactca ggtgtagtta caactgtcat |
| | | 11521 gtttttggcc agaggtattg tttttatgtg tgttgagtat tgccctattt tcttcataac |
| | | 11581 tggtaataca cttcagtgta taatgctagt ttattgtttc ttaggctatt tttgtacttg |
| | | 11641 ttactttggc ctcttttgtt tactcaaccg ctactttaga ctgactcttg gtgtttatga |
| | | 11701 ttacttagtt tctacacagg agtttagata tatgaattca cagggactac toccacccaa |
| | | 11761 gaatagcata gatgccttca aactcaacat taaattgttg ggtgttggtg gcaaaccttg |
| | | 11821 tatcaaagta gccactgtac agtctaaaat gtcagatgta aagtgcacat cagtagtctt |
| | | 11881 actctcagtt ttgcaacaac tcagagtaga atcatcatct aaattgtggg ctcaatgtgt |
| | | 11941 ccagttacac aatgacattc tcttagctaa agatactact gaagcctttg aaaaaatggt |
| | | 12001 ttcactactt tctgttttgc tttccatgca gggtgctgta gacataaaca agctttgtga |
| | | 12061 agaaatgctg gacaacaggg caaccttaca agctatagcc tcagagttta gttcccttcc |
| | | 12121 atcatatgca gottttgcta ctgctcaaga agcttatgag caggctgttg ctaatggtga |
| | | 12181 ttctgaagtt gttcttaaaa agttgaagaa gtctttgaat gtggctaaat ctgaatttga |
| | | 12241 ccgtgatgca gccatgcaac gtaagttgga aaagatggct gatcaagcta tgacccaaat |
| | | 12301 gtataaacag gctagatctg aggacaagag ggcaaaagtt actagtgcta tgcagacaat |
| | | 12361 gcttttcact atgcttagaa agttggataa tgatgcactc aacaacatta tcaacaatgc |
| | | 12421 aagagatggt tgtgttccct tgaacataat acctcttaca acagcagcca aactaatggt |
| | | 12481 tgtcatacca gactataaca catataaaaa tacgtgtgat ggtacaacat ttacttatgc |
| | | 12541 atcagcattg tgggaaatcc aacaggttgt agatgcagat agtaaaattg ttcaacttag |
| | | 12601 tgaaattagt atggacaatt cacctaattt agcatggcct cttattgtaa cagctttaag |
| | | 12661 ggccaattct gctgtcaaat tacagaataa tgagcttagt cctgttgcac tacgacagat |
| | | 12721 gtcttgtgct gccggtacta cacaaactgc ttgcactgat gacaatgcgt tagcttacta |
| | | 12781 caacacaaca aagggaggta ggtttgtact tgcactgtta tccgatttac aggatttgaa |
| | | 12841 atgggctaga ttccctaaga gtgatggaac tggtactate tatacagaac tggaaccacc |
| | | 12901 ttgtaggttt gttacagaca cacctaaagg toctaaagtg aagtatttat actttattaa |
| | | 12961 aggattaaac aacctaaata gaggtatggt acttggtagt ttagctgcca cagtacgtct |
| | | 13021 acaagctggt aatgcaacag aagtgcctgc caattcaact gtattatctt tctgtgcttt |
| | | 13081 tgctgtagat gctgctaaag cttacaaaga ttatctagct agtgggggac aaccaatcac |
| | | 13141 taattgtgtt aagatgttgt gtacacacac tggtactggt caggcaataa cagttacacc |
| | | 13201 ggaagccaat atggatcaag aatcctttgg tggtgcatcg tgttgtctgt actgccgttg |
| | | 13261 ccacatagat catccaaatc ctaaaggatt ttgtgactta aaaggtaagt atgtacaaat |
| | | 13321 acctacaact tgtgctaatg accctgtggg ttttacactt aaaaacacag tctgtaccgt |
| | | 13381 ctgcggtatg tggaaaggtt atggctgtag ttgtgatcaa ctccgcgaac ccatgcttca |
| | | 13441 gtcagctgat gcacaatcgt ttttaaacgg gtttgcggtg taagtgcagc ccgtcttaca |
| | | 13501 ccgtgcggca caggcactag tactgatgtc gtatacaggg cttttgacat ctacaatgat |
| | | 13561 aaagtagctg gttttgctaa attcctaaaa actaattgtt gtcgcttcca agaaaaggac |
| | | 13621 gaagatgaca atttaattga ttcttacttt gtagttaaga gacacacttt ctctaactac |
| | | 13681 caacatgaag aaacaattta taatttactt aaggattgtc cagctgttgc taaacatgac |
| | | 13741 ttctttaagt ttagaataga cggtgacatg gtaccacata tatcacgtca acgtcttact |
| | | 13801 aaatacacaa tggcagacct cgtctatgct ttaaggcatt ttgatgaagg taattgtgac |
| | | 13861 acattaaaag aaatacttgt cacatacaat tgttgtgatg atgattattt caataaaaag |
| | | 13921 gactggtatg attttgtaga aaacccagat atattacgcg tatacgccaa cttaggtgaa |
| | | 13981 cgtgtacgcc aagctttgtt aaaaacagta caattctgtg atgccatgcg aaatgctggt |
| | | 14041 attgttggtg tactgacatt agataatcaa gatctcaatg gtaactggta tgatttcggt |
| | | 14101 gatttcatac aaaccacgcc aggtagtgga gttcctgttg tagattctta ttattcattg |
| | | 14161 ttaatgccta tattaacctt gaccagggct ttaactgcag agtcacatgt tgacactgac |
| | | 14221 ttaacaaagc cttacattaa gtgggatttg ttaaaatatg acttcacgga agagaggtta |
| | | 14281 aaactctttg accgttattt taaatattgg gatcagacat accacccaaa ttgtgttaac |
| | | 14341 tgtttggatg acagatgcat tctgcattgt gcaaacttta atgttttatt ctctacagtg |
| | | 14401 ttcccaccta caagttttgg accactagtg agaaaaatat ttgttgatgg tgttccattt |
| | | 14461 gtagtttcaa ctggatacca cttcagagag ctaggtgttg tacataatca ggatgtaaac |
| | | 14521 ttacatagct ctagacttag ttttaaggaa ttacttgtgt atgctgctga ccctgctatg |
| | | 14581 cacgctgctt ctggtaatct attactagat aaacgcacta cgtgcttttc agtagctgca |
| | | 14641 cttactaaca atgttgcttt tcaaactgtc aaacccggta attttaacaa agacttctat |
| | | 14701 gactttgctg tgtctaaggg tttctttaag gaaggaagtt ctgttgaatt aaaacacttc |
| | | 14761 ttctttgctc aggatggtaa tgctgctatc agcgattatg actactatcg ttataatcta |
| | | 14821 ccaacaatgt gtgatatcag acaactacta tttgtagttg aagttgttga taagtacttt |
| | | 14881 gattgttacg atggtggctg tattaatgct aaccaagtca togtcaacaa cctagacaaa |
| | | 14941 tcagctggtt ttccatttaa taaatggggt aaggctagac tttattatga ttcaatgagt |
| | | 15001 tatgaggatc aagatgcact tttcgcatat acaaaacgta atgtcatccc tactataact |
| | | 15061 caaatgaatc ttaagtatgc cattagtgca aagaatagag ctcgcaccgt agctggtgtc |
| | | 15121 tctatctgta gtactatgac caatagacag tttcatcaaa aattattgaa atcaatagcc |
| | | 15181 gccactagag gagctactgt agtaattgga acaagcaaat tctatggtgg ttggcacaac |
| | | 15241 atgttaaaaa ctgtttatag tgatgtagaa aaccctcacc ttatgggttg ggattatcct |
| | | 15301 aaatgtgata gagccatgcc taacatgctt agaattatgg cctcacttgt tcttgctcgc |
| | | 15361 aaacatacaa cgtgttgtag cttgtcacac cgtttctata gattagctaa tgagtgtgct |
| | | 15421 caagtattga gtgaaatggt catgtgtggc ggttcactat atgttaaacc aggtggaacc |
| | | 15481 tcatcaggag atgccacaac tgcttatgct aatagtgttt ttaacatttg tcaagctgtc |
| | | 15541 acggccaatg ttaatgcact tttatctact gatggtaaca aaattgccga taagtatgtc |
| | | 15601 cgcaatttac aacacagact ttatgagtgt ctctatagaa atagagatgt tgacacagac |
| | | 15661 tttgtgaatg agttttacgc atatttgcgt aaacatttct caatgatgat actctctgac |
| | | 15721 gatgctgttg tgtgtttcaa tagcacttat gcatctcaag gtctagtggc tagcataaag |
| | | 15781 aactttaagt cagttcttta ttatcaaaac aatgttttta tgtctgaagc aaaatgttgg |
| | | 15841 actgagactg accttactaa aggacctcat gaattttgct ctcaacatac aatgctagtt |
| | | 15901 aaacagggtg atgattatgt gtaccttcct tacccagate catcaagaat cctaggggcc |
| | | 15961 ggctgttttg tagatgatat cgtaaaaaca gatggtacac ttatgattga acggttcgtg |
| | | 16021 tctttagcta tagatgctta cccacttact aaacatccta atcaggagta tgctgatgtc |
| | | 16081 tttcatttgt acttacaata cataagaaag ctacatgatg agttaacagg acacatgtta |
| | | 16141 gacatgtatt ctgttatgct tactaatgat aacacttcaa ggtattggga acctgagttt |
| | | 16201 tatgaggcta tgtacacacc gcatacagtc ttacaggctg ttggggcttg tgttctttgc |
| | | 16261 aattcacaga cttcattaag atgtggtgct tgcatacgta gaccattctt atgttgtaaa |
| | | 16321 tgctgttacg accatgtcat atcaacatca cataaattag tcttgtctgt taatccgtat |
| | | 16381 gtttgcaatg ctccaggttg tgatgtcaca gatgtgactc aactttactt aggaggtatg |
| | | 16441 agctattatt gtaaatcaca taaaccaccc attagttttc cattgtgtgc taatggacaa |
| | | 16501 gtttttggtt tatataaaaa tacatgtgtt ggtagcgata atgttactga ctttaatgca |
| | | 16561 attgcaacat gtgactggac aaatgctggt gattacattt tagctaacac ctgtactgaa |
| | | 16621 agactcaagc tttttgcagc agaaacgctc aaagctactg aggagacatt taaactgtct |
| | | 16681 tatggtattg ctactgtacg tgaagtgctg tctgacagag aattacatct ttcatgggaa |
| | | 16741 gttggtaaac ctagaccacc acttaaccga aattatgtct ttactggtta tegtgtaact |
| | | 16801 aaaaacagta aagtacaaat aggagagtac acctttgaaa aaggtgacta tggtgatgct |
| | | 16861 gttgtttacc gaggtacaac aacttacaaa ttaaatgttg gtgattattt tgtgctgaca |
| | | 16921 tcacatacag taatgccatt aagtgcacct acactagtgc cacaagagca ctatgttaga |
| | | 16981 attactggct tatacccaac actcaatatc tcagatgagt tttctagcaa tgttgcaaat |
| | | 17041 tatcaaaagg ttggtatgca aaagtattct acactccagg gaccacctgg tactggtaag |
| | | 17101 agtcattttg ctattggcct agctctctac tacccttctg ctcgcatagt gtatacagct |
| | | 17161 tgctctcatg ccgctgttga tgcactatgt gagaaggcat taaaatattt gcctatagat |
| | | 17221 aaatgtagta gaattatacc tgcacgtgct cgtgtagagt gttttgataa attcaaagtg |
| | | 17281 aattcaacat tagaacagta tgtcttttgt actgtaaatg cattgcctga gacgacagca |
| | | 17341 gatatagttg tctttgatga aatttcaatg gccacaaatt atgatttgag tgttgtcaat |
| | | 17401 gccagattac gtgctaagca ctatgtgtac attggcgacc ctgctcaatt acctgcacca |
| | | 17461 cgcacattgc taactaaggg cacactagaa ccagaatatt tcaattcagt gtgtagactt |
| | | 17521 atgaaaacta taggtccaga catgttcctc ggaacttgtc ggcgttgtcc tgctgaaatt |
| | | 17581 gttgacactg tgagtgcttt ggtttatgat aataagctta aagcacataa agacaaatca |
| | | 17641 gctcaatgct ttaaaatgtt ttataagggt gttatcacgc atgatgtttc atctgcaatt |
| | | 17701 aacaggccac aaataggcgt ggtaagagaa ttccttacac gtaaccctgc ttggagaaaa |
| | | 17761 gctgtcttta tttcacctta taattcacag aatgctgtag cctcaaagat tttgggacta |
| | | 17821 ccaactcaaa ctgttgatto atcacagggc tcagaatatg actatgtcat attcactcaa |
| | | 17881 accactgaaa cagctcactc ttgtaatgta aacagattta atgttgctat taccagagca |
| | | 17941 aaagtaggca tactttgcat aatgtctgat agagaccttt atgacaagtt gcaatttaca |
| | | 18001 agtcttgaaa ttccacgtag gaatgtggca actttacaag ctgaaaatgt aacaggactc |
| | | 18061 tttaaagatt gtagtaaggt aatcactggg ttacatccta cacaggcacc tacacacctc |
| | | 18121 agtgttgaca ctaaattcaa aactgaaggt ttatgtgttg acatacctgg catacctaag |
| | | 18181 gacatgacct atagaagact catctctatg atgggtttta aaatgaatta tcaagttaat |
| | | 18241 ggttacccta acatgtttat cacccgcgaa gaagctataa gacatgtacg tgcatggatt |
| | | 18301 ggcttcgatg tcgaggggtg tcatgctact agagaagctg ttggtaccaa tttaccttta |
| | | 18361 cagctaggtt tttctacagg tgttaaccta gttgctgtac ctacaggtta tgttgataca |
| | | 18421 cctaataata cagatttttc cagagttagt gctaaaccac cgcctggaga tcaatttaaa |
| | | 18481 cacctcatac cacttatgta caaaggactt ccttggaatg tagtgcgtat aaagattgta |
| | | 18541 caaatgttaa gtgacacact taaaaatctc tctgacagag tcgtatttgt cttatgggca |
| | | 18601 catggctttg agttgacatc tatgaagtat tttgtgaaaa taggacctga gcgcacctgt |
| | | 18661 tgtctatgtg atagacgtgc cacatgcttt tccactgctt cagacactta tgcctgttgg |
| | | 18721 catcattcta ttggatttga ttacgtctat aatccgttta tgattgatgt tcaacaatgg |
| | | 18781 ggttttacag gtaacctaca aagcaaccat gatctgtatt gtcaagtcca tggtaatgca |
| | | 18841 catgtagcta gttgtgatgc aatcatgact aggtgtctag ctgtccacga gtgctttgtt |
| | | 18901 aagcgtgttg actggactat tgaatatcct ataattggtg atgaactgaa gattaatgcg |
| | | 18961 gcttgtagaa aggttcaaca catggttgtt aaagctgcat tattagcaga caaattccca |
| | | 19021 gttcttcacg acattggtaa ccctaaagct attaagtgtg tacctcaagc tgatgtagaa |
| | | 19081 tggaagttct atgatgcaca gccttgtagt gacaaagctt ataaaataga agaattattc |
| | | 19141 tattcttatg ccacacattc tgacaaattc acagatggtg tatgcctatt ttggaattgc |
| | | 19201 aatgtcgata gatatcctgc taattccatt gtttgtagat ttgacactag agtgctatct |
| | | 19261 aaccttaact tgcctggttg tgatggtggc agtttgtatg taaataaaca tgcattccac |
| | | 19321 acaccagctt ttgataaaag tgcttttgtt aatttaaaac aattaccatt tttctattac |
| | | 19381 tctgacagtc catgtgagtc tcatggaaaa caagtagtgt cagatataga ttatgtacca |
| | | 19441 ctaaagtctg ctacgtgtat aacacgttgc aatttaggtg gtgctgtctg tagacatcat |
| | | 19501 gctaatgagt acagattgta tctcgatgct tataacatga tgatctcagc tggctttagc |
| | | 19561 ttgtgggttt acaaacaatt tgatacttat aacctctgga acacttttac aagacttcag |
| | | 19621 agtttagaaa atgtggcttt taatgttgta aataagggac actttgatgg acaacagggt |
| | | 19681 gaagtaccag tttctatcat taataacact gtttacacaa aagttgatgg tgttgatgta |
| | | 19741 gaattgtttg aaaataaaac aacattacct gttaatgtag catttgagct ttgggctaag |
| | | 19801 cgcaacatta aaccagtacc agaggtgaaa atactcaata atttgggtgt ggacattgct |
| | | 19861 gctaatactg tgatctggga ctacaaaaga gatgctccag cacatatatc tactattggt |
| | | 19921 gtttgttcta tgactgacat agccaagaaa ccaactgaaa cgatttgtgc accactcact |
| | | 19981 gtcttttttg atggtagagt tgatggtcaa gtagacttat ttagaaatgc ccgtaatggt |
| | | 20041 gttcttatta cagaaggtag tgttaaaggt ttacaaccat ctgtaggtcc caaacaagct |
| | | 20101 agtcttaatg gagtcacatt aattggagaa gccgtaaaaa cacagttcaa ttattataag |
| | | 20161 aaagttgatg gtgttgtcca acaattacct gaaacttact ttactcagag tagaaattta |
| | | 20221 caagaattta aacccaggag tcaaatggaa attgatttct tagaattagc tatggatgaa |
| | | 20281 ttcattgaac ggtataaatt agaaggctat gccttcgaac atatcgttta tggagatttt |
| | | 20341 agtcatagtc agttaggtgg tttacatcta ctgattggac tagctaaacg ttttaaggaa |
| | | 20401 tcaccttttg aattagaaga ttttattcct atggacagta cagttaaaaa ctatttcata |
| | | 20461 acagatgcgc aaacaggttc atctaagtgt gtgtgttctg ttattgattt attacttgat |
| | | 20521 gattttgttg aaataataaa atcccaagat ttatctgtag tttctaaggt tgtcaaagtg |
| | | 20581 actattgact atacagaaat ttcatttatg ctttggtgta aagatggcca tgtagaaaca |
| | | 20641 ttttacccaa aattacaatc tagtcaagcg tggcaaccgg gtgttgctat gcctaatctt |
| | | 20701 tacaaaatgc aaagaatgct attagaaaag tgtgaccttc aaaattatgg tgatagtgca |
| | | 20761 acattaccta aaggcataat gatgaatgtc gcaaaatata ctcaactgtg tcaatattta |
| | | 20821 aacacattaa cattagctgt accctataat atgagagtta tacattttgg tgctggttct |
| | | 20881 gataaaggag ttgcaccagg tacagctgtt ttaagacagt ggttgcctac gggtacgctg |
| | | 20941 cttgtcgatt cagatcttaa tgactttgtc tctgatgcag attcaacttt gattggtgat |
| | | 21001 tgtgcaactg tacatacagc taataaatgg gatctcatta ttagtgatat gtacgaccct |
| | | 21061 aagactaaaa atgttacaaa agaaaatgac tctaaagagg gttttttcac ttacatttgt |
| | | 21121 gggtttatac aacaaaagct agctcttgga ggttccgtgg ctataaagat aacagaacat |
| | | 21181 tcttggaatg ctgatcttta taagctcatg ggacacttcg catggtggac agcctttgtt |
| | | 21241 actaatgtga atgcgtcatc atctgaagca tttttaattg gatgtaatta tcttggcaaa |
| | | 21301 ccacgcgaac aaatagatgg ttatgtcatg catgcaaatt acatattttg gaggaataca |
| | | 21361 aatccaattc agttgtcttc ctattcttta tttgacatga gtaaatttcc ccttaaatta |
| | | 21421 aggggtactg ctgttatgtc tttaaaagaa ggtcaaatca atgatatgat tttatctctt |
| | | 21481 cttagtaaag gtagacttat aattagagaa aacaacagag ttgttatttc tagtgatgtt |
| | | 21541 cttgttaaca actaaacgaa caatgtttgt ttttcttgtt ttattgccac tagtctctag |
| | | 21601 tcagtgtgtt aatcttacaa ceagaactca attaccccct gcatacacta attctttcac |
| | | 21661 acgtggtgtt tattaccctg acaaagtttt cagatcctca gttttacatt caactcagga |
| | | 21721 cttgttctta cctttctttt ccaatgttac ttggttccat gctatacatg tctctgggac |
| | | 21781 caatggtact aagaggtttg ataaccctgt cctaccattt aatgatggtg tttattttgc |
| | | 21841 ttccactgag aagtctaaca taataagagg ctggattttt ggtactactt tagattcgaa |
| | | 21901 gacccagtcc ctacttattg ttaataacgc tactaatgtt gttattaaag tctgtgaatt |
| | | 21961 tcaattttgt aatgatccat ttttgggtgt ttattaccac aaaaacaaca aaagttggat |
| | | 22021 ggaaagtgag ttcagagttt attctagtgc gaataattgc acttttgaat atgtctctca |
| | | 22081 gccttttctt atggaccttg aaggaaaaca gggtaatttc aaaaatctta gggaatttgt |
| | | 22141 gtttaagaat attgatggtt attttaaaat atattctaag cacacgccta ttaatttagt |
| | | 22201 gcgtgatctc cctcagggtt tttcggcttt agaaccattg gtagatttgc caataggtat |
| | | 22261 taacatcact aggtttcaaa ctttacttgc tttacataga agttatttga ctcctggtga |
| | | 22321 ttcttcttca ggttggacag ctggtgctgc agcttattat gtgggttatc ttcaacctag |
| | | 22381 gacttttcta ttaaaatata atgaaaatgg aaccattaca gatgctgtag actgtgcact |
| | | 22441 tgaccctctc tcagaaacaa agtgtacgtt gaaatccttc actgtagaaa aaggaatcta |
| | | 22501 tcaaacttct aactttagag tccaaccaac agaatctatt gttagatttc ctaatattac |
| | | 22561 aaacttgtgc ccttttggtg aagtttttaa cgccaccaga tttgcatctg tttatgcttg |
| | | 22621 gaacaggaag agaatcagca actgtgttgc tgattattct gtcctatata attccgcatc |
| | | 22681 attttccact tttaagtgtt atggagtgtc tcctactaaa ttaaatgato tctgctttac |
| | | 22741 taatgtctat gcagattcat ttgtaattag aggtgatgaa gtcagacaaa tcgctccagg |
| | | 22801 gcaaactgga aagattgctg attataatta taaattacca gatgatttta caggctgcgt |
| | | 22861 tatagcttgg aattctaaca atcttgattc taaggttggt ggtaattata attacctgta |
| | | 22921 tagattgttt aggaagtcta atctcaaacc ttttgagaga gatatttcaa ctgaaatcta |
| | | 22981 tcaggccggt agcacacctt gtaatggtgt tgaaggtttt aattgttact ttcctttaca |
| | | 23041 atcatatggt ttccaaccca ctaatggtgt tggttaccaa ccatacagag tagtagtact |
| | | 23101 ttcttttgaa cttctacatg caccagcaac tgtttgtgga cctaaaaagt ctactaattt |
| | | 23161 ggttaaaaac aaatgtgtca atttcaactt caatggttta acaggcacag gtgttcttac |
| | | 23221 tgagtctaac aaaaagtttc tgcctttcca acaatttggc agagacattg ctgacactac |
| | | 23281 tgatgctgtc cgtgatccac agacacttga gattcttgac attacaccat gttcttttgg |
| | | 23341 tggtgtcagt gttataacac caggaacaaa tacttctaac caggttgctg ttctttatca |
| | | 23401 ggatgttaac tgcacagaag tccctgttgc tattcatgca gatcaactta ctcctacttg |
| | | 23461 gcgtgtttat tctacaggtt ctaatgtttt tcaaacacgt gcaggctgtt taataggggc |
| | | 23521 tgaacatgtc aacaactcat atgagtgtga catacccatt ggtgcaggta tatgcgctag |
| | | 23581 ttatcagact cagactaatt ctcctcggcg ggcacgtagt gtagctagtc aatccatcat |
| | | 23641 tgcctacact atgtcacttg gtgcagaaaa ttcagttgct tactctaata actctattgc |
| | | 23701 catacccaca aattttacta ttagtgttac cacagaaatt ctaccagtgt ctatgaccaa |
| | | 23761 gacatcagta gattgtacaa tgtacatttg tggtgattca actgaatgca gcaatctttt |
| | | 23821 gttgcaatat ggcagttttt gtacacaatt aaaccgtgct ttaactggaa tagctgttga |
| | | 23881 acaagacaaa aacacccaag aagtttttgc acaagtcaaa caaatttaca aaacaccacc |
| | | 23941 aattaaagat tttggtggtt ttaatttttc acaaatatta ccagatccat caaaaccaag |
| | | 24001 caagaggtca tttattgaag atctactttt caacaaagtg acacttgcag atgctggctt |
| | | 24061 catcaaacaa tatggtgatt gccttggtga tattgctgct agagacctca tttgtgcaca |
| | | 24121 aaagtttaac ggccttactg ttttgccacc tttgctcaca gatgaaatga ttgctcaata |
| | | 24181 cacttctgca ctgttagcgg gtacaatcac ttctggttgg acctttggtg caggtgctgc |
| | | 24241 attacaaata ccatttgcta tgcaaatggc ttataggttt aatggtattg gagttacaca |
| | | 24301 gaatgttctc tatgagaacc aaaaattgat tgccaaccaa tttaatagtg ctattggcaa |
| | | 24361 aattcaagac tcactttctt ccacagcaag tgcacttgga aaacttcaag atgtggtcaa |
| | | 24421 ccaaaatgca caagctttaa acacgcttgt taaacaactt agctccaatt ttggtgcaat |
| | | 24481 ttcaagtgtt ttaaatgata tcctttcacg tcttgacaaa gttgaggctg aagtgcaaat |
| | | 24541 tgataggttg atcacaggca gacttcaaag tttgcagaca tatgtgactc aacaattaat |
| | | 24601 tagagctgca gaaatcagag cttctgctaa tcttgctgct actaaaatgt cagagtgtgt |
| | | 24661 acttggacaa tcaaaaagag ttgatttttg tggaaagggc tatcatctta tgtccttccc |
| | | 24721 tcagtcagca cctcatggtg tagtcttctt gcatgtgact tatgtccctg cacaagaaaa |
| | | 24781 gaacttcaca actgctcctg ccatttgtca tgatggaaaa gcacactttc ctcgtgaagg |
| | | 24841 tgtctttgtt tcaaatggca cacactggtt tgtaacacaa aggaattttt atgaaccaca |
| | | 24901 aatcattact acagacaaca catttgtgtc tggtaactgt gatgttgtaa taggaattgt |
| | | 24961 caacaacaca gtttatgatc ctttgcaacc tgaattagac tcattcaagg aggagttaga |
| | | 25021 taaatatttt aagaatcata catcaccaga tgttgattta ggtgacatct ctggcattaa |
| | | 25081 tgcttcagtt gtaaacattc aaaaagaaat tgaccgcctc aatgaggttg ccaagaattt |
| | | 25141 aaatgaatct ctcategate tccaagaact tggaaagtat gagcagtata taaaatggcc |
| | | 25201 atggtacatt tggctaggtt ttatagctgg cttgattgcc atagtaatgg tgacaattat |
| | | 25261 gctttgctgt atgaccagtt gctgtagttg tctcaagggc tgttgttctt gtggatcctg |
| | | 25321 ctgcaaattt gatgaagacg actctgagcc agtgctcaaa ggagtcaaat tacattacac |
| | | 25381 ataaacgaac ttatggattt gtttatgaga atcttcacaa ttggaactgt aactttgaag |
| | | 25441 caaggtgaaa tcaaggatgc tactccttca gattttgttc gegctactgc aacgataccg |
| | | 25501 atacaagcct cactcccttt cggatggctt attgttggcg ttgcacttct tgctgttttt |
| | | 25561 cagagcgctt ccaaaatcat aaccctcaaa aagagatggc aactagcact ctccaagggt |
| | | 25621 gttcactttg tttgcaactt gctgttgttg tttgtaacag tttactcaca ccttttgctc |
| | | 25681 gttgctgctg gccttgaagc cccttttctc tatctttatg ctttagtcta cttcttgcag |
| | | 25741 agtataaact ttgtaagaat aataatgagg ctttggcttt gctggaaatg cogttccaaa |
| | | 25801 aacccattac tttatgatgc caactatttt ctttgctggc atactaattg ttacgactat |
| | | 25861 tgtatacctt acaatagtgt aacttcttca attgtcatta cttcaggtga tggcacaaca |
| | | 25921 agtcctattt ctgaacatga ctaccagatt ggtggttata ctgaaaaatg ggaatctgga |
| | | 25981 gtaaaagact gtgttgtatt acacagttac ttcacttcag actattacca gctgtactca |
| | | 26041 actcaattga gtacagacac tggtgttgaa catgttacct tcttcatcta caataaaatt |
| | | 26101 gttgatgagc ctgaagaaca tgtccaaatt cacacaatcg acggttcatc cggagttgtt |
| | | 26161 aatccagtaa tggaaccaat ttatgatgaa ccgacgacga ctactagcgt gcctttgtaa |
| | | 26221 gcacaagctg atgagtacga acttatgtac tcattcgttt cggaagagac aggtacgtta |
| | | 26281 atagttaata gcgtacttct ttttcttgct ttcgtggtat tcttgctagt tacactagcc |
| | | 26341 atccttactg cgcttcgatt gtgtgcgtac tgctgcaata ttgttaacgt gagtcttgta |
| | | 26401 aaaccttctt tttacgttta ctctcgtgtt aaaaatctga attcttctag agttcctgat |
| | | 26461 cttctggtct aaacgaacta aatattatat tagtttttct gtttggaact ttaattttag |
| | | 26521 ccatggcaga ttccaacggt actattaccg ttgaagagct taaaaagctc cttgaacaat |
| | | 26581 ggaacctagt aataggtttc ctattcctta catggatttg tottctacaa tttgcctatg |
| | | 26641 ccaacaggaa taggtttttg tatataatta agttaatttt cctctggctg ttatggccag |
| | | 26701 taactttagc ttgttttgtg cttgctgctg tttacagaat aaattggatc accggtggaa |
| | | 26761 ttgctatcgc aatggcttgt cttgtaggct tgatgtggct cagctacttc attgcttctt |
| | | 26821 tcagactgtt tgcgcgtacg cgttccatgt ggtcattcaa tccagaaact aacattcttc |
| | | 26881 tcaacgtgcc actccatggc actattctga ccagaccgct tctagaaagt gaactcgtaa |
| | | 26941 tcggagctgt gatccttcgt ggacatcttc gtattgctgg acaccatcta ggacgctgtg |
| | | 27001 acatcaagga cctgcctaaa gaaatcactg ttgctacatc acgaacgctt tcttattaca |
| | | 27061 aattgggagc ttcgcagcgt gtagcaggtg actcaggttt tgctgcatac agtcgctaca |
| | | 27121 ggattggcaa ctataaatta aacacagacc attccagtag cagtgacaat attgctttgc |
| | | 27181 ttgtacagta agtgacaaca gatgtttcat ctogttgact ttcaggttac tatagcagag |
| | | 27241 atattactaa ttattatgag gacttttaaa gtttccattt ggaatcttga ttacatcata |
| | | 27301 aacctcataa ttaaaaattt atctaagtca ctaactgaga ataaatattc tcaattagat |
| | | 27361 gaagagcaac caatggagat tgattaaacg aacatgaaaa ttattctttt cttggcactg |
| | | 27421 ataacacteg ctacttgtga gctttatcac taccaagagt gtgttagagg tacaacagta |
| | | 27481 cttttaaaag aaccttgctc ttctggaaca tacgagggca attcaccatt tcatcctcta |
| | | 27541 gctgataaca aatttgcact gacttgcttt agcactcaat ttgcttttgc ttgtcctgac |
| | | 27601 ggcgtaaaac acgtctatca gttacgtgcc agatcagttt cacctaaact gttcatcaga |
| | | 27661 caagaggaag ttcaagaact ttactctcca atttttctta ttgttgcggc aatagtgttt |
| | | 27721 ataacacttt gcttcacact caaaagaaag acagaatgat tgaactttca ttaattgact |
| | | 27781 tctatttgtg ctttttagcc tttctgctat tccttgtttt aattatgctt attatctttt |
| | | 27841 ggttctcact tgaactgcaa gatcataatg aaacttgtca cgcctaaacg aacatgaaat |
| | | 27901 ttcttgtttt cttaggaatc atcacaactg tagctgcatt tcaccaagaa tgtagtttac |
| | | 27961 agtcatgtac tcaacatcaa ccatatgtag ttgatgaccc gtgtcctatt cacttctatt |
| | | 28021 ctaaatggta tattagagta ggagctagaa aatcagcacc tttaattgaa ttgtgcgtgg |
| | | 28081 atgaggctgg ttctaaatca cccattcagt acatcgatat cggtaattat acagtttcct |
| | | 28141 gtttaccttt tacaattaat tgccaggaac ctaaattggg tagtcttgta gtgcgttgtt |
| | | 28201 cgttctatga agacttttta gagtatcatg acgttcgtgt tgttttagat ttcatctaaa |
| | | 28261 cgaacaaact aaaatgtctg ataatggacc ccaaaatcag cgaaatgcac cccgcattac |
| | | 28321 gtttggtgga ccctcagatt caactggcag taaccagaat ggagaacgca gtggggcgcg |
| | | 28381 atcaaaacaa cgteggcccc aaggtttacc caataatact gcgtcttggt tcaccgctct |
| | | 28441 cactcaacat ggcaaggaag accttaaatt ccctogagga caaggegttc caattaacac |
| | | 28501 caatagcagt ccagatgacc aaattggcta ctaccgaaga gctaccagac gaattcgtgg |
| | | 28561 tggtgacggt aaaatgaaag atctcagtcc aagatggtat ttctactacc taggaactgg |
| | | 28621 gccagaagct ggacttccct atggtgctaa caaagacggc atcatatggg ttgcaactga |
| | | 28681 gggagccttg aatacaccaa aagatcacat tggcacccgc aatcctgcta acaatgctgc |
| | | 28741 aatcgtgcta caacttcctc aaggaacaac attgccaaaa ggcttctacg cagaagggag |
| | | 28801 cagaggcggc agtcaagcct cttctogttc ctcatcacgt agtcgcaaca gttcaagaaa |
| | | 28861 ttcaactcca ggcagcagta ggggaacttc tcctgctaga atggctggca atggcggtga |
| | | 28921 tgctgctctt gctttgctgc tgcttgacag attgaaccag cttgagagca aaatgtctgg |
| | | 28981 taaaggccaa caacaacaag gccaaactgt cactaagaaa tctgctgctg aggcttctaa |
| | | 29041 gaagcctcgg caaaaacgta ctgccactaa agcatacaat gtaacacaag ctttcggcag |
| | | 29101 acgtggtcca gaacaaaccc aaggaaattt tggggaccag gaactaatca gacaaggaac |
| | | 29161 tgattacaaa cattggccgc aaattgcaca atttgccccc agcgcttcag cgttcttcgg |
| | | 29221 aatgtcgcgc attggcatgg aagtcacacc ttcgggaacg tggttgacct acacaggtgc |
| | | 29281 catcaaattg gatgacaaag atccaaattt caaagatcaa gtcattttgc tgaataagca |
| | | 29341 tattgacgca tacaaaacat toccaccaac agagcctaaa aaggacaaaa agaagaaggc |
| | | 29401 tgatgaaact caagccttac cgcagagaca gaagaaacag caaactgtga ctcttcttcc |
| | | 29461 tgctgcagat ttggatgatt tctccaaaca attgcaacaa tocatgagca gtgctgactc |
| | | 29521 aactcaggcc taaactcatg cagaccacac aaggcagatg ggctatataa acgttttcgc |
| | | 29581 ttttccgttt acgatatata gtctactctt gtgcagaatg aattctcgta actacatagc |
| | | 29641 acaagtagat gtagttaact ttaatctcac atagcaatct ttaatcagtg tgtaacatta |
| | | 29701 gggaggactt gaaagagcca ccacattttc accgaggcca cgcggagtac gatcgagtgt |
| | | 29761 acagtgaaca atgctaggga gagctgccta tatggaagag ccctaatgtg taaaattaat |
| | | 29821 tttagtagtg ctatccccat gtgattttaa tagcttctta ggagaatgac aaaaaaaaaa |
| | | 29881 aaaaaaaaaa aaaaaaaaaa aaa |
| Wuhan | 164 | MESLVPGFNEKTHVQLSLPVLQVRDVLVRG |
| seafood | | FGDSVEEVLSEARQHLKDGTCGLVEVEKGV |
| market | | LPQLEQPYVFIKRSDARTAPHGHVMVELVA |
| pneumonia | | ELEGIQYGRSGETLGVLVPHVGEIPVAYRK |
| virus isolate | | VLLRKNGNKGAGGHSYGADLKSFDLGDELG |
| Wuhan-Hu-1 | | TDPYEDFQENWNTKHSSGVTRELMRELNGG |
| genomic | | AYTRYVDNNFCGPDGYPLECIKDLLARAGK |
| sequence | | ASCTLSEQLDFIDTKRGVYCCREHEHEIAW |
| (GenBank: | | YTERSEKSYELQTPFEIKLAKKFDTFNGEC |
| MN908947.3; | | PNFVFPLNSIIKTIQPRVEKKKLDGFMGRI |
| Jan. 23, | | RSVYPVASPNECNQMCLSTLMKCDHCGETS |
| 2020)-amino | | WQTGDFVKATCEFCGTENLTKEGATTCGYL |
| acid | | PQNAVVKIYCPACHNSEVGPEHSLAEYHNE |
| translation | | SGLKTILRKGGRTIAFGGCVFSYVGCHNKC |
| | | AYWVPRASANIGCNHTGVVGEGSEGLNDNL |
| | | LEILQKEKVNINIVGDFKLNEEIAIILASF |
| | | SASTSAFVETVKGLDYKAFKQIVESCGNFK |
| | | VTKGKAKKGAWNIGEQKSILSPLYAFASEA |
| | | ARVVRSIFSRTLETAQNSVRVLQKAAITIL |
| | | DGISQYSLRLIDAMMFTSDLATNNLVVMAY |
| | | ITGGVVQLTSQWLTNIFGTVYEKLKPVLDW |
| | | LEEKFKEGVEFLRDGWEIVKFISTCACEIV |
| | | GGQIVTCAKEIKESVQTFFKLVNKFLALCA |
| | | DSIIIGGAKLKALNLGETFVTHSKGLYRKC |
| | | VKSREETGLLMPLKAPKEIIFLEGETLPTE |
| | | VLTEEVVLKTGDLQPLEQPTSEAVEAPLVG |
| | | TPVCINGLMLLEIKDTEKYCALAPNMMVTN |
| | | NTFTLKGGAPTKVTFGDDTVIEVQGYKSVN |
| | | ITFELDERIDKVLNEKCSAYTVELGTEVNE |
| | | FACVVADAVIKTLQPVSELLTPLGIDLDEW |
| | | SMATYYLFDESGEFKLASHMYCSFYPPDED |
| | | EEEGDCEEEEFEPSTQYEYGTEDDYQGKPL |
| | | EFGATSAALQPEEEQEEDWLDDDSQQTVGQ |
| | | QDGSEDNQTTTIQTIVEVQPQLEMELTPVV |
| | | QTIEVNSFSGYLKLTDNVYIKNADIVEEAK |
| | | KVKPTVVVNAANVYLKHGGGVAGALNKATN |
| | | NAMQVESDDYIATNGPLKVGGSCVLSGHNL |
| | | AKHCLHVVGPNVNKGEDIQLLKSAYENFNQ |
| | | HEVLLAPLLSAGIFGADPIHSLRVCVDTVR |
| | | TNVYLAVFDKNLYDKLVSSFLEMKSEKQVE |
| | | QKIAEIPKEEVKPFITESKPSVEQRKQDDK |
| | | KIKACVEEVTTTLEETKFLTENLLLYIDIN |
| | | GNLHPDSATLVSDIDITFLKKDAPYIVGDV |
| | | VQEGVLTAVVIPTKKAGGTTEMLAKALRKV |
| | | PTDNYITTYPGQGLNGYTVEEAKTVLKKCK |
| | | SAFYILPSIISNEKQEILGTVSWNLREMLA |
| | | HAEETRKLMPVCVETKAIVSTIQRKYKGIK |
| | | IQEGVVDYGARFYFYTSKTTVASLINTLND |
| | | LNETLVTMPLGYVTHGLNLEEAARYMRSLK |
| | | VPATVSVSSPDAVTAYNGYLTSSSKTPEEH |
| | | FIETISLAGSYKDWSYSGQSTQLGIEFLKR |
| | | GDKSVYYTSNPTTFHLDGEVITFDNLKTLL |
| | | SLREVRTIKVFTTVDNINLHTQVVDMSMTY |
| | | GQQFGPTYLDGADVTKIKPHNSHEGKTFYV |
| | | LPNDDTLRVEAFEYYHTTDPSFLGRYMSAL |
| | | NHTKKWKYPQVNGLTSIKWADNNCYLATAL |
| | | LTLQQIELKFNPPALQDAYYRARAGEAANF |
| | | CALILAYCNKTVGELGDVRETMSYLFQHAN |
| | | LDSCKRVLNVVCKTCGQQQTTLKGVEAVMY |
| | | MGTLSYEQFKKGVQIPCTCGKQATKYLVQQ |
| | | ESPFVMMSAPPAQYELKHGTFTCASEYTGN |
| | | YQCGHYKHITSKETLYCIDGALLTKSSEYK |
| | | GPITDVFYKENSYTTTIKPVTYKLDGVVCT |
| | | EIDPKLDNYYKKDNSYFTEQPIDLVPNQPY |
| | | PNASFDNFKFVCDNIKFADDLNQLTGYKKP |
| | | ASRELKVTFFPDLNGDVVAIDYKHYTPSFK |
| | | KGAKLLHKPIVWHVNNATNKATYKPNTWCI |
| | | RCLWSTKPVETSNSFDVLKSEDAQGMDNLA |
| | | CEDLKPVSEEVVENPTIQKDVLECNVKTTE |
| | | VVGDIILKPANNSLKITEEVGHTDLMAAYV |
| | | DNSSLTIKKPNELSRVLGLKTLATHGLAAV |
| | | NSVPWDTIANYAKPFLNKVVSTTTNIVTRC |
| | | LNRVCTNYMPYFFTLLLQLCTFTRSTNSRI |
| | | KASMPTTIAKNTVKSVGKFCLEASFNYLKS |
| | | PNFSKLINIIIWFLLLSVCLGSLIYSTAAL |
| | | GVLMSNLGMPSYCTGYREGYLNSTNVTIAT |
| | | YCTGSIPCSVCLSGLDSLDTYPSLETIQIT |
| | | ISSFKWDLTAFGLVAEWFLAYILFTRFFYV |
| | | LGLAAIMQLFFSYFAVHFISNSWLMWLIIN |
| | | LVQMAPISAMVRMYIFFASFYYVWKSYVHV |
| | | VDGCNSSTCMMCYKRNRATRVECTTIVNGV |
| | | RRSFYVYANGGKGFCKLHNWNCVNCDTFCA |
| | | GSTFISDEVARDLSLQFKRPINPTDQSSYI |
| | | VDSVTVKNGSIHLYFDKAGQKTYERHSLSH |
| | | FVNLDNLRANNTKGSLPINVIVFDGKSKCE |
| | | ESSAKSASVYYSQLMCQPILLLDQALVSDV |
| | | GDSAEVAVKMFDAYVNTFSSTFNVPMEKLK |
| | | TLVATAEAELAKNVSLDNVLSTFISAARQG |
| | | FVDSDVETKDVVECLKLSHQSDIEVTGDSC |
| | | NNYMLTYNKVENMTPRDLGACIDCSARHIN |
| | | AQVAKSHNIALIWNVKDFMSLSEQLRKQIR |
| | | SAAKKNNLPFKLTCATTRQVVNVVTTKIAL |
| | | KGGKIVNNWLKQLIKVTLVFLFVAAIFYLI |
| | | TPVHVMSKHTDFSSEIIGYKAIDGGVTRDI |
| | | ASTDTCFANKHADFDTWFSQRGGSYTNDKA |
| | | CPLIAAVITREVGFVVPGLPGTILRTTNGD |
| | | FLHFLPRVFSAVGNICYTPSKLIEYTDFAT |
| | | SACVLAAECTIFKDASGKPVPYCYDTNVLE |
| | | GSVAYESLRPDTRYVLMDGSIIQFPNTYLE |
| | | GSVRVVTTFDSEYCRHGTCERSEAGVCVST |
| | | SGRWVLNNDYYRSLPGVFCGVDAVNLLTNM |
| | | FTPLIQPIGALDISASIVAGGIVAIVVTCL |
| | | AYYFMRFRRAFGEYSHVVAFNTLLFLMSFT |
| | | VLCLTPVYSFLPGVYSVIYLYLTFYLTNDV |
| | | SFLAHIQWMVMFTPLVPFWITIAYIICIST |
| | | KHFYWFFSNYLKRRVVFNGVSFSTFEEAAL |
| | | CTFLLNKEMYLKLRSDVLLPLTQYNRYLAL |
| | | YNKYKYFSGAMDTTSYREAACCHLAKALND |
| | | FSNSGSDVLYQPPQTSITSAVLQSGFRKMA |
| | | FPSGKVEGCMVQVTCGTTTLNGLWLDDVVY |
| | | CPRHVICTSEDMLNPNYEDLLIRKSNHNFL |
| | | VQAGNVQLRVIGHSMQNCVLKLKVDTANPK |
| | | TPKYKFVRIQPGQTFSVLACYNGSPSGVYQ |
| | | CAMRPNFTIKGSFLNGSCGSVGFNIDYDCV |
| | | SFCYMHHMELPTGVHAGTDLEGNFYGPFVD |
| | | RQTAQAAGTDTTITVNVLAWLYAAVINGDR |
| | | WFLNRFTTTLNDFNLVAMKYNYEPLTQDHV |
| | | DILGPLSAQTGIAVLDMCASLKELLQNGMN |
| | | GRTILGSALLEDEFTPFDVVRQCSGVTFQS |
| | | AVKRTIKGTHHWLLLTILTSLLVLVQSTQW |
| | | SLFFFLYENAFLPFAMGIIAMSAFAMMFVK |
| | | HKHAFLCLFLLPSLATVAYFNMVYMPASWV |
| | | MRIMTWLDMVDTSLSGFKLKDCVMYASAVV |
| | | LLILMTARTVYDDGARRVWTLMNVLTLVYK |
| | | VYYGNALDQAISMWALIISVTSNYSGVVTT |
| | | VMFLARGIVFMCVEYCPIFFITGNTLQCIM |
| | | LVYCFLGYFCTCYFGLFCLLNRYFRLTLGV |
| | | YDYLVSTQEFRYMNSQGLLPPKNSIDAFKL |
| | | NIKLLGVGGKPCIKVATVQSKMSDVKCTSV |
| | | VLLSVLQQLRVESSSKLWAQCVQLHNDILL |
| | | AKDTTEAFEKMVSLLSVLLSMQGAVDINKL |
| | | CEEMLDNRATLQAIASEFSSLPSYAAFATA |
| | | QEAYEQAVANGDSEVVLKKLKKSLNVAKSE |
| | | FDRDAAMQRKLEKMADQAMTQMYKQARSED |
| | | KRAKVTSAMQTMLFTMLRKLDNDALNNIIN |
| | | NARDGCVPLNIIPLTTAAKLMVVIPDYNTY |
| | | KNTCDGTTFTYASALWEIQQVVDADSKIVQ |
| | | LSEISMDNSPNLAWPLIVTALRANSAVKLQ |
| | | NNELSPVALRQMSCAAGTTQTACTDDNALA |
| | | YYNTTKGGRFVLALLSDLQDLKWARFPKSD |
| | | GTGTIYTELEPPCRFVTDTPKGPKVKYLYF |
| | | IKGLNNLNRGMVLGSLAATVRLQAGNATEV |
| | | PANSTVLSFCAFAVDAAKAYKDYLASGGQP |
| | | ITNCVKMLCTHTGTGQAITVTPEANMDQES |
| | | FGGASCCLYCRCHIDHPNPKGFCDLKGKYV |
| | | QIPTTCANDPVGFTLKNTVCTVCGMWKGYG |
| | | CSCDQLREPMLQSADAQSFLNRVCGVSAAR |
| | | LTPCGTGTSTDVVYRAFDIYNDKVAGFAKF |
| | | LKTNCCRFQEKDEDDNLIDSYFVVKRHTFS |
| | | NYQHEETIYNLLKDCPAVAKHDFFKFRIDG |
| | | DMVPHISRQRLTKYTMADLVYALRHFDEGN |
| | | CDTLKEILVTYNCCDDDYFNKKDWYDFVEN |
| | | PDILRVYANLGERVRQALLKTVQFCDAMRN |
| | | AGIVGVLTLDNQDLNGNWYDFGDFIQTTPG |
| | | SGVPVVDSYYSLLMPILTLTRALTAESHVD |
| | | TDLTKPYIKWDLLKYDFTEERLKLFDRYFK |
| | | YWDQTYHPNCVNCLDDRCILHCANFNVLFS |
| | | TVFPPTSFGPLVRKIFVDGVPFVVSTGYHF |
| | | RELGVVHNQDVNLHSSRLSFKELLVYAADP |
| | | AMHAASGNLLLDKRTTCFSVAALTNNVAFQ |
| | | TVKPGNFNKDFYDFAVSKGFFKEGSSVELK |
| | | HFFFAQDGNAAISDYDYYRYNLPTMCDIRQ |
| | | LLFVVEVVDKYFDCYDGGCINANQVIVNNL |
| | | DKSAGFPFNKWGKARLYYDSMSYEDQDALF |
| | | AYTKRNVIPTITQMNLKYAISAKNRARTVA |
| | | GVSICSTMTNRQFHQKLLKSIAATRGATVV |
| | | IGTSKFYGGWHNMLKTVYSDVENPHLMGWD |
| | | YPKCDRAMPNMLRIMASLVLARKHTTCCSL |
| | | SHRFYRLANECAQVLSEMVMCGGSLYVKPG |
| | | GTSSGDATTAYANSVFNICQAVTANVNALL |
| | | STDGNKIADKYVRNLQHRLYECLYRNRDVD |
| | | TDFVNEFYAYLRKHFSMMILSDDAVVCFNS |
| | | TYASQGLVASIKNFKSVLYYQNNVFMSEAK |
| | | CWTETDLTKGPHEFCSQHTMLVKQGDDYVY |
| | | LPYPDPSRILGAGCFVDDIVKTDGTLMIER |
| | | FVSLAIDAYPLTKHPNQEYADVFHLYLQYI |
| | | RKLHDELTGHMLDMYSVMLINDNTSRYWEP |
| | | EFYEAMYTPHTVLQAVGACVLCNSQTSLRC |
| | | GACIRRPFLCCKCCYDHVISTSHKLVLSVN |
| | | PYVCNAPGCDVTDVTQLYLGGMSYYCKSHK |
| | | PPISFPLCANGQVFGLYKNTCVGSDNVTDF |
| | | NAIATCDWTNAGDYILANTCTERLKLFAAE |
| | | TLKATEETFKLSYGIATVREVLSDRELHLS |
| | | WEVGKPRPPLNRNYVFTGYRVTKNSKVQIG |
| | | EYTFEKGDYGDAVVYRGTTTYKLNVGDYFV |
| | | LTSHTVMPLSAPTLVPQEHYVRITGLYPTL |
| | | NISDEFSSNVANYQKVGMQKYSTLQGPPGT |
| | | GKSHFAIGLALYYPSARIVYTACSHAAVDA |
| | | LCEKALKYLPIDKCSRIIPARARVECFDKF |
| | | KVNSTLEQYVFCTVNALPETTADIVVFDEI |
| | | SMATNYDLSVVNARLRAKHYVYIGDPAQLP |
| | | APRTLLTKGTLEPEYFNSVCRLMKTIGPDM |
| | | FLGTCRRCPAEIVDTVSALVYDNKLKAHKD |
| | | KSAQCFKMFYKGVITHDVSSAINRPQIGVV |
| | | REFLTRNPAWRKAVFISPYNSQNAVASKIL |
| | | GLPTQTVDSSQGSEYDYVIFTQTTETAHSC |
| | | NVNRFNVAITRAKVGILCIMSDRDLYDKLQ |
| | | FTSLEIPRRNVATLQAENVTGLFKDCSKVI |
| | | TGLHPTQAPTHLSVDTKFKTEGLCVDIPGI |
| | | PKDMTYRRLISMMGFKMNYQVNGYPNMFIT |
| | | REEAIRHVRAWIGFDVEGCHATREAVGTNL |
| | | PLQLGFSTGVNLVAVPTGYVDTPNNTDFSR |
| | | VSAKPPPGDQFKHLIPLMYKGLPWNVVRIK |
| | | IVQMLSDTLKNLSDRVVFVLWAHGFELTSM |
| | | KYFVKIGPERTCCLCDRRATCFSTASDTYA |
| | | CWHHSIGFDYVYNPFMIDVQQWGFTGNLQS |
| | | NHDLYCQVHGNAHVASCDAIMTRCLAVHEC |
| | | FVKRVDWTIEYPIIGDELKINAACRKVQHM |
| | | VVKAALLADKFPVLHDIGNPKAIKCVPQAD |
| | | VEWKFYDAQPCSDKAYKIEELFYSYATHSD |
| | | KFTDGVCLFWNCNVDRYPANSIVCRFDTRV |
| | | LSNLNLPGCDGGSLYVNKHAFHTPAFDKSA |
| | | FVNLKQLPFFYYSDSPCESHGKQVVSDIDY |
| | | VPLKSATCITRCNLGGAVCRHHANEYRLYL |
| | | DAYNMMISAGFSLWVYKQFDTYNLWNTFTR |
| | | LQSLENVAFNVVNKGHFDGQQGEVPVSIIN |
| | | NTVYTKVDGVDVELFENKTTLPVNVAFELW |
| | | AKRNIKPVPEVKILNNLGVDIAANTVIWDY |
| | | KRDAPAHISTIGVCSMTDIAKKPTETICAP |
| | | LTVFFDGRVDGQVDLFRNARNGVLITEGSV |
| | | KGLQPSVGPKQASLNGVTLIGEAVKTQFNY |
| | | YKKVDGVVQQLPETYFTQSRNLQEFKPRSQ |
| | | MEIDFLELAMDEFIERYKLEGYAFEHIVYG |
| | | DFSHSQLGGLHLLIGLAKRFKESPFELEDF |
| | | IPMDSTVKNYFITDAQTGSSKCVCSVIDLL |
| | | LDDFVEIIKSQDLSVVSKVVKVTIDYTEIS |
| | | FMLWCKDGHVETFYPKLQSSQAWQPGVAMP |
| | | NLYKMQRMLLEKCDLQNYGDSATLPKGIMM |
| | | NVAKYTQLCQYLNTLTLAVPYNMRVIHFGA |
| | | GSDKGVAPGTAVLRQWLPTGTLLVDSDLND |
| | | FVSDADSTLIGDCATVHTANKWDLIISDMY |
| | | DPKTKNVTKENDSKEGFFTYICGFIQQKLA |
| | | LGGSVAIKITEHSWNADLYKLMGHFAWWTA |
| | | FVTNVNASSSEAFLIGCNYLGKPREQIDGY |
| | | VMHANYIFWRNTNPIQLSSYSLFDMSKFPL |
| | | KLRGTAVMSLKEGQINDMILSLLSKGRLII |
| | | RENNRVVISSDVLVNN |
| surface | 165 | mfvflvllpl vssqcvnltt rtqlppaytn sftrgvyypd kvfrssvlhs tqdlflpffs 61 |
| glycoprotein | | nvtwfhaihv sgtngtkrfd npvlpfndgv yfasteksni irgwifgttl dsktqslliv 121 |
| [Wuhan | | nnatnvvikv cefqfondpf lgvyyhknnk swmesefrvy ssannctfey vsqpflmdle 181 |
| seafood | | gkqgnfknlr efvfknidgy fkiyskhtpi nlvrdlpqgf saleplvdlp iginitrfqt 241 |
| market | | llalhrsylt pgdsssgwta gaaayyvgyl qprtfllkyn engtitdavd caldplsetk 301 |
| pneumonia | | ctlksftvek giyqtsnfrv qptesivrfp nitnlcpfge vfnatrfasv yawnrkrisn 361 |
| virus]; | | cvadysvlyn sasfstfkcy gvsptklndl cftnvyadsf virgdevrqi apgqtgkiad 421 |
| GenBank: | | ynyklpddft gcviawnsnn ldskvggnyn ylyrlfrksn lkpferdist eiyqagstpc 481 |
| QHD43416.1; | | ngvegfncyf plqsygfqpt ngvgyqpyrv vvlsfellha patvcgpkks tnlvknkcvn 541 |
| Jan. 23, | | fnfngltgtg vltesnkkfl pfqqfgrdia dttdavrdpq tleilditpc sfggvsvitp 601 |
| 2020 | | gtntsnqvav lyqdvnctev pvaihadqlt ptwrvystgs nvfqtragcl igaehvnnsy 661 |
| | | ecdipigagi casyqtqtns prrarsvasq siiaytmslg aensvaysnn siaiptnfti 721 |
| | | svtteilpvs mtktsvdctm yicgdstecs nlllqygsfc tqlnraltgi aveqdkntqe 781 |
| | | vfaqvkqiyk tppikdfggf nfsqilpdps kpskrsfied llfnkvtlad agfikqygdc 841 |
| | | lgdiaardli caqkfngltv lpplltdemi aqytsallag titsgwtfga gaalqipfam 901 |
| | | qmayrfngig vtqnvlyenq klianqfnsa igkiqdslss tasalgklqd vvnqnaqaln 961 |
| | | tlvkqlssnf gaissvlndi lsrldkveae vqidrlitgr lqslqtyvtq qliraaeira 1021 |
| | | sanlaatkms ecvlgqskrv dfcgkgyhlm sfpqsaphgv vflhvtyvpa qeknfttapa 1081 |
| | | ichdgkahfp regvfvsngt hwfvtqrnfy epqiittdnt fvsgncdvvi givnntvydp 1141 |
| | | lqpeldsfke eldkyfknht spdvdlgdis ginasvvniq keidrlneva knlneslidl 1201 |
| | | qelgkyeqyi kwpwyiwlgf iagliaivmv timlccmtsc csclkgccsc gscckfdedd 1261 |
| | | sepvlkgvkl hyt |
| surface | 166 | nitnlcpfgevfnatrfasvyawnrkrisn |
| glycoprotein | | cvadysvlynsasfstfkcygvsptklndl |
| RBD [Wuhan | | cftnvyadsfvirgdevrqiapgqtgkiad |
| seafood | | ynyklpddftgcviawnsnnldskvggnyn |
| market | | ylyrlfrksnlkpferdisteiyqagstpc |
| pneumonia | | ngvegfncyfplqsygfqptngvgyqpyrv |
| virus]; | | vvlsfellhapatvcgpkkstnlvknkcvn |
| GenBank: | | fnfngltgtg |
| QHD43416.1; | | |
| Jan. 23, | | |
| 2020 | | |
| Receptor | 167 | Nsnnldskvggnynylyrlfrksnlkpfer |
| Binding Motif | | disteiyqagstpcngvegfncyfplqsyg |
| (RBM) in | | fqptngvgyqpy |
| surface | | |
| glycoprotein | | |
| RBD [Wuhan | | |
| seafood | | |
| market | | |
| pneumonia | | |
| virus]; | | |
| GenBank: | | |
| QHD43416.1; | | |
| Jan. 23, | | |
| 2020 | | |
| SARS-CoV-2 | 168 | EIVLTQSPGTLSLSPGERATLSCRASQTVS |
| S309-v13 | | STSLAWYQQKPGQAPRLLIYGASSRATGIP |
| mAb VL | | DRFSGSGSGTDFTLTISRLEPEDFAVYYCQ |
| (VK) (aa) | | QHDTSLTFGGGTKVEIK |
| SARS-CoV-2 | 169 | QTVSSTS |
| S309-v13 | | |
| mAb CDRL1 | | |
| (aa) | | |
| SARS-CoV-2 | 170 | GAS |
| S309-v13 | | |
| mAb CDRL2 | | |
| (aa) | | |
| SARS-CoV-2 | 171 | QQHDTSLT |
| S309-v13 | | |
| mAb CDRL3 | | |
| (aa) | | |
| SARS-CoV-2 | 172 | QVQLVQSGAEVKKPGASVKVSCKASGYPFT |
| S309-v2.9 | | SYGISWVRQAPGQGLEWMGFISTYNANTNY |
| mAb VH (aa) | | AQKFQGRVTMTTDTSTTTGYMELRRLRSDD |
| | | TAVYYCARDYTRGAFFGESLIGGFDNWGQG |
| | | TLVTVSS |
| SARS-CoV-2 | 173 | ASTKGPSVFPLAPSSKSTSGGTAALGCLVK |
| CH1-CH3 | | DYFPEPVTVSWNSGALTSGVHTFPAVLQSS |
| G1m17; | | GLYSLSSVVTVPSSSLGTQTYICNVNHKPS |
| IgG1*01 LS | | NTKVDKKVEPKSCDKTHTCPPCPAPELLGG |
| (aa) | | PSVFLFPPKPKDTLMISRTPEVTCVVVDVS |
| | | HEDPEVKFNWYVDGVEVHNAKTKPREEQYN |
| | | STYRVVSVLTVLHQDWLNGKEYKCKVSNKA |
| | | LPAPIEKTISKAKGQPREPQVYTLPPSRDE |
| | | LTKNQVSLTCLVKGFYPSDIAVEWESNGQP |
| | | ENNYKTTPPVLDSDGSFFLYSKLTVDKSRW |
| | | QQGNVFSCSVLHEALHSHYTQKSLSLSPGK |
| SARS-CoV-2 | 174 | RTVAAPSVFIFPPSDEQLKSGTASVVCLLN |
| mAb CL (Ck) | | NFYPREAKVQWKVDNALQSGNSQESVTEQD |
| IgKC*01 | | SKDSTYSLSSTLTLSKADYEKHKVYACEVT |
| klm3 (aa) | | HQGLSSPVTKSFNRGEC |
| SARS-CoV-2 | 175 | ASTKGPSVFPLAPSSKSTSGGTAALGCLV |
| CH1-CH3 | | KDYFPEPVTVSWNSGALTSGVHTFPAVLQS |
| G1m17; | | SGLYSLSSVVTVPSSSLGTQTYICNVNHKP |
| IgG1*01 LS | | SNTKVDKKVEPKSCDKTHTCPPCPAPELLA |
| GAALIE (aa) | | GPSVFLFPPKPKDTLMISRTPEVTCVVVDV |
| | | SHEDPEVKFNWYVDGVEVHNAKTKPREEQY |
| | | NSTYRVVSVLTVLHQDWLNGKEYKCKVSNK |
| | | ALPLPEEKTISKAKGQPREPQVYTLPPSRD |
| | | ELTKNQVSLTCLVKGFYPSDIAVEWESNG |
| | | QPENNYKTTPPVLDSDGSFFLYSKLTVDKS |
| | | RWQQGNVFSCSVLHEALHSHYTQKSLSLSP |
| | | GK |
| SARS-CoV-2 | 176 | QVQLVQSGAEVKKPGASVKVSCKASGYTFT |
| S300-v2.10 | | DYYIHWVRQAPGQGPEWLGFVNAYSGATRY |
| mAb VH (aa) | | AQKYQGRVTMTRDTSISTAYMQLSRLRPDD |
| | | TAVYYCARDRPSHEFAMYFFDNWGQGTLVT |
| | | VSS |
| SARS-CoV-2 | 177 | QVQLVQSGAEVKKPGASVKVSCKASGYTFT |
| S300-v2.11 | | DYYIHWVRQAPGQGPEWLGFVQGYSGATRY |
| mAb VH (aa) | | AQKYQGRVTMTRDTSISTAYMQLSRLRPDD |
| | | TAVYYCARDRPSHEFAMYFFDNWGQGTLVT |
| | | VSS |
| SARS-CoV-2 | 178 | EVQLVESGGGLVQPGGSLRLSCAASGFTFS |
| S315-v1 mAb | | NYWMTWVRQAPGKGLEWVANIKQDGSEKYY |
| VH (aa) | | VDSVKGRFTISRDNAKNSLYLQMNSLRAED |
| | | TAVYYCARDLWWNDQAHYYGMDVWGQGTTV |
| | | TVSS |
| SARS-CoV-2 | 179 | GFTFSNYW |
| S315-v1 mAb | | |
| CDRH1 (aa) | | |
| SARS-CoV-2 | 180 | IKQDGSEK |
| S315-v1 mAb | | |
| CDRH2 (aa) | | |
| SARS-CoV-2 | 181 | ARDLWWNDQAHYYGMDV |
| S315-v1 mAb | | |
| CDRH3 (aa) | | |
| SARS-CoV-2 | 182 | SYELTQPPSVSVSPGQTARITCSGDAFPNQ |
| S315-v1 mAb | | YAYWYQQKPGQAPVMLIYKDSERPSGIPER |
| VL (aa) | | FFGSSSGTTVTLTIRGVQAEDEADYYCQSA |
| | | DSSGTVFGGGTKLTVL |
| SARS-CoV-2 | 183 | AFPNQY |
| S315-v1 mAb | | |
| CDRL1 (aa) | | |
| SARS-CoV-2 | 184 | KDS |
| S315-v1 mAb | | |
| CDRL2 (aa) | | |
| SARS-CoV-2 | 185 | QSADSSGTV |
| S315-v1 mAb | | |
| CDRL3 (aa) | | |
| SARS-CoV-2 | 186 | gaggtgcagctggtggagtctgggggaggc |
| S315-v1 mAb | | ttggtccagcctggggggtccctgagactc |
| VH (nt-wt) | | tcctgtgcagcctctggattcacctttagt |
| | | aattattggatgacctgggtccgccaggct |
| | | ccagggaaggggctggagtgggtggccaac |
| | | ataaagcaagatggaagtgagaaatactat |
| | | gtggactctgtgaagggccgattcaccatc |
| | | tccagagacaacgccaagaactcactgtat |
| | | ctgcaaatgaacagcctgagagccgaggac |
| | | acggctgtgtattactgtgcgagagatctt |
| | | tggtggaacgaccaggctcactactacggt |
| | | atggacgtctggggccaagggaccacggtc |
| | | accgtctcctcag |
| SARS-CoV-2 | 187 | tcctatgagctgacacagccaccctcggtg |
| S315-v1 mAb | | tcagtgtccccaggacagacggccaggatc |
| VL (nt-wt) | | acctgctctggagatgcattcccaaaccaa |
| | | tatgcttattggtaccagcagaagccaggc |
| | | caggcccctgtgatgctgatctataaagac |
| | | agtgagaggccctcagggatccctgagcga |
| | | ttctttggctccagctcagggacaacagtc |
| | | acgttgaccatcagaggagtccaggcagaa |
| | | gacgaggctgactattactgtcaatcagca |
| | | gacagcagtggtaccgtgttcggcggaggg |
| | | accaagctgaccgtcctag |
| SARS-CoV-2 | 188 | GAAGTGCAGCTTGTCGAGAGCGGCGGAGGC |
| S315-v1 mAb | | CTCGTTCAGCCAGGTGGGAGTCTCCGTCTT |
| VH (nt- | | TCATGCGCCGCTTCAGGATTTACGTTCTCC |
| codon | | AACTACTGGATGACATGGGTGAGGCAGGCA |
| optimized) | | CCTGGGAAGGGGCTGGAGTGGGTGGCTAAC |
| | | ATCAAGCAGGACGGATCTGAAAAATATTAT |
| | | GTAGATTCTGTGAAGGGGCGGTTTACCATC |
| | | TCAAGGGATAATGCCAAAAACTCTTTGTAT |
| | | TTACAGATGAACTCTCTTCGAGCCGAGGAC |
| | | ACCGCCGTTTACTACTGTGCCCGAGATCTA |
| | | TGGTGGAATGACCAGGCTCACTATTATGGA |
| | | ATGGACGTGTGGGGCCAGGGTACTACCGTT |
| | | ACCGTCTCCTCA |
| SARS-CoV-2 | 189 | TCTTACGAGCTCACCCAGCCACCCTCAGTG |
| S315-v1 mAb | | TCAGTGAGCCCTGGCCAAACAGCTCGCATC |
| VL (nt-codon | | ACCTGTTCAGGTGACGCCTTTCCAAATCAG |
| optimized) | | TACGCCTACTGGTATCAGCAGAAACCCGGC |
| | | CAGGCACCCGTTATGCTCATCTACAAAGAT |
| | | TCTGAGCGGCCATCCGGTATCCCCGAACGC |
| | | TTTTTCGGAAGCTCCAGTGGGACTACAGTT |
| | | ACACTTACTATCCGGGGAGTGCAAGCTGAA |
| | | GATGAGGCCGACTATTATTGCCAGAGCGCA |
| | | GACTCCTCAGGCACAGTGTTTGGGGGCGGG |
| | | ACTAAACTAACTGTGCTG |
| SARS-CoV-2 | 190 | SYELTQPPSVSVSPGQTARITCSGDAFPNQ |
| S315-v2 mAb | | YAYWYQQKPGQAPVMLIYKDSERPSGIPER |
| VL (aa) | | FFGSSSGTTVTLTISGVQAEDEADYYCQSA |
| | | DSSGTVFGGGTKLTVL |
| SARS-CoV-2 | 191 | tcctatgagctgacacagccaccctcggtg |
| S315-v2 mAb | | tcagtgtccccaggacagacggccaggatc |
| VL (nt-wt) | | acctgctctggagatgcattcccaaaccaa |
| | | tatgcttattggtaccagcagaagccaggc |
| | | caggcccctgtgatgctgatctataaagac |
| | | agtgagaggccctcagggatccctgagcga |
| | | ttctttggctccagctcagggacaacagtc |
| | | acgttgaccatcagtggagtccaggcagaa |
| | | gacgaggctgactattactgtcaatcagca |
| | | gacagcagtggtaccgtgttcggcggaggg |
| | | accaagctgaccgtcctag |
| SARS-CoV-2 | 192 | TCCTACGAGCTCACCCAGCCCCCCTCAGTC |
| S315-v2 mAb | | TCTGTGTCTCCTGGACAGACAGCCAGAATC |
| VL (nt- | | ACCTGCTCGGGAGATGCTTTTCCCAACCAA |
| codon | | TACGCCTACTGGTACCAACAGAAACCAGGT |
| optimized) | | CAGGCGCCTGTCATGCTGATTTATAAAGAC |
| | | TCAGAGCGGCCTTCAGGAATTCCCGAAAGA |
| | | TTCTTCGGGAGTTCAAGCGGAACTACCGTG |
| | | ACCTTAACCATAAGCGGGGTGCAGGCCGAA |
| | | GATGAAGCAGACTATTATTGCCAGAGTGCC |
| | | GATAGTAGTGGCACAGTCTTTGGGGGGGGG |
| | | ACAAAGCTGACAGTACTC |
| SARS-CoV-2 | 193 | GQPKAAPSVTLFPPSSEELQANKATLVCLI |
| mAb CL | | SDFYPGAVTVAWKADSSPVKAGVETTTPSK |
| IgLC*01 | | QSNNKYAASSYLSLTPEQWKSHRSYSCQVT |
| | | HEGSTVEKTVAPTECS |
| SARS-CoV-2 | 194 | EVQLVESGGGLVQPGGSLRLSCAASGFTFS |
| S315-v3 mAb | | NYFMTWVRQAPGKGLEWVANIKQDGSEKYY |
| VH (aa) | | VDSVKGRFTISRDNAKNSLYLQMNSLRAED |
| | | TAVYYCARDLWWNDQAHYYGMDVWGQGTTV |
| | | TVSS |
| SARS-CoV-2 | 195 | GFTFSNYF |
| S315-v3 mAb | | |
| CDRH1 (aa) | | |
| SARS-CoV-2 | 196 | EVQLVESGGGLVQPGGSLRLSCAASGFTFS |
| S315-v4 mAb | | NYWMTWVRQAPGKGLEWVANIKQDASEKYY |
| VH (aa) | | VDSVKGRFTISRDNAKNSLYLQMNSLRAED |
| | | TAVYYCARDLWWNDQAHYYGMDVWGQGTTV |
| | | TVSS |
| SARS-CoV-2 | 197 | IKQDASEK |
| S315-v4 mAb | | |
| CDRH2 (aa) | | |
| SARS-CoV-2 | 198 | EVQLVESGGGLVQPGGSLRLSCAASGFTFS |
| S315-v5 mAb | | NYWMTWVRQAPGKGLEWVANIKQEGSEKYY |
| VH (aa) | | VDSVKGRFTISRDNAKNSLYLQMNSLRAED |
| | | TAVYYCARDLWWNDQAHYYGMDVWGQGTTV |
| | | TVSS |
| SARS-CoV-2 | 199 | IKQEGSEK |
| S315-v5 mAb | | |
| CDRH2 (aa) | | |
| SARS-CoV-2 | 200 | EVQLVESGGGLVQPGGSLRLSCAASGFTFS |
| S315-v6 mAb | | NYWMTWVRQAPGKGLEWVANIKQDGSEKYY |
| VH (aa) | | VDSVKGRFTISRDNAKNSLYLQMNSLRAED |
| | | TAVYYCARDLFWNDQAHYYGMDVWGQGTTV |
| | | TVSS |
| SARS-CoV-2 | 201 | ARDLFWNDQAHYYGMDV |
| S315-v6 mAb | | |
| CDRH3 (aa) | | |
| SARS-CoV-2 | 202 | EVQLVESGGGLVQPGGSLRLSCAASGFTFS |
| S315-v7 mAb | | NYWMTWVRQAPGKGLEWVANIKQDGSEKYY |
| VH (aa) | | VDSVKGRFTISRDNAKNSLYLQMNSLRAED |
| | | TAVYYCARDLWFNDQAHYYGMDVWGQGTTV |
| | | TVSS |
| SARS-CoV-2 | 203 | ARDLWFNDQAHYYGMDV |
| S315-v7 mAb | | |
| CDRH3 (aa) | | |
| SARS-CoV-2 | 204 | QVQLVQSGAEVKKPGASVKVSCKASGYPFT |
| Heavy Chain | | SYGISWVRQAPGQGLEWMGWISTYNGNTNY |
| IgHG1*01 Fd | | AQKFQGRVTMTTDTSTTTGYMELRRLRSDD |
| (aa) | | TAVYYCARDYTRGAWFGESLIGGFDNWGQG |
| | | TLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYIC |
| | | NVNHKPSNTKVDKRVEPKSC |
| SARS-CoV-2 | 205 | EIVLTQSPGTLSLSPGERATLSCRASQTVS |
| Light Chain | | STSLAWYQQKPGQAPRLLIYGASSRATGIP |
| IgKC*01 (aa) | | DRFSGSGSGTDFTLTISRLEPEDFAVYYCQ |
| | | QHDTSLTFGGGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| Linker (aa) | 206 | GSTSGSGKPGSGEGSTKG |
| Linker (aa) | 207 | GSGKPGSGEG |
| Linker (aa) | 208 | GKPGSGEG |
| Linker (aa) | 209 | SGKPGSGE |
| Linker (aa) | 210 | BPXXXZ, wherein each X is independently a |
| | | glycine (G) or serine (S), B is a positively charged |
| | | amino acid and Z is glycine (G) or a negatively |
| | | charged amino acid |
| Linker (aa) | 211 | (GxS)y, wherein x is 1-10 and y is 1-10 |
| Linker (aa) | 212 | GGGGSGGGGSGGGGS |
| Linker (aa) | 213 | GGGGSGGGGSGGGGSGGGGSGGGGS |
| | | GGGGSGGGGSGGGGSGGGGSGGGGS |
| Linker (aa) | 214 | GSTSGGGSGGGSGGGGSS |
| Linker (aa) | 215 | EGKSSGSGSESKVD |
| Linker (aa) | 216 | KESGSVSSEQLAQFRSLD |
| Linker (aa) | 217 | GGGGS |
| SARS-CoV-2 | 218 | QVQLVQSGAEVKKPGASVKVSCKASGYPFT |
| S309-scFab | | SYGISWVRQAPGQGLEWMGWISTYNGNTNY |
| (H-L) (aa) | | AQKFQGRVTMTTDTSTTTGYMELRRLRSDD |
| | | TAVYYCARDYTRGAWFGESLIGGFDNWGQG |
| | | TLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYIC |
| | | NVNHKPSNTKVDKRVEPKSCGGGGSGGGGS |
| | | GGGGSGGGGSGGGGSGGGGSGGGGSGGGGS |
| | | GGGGSGGGGSEIVLTQSPGTLSLSPGERAT |
| | | LSCRASQTVSSTSLAWYQQKPGQAPRLLIY |
| | | GASSRATGIPDRFSGSGSGTDFTLTISRLE |
| | | PEDFAVYYCQQHDTSLTFGGGTKVEIKRTV |
| | | AAPSVFIFPPSDEQLKSGTASVVCLLNNFY |
| | | PREAKVQWKVDNALQSGNSQESVTEQDSKD |
| | | STYSLSSTLTLSKADYEKHKVYACEVTHQG |
| | | LSSPVTKSFNRGEC |
| SARS-CoV-2 | 219 | EIVLTQSPGTLSLSPGERATLSCRASQTVS |
| S309-scFab | | STSLAWYQQKPGQAPRLLIYGASSRATGIP |
| (L-H) (aa) | | DRFSGSGSGTDFTLTISRLEPEDFAVYYCQ |
| | | QHDTSLTFGGGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGECGGGGSGGGGSGGGGSGGGGSGGGGSG |
| | | GGGSGGGGSGGGGSGGGGSGGGGSQVQLVQ |
| | | SGAEVKKPGASVKVSCKASGYPFTSYGISW |
| | | VRQAPGQGLEWMGWISTYNGNTNYAQKFQG |
| | | RVTMTTDTSTTTGYMELRRLRSDDTAVYYC |
| | | ARDYTRGAWFGESLIGGFDNWGQGTLVTVS |
| | | SASTKGPSVFPLAPSSKSTSGGTAALGCLV |
| | | KDYFPEPVTVSWNSGALTSGVHTFPAVLQS |
| | | SGLYSLSSVVTVPSSSLGTQTYICNVNHKP |
| | | SNTKVDKRVEPKSC |
| SARS-CoV-2 | 220 | QVQLVQSGAEVKKPGASVKVSCKASGYPFT |
| S309-scFv | | SYGISWVRQAPGQGLEWMGWISTYNGNTNY |
| (VH-VL) (aa) | | AQKFQGRVTMTTDTSTTTGYMELRRLRSDD |
| | | TAVYYCARDYTRGAWFGESLIGGFDNWGQG |
| | | TLVTVSSGGGGSGGGGSGGGGSEIVLTQSP |
| | | GTLSLSPGERATLSCRASQTVSSTSLAWYQ |
| | | QKPGQAPRLLIYGASSRATGIPDRFSGSGS |
| | | GTDFTLTISRLEPEDFAVYYCQQHDTSLTF |
| | | GGGTKVEIK |
| SARS-CoV-2 | 221 | EIVLTQSPGTLSLSPGERATLSCRASQTVS |
| S309-scFv | | STSLAWYQQKPGQAPRLLIYGASSRATGIP |
| (VL-VH) (aa) | | DRFSGSGSGTDFTLTISRLEPEDFAVYYCQ |
| | | QHDTSLTFGGGTKVEIKGGGGSGGGGSGGG |
| | | GSQVQLVQSGAEVKKPGASVKVSCKASGYP |
| | | FTSYGISWVRQAPGQGLEWMGWISTYNGNT |
| | | NYAQKFQGRVTMTTDTSTTTGYMELRRLRS |
| | | DDTAVYYCARDYTRGAWFGESLIGGFDNWG |
| | | QGTLVTVSS |
| SARS-CoV-2 | 222 | QVQLVQSGAEVKKPGASVKVSCKASGYPFT |
| S309-scFv | | SYGISWVRQAPGQGLEWMGWISTYNGNTNY |
| (VH-VL)- | | AQKFQGRVTMTTDTSTTTGYMELRRLRSDD |
| (VH-VL) (aa) | | TAVYYCARDYTRGAWFGESLIGGFDNWGQG |
| | | TLVTVSSGGGGSGGGGSGGGGSEIVLTQSP |
| | | GTLSLSPGERATLSCRASQTVSSTSLAWYQ |
| | | QKPGQAPRLLIYGASSRATGIPDRFSGSGS |
| | | GTDFTLTISRLEPEDFAVYYCQQHDTSLTF |
| | | GGGTKVEIKGGGGSGGGGSGGGGSGGGGSQ |
| | | VQLVQSGAEVKKPGASVKVSCKASGYPFTS |
| | | YGISWVRQAPGQGLEWMGWISTYNGNTNYA |
| | | QKFQGRVTMTTDTSTTTGYMELRRLRSDDT |
| | | AVYYCARDYTRGAWFGESLIGGFDNWGQGT |
| | | LVTVSSGGGGSGGGGSGGGGSEIVLTQSPG |
| | | TLSLSPGERATLSCRASQTVSSTSLAWYQQ |
| | | KPGQAPRLLIYGASSRATGIPDRFSGSGSG |
| | | TDFTLTISRLEPEDFAVYYCQQHDTSLTFG |
| | | GGTKVEIK |
| SARS-CoV-2 | 223 | QVQLVQSGAEVKKPGASVKVSCKASGYPFT |
| S309-scFv- | | SYGISWVRQAPGQGLEWMGWISTYNGNTNY |
| (VH-VL)- | | AQKFQGRVTMTTDTSTTTGYMELRRLRSDD |
| (VL-VH) (aa) | | TAVYYCARDYTRGAWFGESLIGGFDNWGQG |
| | | TLVTVSSGGGGSGGGGSGGGGSEIVLTQSP |
| | | GTLSLSPGERATLSCRASQTVSSTSLAWYQ |
| | | QKPGQAPRLLIYGASSRATGIPDRFSGSGS |
| | | GTDFTLTISRLEPEDFAVYYCQQHDTSLTF |
| | | GGGTKVEIKGGGGSGGGGSGGGGSGGGGSE |
| | | IVLTQSPGTLSLSPGERATLSCRASQTVSS |
| | | TSLAWYQQKPGQAPRLLIYGASSRATGIPD |
| | | RFSGSGSGTDFTLTISRLEPEDFAVYYCQQ |
| | | HDTSLTFGGGTKVEIKGGGGSGGGGSGGGG |
| | | SQVQLVQSGAEVKKPGASVKVSCKASGYPF |
| | | TSYGISWVRQAPGQGLEWMGWISTYNGNTN |
| | | YAQKFQGRVTMTTDTSTTTGYMELRRLRSD |
| | | DTAVYYCARDYTRGAWFGESLIGGFDNWGQ |
| | | GTLVTVSS |
| SARS-CoV-2 | 224 | EIVLTQSPGTLSLSPGERATLSCRASQTVS |
| S309-scFv- | | STSLAWYQQKPGQAPRLLIYGASSRATGIP |
| (VL-VH)- | | DRFSGSGSGTDFTLTISRLEPEDFAVYYCQ |
| (VH-VL) (aa) | | QHDTSLTFGGGTKVEIKGGGGSGGGGSGGG |
| | | GSQVQLVQSGAEVKKPGASVKVSCKASGYP |
| | | FTSYGISWVRQAPGQGLEWMGWISTYNGNT |
| | | NYAQKFQGRVTMTTDTSTTTGYMELRRLRS |
| | | DDTAVYYCARDYTRGAWFGESLIGGFDNWG |
| | | QGTLVTVSSGGGGSGGGGSGGGGSGGGGS |
| | | QVQLVQSGAEVKKPGASVKVSCKASGYPFT |
| | | SYGISWVRQAPGQGLEWMGWISTYNGNTNY |
| | | AQKFQGRVTMTTDTSTTTGYMELRRLRSDD |
| | | TAVYYCARDYTRGAWFGESLIGGFDNWGQG |
| | | TLVTVSSGGGGSGGGGSGGGGSEIVLTQSP |
| | | GTLSLSPGERATLSCRASQTVSSTSLAWYQ |
| | | QKPGQAPRLLIYGASSRATGIPDRFSGSGS |
| | | GTDFTLTISRLEPEDFAVYYCQQHDTSLTF |
| | | GGGTKVEIK |
| SARS-CoV-2 | 225 | EIVLTQSPGTLSLSPGERATLSCRASQTVS |
| S309-scFv- | | STSLAWYQQKPGQAPRLLIYGASSRATGIP |
| (VL-VH)- | | DRFSGSGSGTDFTLTISRLEPEDFAVYYCQ |
| (VL-VH) (aa) | | QHDTSLTFGGGTKVEIKGGGGSGGGGSGGG |
| | | GSQVQLVQSGAEVKKPGASVKVSCKASGYP |
| | | FTSYGISWVRQAPGQGLEWMGWISTYNGNT |
| | | NYAQKFQGRVTMTTDTSTTTGYMELRRLRS |
| | | DDTAVYYCARDYTRGAWFGESLIGGFDNWG |
| | | QGTLVTVSSGGGGSGGGGSGGGGSGGGGS |
| | | EIVLTQSPGTLSLSPGERATLSCRASQTVS |
| | | STSLAWYQQKPGQAPRLLIYGASSRATGIP |
| | | DRFSGSGSGTDFTLTISRLEPEDFAVYYCQ |
| | | QHDTSLTFGGGTKVEIKGGGGSGGGGSGGG |
| | | GSQVQLVQSGAEVKKPGASVKVSCKASGYP |
| | | FTSYGISWVRQAPGQGLEWMGWISTYNGNT |
| | | NYAQKFQGRVTMTTDTSTTTGYMELRRLRS |
| | | DDTAVYYCARDYTRGAWFGESLIGGFDNWG |
| | | QGTLVTVSS |
| SARS-CoV-2 | 226 | QVQLVQSGAEVKKPGASVKVSCKASGYPFT |
| S309-scFab- | | SYGISWVRQAPGQGLEWMGWISTYQGNTNY |
| (H-L) v1.1 | | AQKFQGRVTMTTDTSTTTGYMELRRLRSDD |
| (aa) | | TAVYYCARDYTRGAWFGESLIGGFDNWGQG |
| | | TLVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYIC |
| | | NVNHKPSNTKVDKRVEPKSCGGGGSGGGGS |
| | | GGGGSGGGGSGGGGSGGGGSGGGGSGGGGS |
| | | GGGGSGGGGSEIVLTQSPGTLSLSPGERAT |
| | | LSCRASQTVSSTSLAWYQQKPGQAPRLLIY |
| | | GASSRATGIPDRFSGSGSGTDFTLTISRLE |
| | | PEDFAVYYCQQHDTSLTFGGGTKVEIKRTV |
| | | AAPSVFIFPPSDEQLKSGTASVVCLLNNFY |
| | | PREAKVQWKVDNALQSGNSQESVTEQDSKD |
| | | STYSLSSTLTLSKADYEKHKVYACEVTHQG |
| | | LSSPVTKSFNRGEC |
| SARS-CoV-2 | 227 | EIVLTQSPGTLSLSPGERATLSCRASQTVS |
| S309-scFab- | | STSLAWYQQKPGQAPRLLIYGASSRATGIP |
| (L-H) v1.1 | | DRFSGSGSGTDFTLTISRLEPEDFAVYYCQ |
| (aa) | | QHDTSLTFGGGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGECGGGGGGGGSGGGGSGGGGSGGGGSGG |
| | | GGSGGGGSGGGGSGGGGSGGGGSQVQLVQS |
| | | GAEVKKPGASVKVSCKASGYPFTSYGISWV |
| | | RQAPGQGLEWMGWISTYQGNTNYAQKFQGR |
| | | VTMTTDTSTTTGYMELRRLRSDDTAVYYCA |
| | | RDYTRGAWFGESLIGGFDNWGQGTLVTVSS |
| | | ASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPS |
| | | NTKVDKRVEPKSC |
| SARS-CoV-2 | 228 | QVQLVQSGAEVKKPGASVKVSCKASGYPFT |
| S309-scFv- | | SYGISWVRQAPGQGLEWMGWISTYQGNTNY |
| (VH-VL) v1.1 | | AQKFQGRVTMTTDTSTTTGYMELRRLRSDD |
| (aa) | | TAVYYCARDYTRGAWFGESLIGGFDNWGQG |
| | | TLVTVSSGGGGSGGGGSGGGGSEIVLTQSP |
| | | GTLSLSPGERATLSCRASQTVSSTSLAWYQ |
| | | QKPGQAPRLLIYGASSRATGIPDRFSGSGS |
| | | GTDFTLTISRLEPEDFAVYYCQQHDTSLTF |
| | | GGGTKVEIK |
| SARS-CoV-2 | 229 | EIVLTQSPGTLSLSPGERATLSCRASQTVS |
| S309-scFv- | | STSLAWYQQKPGQAPRLLIYGASSRATGIP |
| (VL-VH) v1.1 | | DRFSGSGSGTDFTLTISRLEPEDFAVYYCQ |
| (aa) | | QHDTSLTFGGGTKVEIKGGGGSGGGGSGGG |
| | | GSQVQLVQSGAEVKKPGASVKVSCKASGYP |
| | | FTSYGISWVRQAPGQGLEWMGWISTYQGNT |
| | | NYAQKFQGRVTMTTDTSTTTGYMELRRLRS |
| | | DDTAVYYCARDYTRGAWFGESLIGGFDNWG |
| | | QGTLVTVSS |
| SARS-CoV-2 | 230 | QVQLVQSGAEVKKPGASVKVSCKASGYPFT |
| S309-scFv- | | SYGISWVRQAPGQGLEWMGWISTYQGNTNY |
| (VH-VL)- | | AQKFQGRVTMTTDTSTTTGYMELRRLRSDD |
| (VH-VL) v1.1 | | TAVYYCARDYTRGAWFGESLIGGFDNWGQG |
| (aa) | | TLVTVSSGGGGSGGGGSGGGGSEIVLTQSP |
| | | GTLSLSPGERATLSCRASQTVSSTSLAWYQ |
| | | QKPGQAPRLLIYGASSRATGIPDRFSGSGS |
| | | GTDFTLTISRLEPEDFAVYYCQQHDTSLTF |
| | | GGGTKVEIKGGGGSGGGGSGGGGSGGGGSQ |
| | | VQLVQSGAEVKKPGASVKVSCKASGYPFTS |
| | | YGISWVRQAPGQGLEWMGWISTYQGNTNYA |
| | | QKFQGRVTMTTDTSTTTGYMELRRLRSDDT |
| | | AVYYCARDYTRGAWFGESLIGGFDNWGQGT |
| | | LVTVSSGGGGSGGGGSGGGGSEIVLTQSPG |
| | | TLSLSPGERATLSCRASQTVSSTSLAWYQQ |
| | | KPGQAPRLLIYGASSRATGIPDRFSGSGSG |
| | | TDFTLTISRLEPEDFAVYYCQQHDTSLTFG |
| | | GGTKVEIK |
| SARS-CoV-2 | 231 | QVQLVQSGAEVKKPGASVKVSCKASGYPFT |
| S309-scFv- | | SYGISWVRQAPGQGLEWMGWISTYQGNTNY |
| (VH-VL)- | | AQKFQGRVTMTTDTSTTTGYMELRRLRSDD |
| (VL-VH) v1.1 | | TAVYYCARDYTRGAWFGESLIGGFDNWGQG |
| (aa) | | TLVTVSSGGGGSGGGGSGGGGSEIVLTQSP |
| | | GTLSLSPGERATLSCRASQTVSSTSLAWYQ |
| | | QKPGQAPRLLIYGASSRATGIPDRFSGSGS |
| | | GTDFTLTISRLEPEDFAVYYCQQHDTSLTF |
| | | GGGTKVEIKGGGGSGGGGSGGGGSGGGGSE |
| | | IVLTQSPGTLSLSPGERATLSCRASQTVSS |
| | | TSLAWYQQKPGQAPRLLIYGASSRATGIPD |
| | | RFSGSGSGTDFTLTISRLEPEDFAVYYCQQ |
| | | HDTSLTFGGGTKVEIKGGGGSGGGGSGGGG |
| | | SQVQLVQSGAEVKKPGASVKVSCKASGYPF |
| | | TSYGISWVRQAPGQGLEWMGWISTYQGNTN |
| | | YAQKFQGRVTMTTDTSTTTGYMELRRLRSD |
| | | DTAVYYCARDYTRGAWFGESLIGGFDNWGQ |
| | | GTLVTVSS |
| SARS-CoV-2 | 232 | EIVLTQSPGTLSLSPGERATLSCRASQTVS |
| S309-scFv- | | STSLAWYQQKPGQAPRLLIYGASSRATGIP |
| (VL-VH)- | | DRFSGSGSGTDFTLTISRLEPEDFAVYYCQ |
| (VH-VL) v1.1 | | QHDTSLTFGGGTKVEIKGGGGSGGGGSGGG |
| (aa) | | GSQVQLVQSGAEVKKPGASVKVSCKASGYP |
| | | FTSYGISWVRQAPGQGLEWMGWISTYQGNT |
| | | NYAQKFQGRVTMTTDTSTTTGYMELRRLRS |
| | | DDTAVYYCARDYTRGAWFGESLIGGFDNWG |
| | | QGTLVTVSSGGGGSGGGGSGGGGSGGGGSQ |
| | | VQLVQSGAEVKKPGASVKVSCKASGYPFTS |
| | | YGISWVRQAPGQGLEWMGWISTYQGNTNYA |
| | | QKFQGRVTMTTDTSTTTGYMELRRLRSDDT |
| | | AVYYCARDYTRGAWFGESLIGGFDNWGQGT |
| | | LVTVSSGGGGSGGGGSGGGGSEIVLTQSPG |
| | | TLSLSPGERATLSCRASQTVSSTSLAWYQQ |
| | | KPGQAPRLLIYGASSRATGIPDRFSGSGSG |
| | | TDFTLTISRLEPEDFAVYYCQQHDTSLTFG |
| | | GGTKVEIK |
| SARS-CoV-2 | 233 | EIVLTQSPGTLSLSPGERATLSCRASQTVS |
| S309-scFv- | | STSLAWYQQKPGQAPRLLIYGASSRATGIP |
| (VL-VH)- | | DRFSGSGSGTDFTLTISRLEPEDFAVYYCQ |
| (VL-VH) v1.1 | | QHDTSLTFGGGTKVEIKGGGGSGGGGSGGG |
| (aa) | | GSQVQLVQSGAEVKKPGASVKVSCKASGYP |
| | | FTSYGISWVRQAPGQGLEWMGWISTYQGNT |
| | | NYAQKFQGRVTMTTDTSTTTGYMELRRLRS |
| | | DDTAVYYCARDYTRGAWFGESLIGGFDNWG |
| | | QGTLVTVSSGGGGSGGGGSGGGGSGGGGSE |
| | | IVLTQSPGTLSLSPGERATLSCRASQTVSS |
| | | TSLAWYQQKPGQAPRLLIYGASSRATGIPD |
| | | RFSGSGSGTDFTLTISRLEPEDFAVYYCQQ |
| | | HDTSLTFGGGTKVEIKGGGGGGGGSGGGGS |
| | | QVQLVQSGAEVKKPGASVKVSCKASGYPFT |
| | | SYGISWVRQAPGQGLEWMGWISTYQGNTNY |
| | | AQKFQGRVTMTTDTSTTTGYMELRRLRSDD |
| | | TAVYYCARDYTRGAWFGESLIGGFDNWGQG |
| | | TLVTVSS |
| SARS-CoV-2 | 234 | DIVMTQSPDSLAVSLGERATINCKSSQSVL |
| S300-v14 | | YSSNNKNYLAWYQQKPGQPPKLLISWASTR |
| mAb VL | | ESGVPDRFSGSGSGTDFTLTISSLQAEDVA |
| (VK) (aa) | | VYYCQQYYSAPGITFGQGTRLEIK |
| SARS-CoV-2 | 235 | QSVLYSSNNKNY |
| S300-v14 | | |
| mAb CDRL1 | | |
| (aa) | | |
| SARS-CoV-2 | 236 | WAS |
| S300-v14 | | |
| mAb CDRL2 | | |
| (aa) | | |
| SARS-CoV-2 | 237 | QQYYSAPGIT |
| S300-v14 | | |
| mAb CDRL3 | | |
| (aa) | | |
| SARS-CoV2 | 238 | GACATCGTGATGACCCAGTCTCCAGACTCA |
| S300-v14 | | CTGGCTGTGTCTCTGGGCGAGAGGGCCACC |
| mAb VL | | ATCAACTGTAAGTCCAGCCAGAGTGTTTTA |
| (VK) (nt) | | TACAGCTCCAACAATAAGAACTACTTAGCT |
| | | TGGTACCAGCAGAAACCAGGACAGCCTCCT |
| | | AAGCTGCTCATTTCCTGGGCTTCTACCCGG |
| | | GAATCCGGGGTCCCTGACCGATTCAGTGGC |
| | | AGCGGGTCTGGGACAGATTTCACTCTCACC |
| | | ATCAGCAGCCTGCAGGCTGAAGATGTGGCA |
| | | GTTTATTACTGTCAACAATATTATAGTGCT |
| | | CCCGGGATCACCTTCGGCCAGGGGACACGA |
| | | CTGGAGATTAAAC |
| SARS-CoV2 | 239 | QVQLQESGPGLVKPSETLSLTCTVSGGSVT |
| S307 mAb | | SGSYYWSWIRQPPGKGLEWIGYMYYSGSTN |
| VH (aa) | | YNPSLKSRVTISVDTSKNQFSLKLSSVTAA |
| | | DTAVYYCARAGCTGITCLRYDYYYGLDVWG |
| | | QGTTVTVSS |
| SARS-CoV2 | 240 | GGSVTSGSYY |
| S307 mAb | | |
| CDRH1 (aa) | | |
| SARS-CoV2 | 241 | MYYSGST |
| S307 mAb | | |
| CDRH2 (aa) | | |
| SARS-CoV2 | 242 | ARAGCTGITCLRYDYYYGLDV |
| S307 mAb | | |
| CDRH3 (aa) | | |
| SARS-CoV2 | 243 | EIVLTQSPGTLSLSPGKRATLSCRASQSVS |
| S307 mAb | | SSYLAWYQQRPGQAPRLLIYGASSRAAGIP |
| VL(VK) (aa) | | DRESGSGSGTDFTLTISRLEPEDFAVYYCQ |
| | | QYGSSSWTFGQGTKVEIK |
| SARS-CoV2 | 244 | QSVSSSY |
| S307 mAb | | |
| CDRL1 (aa) | | |
| SARS-CoV2 | 245 | GAS |
| S307 mAb | | |
| CDRL2 (aa) | | |
| SARS-CoV2 | 246 | QQYGSSSWT |
| S307 mAb | | |
| CDRL3 (aa) | | |
| SARS-CoV2 | 247 | CAGGTGCAGCTGCAGGAGTCGGGCCCAGGA |
| S307 mAb | | CTGGTGAAGCCTTCGGAGACCCTGTCCCTC |
| VH (nt) | | ACCTGCACTGTCTCTGGTGGCTCCGTCACC |
| | | AGTGGTAGTTACTACTGGAGCTGGATCCGG |
| | | CAGCCCCCAGGGAAGGGACTGGAGTGGATT |
| | | GGGTATATGTATTACAGTGGGAGCACCAAT |
| | | TACAACCCCTCCCTCAAGAGTCGAGTCACC |
| | | ATATCAGTAGACACGTCCAAGAACCAGTTC |
| | | TCCCTGAAGCTGAGCTCTGTGACCGCTGCG |
| | | GACACGGCCGTGTATTACTGTGCGAGGGCA |
| | | GGTTGTACTGGTATCACCTGCTTACGTTAC |
| | | GACTACTACTACGGTCTGGACGTCTGGGGC |
| | | CAAGGGACCACGGTCACCGTCTCCTCA |
| SARS-CoV2 | 248 | GAAATTGTGTTGACGCAGTCTCCAGGCACC |
| S307 mAb | | CTGTCTTTGTCTCCAGGGAAAAGAGCCACC |
| VL(VK) (nt) | | CTCTCCTGCAGGGCCAGTCAGAGTGTTAGC |
| | | AGCAGCTACTTAGCCTGGTACCAGCAGAGA |
| | | CCTGGCCAGGCTCCCAGGCTCCTCATCTAT |
| | | GGTGCATCCAGCAGGGCCGCTGGCATCCCA |
| | | GACAGGTTCAGTGGCAGTGGGTCTGGGACA |
| | | GACTTCACTCTCACCATCAGCAGACTGGAG |
| | | CCTGAAGATTTTGCAGTGTATTACTGTCAG |
| | | CAGTATGGTAGCTCATCGTGGACGTTCGGC |
| | | CAAGGGACCAAGGTGGAAATCAAAC |
| SARS-CoV-2 | 249 | CAGGTTCAGCTGGTGCAGTCTGGAGCTGAG |
| S309-v1 mAb | | GTGAAGAAGCCTGGGGCCTCAGTGAAGGTC |
| VH (nt) | | TCCTGCAAGGCTTCTGGTTACCCCTTTACC |
| | | AGTTATGGTATCAGCTGGGTGCGACAGGCC |
| | | CCTGGACAAGGGCTTGAGTGGATGGGATGG |
| | | ATCAGCACTTACAATGGTAACACAAATTAT |
| | | GCACAGAAGTTCCAGGGCAGAGTCACCATG |
| | | ACCACAGACACATCCACGACCACAGGCTAC |
| | | ATGGAGCTGAGGAGGCTGAGATCTGACGAC |
| | | ACGGCCGTGTATTACTGTGCGAGAGATTAT |
| | | ACTCGTGGTGCTTGGTTCGGGGAGTCATTG |
| | | ATAGGGGGCTTTGACAACTGGGGCCAGGGA |
| | | ACCCTGGTCACCGTCTCCTCA |
| SAES-CoV-2 | 250 | GAAATTGTGTTGACGCAGTCTCCAGGCACC |
| S309-v13 | | CTGTCTTTGTCTCCAGGGGAAAGAGCCACC |
| mAb VL(VK) | | CTCTCCTGCAGGGCCAGTCAGACTGTTAGC |
| (nt) | | AGCACCTCCTTAGCCTGGTACCAGCAGAAA |
| | | CCTGGCCAGGCTCCCAGGCTCCTCATCTAT |
| | | GGTGCATCCAGCAGGGCCACTGGCATCCCA |
| | | GACAGGTTCAGCGGCAGTGGGTCTGGGACA |
| | | GACTTCACTCTCACCATCAGCAGACTGGAG |
| | | CCTGAAGATTTTGCAGTGTATTACTGTCAG |
| | | CAGCATGATACCTCACTCACTTTCGGCGGA |
| | | GGGACCAAGGTGGAGATCAAAC |
| CMV | 251 | GACATTGATTATTGACTAGTTATTAATAGT |
| promoter (nt) | | AATCAATTACGGGGTCATTAGTTCATAGCC |
| | | CATATATGGAGTTCCGCGTTACATAACTTA |
| | | CGGTAAATGGCCCGCCTGGCTGACCGCCCA |
| | | ACGACCCCCGCCCATGACGTCAATAATGAC |
| | | GTATGTTCCCATAGTAACGCCAATAGGGAC |
| | | TTTCCATTGACGTCAATGGGTGGAGTATTT |
| | | ACGGTAAACTGCCCACTTGGCAGTACATCA |
| | | AGTGTATCATATGCCAAGTACGCCCCCTAT |
| | | TGACGTCAATGACGGTAAATGGCCCGCCTG |
| | | GCATTATGCCCAGTACATGACCTTATGGGA |
| | | CTTTCCTACTTGGCAGTACATCTACGTATT |
| | | AGTCATCGCTATTACCATGGTGATGCGGTT |
| | | TTGGCAGTACATCAATGGGCGTGGATAGCG |
| | | GTTTGACTCACGGGGATTTCCAAGTCTCCA |
| | | CCCCATTGACGTCAATGGGAGTTTGTTTTG |
| | | GCACCAAAATCAACGGGACTTTCCAAAATG |
| | | TCGTAACAACTCCGCCCCATTGACGCAAAT |
| | | GGGCGGTAGGCGTGTACGGTGGGAGGTCTA |
| | | TATAAGCAGAGCTCGTTTAGTGAACCGTCA |
| | | GATCGCCTGGAGACGCCATCCACGCTGTTT |
| | | TGACCTCCATAGAAGACACCGGGACCGATC |
| | | CAGCCTCCGCGGCCGGGAACGGTGCATTGG |
| | | AACGCGGATTCCCCGTGCCAAGAGTGACGT |
| | | AAGTACCGCCTATAGAGTCTATAGGCCCAC |
| | | CCCCTTGGCTTCGTTAG |
| Signal | 252 | ATGGGATGGTCATGTATCATCCTTTTTCTA |
| peptide | | GTAGCAACTGCAACCGGTGT |
| (nt) | | |
| Poly- | 253 | AACTTGTTTATTGCAGCTTATAATGGTTAC |
| adenylation | | AAATAAAGCAATAGCATCACAAATTTCACA |
| signal | | AATAAAGCATTTTTTTCACTGCATTCTAGT |
| sequence (nt) | | TGTGGTTTGTCCAAACTCATCAATGTATCT |
| | | TATCATGTCTGGATC |
| SARS-CoV-2 | 254 | GTACGGTGGCTGCACCATCTGTCTTCATCT |
| Light Chain | | TCCCGCCATCTGATGAGCAGTTGAAATCTG |
| IgKC*01 (nt) | | GAACTGCCTCTGTTGTGTGCCTGCTGAATA |
| | | ACTTCTATCCCAGAGAGGCCAAAGTACAGT |
| | | GGAAGGTGGATAACGCCCTCCAATCGGGTA |
| | | ACTCCCAGGAGAGTGTCACAGAGCAGGACA |
| | | GCAAGGACAGCACCTACAGCCTCAGCAGCA |
| | | CCCTGACGCTGAGCAAAGCAGACTACGAGA |
| | | AACACAAAGTCTACGCCTGCGAAGTCACCC |
| | | ATCAGGGCCTGAGCTCGCCCGTCACAAAGA |
| | | GCTTCAACAGGGGAGAGTGTTAG |
| SARS-CoV-2 | 255 | GCGTCGACCAAGGGCCCATCGGTCTTCCCC |
| CH1-CH3 | | CTGGCACCCTCCTCCAAGAGCACCTCTGGG |
| G1m17; | | GGCACAGCGGCCCTGGGCTGCCTGGTCAAG |
| IgG1*01 (nt) | | GACTACTTCCCCGAACCTGTGACGGTCTCG |
| | | TGGAACTCAGGCGCCCTGACCAGCGGCGTG |
| | | CACACCTTCCCGGCTGTCCTACAGTCCTCA |
| | | GGACTCTACTCCCTCAGCAGCGTGGTGACC |
| | | GTGCCCTCCAGCAGCTTGGGCACCCAGACC |
| | | TACATCTGCAACGTGAATCACAAGCCCAGC |
| | | AACACCAAGGTGGACAAGAGAGTTGAGCCC |
| | | AAATCTTGTGACAAAACTCACACATGCCCA |
| | | CCGTGCCCAGCACCTGAACTCCTGGGGGGA |
| | | CCGTCAGTCTTCCTCTTCCCCCCAAAACCC |
| | | AAGGACACCCTCATGATCTCCCGGACCCCT |
| | | GAGGTCACATGCGTGGTGGTGGACGTGAGC |
| | | CACGAAGACCCTGAGGTCAAGTTCAACTGG |
| | | TACGTGGACGGCGTGGAGGTGCATAATGCC |
| | | AAGACAAAGCCGCGGGAGGAGCAGTACAAC |
| | | AGCACGTACCGTGTGGTCAGCGTCCTCACC |
| | | GTCCTGCACCAGGACTGGCTGAATGGCAAG |
| | | GAGTACAAGTGCAAGGTCTCCAACAAAGCC |
| | | CTCCCAGCCCCCATCGAGAAAACCATCTCC |
| | | AAAGCCAAAGGGCAGCCCCGAGAACCACAG |
| | | GTGTACACCCTGCCCCCATCCCGGGAGGAG |
| | | ATGACCAAGAACCAGGTCAGCCTGACCTGC |
| | | CTGGTCAAAGGCTTCTATCCCAGCGACATC |
| | | GCCGTGGAGTGGGAGAGCAATGGGCAGCCG |
| | | GAGAACAACTACAAGACCACGCCTCCCGTG |
| | | CTGGACTCCGACGGCTCCTTCTTCCTCTAT |
| | | AGCAAGCTCACCGTGGACAAGAGCAGGTGG |
| | | CAGCAGGGGAACGTCTTCTCATGCTCCGTG |
| | | ATGCATGAGGCTCTGCACAACCACTACACG |
| | | CAGAAGAGCCTCTCCCTGTCCCCGGGTAAA |
| | | TGA |
| Signal | 256 | MGWSCIILFLVATATG |
| peptide | | |
| (aa) | | |
| SARS-CoV-2 | 257 | atgggctggtcctgtatcatcctgttcctg |
| S309-v1.1 | | gtcgccacagccaccggagtgcacagccaa |
| mAb CH1- | | gtgcagctggtccagagcggcgccgaggtg |
| CH3 G1m17; | | aagaagcccggcgctagcgtgaaggtgtcc |
| IgGHG1*01 | | tgtaaagccagcggatatccttttaccagc |
| LS; GAALIE; | | tacggcatctcctgggtgcggcaggcccct |
| signal | | ggccagggcctggaatggatgggctggatc |
| peptide | | agcacctaccagggaaataccaactacgcc |
| (nt-CO) | | cagaagttccagggaagagtgacaatgacc |
| | | acagatacatctacaaccaccggctacatg |
| | | gaactgaggcggctgagaagcgacgacacc |
| | | gccgtgtactactgcgccagagattacacc |
| | | agaggcgcttggttcggcgagagcctgatc |
| | | ggcggcttcgacaactggggccagggaacc |
| | | ctggtgacagtgtctagcgcttctaccaaa |
| | | ggcccttctgtctttcctctggccccttct |
| | | agcaagtctacaagcggaggcaccgccgcc |
| | | ctgggctgcctggtgaaggactacttcccc |
| | | gagcccgtgaccgtgagctggaatagcggc |
| | | gccctgacaagcggcgtgcacaccttccca |
| | | gctgtgctgcagagcagcggcctgtatagc |
| | | ctgagcagcgtggtcaccgtgcccagcagc |
| | | agcctgggaacacagacctacatctgcaac |
| | | gtgaaccacaagccttctaataccaaggtg |
| | | gataagaaggtggaacctaagagctgcgac |
| | | aaaacacacacatgccctccatgtcctgct |
| | | ccagagctgctggccggccccagcgttttt |
| | | ctgttcccccccaaacctaaagacaccctg |
| | | atgatcagcagaacccctgaggtgacctgt |
| | | gtggtggtggacgtgtcccacgaagatcct |
| | | gaggtgaagttcaactggtacgtggatgga |
| | | gtggaagtgcacaacgccaagaccaaacct |
| | | agagaagagcagtacaacagcacatataga |
| | | gtcgtgtccgtgcttacagtgctgcaccag |
| | | gactggctgaatggaaaggaatacaagtgc |
| | | aaggtgtccaacaaggccctgcctctgcct |
| | | gaggagaagacaatctctaaagccaagggc |
| | | caacctcgggaacctcaggtgtacacactg |
| | | ccccccagccgggacgagctgaccaagaac |
| | | caggtgtccctgacctgcctggtcaagggc |
| | | ttctacccctctgatatcgccgtggaatgg |
| | | gagagcaacggccaacctgagaacaactac |
| | | aagaccacccctccagtgctggacagcgac |
| | | ggcagcttcttcctgtacagcaagctgacc |
| | | gttgacaagtccagatggcagcagggcaac |
| | | gtgttcagctgtagcgtcctgcacgaggcc |
| | | ctgcattctcactacacccagaagagcctg |
| | | tccctcagccctggcaagtga |
| SARS-CoV-2 | 258 | caagtgcagctggtccagagcggcgccgag |
| S309-v1.1 | | gtgaagaagcccggcgctagcgtgaaggtg |
| mAb CH1- | | tcctgtaaagccagcggatatccttttacc |
| CH3 G1m17; | | agctacggcatctcctgggtgcggcaggcc |
| IgGHG1*01 | | cctggccagggcctggaatggatgggctgg |
| LS; GAALIE; | | atcagcacctaccagggaaataccaactac |
| (nt-CO) | | gcccagaagttccagggaagagtgacaatg |
| | | accacagatacatctacaaccaccggctac |
| | | atggaactgaggcggctgagaagcgacgac |
| | | accgccgtgtactactgcgccagagattac |
| | | accagaggcgcttggttcggcgagagcctg |
| | | atcggcggcttcgacaactggggccaggga |
| | | accctggtgacagtgtctagcgcttctacc |
| | | aaaggcccttctgtctttcctctggcccct |
| | | tctagcaagtctacaagcggaggcaccgcc |
| | | gccctgggctgcctggtgaaggactacttc |
| | | cccgagcccgtgaccgtgagctggaatagc |
| | | ggcgccctgacaagcggcgtgcacaccttc |
| | | ccagctgtgctgcagagcagcggcctgtat |
| | | agcctgagcagcgtggtcaccgtgcccagc |
| | | agcagcctgggaacacagacctacatctgc |
| | | aacgtgaaccacaagccttctaataccaag |
| | | gtggataagaaggtggaacctaagagctgc |
| | | gacaaaacacacacatgccctccatgtcct |
| | | gctccagagctgctggccggccccagcgtt |
| | | tttctgttcccccccaaacctaaagacacc |
| | | ctgatgatcagcagaacccctgaggtgacc |
| | | tgtgtggtggtggacgtgtcccacgaagat |
| | | cctgaggtgaagttcaactggtacgtggat |
| | | ggagtggaagtgcacaacgccaagaccaaa |
| | | cctagagaagagcagtacaacagcacatat |
| | | agagtcgtgtccgtgcttacagtgctgcac |
| | | caggactggctgaatggaaaggaatacaag |
| | | tgcaaggtgtccaacaaggccctgcctctg |
| | | cctgaggagaagacaatctctaaagccaag |
| | | ggccaacctcgggaacctcaggtgtacaca |
| | | ctgccccccagccgggacgagctgaccaag |
| | | aaccaggtgtccctgacctgcctggtcaag |
| | | ggcttctacccctctgatatcgccgtggaa |
| | | tgggagagcaacggccaacctgagaacaac |
| | | tacaagaccacccctccagtgctggacagc |
| | | gacggcagcttcttcctgtacagcaagctg |
| | | accgttgacaagtccagatggcagcagggc |
| | | aacgtgttcagctgtagcgtcctgcacgag |
| | | gccctgcattctcactacacccagaagagc |
| | | ctgtccctcagccctggcaagtga |
| SARS-CoV-2 | 259 | atgggctggtcctgtatcatcctgttcctg |
| S309-v1.1 | | gtcgccacagccaccggagtgcacagccaa |
| mAb CH1- | | gtgcagctggtccagagcggcgccgaggtg |
| CH3 G1m17; | | aagaagcccggcgctagcgtgaaggtgtcc |
| IgGHG1*01 | | tgtaaagccagoggatatccttttaccagc |
| LS; signal | | tacggcatctcctgggtgcggcaggcccct |
| peptide (nt- | | ggccagggcctggaatggatgggctggatc |
| CO) | | agcacctaccagggaaataccaactacgcc |
| | | cagaagttccagggaagagtgacaatgacc |
| | | acagatacatctacaaccaccggctacatg |
| | | gaactgaggcggctgagaagcgacgacacc |
| | | gccgtgtactactgcgccagagattacacc |
| | | agaggcgcttggttcggcgagagcctgatc |
| | | ggcggcttcgacaactggggccagggaacc |
| | | ctggtgacagtgtctagcgcttctaccaaa |
| | | ggcccttctgtctttcctctggccccttct |
| | | agcaagtctacaagcggaggcaccgccgcc |
| | | ctgggctgcctggtgaaggactacttcccc |
| | | gagcccgtgaccgtgagctggaatagcggc |
| | | gccctgacaagcggcgtgcacaccttccca |
| | | gctgtgctgcagagcagcggcctgtatagc |
| | | ctgagcagcgtggtcaccgtgcccagcagc |
| | | agcctgggaacacagacctacatctgcaac |
| | | gtgaaccacaagccttctaataccaaggtg |
| | | gataagaaggtggaacctaagagctgcgac |
| | | aaaacacacacatgccctccatgtcctgct |
| | | ccagagctgctgggcggccccagcgttttt |
| | | ctgttcccccccaaacctaaagacaccctg |
| | | atgatcagcagaacccctgaggtgacctgt |
| | | gtggtggtggacgtgtcccacgaagatcct |
| | | gaggtgaagttcaactggtacgtggatgga |
| | | gtggaagtgcacaacgccaagaccaaacct |
| | | agagaagagcagtacaacagcacatataga |
| | | gtcgtgtccgtgcttacagtgctgcaccag |
| | | gactggctgaatggaaaggaatacaagtgc |
| | | aaggtgtccaacaaggccctgcctgcccct |
| | | atcgagaagacaatctctaaagccaagggc |
| | | caacctcgggaacctcaggtgtacacactg |
| | | ccccccagccgggacgagctgaccaagaac |
| | | caggtgtccctgacctgcctggtcaagggc |
| | | ttctacccctctgatatcgccgtggaatgg |
| | | gagagcaacggccaacctgagaacaactac |
| | | aagaccacccctccagtgctggacagcgac |
| | | ggcagcttcttcctgtacagcaagctgacc |
| | | gttgacaagtccagatggcagcagggcaac |
| | | gtgttcagctgtagcgtcctgcacgaggcc |
| | | ctgcattctcactacacccagaagagcctg |
| | | tccctcagccctggcaagtga |
| SARS-CoV-2 | 260 | caagtgcagctggtccagagcggcgccgag |
| S309-v1.1 | | gtgaagaagcccggcgctagcgtgaaggtg |
| mAb CH1- | | tcctgtaaagccagcggatatccttttacc |
| CH3 G1m17; | | agctacggcatctcctgggtgcggcaggcc |
| IgGHG1*01 | | cctggccagggcctggaatggatgggctgg |
| LS (nt-CO) | | atcagcacctaccagggaaataccaactac |
| | | gcccagaagttccagggaagagtgacaatg |
| | | accacagatacatctacaaccaccggctac |
| | | atggaactgaggcggctgagaagcgacgac |
| | | accgccgtgtactactgcgccagagattac |
| | | accagaggcgcttggttcggcgagagcctg |
| | | atcggcggcttcgacaactggggccaggga |
| | | accctggtgacagtgtctagcgcttctacc |
| | | aaaggcccttctgtctttcctctggcccct |
| | | tctagcaagtctacaagcggaggcaccgcc |
| | | gccctgggctgcctggtgaaggactacttc |
| | | cccgagcccgtgaccgtgagctggaatagc |
| | | ggcgccctgacaagcggcgtgcacaccttc |
| | | ccagctgtgctgcagagcagcggcctgtat |
| | | agcctgagcagcgtggtcaccgtgcccagc |
| | | agcagcctgggaacacagacctacatctgc |
| | | aacgtgaaccacaagccttctaataccaag |
| | | gtggataagaaggtggaacctaagagctgc |
| | | gacaaaacacacacatgccctccatgtcct |
| | | gctccagagctgctgggcggccccagcgtt |
| | | tttctgttcccccccaaacctaaagacacc |
| | | ctgatgatcagcagaacccctgaggtgacc |
| | | tgtgtggtggtggacgtgtcccacgaagat |
| | | cctgaggtgaagttcaactggtacgtggat |
| | | ggagtggaagtgcacaacgccaagaccaaa |
| | | cctagagaagagcagtacaacagcacatat |
| | | agagtcgtgtccgtgcttacagtgctgcac |
| | | caggactggctgaatggaaaggaatacaag |
| | | tgcaaggtgtccaacaaggccctgcctgcc |
| | | cctatcgagaagacaatctctaaagccaag |
| | | ggccaacctcgggaacctcaggtgtacaca |
| | | ctgccccccagccgggacgagctgaccaag |
| | | aaccaggtgtccctgacctgcctggtcaag |
| | | ggcttctacccctctgatatcgccgtggaa |
| | | tgggagagcaacggccaacctgagaacaac |
| | | tacaagaccacccctccagtgctggacagc |
| | | gacggcagcttcttcctgtacagcaagctg |
| | | accgttgacaagtccagatggcagcagggc |
| | | aacgtgttcagctgtagcgtcctgcacgag |
| | | gccctgcattctcactacacccagaagagc |
| | | ctgtccctcagccctggcaagtga |
| S309-v13 | 261 | tgggctggtcctgcatcatcctgttcctgg |
| Light chain | | tggccacagccaccggcgtgcacagcgaga |
| k1m3; | | tcgtcctgacacagagccccggcacactga |
| IgKC*01; | | gcctctccccaggcgagcgggctacactgt |
| signal | | cttgtagagcttctcagaccgtgtccagca |
| peptide | | ccagcctggcctggtatcagcagaaacctg |
| (nt-CO) | | gccaggcccctagactgctgatctacggcg |
| | | ccagcagcagagccaccggcatccctgata |
| | | gattcagcggcagcggatctggaaccgact |
| | | tcaccctgaccatcagccggctggaacccg |
| | | aggactttgccgtgtactactgccagcaac |
| | | acgacaccagcctgaccttcggcggcggaa |
| | | caaaggtggaaatcaagagaaccgtggccg |
| | | cccctagcgtgttcatcttcccccccagcg |
| | | acgagcagctgaagagcggtacagcttctg |
| | | tggtgtgcctgctgaacaacttctacccgc |
| | | gggaagccaaggtgcagtggaaggtggaca |
| | | acgccctgcagagcggcaacagccaggaga |
| | | gcgtgacagagcaggacagcaaggacagca |
| | | cctacagcctgagcagcaccctgaccctga |
| | | gcaaggccgactacgagaagcacaaggtgt |
| | | acgcctgtgaagtgacccaccagggcctgt |
| | | ctagccctgtgaccaagtcttttaacagag |
| | | gcgagtgctga |
| S309-v13 | 262 | Gagatcgtcctgacacagagccccggcaca |
| Light chain | | ctgagcctctccccaggcgagcgggctaca |
| k1m3; | | ctgtcttgtagagcttctcagaccgtgtcc |
| IgKC*01; (nt- | | agcaccagcctggcctggtatcagcagaaa |
| CO) | | cctggccaggcccctagactgctgatctac |
| | | ggcgccagcagcagagccaccggcatccct |
| | | gatagattcagcggcagcggatctggaacc |
| | | gacttcaccctgaccatcagccggctggaa |
| | | cccgaggactttgccgtgtactactgccag |
| | | caacacgacaccagcctgaccttcggcggc |
| | | ggaacaaaggtggaaatcaagagaaccgtg |
| | | gccgcccctagcgtgttcatcttccccccc |
| | | agcgacgagcagctgaagagcggtacagct |
| | | tctgtggtgtgcctgctgaacaacttctac |
| | | ccgcgggaagccaaggtgcagtggaaggtg |
| | | gacaacgccctgcagagcggcaacagccag |
| | | gagagcgtgacagagcaggacagcaaggac |
| | | agcacctacagcctgagcagcaccctgacc |
| | | ctgagcaaggccgactacgagaagcacaag |
| | | gtgtacgcctgtgaagtgacccaccagggc |
| | | ctgtctagccctgtgaccaagtcttttaac |
| | | agaggcgagtgctga |
| Signal | 263 | Atgggctggtcctgcatcatcctgttcctg |
| peptide | | gtggccacagccaccggcgtgcacagc |
| (nt-CO) | | |
| Signal | 264 | MGWSCIILFLVATATGVHS |
| peptide | | |
| (aa) | | |
| SARS-CoV-2 | 265 | ASTKGPSVFPLAPSSKSTSGGTAALGCLVK |
| CH1-CH3 | | DYFPEPVTVSWNSGALTSGVHTFPAVLQSS |
| G1m17; | | GLYSLSSVVTVPSSSLGTQTYICNVNHKPS |
| IgG1*01 LS | | NTKVDKKVEPKSCDKTHTCPPCPAPELLGG |
| no C-term Lys | | PSVFLFPPKPKDTLMISRTPEVTCVVVDVS |
| (aa) | | HEDPEVKFNWYVDGVEVHNAKTKPREEQYN |
| | | STYRVVSVLTVLHQDWLNGKEYKCKVSNKA |
| | | LPAPIEKTISKAKGQPREPQVYTLPPSRDE |
| | | LTKNQVSLTCLVKGFYPSDIAVEWESNGQP |
| | | ENNYKTTPPVLDSDGSFFLYSKLTVDKSRW |
| | | QQGNVFSCSVLHEALHSHYTQKSLSLSPG |
| SARS-CoV-2 | 266 | ASTKGPSVFPLAPSSKSTSGGTAALGCLVK |
| CH1-CH3 | | DYFPEPVTVSWNSGALTSGVHTFPAVLQSS |
| G1m17; | | GLYSLSSVVTVPSSSLGTQTYICNVNHKPS |
| IgG1*01 LS | | NTKVDKKVEPKSCDKTHTCPPCPAPELLAG |
| GAALIE no | | PSVFLFPPKPKDTLMISRTPEVTCVVVDVS |
| C-term Lys | | HEDPEVKFNWYVDGVEVHNAKTKPREEQYN |
| (aa) | | STYRVVSVLTVLHQDWLNGKEYKCKVSNKA |
| | | LPLPEEKTISKAKGQPREPQVYTLPPSRDE |
| | | LTKNQVSLTCLVKGFYPSDIAVEWESNGQP |
| | | ENNYKTTPPVLDSDGSFFLYSKLTVDKSRW |
| | | QQGNVFSCSVLHEALHSHYTQKSLSLSPG |

### EXAMPLES

### Example 1

### Human Monoclonal Antibodies that Bind Spike Protein of SARS-CoV-2

B cells from a donor with previous SARS-CoV infection were sorted and immortalized with EBV and screened in 384-well plates, as described in European patent EP1597280B1.

Two weeks after immortalization, supernatants of immortalized B cells were tested for antibodies binding to SARS-CoV-2 Spike protein using a flow cytometry-based method. ExpiCHO cells were transfected with S protein of SARS-Cov-2 (strain BetaCoV/Wuhan-Hu-1/2019), or with an empty plasmid as a negative control. Fourteen monoclonal antibodies were identified that bind SARS-CoV-2 S, and were termed SARS-CoV-2 S300 through SARS-CoV-2 S312 and SARS-CoV-2 S315.

### Example 2

### Binding of Human Monoclonal Antibodies to RBD of SARS-CoV-2 Using Octet

Strepavidin biosensors (Pall ForteBio) were used to immobilize anti-Strep Tag II antibody at 3 μg/ml (clone 5A9F9, Biotin, LabForce AG, Muttenz CH), after a hydration step for 10 min with Kinetics Buffer (KB; 0.01% endotoxin-free BSA, 0.002\hat{ } Tween-20, 0.005% NaN3 in PBS). SARS-CoV-2 RBD with a Strep Tag II (produced in-house) was then loaded for 6 min at a concentration of 4 μg/ml in KB. Antibodies from B cell supernatant were allowed to associate for a period of time. To observe dissociation, sensors were moved from the antibody solution into KB and antibody dissociation was monitored.

The S309 mAb comprises the S309-v1 VH and S309-v13 VL amino acid sequences provided in Table 1 (SEQ ID Nos.: 105 and 168, respectively). Comparison of the binding curves for S303 and S309 indicates that S303 has both a faster on-rate and a faster off-rate than S309, suggesting that S309 may bind with higher affinity.

### Example 3

### Binding of Human Monoclonal Antibodies to RBD of SARS-CoV-2 and SARS-CoV-1 Using Octet

The affinity of three cross-reactive recombinant antibodies (S303 rIgG1, S304 rIgG1, S309 rIgG1) and two SARS-CoV-1 specific antibodies was tested using Octet. The affinity was measured by immobilizing the antibody on sensors and dipping the sensors into wells with different concentrations of RBD.

The kinetics of antibody binding to RBD was recorded during the association phase, after which the sensors were dipped into buffer without antibody to observe the kinetics of antibody detaching from the RBD during the dissociation phase. Briefly, protein A biosensors (Pall ForteBio) were used to immobilize recombinant antibodies at 2.7 μg/ml for 1 minute, after a hydration step for 10 minutes with Kinetics Buffer (KB; 0.01% endotoxin-free BSA, 0.002\hat{ }Tween-20, 0.005% NaN3 in PBS). Association curves were recorded for 5 minutes by incubating the antibody-coated sensors with different concentration of SARS-CoV-1 RBD (Sino Biological) or SARS-CoV-2 RBD (produced in house in Expi-CHO cells; residues 331-550 of spike from BetaCoV/Wuhan-Hu-1/2019, accession number MN908947). The highest RBD concentration tested was 10 ug/ml, then 1:2.5 serially diluted. Dissociation was recorded for 9 minutes by moving the sensors to wells containing KB. Affinities, represented by KD values, were calculated using a global fit model (Octet). Octet Red96 (ForteBio) equipment was used.

Three cross-reactive antibodies (S303 rIgG1, S304 rIgG1, S309 rIgG1) and two SARS-CoV-1 specific antibodies (S230 and S109) were tested. All antibodies showed strong binding to SARS-CoV-1 RBD. S230 and S109 did not bind to SARS-CoV-2 RBD. Binding of S303 rIgG1, S304 rIgG1, and S309 rIGg1 to SARS-CoV-2 RBD was in the nanomolar range, with S309 rIgG1 showing the strongest affinity. KD values are indicated below the graphs. KD values are only estimates (KD=<1.0×10⁻¹²M) if the antibody binding is very strong and dissociation is slow. An exact KD for S309 rIgG1 could not be measured by this assay since the dissociation was too slow.

### Example 4

### Neutralization of SARS-CoV-2 by Human Monoclonal Antibodies

Replication-incompetent viruses pseudotyped with the SARS-CoV-2 S gene (isolate BetaCoV/Wuhan-Hu-1/2019; accession number MN908947) were produced using methods as previously described (Temperton NJ, et al. (2005) Longitudinally profiling neutralizing antibody response to SARS coronavirus with pseudotypes. Emerg Infect Dis 11 (3): 411-416.). Briefly, HEK293T/17 was cotransfected with a SARS-CoV-2 S-expressing plasmid (phCMV1, Genlantis) and with a complementing viral-genome reporter gene vector, pNL4-3. Luc+.E-R+. A single-cycle infectivity assay was used to measure the neutralization of luciferase-encoding virions pseudotyped with the SARS-CoV-2 S protein, as previously described (Temperton NJ, et al. (2007) A sensitive retroviral pseudotype assay for influenza H5N1-neutralizing antibodies. Influenza Other Respi Viruses 1(3): 105-112.). Briefly, appropriate dilutions of the virion-containing culture supernatants were preincubated at 37° C. for 1 h with antibodies at various concentrations and the virus-mAb mixtures was then added to Vero E6 cells that were seeded the day before infection. The cells were then lysed with Steady-Glo reagent (Promega, E2520), and the relative luminescence units (RLU) in the cell lysates was determined on a luminometer microplate reader (Synergy H1 Hybrid Multi-Mode Reader; Biotek). The reduction of infectivity was determined by comparing the RLU in the presence and absence of antibody and expressed as percentage of neutralization.

Antibodies SARS-CoV-2 S300-v1, S301, S302, S303-v1, S304, S306, S307, S308-v1, S309 (comprising the S309-v1 VH sequence and the S309-v13 VL (VK) sequence), and S310 were tested for neutralization capacity. Antibodies SARS-CoV-2 S300-v1 and SARS-CoV-2 S309 were shown to neutralize SARS-CoV-2.

Further neutralization assays were carried out using SARS donor plasma and antibodies SARS-CoV-2 S309, S311, S312, S303-v1 (rIgG1), S304 (rIgG1), S306 (rIgG1), S310 (rIgG1), and S315. Using this assay, antibodies S309, S311, S312, and S315 were shown to neutralize SARS-CoV-2.

Additional neutralization assays were carried out using monoclonal antibodies S303, S304, S306, S309, S310, and S315.

### Example 5

### Neutralization of SARS-CoV-2 by Recombinant Human Monoclonal Antibodies

The neutralizing activity of two recombinant SARS-CoV-1 and SARS-Cov-2 cross-neutralizing antibodies, S304 rIgG1 and S309 rIgG1, against SARS-CoV-2 pseudotyped viruses (SARS-CoV-2pp) was determined.

Murine leukemia virus (MLV) pseudotyped with SARS-CoV-2 Spike protein (SARS-CoV-2pp) were used. DBT cells stably transfected with ACE2 (DBT-ACE2) were used as target cells. SARS-CoV-2pp was activated with trypsin TPCK at 10 ug/ml. Activated SARS-CoV-2pp was added to a dilution series of antibodies (starting with 50 ug/ml final concentration per antibody, 3-fold dilution). Antibodies were tested at concentrations between 50 ug/ml and 0.02 ug/ml. for the combination of S304 rIgG1 and S309 rIgG1, starting concentrations were 50 ug/ml for each antibody, i.e. the total starting antibody amount was 100 ug/ml. DBT-ACE2 cells were added to the antibody-virus mixtures and incubated for 48 hours. Luminescence was measured after aspirating cell culture supernatant and adding steady-GLO substrate (Promega). S309 rIgG1 showed an IC50 of 0.37 ug/ml, and S304 rIgG1 showed an IC50 of approximately 17 ug/ml. A combination of both antibodies was strongly neutralizing, with an IC50 of 0.077 μg/ml. Further neutralization assays were carried out using the same procedure for recombinant monoclonal antibodies S309 and S315, singly and in combination. S309 showed an IC50 of 1.091 ug/ml, and S315 showed an IC50 of 25.1 ug/ml. The combination of both antibodies showed an IC50 of 0.3047 ug/ml.

### Example 6

### Reactivity of Human Monoclonal Antibodies to SARS-CoV and SARS-CoV-2

Reactivity of additional human mAbs S311 and S312 with the spike S1 subunit protein and the RBD of SARS-CoV and SARS-CoV-2 protein was determined by enzyme-linked immunosorbent assays (ELISA).

96-well plates were coated with recombinant SARS-CoV-2 Spike S1 Subunit Protein (Sino Biological), SARS-CoV-2 RBD (Sino Biological or produced in house; residues 331-550 of spike from BetaCoV/Wuhan-Hu-1/2019, accession number MN908947), recombinant SARS-CoV Spike S1 Subunit Protein (Sino Biological), or SARS-CoV RBD (Sino Biological).

Wells were washed and blocked with PBS+1% BSA for 1 hour at room temperature and were then incubated with serially diluted mAbs for 1 hour at room temperature. Bound mAbs were detected by incubating alkaline phosphatase-conjugated goat anti-human IgG (Southern Biotechnology: 2040-04) for 1 hour at room temperature and were developed by 1 mg/ml p-nitrophenylphosphate substrate in 0.1 M glycine buffer (pH 10.4) for 30 min at room temperature. The optical density (OD) values were measured at a wavelength of 405 nm in an ELISA reader (Powerwave 340/96 spectrophotometer, BioTek).

Further assays were carried out to determine reactivity of human monoclonal antibodies S300, S307, and S309 to RBD of SARS-CoV-2 and SARS-CoV-1 using the same procedure.

### Example 7

### Neutralization of SARS-CoV-2 by Recombinant Human Monoclonal Antibodies S309 and S315

Neutralizing activity of recombinant antibodies S309 rIgG1-LS and S315 rIgG1-LS against SARS-CoV-2 pseudotyped viruses (SARS-CoV-2pp) was determined.

Murine leukemia virus (MLV) pseudotyped with SARS-CoV-2 Spike protein (SARS-CoV-2pp) were used. DBT cells stably transfected with ACE2 (DBT-ACE2) were used as target cells. SARS-CoV-2pp was activated with trypsin TPCK at 10 ug/ml. Activated SARS-CoV-2pp was added to a dilution series of antibodies. DBT-ACE2 cells were added to the antibody-virus mixtures and incubated for 48 hours. Luminescence was measured after aspirating cell culture supernatant and adding steady-GLO substrate (Promega). Luciferase signal of infected cells was used to calculate the percentage of neutralization relative to a no-antibody control.

S309 rIgG1-LS showed an IC50 of approximately 3.9 nM, and S315 rIgG1-LS showed an IC50 of approximately 111.7 mM.

The neutralizing activity of S309-rFab was compared to that of full length S309 rIgG1-LS. Full length S309 rIgG-LS showed an IC50 of 3.821 nM, while S309-rFab showed in IC50 of 3.532 nM.

### Example 8

### Reactivity of Human Monoclonal Antibodies to RBD of SARS-CoV-1, RBD of SARS-CoV-2, and Ectodomains of Various Coronaviruses

The reactivity of monoclonal antibodies with the RBD of SARS-CoV-1 and SARS-CoV-2 and the Spike protein of SARS-CoV-1, SARS-CoV-2, OC43, and MERS was determined by enzyme-linked immunosorbent assays (ELISA).

384-well shallow ELISA plates were coated with stabilized prefusion Spike protein trimer of SARS-CoV-1, SARS-CoV-2, OC43, or MERS at 1 μg/ml, or with SARS-CoV-2 RBD (produced in house; residues 331-550 of spike from BetaCoV/Wuhan-Hu-1/2019, accession number MN908947) at 10 μg/ml, or SARS-CoV-1 RBD (Sino Biological) at 1 μg/ml.

Wells were washed and blocked with PBS+1% BSA for 1 hour at room temperature and were then incubated with serially diluted antibodies for 1-2 hours at room temperature. Antibodies were tested at a concentration range of 5 to 0.00028 μg/ml. Plates were washed and bound antibodies were detected by incubating alkaline phosphatase-conjugated goat anti-human IgG (Southern Biotechnology: 2040-04) for 1 hour at room temperature followed by color development using 1 mg/ml p-nitrophenylphosphate substrate (Sigma-Aldrich 71768) in 0.1 M glycine buffer (pH 10.4) for 30 min at room temperature. The optical density (OD) values were measured at a wavelength of 405 nm in an ELISA reader (Powerwave 340/96 spectrophotometer, BioTek).

### Example 9

### Binding of Monoclonal Antibodies to SARS-CoV-1 and SARS-CoV-2 Spike Protein

ExpiCHO cells were transfected with phCMV1-SARS-CoV-2-S, SARS-spike_pcDNA.3 (strain SARS), or empty phCMV1 using Expifectamine CHO Enhancer. Two days after transfection, cells were collected for immunostaining with antibody. An Alexa647-labelled secondary antibody anti-human IgG Fc was used for detection. Binding of monoclonal antibody to transfected cells was analyzed by flow cytometry using a ZE5 Cell Analyzer (Biorard) and FlowJo software (TreeStar). Positive binding was defined by differential staining of CoV-S transfectants versus mock transfectants. Monoclonal antibody S309 was tested by flow-cytometry at 10 μg/ml for the ability to stain ExpiCHO cells expressing the S protein of SARS-CoV-1 or SARS-CoV-2.

Binding to SARS-CoV-1 S protein or SARS-CoV-2 S protein was measured by flow cytometry for monoclonal antibodies S303, S304, S306, S309, S310, S315, S110, S124, S230, and S109, and the EC50 values were calculated. Eight of these antibodies was calculated to have EC50 values ranging between 1.4 ng/ml and 6,100 ng/ml for SARS-CoV-2 S protein binding and between 0.8 ng/ml and 254 ng/ml for SARS-CoV-1 S protein binding.

Further binding assays using the same procedure were carried out on recombinant monoclonal antibody S309 and four S309 variants. The EC50 values for each antibody are shown in Table 2. The VH and VL amino acid SEQ ID NOs.: are summarized in Example 17.

TABLE 2

| Antibody | EC50 (ng/ml) - Exp. 1 | EC50 (ng/ml) - Exp. 2 |
|---|---|---|
| S309-11 (“11”) | 23.1 | 11.5 |
| S309-12 (“12”) | 22.3 | 9.6 |
| S309-13 (“13”) | 21.8 | 8.9 |
| S309-14 (“14”) | 21.4 | 8.4 |
| S309-15 (“15”) | 18.8 | 7.8 |

Additional assays using the same procedure were carried out using monoclonal antibodies S303, S304, S306, S309, S310, S315, and comparator antibodies S109, S110, S124, and S230.

Assays using the same procedure were also carried out using recombinant monoclonal antibodies S300 and S307.

### Example 10

### Binding of Human Monoclonal Antibodies S309, S303, S304, and S315 to RBD of SARS-CoV-2 and SARS-CoV-1 Using Octet

Affinity of recombinant monoclonal antibodies S309, S303, S304, and S315 was tested using Octet.

His-tagged RBD of SARS-CoV-1 or SARS-CoV-2 were loaded at 3 μg/ml in kinetics buffer (KB) for 15 minutes onto anti-HIS (HIS2) biosensors (Molecular Devices, ForteBio). Association of full-length antibodies was performed in KB at 15 μg/ml for 5 minutes. Association of Fab fragments was performed in KB at 5 μg/ml for 5 minutes. Dissociation in KB was measured for 10 minutes.

Affinities, represented by KD values, were calculated using a global fit model (Octet). Octet Red96 (ForteBio) equipment was used.

Each of these antibodies bound SARS-CoV-2 and SARS-CoV-1 RBD with nanomolar to sub-picomolar affinity.

### Example 11

### Binding of Human Monoclonal Antibody S309 IGG and S309 FAB to SARS-CoV-2 S Protein Ectodomain Trimer and RBD

Affinity and avidity determination of IgG1 compared to Fab fragment: biotinylated RBD of SARS-CoV-2 (produced in house; residues 331-550 of spike protein from BetaCoV/Wuhan-Hu-1/2019, accession number MN908947, biotinylated with EZ-Link NHS-PEG4-Biotin from ThermoFisher) and biotinylated SARS-CoV-2 2P S avi-tagged were loaded at 7.5 μg/ml in Kinetics Buffer (KB; 0.01% endotoxin-free BSA, 0.002% Tween-20, 0.005% NaN3 in PBS) for 8 minutes onto Streptavidin biosensors (Molecular Devices, ForteBio). Association of IgG1 and Fab was performed in KB at 100, 33, 11, 3.6, 1.2 nM for 5 minutes. Dissociation in KB was measured for 10 minutes. KD values were calculated using a 1:1 global fit model (Octet). S309 IgG bound to the SARS-CoV-2 RBD and to the S ectodomain trimer with sub-picomolar and picomolar avidities, respectively. S309 Fab bound to both the SARS-CoV-2 RBD and the S ectodomain trimer with nanomolar to sub-nanomolar affinities.

### Example 12

### Competitive Binding of Human Monoclonal Antibodies to RBD of SARS-CoV-1 OR SARS-CoV-2

Competitive binding of pairs of monoclonal antibodies to SARS-CoV-1 RBD or SARS-CoV-2 RBD was measured to define the binding sites of the antibodies.

Strepavidin biosensors (Pall ForteBio) were used to immobilize anti-Strep Tag II antibody at 3 ug/ml (clone 5A9F9, Biotin, LabForce AG, Muttenz CH), after a hydration step for 10 min with Kinetics Buffer (KB; 0.01% endotoxin-free BSA, 0.002\hat{ } Tween-20, 0.005% NaN3 in PBS). Either SARS-CoV-1 or SARS-CoV-2 RBD with a Strep Tag II (produced in-house) was then loaded for 6 min at a concentration of 4 μg/ml in KB. The first antibody was allowed to associate for a period of time, then the second antibody was allowed to associate for a period of time.

### Example 13

### Competitive Binding of Human Monoclonal Antibody S309 and RBD to Human ACE2

Competitive binding of monoclonal antibodies and RBD to human ACE2 was measured. ACE2-His (Bio-Techne AG) was loaded for 30 minutes at 5 μg/ml in kinetics buffer (KB) onto anti-HIS (HIS2) biosensors (molecular Devices-ForteBio) SARS-CoV-1 RBD-rabbit Fc or SARS-CoV-2 RBD-mouse Fc (Sino Biological Europe GmbH) at 1 μg/ml was associated for 15 minutes, after a preincubation with or without antibody at 30 μg/ml for 30 minutes. Dissociation was monitored for 5 minutes.

### Example 14

### Antibody-Dependent Cytotoxicity and Antibody-Dependent Cellular Phagocytosis of Human Monoclonal Antibodies

Natural killer (NK)-mediated antibody-dependent cell cytotoxicity (ADCC) can contribute to viral control by killing infected cells displaying viral protein on their surface. To investigate the ability of the Abs isolated to leverage this function, ADCC was investigated in vitro using freshly isolated human NK cells as effector cells and SARS-CoV-2 S-transfected ExpiCHO cells as target cells. Target cells were incubated with different amounts of antibody and after 10 minutes were incubated with primary human NK cells as effector cells at a target:effector ratio of 9:1. NK cells were isolated from fresh blood of healthy donors using the MACSxpress NK Isolation Kit (Miltenyi Biotec, Cat. Nr.: 130-098-185). Antibody-dependent cell killing was measured using an LDH release assay (Cytotoxicity Detection Kit (LDH) (Roche; Cat. Nr.: 11644793001) after 4 hours of incubation at 37° C.

Macrophage or dendritic cell-mediated antibody-dependent cellular phagocytosis (ADCP) can also contribute to viral control by clearing infected cells and by potentially stimulating T cell response with viral antigen presentation. ADCP was tested with peripheral blood mononuclear cells as phagocytes and ExpiCHO transfected with SARS-CoV-2 S fluorescently labeled with PKH67 Fluorescent Cell Linker Kits (Sigma Aldrich, Cat. Nr.: MINI67) as target cells. Target cells were incubated with different amounts of antibody for 10 minutes, followed by incubation with human PBMCs isolated from healthy donors that were fluorescently labeled with Cell Trace Violet (Invitrogen, Cat. Nr.: C34557) at an effector: target ratio of 20:1. After an overnight incubation at 37° C., cells were stained with anti-human CD14-APC antibody (BD Pharmingen, Cat. Nr.: 561708, Clone M5E2) to stain phagocytic cells. Antibody-mediated phagocytosis was determined by flow cytometry, measuring the % of monocytes that were positive for PKH67 fluorescence.

Human monoclonal antibodies S309, S304, S306, S315, S230, and the combination of S309 and S304 were assayed for antibody-dependent cell-mediated cytotoxicity (ADCC) and antibody-dependent cellular phagocytosis (ADCP).

Fc variants of monoclonal antibody S309 were tested for ADCC. S309-LS includes the MLNS Fc mutation. S309-GRLR includes the G236R/L328R Fc mutation, which exhibits minimal binding to FcγRs. S309-LS-GAALIE includes both the MLNS and GAALIE Fc mutations.

Human monoclonal antibodies S303, S304, S306, S309, S315, and the combination of S309 and S315 were assayed for ADCC and ADCP. At least the non-GRLR S309 antibodies (alone or in combination with S304) demonstrated ADCC and ADCP.

### Example 15

### Reactivity of Monoclonal Antibodies to Cell Lysate of SARS-CoV-2-Infected Cells

Reactivity of monoclonal antibodies S304, S306, S309, and S310 to cell lysate of SARS-CoV-2-infected VeroE6 cells was measured.

### Example 16

### Neutralization of SARS-CoV-2 Infection by Monoclonal Antibodies S304 and S309, Alone and in Combination

Neutralization of SARS-CoV-2 infection by monoclonal antibodies S304 and S309 was assessed using a SARS-CoV-2 live virus assay. The live virus neutralization assay quantifies the number of infected cells by staining for viral nucleoprotein (NP) with an NP-specific polyclonal rabbit serum. Inhibition was assessed by measuring NP expression at 24 and 45 hours post infection. Enzyme immunoassay (EIA) was used to quantify the level of infection for each antibody dilution tested.

Neutralization was carried out for one hour at room temperature at the indicated antibody concentrations using Vero E6 cells in monolayer in 96-well plates. Wells were infected with 100 TCID50 of virus. After 24 or 45 hours, monolayers were fixed and stained for inhibition of NP expression. Monoclonal antibodies S304 and S309 show a synergistic enhancement of neutralization.

### Example 17

### Production of S309 rIgG Variants

Recombinant IgG1 antibodies were produced using the VH and VL sequences of monoclonal antibody S309. S309-11 comprises the wild-type VH sequence (SEQ ID NO: 105) and wild-type VL sequence (SEQ ID NO: 168) of S309. S309-12 comprises an N55Q VH variant sequence (SEQ ID NO: 113) and the wild-type VL sequence (SEQ ID NO: 168) of S309. S309-13 comprises a W50F variant sequence (SEQ ID NO: 129) and the wild-type VL sequence (SEQ ID NO: 168) of S309. S309-14 comprises a W105F variant sequence (SEQ ID NO: 119) and the wild-type VL sequence (SEQ ID NO: 168) of S309. S309-15 comprises a W50F/G56A/W105F VH variant sequence (SEQ ID NO: 172) and the wild-type VL sequence of S309. S309 recombinant antibody and each of the four variants were produced by transient transfection and expression of a plasmid vector encoding the recombinant antibody in HD 293F cells (GenScript). Cells were harvested on day 4 and IgG expression was validated by Western blot and protein A titer analysis.

### Example 18

### Binding of S309 RIGG and Variants to SARS-CoV-2 RBD Measured by SPR

Binding of recombinant monoclonal antibody S309 and four variants (see Example 17) to RBD was measured using surface plasmon resonance (SPR). SPR experiments were carried out with a Biacore T200 instrument using a single-cycle kinetics approach. S309 IgG was captured on the surface and increasing concentrations of purified SARS-CoV-2 RBD, either glycosylated or deglycosylated, were injected. SPR was conducted using a sensor chip with anti-human Fc covalently immobilized (GE). Buffer used was 10 mM HEPES pH 7.4, 150 mM NaCl, 3 mM EDTA, and 0.05% P20 detergent. Assays were conducted at 25° C. Recombinant antibodies were diluted from supernatant to approximately 2 μg/ml. RBD concentrations were 0.8 nM, 3.1 nM, 12.5 nM, 50 nM, and 200 nM. Glycosylated RBD was obtained by expression in HEK293 cells and purified using one-step Ni affinity purification. Deglycosylated RBD was obtained by expression in-house in Expi293 cells grown in the presence of kifunensine, purification using one-step Ni affinity purification, and treatment with endoglycosidase H. Single-cycle kinetics assays were carried out with 3 minute injections and 20 minute dissociation periods. Association and dissociation kinetics were monitored and fit to a binding model to determine affinity. The results are summarized in Table 3.

TABLE 3

| | Glycosylated RBD | | | Deglycosylated RBD | | | |
|---|---|---|---|---|---|---|---|
| S309 variant | K_{D} | Kₐ (1/Ms) | K_{d} (1/s) | K_{D} | | Kₐ (1/Ms) | K_{d} (1/s) |
| S309-11 (WT) | 0.50 nM | 10.0e4 | 5.0e−5 | 0.91 | nM | 3.0e5 | 2.8e−4 |
| S309-11 (WT) | 0.68 nM | 9.5e4 | 6.5e−5 | 0.98 | nM | 2.9e5 | 2.9e−4 |
| replicate | | | | | | | |
| S309-12 (N55Q) | 0.46 nM | 9.2e4 | 4.2e−5 | 1.3 | nM | 2.7e5 | 3.6e−4 |
| S309-13 (W50F) | 0.51 nM | 9.9e4 | 5.0e−5 | 1.8 | nM | 3.0e5 | 5.3e−4 |
| S309-14 | 0.38 nM | 1.0e5 | 3.9e−5 | 7.9 | nM | 9.8e5 | 7.7e−3 |
| (W105F) | | | | | | | |
| S309-15 | 1.7 nM | 9.9e4 | 1.6e−4 | >10 | nM | estimate Kd with | |
| (W50F/G56A/ | | | | | | steady-state fit | |
| W105F) | | | | | | | |

Binding to deglycosylated RBD was measured in two different SPR assays using different parameters. Experiment 1 used 10 minute injections and an RBD concentration series of 4-fold dilutions from 100 nM. Experiment 2 used 3 minute injections and a concentration series of 4-fold dilutions from 200 nM, as described 5 above. Results are shown in Table 4.

TABLE 4

| | Experiment 1 | | | Experiment 2 | | |
|---|---|---|---|---|---|---|
| S309 variant | K_{D} | Kₐ (1/Ms) | K_{d} (1/s) | K_{D} | Kₐ (1/Ms) | K_{d} (1/s) |
| S309-11 (WT) | 0.83 nM | 3.0e5 | 2.5e−4 | 0.91 nM | 3.0e5 | 2.8e−4 |
| S309-11 (WT) | 0.91 nM | 3.0e5 | 2.7e−4 | 0.98 nM | 2.9e5 | 2.9e−4 |
| replicate | | | | | | |
| S309-12 (N55Q) | 1.2 nM | 2.7e5 | 3.2e−4 | 1.3 nM | 2.7e5 | 3.6e−4 |
| S309-13 (W50F) | 1.7 nM | 2.8e5 | 4.6e−4 | 1.8 nM | 3.0e5 | 5.3e−4 |
| S309-14 | 14 nM | Fit to steady state | | 7.9 nM | 9.8e5 | 7.7e−3 |
| (W105F) | | | | | | |
| S309-15 | 37 nM | Fit to steady state | | Steady-state fit not possible | | |
| (W50F/G56A/ | | | | | | |
| W105F) | | | | | | |

Binding of recombinant monoclonal antibody S309 and five variants to RBD was measured by surface plasmon resonance (SPR) using the same procedure described above, except using purified recombinant antibodies rather than cell culture supernatant. Results are shown in Table 5.

TABLE 5

| | Glycosylated RBD | | | Deglycosylated RBD | | | |
|---|---|---|---|---|---|---|---|
| S309 variant | K_{D} | Kₐ (1/Ms) | K_{d} (1/s) | K_{D} | | Kₐ (1/Ms) | K_{d} (1/s) |
| S309 WT | 0.26 nM | 9.3e4 | 2.4e−5 | 0.67 | nM | 3.4e5 | 2.3e−4 |
| S309 N55Q | 0.39 nM | 8.5e4 | 3.3e−5 | 1.1 | nM | 3.1e5 | 3.2e−4 |
| S309 W50F | 0.39 nM | 9.2e4 | 3.6e−5 | 1.4 | nM | 3.5e5 | 4.9e−4 |
| S309 W105F | 0.35 nM | 9.6e5 | 3.4e−5 | 5.1 | nM | 1.5e6 | 7.9e−3 |
| S309 | 1.6 nM | 9.4e4 | 1.5e−4 | >10 | nM | estimate Kd with | |
| W50F/G56A/ | | | | | | steady-state fit | |
| W105F | | | | | | | |
| S306 G56A | 0.54 nM | 9.3e4 | 5.1e−5 | 0.70 | nM | 3.4e5 | 2.4e−4 |

### Example 19

### Neutralization of SARS-CoV-2 by Recombinant Monoclonal Antibody S309 and Four Variants

Neutralizing activity of recombinant monoclonal antibody S309 and four variants (Example 17) was determined using a VSV-based luciferase reporter pseudotyping system (Kerafast). VSV pseudoparticles and antibody are mixed in DMEM and allowed to incubate for 30 minutes at 37C. The infection mixture is then allowed to incubate with Vero E6 cells for 1 h at 37C, followed by the addition of DMEM with Pen-Strep and 10% FBS (infection mixture is not removed). The cells are incubated at 37 C for 18-24 hours. Luciferase is measured using an Ensight Plate Reader (Perkin Elmer) after the addition of Bio-Glo reagent (Promega). Calculated EC50 values based on this experiment are shown in Table 6.

TABLE 6

| Recombinant Antibody | EC50 |
|---|---|
| S309-11 (WT) | 109 |
| S309-12 (N55Q) | 103 |
| S309-13 (W50F) | 97 |
| S309-14 (W105F) | 65 |
| S309-15 | 53 |
| (W50F/G56A/W105F) | |

### Example 20

### Determination of Antibody-Dependent Activation of Human FcγRIIIa OR FcγRIIa

Determination of antibody-dependent activation of human FcgRIIIa or FcgRIIa was performed using ExpiCHO cells transiently transfected with SARS-CoV-2 S (BetaCoV/Wuhan-Hu-1/2019), incubated with titrated concentrations of antibody for 10 minutes. ExpiCHO cells then were incubated with Jurkat cells expressing FcγRIIIa receptor or FcgRIIa on their surface and stably transfected with NFAT-driven luciferase gene (Promega, Cat. Nr.: G9798 and G7018) at an effector to target ratio of 6:1 for FcγRIIIa and 5:1 for FcγRIIa. Activation of human FcγRs in this bioassay results in the NFAT-mediated expression of the luciferase reporter gene. Luminescence was measured after 21 hours of incubation at 37° C. with 5% CO2, using the Bio-Glo-TM Luciferase Assay Reagent according to the manufacturer's instructions. Monoclonal antibodies S303, S304, S306, S309, S315, and a combination of S309 and S315 were assayed, along with comparator antibody S320. S309, alone or in combination with S315, activated FcγRIIIa (V158) and FcγRIIa (H131)

### Example 21

### Analysis of SARS-CoV-2 S Glycoprotein Sequences

Analysis of the S glycoprotein sequences of 2,229 SARS-CoV-2 isolates was carried out, and indicates that several mutations have occurred with variable frequency on the SARS-CoV-2 S ectodomain.

Further analysis of the S glycoprotein sequences was carried out using 11,839 SARS-CoV-2 isolates. The epitope bound by S309 is conserved in all but four isolates, and those isolates contained N354D or S359N substitutions that are not expected to affect S309 recognition.

### Example 22

### Competition of Antibody S309 with Antibodies Isolated from SARS-CoV-2 Patients

Human monoclonal antibodies isolated from patients who recovered from SARS-CoV-2 infection were tested for overlapping RBD binding sites with monoclonal antibody S309. Competition assays were performed using Octet (instrument: Octet Red96, ForteBio). Anti-His sensors (BIOSENSOR ANTI-PENTA-HIS (HIS1K) 1*1ST) were used to immobilize in house produced HIS-tagged RBD of SARS-CoV-2 (residues 331-550 of Spike protein from BetaCoV/Wuhan-Hu-1/2019, accession number MN908947) at a concentration of 3 μg/ml. Antibodies were associated for 6 min at 15 μg/ml. All proteins were dilated in kinetics buffer (KB). Competing antibodies were then associated at the same concentration for additional 6 mins. Two antibodies were shown to compete with S309 for binding to RBD but, unlike S309, they were not neutralizing for SARS-CoV-2. Data not shown.

### Example 23

### Resistance Selection of SARS-CoV-2 Against Monoclonal Antibody S309-12-MLNS

To examine resistance selection, SARS CoV-2 was passaged for over one month in the presence of Vero E6 cells and fixed concentrations of monoclonal antibody S309-12-MLNS (i.e. S309 N55Q (VH) with MLNS mutations in Fc). Cytopathogenic effect (CPE) was evaluated by visual inspection of plates. Even when no CPE was observed, viral titers were evaluated by focus-forming assay with a methylcellulose overlay. No evidence of viral breakthrough in antibody-treated wells was observed, even at the minimum antibody concentration tested. Data are representative of wells in triplicate.

### Example 24

### Neutralization of SARS-CoV-2 Infection of CALU-3 Human Lung Cells by Monoclonal Antibody S309

Monoclonal antibody S309 was tested for its ability to neutralize live SARS-CoV-2 virus infection of Calu-3 human lung cells (which are positive for the transmembrane protease TMPRSS2) and VeroE6 cells using a nano luciferase assay. In this assay, S309 had an IC50 in Calu-3 cells of 97.70 μg/mL and in VeroE6 cells of 158.5 μg/mL

### Example 25

### Neutralization of SARS-CoV-2 Infection by Monoclonal Antibody S309

Monoclonal antibody S309 was tested neutralization of live SARS-CoV-2 virus infection using both a nano luciferase assay and IFA assay. Briefly, model cells were infected with live SARS-CoV-2 luciferase virus for six hours. Data were collected using three different antibody concentrations: 1, 0.1, and 0.01 MOI.

Results from the nano-luciferase assay were as follows: at 1 MOI, S309 IC50 was 240.6 μg/mL; at 0.1 MOI, S309 IC50 was 235.3 μg/mL, and at 0.01 MOI, S309 IC50 was 206.6 μg/mL.

Results from the IFA assay were as follows: at 1 MOI, S309 IC50 was 233.0 μg/mL; at 0.1 MOI, S309 IC50 was 156.5 μg/mL, and at 0.01 MOI, S309 IC50 was 142.8 μg/mL. Notably, no clusters of infection (or foci) were observed in this infection format.

### Example 26

### Neutralization of SARS-CoV-2 Infection by Monoclonal Antibodies S309 N55Q LS and S309 N55Q LS GAALIE

Monoclonal antibodies S309 N55Q LS (also referred to herein as S309 N55Q MLNS, comprising M428L/N434S Fc mutations) and S309 N55Q LS GAALIE (also referred to herein as S309 N55Q MLNS GAALIE, comprising G236A, A330L, I332E, M428L, and N434S Fc mutations) were assayed for ability to neutralize live SARS-CoV-2 virus infection. Each of S309 N55Q LS and S309 N55Q LS GAALIE comprises a VH having the sequence set forth in SEQ ID NO.: 113 and a VL having the sequence set forth in SEQ ID NO.: 168. The calculated EC50 for S309 N55Q LS was 100.1 ng/ml. The calculated EC50 for S309 N55Q LS GAALIE was 78.3 ng/ml.

### Example 27

### Neutralization of SARS-CoV-2 Pseudotyped Virus by Monoclonal Antibodies S309 N55Q LS and S309 N55Q LS GAALIE

Neutralization of SARS-CoV-2 pseudotyped virus by monoclonal antibodies S309 N55Q LS and S309 N55Q LS GAALIE was tested. The pseudotyped virus was VSV pseudotyped with SARS-CoV-2 Spike protein. The calculated EC50 value for S309 N55Q LS was 24.06 ng/ml. The calculated EC50 value for S309 N55Q LS GAALIE was 22.09 ng/ml.

### Example 28

### Binding of Monoclonal Antibodies S309 N55Q LS and S309 N55Q LS GAALIE to SARS-CoV-2 RBD

Binding of monoclonal antibodies S309 N55Q LS and S309 N55Q LS GAALIE to SARS-CoV-2 RBD was measured by surface plasmon resonance (SPR). Both antibodies exhibited strong binding.

### Example 29

### Binding of Monoclonal Antibodies S309 N55Q LS and S309 N55Q LS GAALIE to SARS-CoV-2 Spike Protein

Binding of monoclonal antibodies S309 N55Q LS and S309 N55Q LS GAALIE to SARS-CoV-2 to SARS-CoV-2 Spike protein was measured by flow cytometry. Both antibodies bound to Spike protein at concentrations as low as approximately 10 ng/mL, and binding to more than 60% of cells at 10,000 ng/ml antibody.

### Example 30

### Binding of Monoclonal Antibodies S309 N55Q LS and S309 N55Q LS GAALIE to Human FCγ Receptors

Binding of monoclonal antibodies S309 N55Q LS and S309 N55Q LS GAALIE to human Fcγ receptors was assayed using SPR. Binding to FcγRIIa (both low affinity R131 and high affinity H131 alleles), FcγRIIIa (both low affinity F158 and high affinity V158 alleles), and FCγRIIb was measured. Biotin CAPture Reagent (modified streptavidin) was injected across all flow cells of a CAP sensor chip docked in a Biacore T200 (Cytiva). Biotinylated Fc receptors at 1 μg/mL were injected across a single flow cell at 10 μL/min for 60 seconds (one receptor per flow cell), with one flow cell reserved as a reference surface. Antibody at 100 μg/mL (diluted in HBS-EP+) was injected across all flow cells for 200 seconds using a flow rate of 30 μL/min and association was monitored. Dissociation was monitored for another 200 seconds after injection. Data was collected at 10 Hz. After each binding measurement, CAP Regeneration reagent was injected to prepare the surface for a new cycle. Experiments were performed at 25° C., with the samples held at 15° C. in the instrument prior to injection. Both antibodies had at least measurable binding to all Fcγ receptors tested. S309 N55Q LS GAALIE had increased binding (as compared to S309 N55Q LS) to all Fcγ receptors except for FcγRIIb.

### Example 31

### Binding of Monoclonal Antibodies S309 LS, S309 N55Q LS, and S309 N55Q LS GAALIE to Complement Component C1q

Binding of monoclonal antibodies S309 LS, S309 N55Q LS, and S309 N55Q LS GAALIE to complement component C1q was measured by biolayer interferometry (BLI) on an Octet instrument. Anti-human Fab (CH1-specific) sensors were used to capture antibody at 10 μg/ml for 10 minutes. The IgG-loaded sensors were then exposed to kinetics buffer containing 3 μg/ml of purified human C1q for 4 minutes, followed by a dissociation step in the same buffer for additional 4 minutes. Association and dissociation profiles were measured in real time as changes in the interference pattern. S309 LS and S309 N55Q LS, but not S309 N55Q LS GAALIE, bound strongly to C1q.

### Example 32

### In Vitro Activation of Human Fc Gamma Receptors by Monoclonal Antibodies S309 LS, S309 N55Q LS, and S309 N55Q LS GAALIE

The ability of monoclonal antibodies S309 LS, S309 N55Q LS, and S309 N55Q LS GAALIE to elicit antibody-dependent activation of human Fcγ receptors was assayed in vitro. Each of S309 LS, S309 N55Q LS, S309 N55Q LS GAALIE, and negative control antibody S309-GRLR was serially diluted 6-fold in assay buffer from 10,000 ng/ml to 0.006 ng/ml. Nine point serial dilutions of antibody were incubated with 12,500 (for FcγRIIIa and FcγRIIb) or 10,000 (for FcγRIIa) CHO-CoV-2-Spike cells per 96-plate well in a white, flat-bottom plate for 15 minutes at room temperature. Jurkat effector cells expressing the indicated FcγRs and stably transfected with an NFAT-driven luciferase gene were thawed, diluted in assay buffer, and added to the plate at an effector to target cell ratio of 6:1 for FcRγIIIa and FcγRIIb or 5:1 for FcγRIIa. Control wells were included to measure antibody-independent activation (containing target cells and effector cells but no antibody) and background luminescence of the plate (wells containing assay buffer only). Plates were incubated for 18 hours at 37° C. with 5% CO2. Activation of human FcγRs in this bioassay results in the NFAT-mediated expression of the luciferase reporter gene. Luminescence was measured with a luminometer after adding the Bio-Glo™ Luciferase Assay Reagent according to the manufacturer's instructions. The negative control showed low-level activation of FcγRIIb and did not activate any of the other FcγRs. S309 N55Q LS GAALIE was the only antibody to strongly activate FcγRIIIa (F158). All non-GRLR S309 antibodies activated FcγRIIa, FcγRIIb, and FcγRIIIa (V158),

### Example 33

### Effector Function of Human Monoclonal Antibodies S309 LS, S309 N55Q LS, and S309 N55Q LS GAALIE

Human monoclonal antibodies S309 LS, S309 N55Q LS, and S309 N55Q LS GAALIE were assayed for their ability to promote NK-cell mediated antibody-dependent cell-mediated cytotoxicity (ADCC) and monocyte-mediated antibody-dependent cellular phagocytosis (ADCP) against cells expressing CoV2-spike protein.

ADCC was measured in vitro by exposing freshly isolated human NK cells from two genotyped donors expressing homozygous low-affinity (F/F158) or high-affinity (V/V158) FcγRIIIa to antibody pre-incubated with CHO-CoV-2-Spike cells and measuring LDH release as a readout according to the manufacturer's instructions (Cytotoxicity Detection Kit (LDH), Roche) after 4 hours of incubation at 37° C. In brief, plates were centrifuged for 4 minutes at 400× g, and 35 μl of supernatant was transferred to a flat 384-well plate. LDH reagent was prepared and 35 μl were added to each well. Using a kinetic protocol, the absorbance at 490 nm and 650 nm was measured once every 2 minutes for 8 minutes, and the slope of the kinetics curve was used as result. The percent specific lysis was determined by applying the following formula: (specific release−spontaneous release)/(maximum release−spontaneous release)×100. S309 GRLR was used as a negative control. All non-GRLR S309 antibodies elicited ADCC.

The ability of monoclonal antibodies S309 LS, S309 N55Q LS, S309 N55Q LS GAALIE, and control antibody S309-GRLR to promote ADCP by primary CD14+ monocytes was measured in vitro by exposing freshly isolated human PBMCs (labeled with cell trace violet) to CHO-CoV-2-Spike expressing cells (labeled with PKH67 Fluorescent Cell Linker Kit (Sigma Aldrich)) that were pre-incubated with antibody. Serial dilutions of mAbs (serially diluted 5-fold from 5′,000 ng/ml to 0.32 ng/ml in RPMI-1640+L-glutamine supplemented with 10% Hyclone FBS+2× anti-anti (antibiotic-antimycotic)) were incubated with 10′000 CHO-CoV-2-Spike cells per well of a 96 well polypropylene plate for 10 minutes. Primary PBMCs were fluorescently labeled with Cell Trace Violet according to the manufacturer's instructions. Target cell and antibody mixtures were then incubated with labeled PBMCs at an effector-to-target ratio of 16:1. ADCP activity was measured after overnight incubation by labeling the monocyte population for CD14, and measuring the percentage of cell trace violet+ PKH67+ cells amongst CD14+ monocytes by flow cytometry.

### Example 34

Effect of Monoclonal Antibody S309 on SARS-CoV-2 Spike Protein-Mediated Cell Fusion

The effect of monoclonal antibody S309 on SARS-CoV-2 Spike protein-mediated fusion was tested using cells engineered to over-express Spike protein on the cell surface. The addition of S309 these cell cultures inhibited S protein-mediated cell-cell fusion.

### Example 35

### Effect of Monoclonal Antibodies S309 N55Q LS and S309 N55Q LS GAALIE on SARS-CoV-2 Replication

The effect of monoclonal antibodies S309 N55Q LS and S309 N55Q LS GAALIE on SARS-CoV-2 replication was tested in VeroE6 cells, PBMCs, and dendritic cells. SARS-CoV-2 virus was incubated for one hour with antibody S309 N55Q LS or S309 N55Q LS GAALIE. The virus/antibody mixture was then added to plated VeroE6, PBMC, or monocyte-derived dendritic (MoDC) cells. After incubating the cells with the virus/antibody mixture for one hour at 37° C., the cells were washed and incubated for a further 72 hours in fresh medium. The supernatant from the cultured cells then assayed for focus-forming units (FFU). The supernatant was diluted 1:5 and added to VeroE6 cells. After one hour at 37° C., the VeroE6 cells were overlaid with methylcellulose. After 24 hours' further incubation, the VeroE6 cell cultures were stained for SARS-CoV-2 nucleoprotein. The results showed that S309 variant antibodies do not cause antibody-mediated enhancement of SARS-CoV-2 replication in human donor-derived PBMCs or dendritic cells.

### Example 36

### RBD Epitope-Specific Antibodies in Sera of Individuals Who Recovered from SARS-CoV-2 Infection

Sera from hospitalized, symptomatic, and asymptomatic individuals diagnosed with COVID-19 were tested for competition for binding with monoclonal antibodies that bind identified antigenic sites of SARS-CoV-2 Spike protein RBD.

Six antibodies (S2H13, S2H14, S2A4, S2X35, S304 and S309), whose antigenic binding sites were identified on RBD molecule by X-crystallography or Cryo-EM, were produced, purified and biotinylated using EZ-Link™ NHS-PEO solid phase biotinylation kit (Thermo Scientific #21450) according to manufacturer's instructions. Binding to RBD was measured by ELISA. Briefly, 96-well half-area plates (corning, cat.3690) were coated with 1 ug/ml RBD (mouse Fc Tag, Sino Biological Europa GmbH, cat. no 40592-V05H) and blocked with Blocker buffer (Casein 1%, Thermo Fisher Scientific, cat. no 37528, +0.05% Tween 20). Biotinylated monoclonal antibodies were titrated and added to the plate, followed by incubation with streptavidin-AP and pNPP substrate (Sigma N2765-100TAB). Plates were read by spectrophotometer at 405 nm to determine optical densities (OD). Concentration of biotinylated antibody that determines 80% of binding to RBD (EC80) was calculated by non-linear regression analysis using Graph Prism software. Most sera (58-62%) contained antibodies to RBD Site Ia, which is one of the main portions of the receptor binding motif that interacts with ACE2 receptor. This portion in accessible only in the open conformation of RBD. A smaller fraction of sera (25-41%) contained antibodies recognizing the other RBD sites.

To determine the amount of RBD antigenic site-specific antibodies, titrated sera were tested by ELISA as described above with the additional step whereby a 2× BC80 concentration of biotinylated antibody was added on top of the sera after 20 min of incubation. ODs reflecting residual binding of biotinylated antibodies were measured and percentage of inhibition of binding was measured compared to control wells (100% in wells without sera and biotinylated antibodies and 0% in wells without sera). Dilution of sera that determine 80% of blocking of binding (BD80) was calculated by non-linear regression analysis using Graph Prism software. Similarly, 80% of self-blocking of binding of the 6 monoclonal antibodies was determined (BC80) by testing titrations of purified monoclonal antibodies instead of sera. The estimated amount of RBD antigenic site-specific antibodies was determined for each serum as concentration equivalents (ug/ml) sample with the following formula: BD80×BC80. The antibody response to the six RBD sites varied among individuals, with some individuals showing a poor antibody response.

### Example 37

### Materials and Methods

### Flow-Cytometry Based Screening for Binding to CoV S Protein Expressed on Mammalian Cells

ExpiCHO cells were transfected with S protein of SARS-CoV-2, SARS-CoV and MERS-CoV, or with an empty plasmid as a negative control. The monoclonal antibodies were then tested by flow-cytometry at 10 μg/ml for their ability to stain ExpiCHO cells expressing the S protein of 2019-nCOV, SARS-CoV, MERS-CoV or Mock cell transfectants.

### Transient Expression of Recombinant SARS-CoV-2 Protein

The full-length S gene of SARS-CoV-2 strain (2019-nCoV-S) isolate BetaCoV/Wuhan-Hu-1/2019 (accession number MN908947) was codon optimized for human cell expression and cloned into the phCMV1 expression vector (Genlantis). Expi-CHO cells were transiently transfected with phCMV1-SARS-CoV-2-S, phCMV1-MERS-CoV-S (London1/2012), SARS-spike_pcDNA.3 (strain SARS) or the empty phCMV1 (Mock) using Expifectamine CHO Enhancer. Two days after transfection, cells were collected, fixed, or fixed and permeabilized with saponin for immunostaining with a panel of monoclonal antibodies reactive to SARS-CoV Receptor Binding Domain (RBD). An Alexa647-labelled secondary antibody anti-human IgG Fc was used for detection. Binding of antibodies to transfected cells was analyzed by flow-cytometry using a ZE5 Cell Analyzer (Biorard) and FlowJo software (TreeStar). Positive binding was defined by differential staining of CoV-S-transfectants versus mock-transfectants.

### Competition Experiments Using Octet (BLI, Biolayer Interferometry)

Unless otherwise indicated herein, anti-His sensors (BIOSENSOR ANTI-PENTA-HIS (HIS1K)) were used to immobilize the S1 subunit protein of SARS-CoV (Sino Biological Europe GmbH). Sensors were hydrated for 10 min with Kinetics Buffer (KB; 0.01% endotoxin-free BSA, 0.002\hat{ }Tween-20, 0.005% NaN3 in PBS). SARS-CoV S1 subunit protein was then loaded for 8 min at a concentration of 10 μg/ml in KB. Antibodies were associated for 6 min at 15 μg/ml for full length mAbs nCOV-10 and nCov-6 mAbs or 5 μg/ml for Fab nCOV-4, and in a subsequent experiment comprising nCoV-1 all at 10 μg/ml. Competing antibodies were then associated at the same concentration for additional 6 mins.

### Competition Experiments Using Octet (BLI, Biolayer Interferometry)

For ACE2 competition experiments, ACE2-His (Bio-Techne AG) was loaded for 30 minutes at 5 g/ml in KB onto anti-HIS (HIS2) biosensors (Molecular Devices-ForteBio). SARS-CoV-1 RBD-rabbitFc or SARS-CoV-2 RBD-mouseFc (Sino Biological Europe GmbH) at 1 μg/ml was associated for 15 minutes, after a preincubation with or without antibody (30 μg/ml, 30 minutes). Dissociation was monitored for 5 minutes.

### Affinity Determination Using Octet (BLI, Biolayer Interferometry)

For K_{D} determination of full-length antibodies, protein A biosensors (Pall ForteBio) were used to immobilize recombinant antibodies at 2.7 μg/ml for 1 minute, after a hydration step for 10 minutes with Kinetics Buffer. Association curves were recorded for 5 min by incubating the antibody-coated sensors with different concentration of SARS-CoV-1 RBD (Sino Biological) or SARS-CoV-2 RBD (produced in house; residues 331-550 of spike from BetaCoV/Wuhan-Hu-1/2019, accession number MN908947). Highest RBD concentration tested was 10 ug/ml, then 1:2.5 serially diluted. Dissociation was recorded for 9 min by moving the sensors to wells containing KB. K_{D} values were calculated using a global fit model (Octet). Octet Red96 (ForteBio) equipment was used.

For K_{D} determination of full-length antibodies compared to Fab fragments, His-tagged RBD of SARS-CoV-1 or SARS-CoV-2 were loaded at 3 μg/ml in KB for 15 minutes onto anti-HIS (HIS2) biosensors (Molecular Devices, ForteBio). Association of full-length antibody and Fab was performed in KB at 15 ug/ml and 5 ug/ml respectively for 5 minutes. Dissociation in KB was measured for 10 min.

### ELISA Binding

The reactivities of mAbs with SARS-CoV Spike S1 Subunit Protein (strain WH20) protein were determined by enzyme-linked immunosorbent assays (ELISA). Briefly, 96-well plates were coated with 3 μg/ml of recombinant SARS-CoV Spike S1 Subunit Protein (Sino. Biological). Wells were washed and blocked with PBS+1% BSA for 1 h at room temperature and were then incubated with serially diluted mAbs for 1 h at room temperature. Bound mAbs were detected by incubating alkaline phosphatase-conjugated goat anti-human IgG (Southern Biotechnology: 2040-04) for 1 h at room temperature and were developed by 1 mg/ml p-nitrophenylphosphate substrate in 0.1 M glycine buffer (pH 10.4) for 30 min at room temperature. The optical density (OD) values were measured at a wavelength of 405 nm in an ELISA reader (Powerwave 340/96 spectrophotometer, BioTek).

### Neutralization Assay

Unless otherwise indicated, Murine leukemia virus (MLV) pseudotyped with SARS-CoV-2 Spike protein (SARS-CoV-2pp) or SARS-CoV-1 Spike protein (SARS-CoV-1pp) were used. DBT cells stably transfected with ACE2 (DBT-ACE2) were used as target cells. SARS-CoV-2pp or SARS-CoV-1pp was activated with trypsin TPCK at 10 ug/ml. Activated SARS-CoV-2pp or SARS-CoV-1pp was added to a dilution series of antibodies (starting 50 ug/ml final concentration per antibody, 3-fold dilution). DBT-ACE2 cells were added to the antibody-virus mixtures and incubated for 48 h. Luminescence was measured after aspirating cell culture supernatant and adding steady-GLO substrate (Promega).

Unless otherwise indicated, pseudoparticle neutralization assays use a VSV-based luciferase reporter pseudotyping system (Kerafast). VSV pseudoparticles and antibody are mixed in DMEM and allowed to incubate for 30 minutes at 37C. The infection mixture is then allowed to incubate with Vero E6 cells for 1 h at 37C, followed by the addition of DMEM with Pen-Strep and 10% FBS (infection mixture is not removed). The cells are incubated at 37 C for 18-24 hours. Luciferase is measured using an Ensight Plate Reader (Perkin Elmer) after the addition of Bio-Glo reagent (Promega).

### SPR Single-Cycle Kinetics

SPR experiments were carried out with a Biacore T200 instrument using a single-cycle kinetics approach. S309 IgG was captured on the surface and increasing concentrations of purified SARS-CoV-2 RBD, either glycosylated or deglycosylated, were injected. Association and dissociation kinetics were monitored and fit to a binding model to determine affinity.

### Expression of Recombinant Antibodies

Recombinant antibodies were expressed in ExpiCHO cells transiently cotransfected with plasmids expressing the heavy and light chain as previously described. (Stettler et al. (2016) Specificity, cross-reactivity, and function of antibodies elicited by Zika virus infection. Science, 353 (6301), 823-826) Monoclonal antibodies S303, S304, S306, S309, S310, and S315 were expressed as rIgG-LS antibodies. The LS mutation confers a longer half-life in vivo. (Zalevsky et al. (2010) Enhanced antibody half-life improves in vivo activity. Nature Biotechnology, 28 (2), 157-159)

### Sequence Alignment

SARS-CoV-2 genomics sequences were downloaded from GISAID on Mar. 29, 2020, using the “complete (>29,000 bp)” and “low coverage exclusion” filters. Bat and pangolin sequences were removed to yield human-only sequences. The spike ORF was localized by performing reference protein (YP_009724390.1)-genome alignments with GeneWise2. Incomplete matches and indel-containing ORFs were rescued and included in downstream analysis. Nucleotide sequences were translated in silico using seqkit. Sequences with more than 10% undetermined aminoacids (due to N basecalls) were removed. Multiple sequence alignment was performed using MAFFT. Variants were determined by comparison of aligned sequences (n=2,229) to the reference sequence using the R/Bioconductor package Biostrings. A similar strategy was used to extract and translate spike protein sequences from SARS-CoV genomes sourced from ViPR (search criteria: SARS-related coronavirus, full-length genomes, human host, deposited before December 2019 to exclude SARS-CoV-2, n=53). Sourced SARS-CoV genome sequences comprised all the major published strains, such as Urbani, Tor2, TW1, P2, Frankfurt1, among others. Pangolin sequences as shown by Tsan-Yuk Lam et al were sourced from GISAID. Bat sequences from the three clades of Sarbecoviruses as shown by Lu et al (Lancet 2020) were sourced from Genbank. Civet and raccoon dog sequences were similarly sourced from Genbank.

### Example 38

### Phase 1/2 Clinical Study of Sotrovimab (S309 N55Q LS) and VIR-7832 (S309 N55Q LS GAALIE) for Treatment of Mild to Moderate COVID-19 Disease

In one arm of a multi-center, multi-arm, multi-dose, multi-stage open-label, adaptive, seamless phase I/II Bayesian randomised platform trial (600 patients) to determine the optimal dose, activity and safety of multiple candidate agents for the treatment of COVID-19, sotrovimab (S309 N55Q LS; in more detail, sotrovimab is an engineered monoclonal antibody (IgG1*01 G1m17; VH of SEQ ID NO.: 113, M428L and N434S Fc mutations; VL of SEQ ID NO.: 168 (kappa light chain IgKC*01 k1m3)) and VIR-7832 ((IgG1*01 G1m17; VH of SEQ ID NO.: 113, G236A, A330L, I332E, M428L, and N434S Fc mutations; VL of SEQ ID NO.: 168 (kappa light chain IgKC*01 k1m3)) are evaluated, randomizing between candidate and control with 2:1 allocation in favor of the candidate. Each dose is assessed for safety sequentially in cohorts of 6 patients. This arm comprises 3:1 randomised, blinded, placebo-controlled phase I of VIR-7832, followed by a 2:2:1 blinded, parallel group Phase II trial of VIR-7832 versus sotrovimab versus placebo. Single doses of VIR-7832 are administered by intravenous (IV) infusion. The starting dose is 50 mg, and dose escalations of 150 and 500 mg may be used. The active comparator is sotrovimab (500 mg by i.v. infusion over 1 hour). Placebo is given by i.v. infusion over 1 hour.

### Primary Outcome Measures:

1 Dose-finding/Phase I [Time Frame: 29 days from randomisation]
Determination of a dose(s) for efficacy evaluation. Dose limiting toxicities (Safety and Tolerability of drug under study—CTCAE v5 Grade ≥3 adverse events)
2. Efficacy evaluation/Phase II-Severe patients (Group A) [Time Frame: 29 days from randomisation]
Determination of activity and safety.
In severe patients (Group A): time to clinical improvement. Improvement will be determined according to the WHO Clinical Progression Scale; improvement is defined as a minimum 2-step change from randomisation in the scale up to day 29 post-randomisation.
3. Efficacy evaluation/Phase II-Mild to moderate patients (Group B) [Time Frame: 15 days from randomisation]
Determination of activity and safety.

In mild to moderate patients (Group B): pharmacodynamics of drug under study, defined as time to negative viral titres in nose and/or throat swab, measured up to 15 days post-randomisation.

### Secondary Outcome Measures:

1. Safety assessed by rate of adverse events [Time Frame: Up to 29 days from randomisation]
Adverse event rate according to CTCAE v5
2. To evaluate clinical improvement [Time Frame: From randomisation to day 29]
Proportion of patients with clinical improvement (as defined above) at day 8, 15 and day 29.
3. To evaluate clinical improvement using WHO clinical progression scale [Time Frame: From randomisation to day 15]
Change at day 8 and 15 from randomisation in the WHO Clinical Progression Scale
4. To evaluate clinical improvement using WHO clinical progression scale [Time Frame: From randomisation to day 29]
Time to a one point change on the WHO Clinical Progression Scale
5. To evaluate clinical improvement using SpO2/FiO2 [Time Frame: From randomisation to day 29]
The ratio of the oxygen saturation to fractional inspired oxygen concentration (SpO2/FiO2)
6. To evaluate discharge [Time Frame: From randomisation to day 29]
Proportion of patient discharged at days 8, 15 and 29
7. To evaluate admission to ICU [Time Frame: From randomisation to day 29]
Admission rate to ICU
8 To evaluate safety further (WCC) [Time Frame: From randomisation to day 29]
White cell count on day 1, 3, 5, 8, 11 (while hospitalised); and Day 15 and 29
9. To evaluate safety further (Hg) [Time Frame: From randomisation to day 29]
Haemoglobin on day 1, 3, 5, 8, 11 (while hospitalised); and Day 15 and 29
10. To evaluate safety further (platelets) [Time Frame: From randomisation to day 29]
Platelets on day 1, 3, 5, 8, 11 (while hospitalised); and Day 15 and 29
11. To evaluate safety further (creatinine) [Time Frame: From randomisation to day 29]
Creatinine on day 1, 3, 5, 8, 11 (while hospitalised); and Day 15 and 29
12. To evaluate safety further (ALT) [Time Frame: From randomisation to day 29]
ALT on day 1, 3, 5, 8, 11 (while hospitalised); and Day 15 and 29
13. To evaluate overall mortality [Time Frame: From randomisation to day 29]
Mortality at Days 8, 15 and 29. Time to death from randomisation
14. To evaluate the number of oxygen-free days [Time Frame: From randomisation to day 29]
Duration (days) of oxygen use and oxygen-free days
15. To evaluate ventilator-free days [Time Frame: From randomisation to day 29]
Duration (days) of mechanical ventilation and mechanical ventilation-free days
16. To evaluate incidence of new mechanical ventilation use [Time Frame: From randomisation to day 29]
Incidence of new mechanical ventilation use
17. To evaluate National Early Warning Score (NEWS) 2/qSOFA [Time Frame: From randomisation to day 29]
NEWS2/qSOFA assessed daily while hospitalised
18. To evaluate translational outcomes (Viral Load) [Time Frame: From randomisation to day 29]
Change in viral load over time
19. To evaluate translational outcomes (Baseline SARS-CoV-2) [Time Frame: From randomisation to day 29]
Change in viral load over time

### Eligibility

Ages Eligible for Study: 18 Years and older (Adult, Older Adult)
Sexes Eligible for Study: A11
Accepts Healthy Volunteers: No

### Inclusion Criteria

1. Adults (≥18 years) with laboratory-confirmed SARS-CoV-2 infection (PCR)
2. Ability to provide informed consent signed by study patient or legally acceptable representative
3. Women of childbearing potential (WOCBP) and male patients who are sexually active with WOCBP agree to use a highly effective method of contraception (as outlined in the protocol) from the first administration of trial treatment, throughout trial treatment and for the duration outlined in the candidate-specific trial protocol after the last dose of trial treatment

### Additional Criteria May be Applied:

Group A (severe disease) 4a. Patients with clinical status of Grades 4 (hospitalised, oxygen by mask or nasal prongs), 5 (hospitalised, on non-invasive ventilation, or high flow oxygen), 6 (hospitalised, intubation and mechanical ventilation) or 7 (ventilation and additional organ support-pressors, renal replacement therapy (RRT), extracorporeal membrane oxygenation (ECMO)), as defined by the WHO clinical severity score, 9-point ordinal scale.

Group B (mild-moderate disease) 4b. Ambulant or hospitalised patients with the following characteristics peripheral capillary oxygen saturation (SpO2) >94% RA N.B.

The main trial exclusion criteria are outlined in the master protocol as:
1. *
2. *
3. Pregnant or breast feeding
4. *
5. Allergy to any study medication
6. Patients taking other prohibited drugs (as outline in CST protocol) within 30 days or 5 times the half-life (whichever is longer) of enrolment
7. Patients participating in another clinical trial of an investigational medicinal product (CTIMP)
   * ′The master protocol stipulates the following for ‘1’, ‘2’ and ‘4’:
   ′1. Alanine aminotransferase (ALT) and/or aspartate aminotransferase (AST) >5 times the upper limit of normal (ULN)
   2. Stage 4 severe chronic kidney disease or requiring dialysis (i.e., estimated glomerular filtration rate <30 mL/min/1.73 m²)
   4. ‘anticipated transfer to another hospital which is not a study site within 72 hours’
   Those criteria are not applicable to this protocol as the IMP is a monoclonal antibody, which is not cleared from the body by the liver or kidneys (1 and 2) and patients who are hospitalised are excluded from the study.
   For the purpose of the VIR-7832 and sotrovimab candidate-specific trial the following exclusion criteria also apply:
8. Currently hospitalised or judged by the investigator as likely to require hospitalisation for acute medical care in the next 24 hours
9. Symptoms consistent with severe COVID-19 as defined by shortness of breath at rest or respiratory distress or requiring supplemental oxygen.
10. Participants who, in the judgement of the investigator are likely to die in the next 7 days
11. Severely immunocompromised participants including but not limited to cancer patients receiving immunosuppressive chemotherapy or immunotherapy, those with a solid organ transplant or allogeneic stem cell transplant within the last 3 months, any history of heart or lung transplant or high dose long-term systemic corticosteroids (equivalent to ≥20 mg a day of prednisone or the systemic equivalent for over 2 weeks)
12. Phase I only: Diabetes (requiring medication), chronic kidney disease (i.e., eGFR <60 as determined by the Modification of Diet in Renal Disease (MDRD) study), chronic liver disease (e.g., cirrhosis), congestive heart failure (New York Heart Association (NYHA) class II or more), chronic obstructive pulmonary disease (history of chronic bronchitis, chronic obstructive lung disease, or emphysema with dyspnoea on physical exertion), and moderate to severe asthma (participant requires an inhaled steroid to control symptoms or has been prescribed a course of oral steroids in the past year).
13. Any current medical condition that, in the Investigator's judgment, precludes the participant's safe participation in and completion of the study.
14. Known hypersensitivity to any constituent present in the investigational product
15. Previous anaphylaxis or hypersensitivity to a monoclonal antibody
16. Have ever received a vaccine for SARS-CoV-2, as part of clinical trial or otherwise.
17. Receipt of any vaccine within 48 hours prior to enrolment. Vaccination will not be allowed for 4 weeks after dosing.
18. Receipt of convalescent plasma from a recovered COVID-19 patient or anti SARS-CoV-2 mAb within the last 3 months.
19. Participants who, in the judgment of the investigator, will be unlikely or unable to comply with the requirements of the protocol

### Example 39

### Phase 2/3 Clinical Study of Sotrovimab for Treatment of Mild to Moderate COVID-19 Disease

A randomized, multi-center, double-blind, placebo-controlled study is performed to assess safety and efficacy of sotrovimab (S309 N55Q LS) for the early treatment of COVID-19 in outpatients. Sotrovimab is an engineered monoclonal antibody (IgG1*01 G1m¹⁷; VH of SEQ ID NO.: 113, M428L and N434S Fc mutations; VL of SEQ ID NO.: 168 (kappa light chain IgKC*01 k1m3)).

A primary objective is to evaluate efficacy of sotrovimab versus placebo in preventing the progression of mild/moderate to severe or critical COVID-19 disease. Primary endpoints are the proportion of participants to develop severe disease, critical disease, or death, with a primary analysis up to Day 29.

Key secondary objectives are to determine safety and tolerability of sotrovimab compared to placebo, evaluate efficacy of sotrovimab versus placebo in preventing COVID-19 disease progression by days 8, 15, and 22, evaluate efficacy of sotrovimab versus placebo in preventing mortality, evaluate efficacy of sotrovimab versus placebo in shortening ventilator days, ICU length of stay (LOS), and total hospital LOS, evaluate efficacy of sotrovimab versus placebo in shortening the duration and decreasing the severity of COVID-19 clinical signs and symptoms, evaluate efficacy of sotrovimab versus placebo in preventing non-respiratory complications of COVID-19, evaluate efficacy of sotrovimab versus placebo in reducing duration of SARS-CoV-2 viral shedding, evaluate efficacy of sotrovimab versus placebo in inducing an anti-viral response, evaluate efficacy of sotrovimab versus placebo in facilitating a return to normal living, assess pharmacokinetics (PK) of sotrovimab in serum, and assess immunogenicity of sotrovimab.

Exploratory objectives are to evaluate efficacy of sotrovimab against versus placebo in preventing COVID-19 disease progression by days 8, 15, and 22, monitor on-treatment emergence of SARS-CoV-2 resistant mutants against sotrovimab, evaluate efficacy of sotrovimab versus placebo in reducing SARS-CoV-2 viral load, evaluate effect of sotrovimab versus placebo on potential biomarkers of host response to SARS-CoV-2, evaluate potential relationships between subject genetic polymorphisms and sotrovimab mechanisms of action and/or PK, and measure impact of sotrovimab treatment on time away from work and work productivity due to COVID-19 illness.

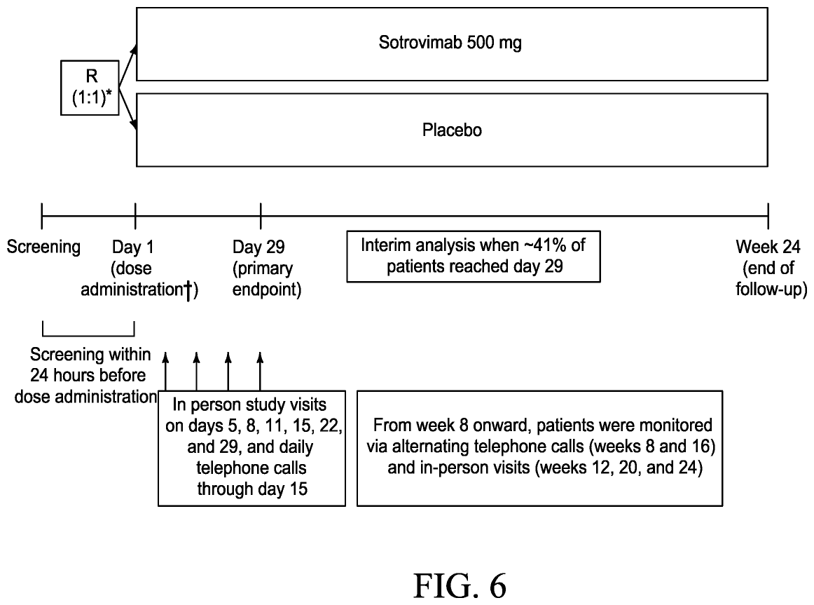

Further study details are shown in FIG. 6.

### Details of Criteria for Evaluation

Primary Endpoints of this Study are as Follows:
Proportion of subjects who progress to severe or critical COVID-19 or die through Day 29 as defined by:
   1. Severe disease: hypoxemia (O2 saturation ≤93% on room air or PaO2/FiO2<300) requiring oxygen supplementation >1 day OR subject requires ≥4 L/min oxygen supplementation or equivalent AND the investigator or designee determine that the measurement of O2 saturation on room air is medically unsafe.
      OR
   2. Critical disease: Respiratory failure requiring at least one of the following: invasive mechanical ventilation, ECMO; OR shock; OR multi-organ dysfunction/failure
      OR
   3. Death
      Secondary Endpoints of this Study are as Follows:
1. Occurrence of adverse events (AEs)
2. Occurrence of serious adverse events (SAEs)
3. Occurrence of adverse events of special interest (AESI)
4. Proportion of subjects who progress to develop severe or critical COVID-19 at Day 8, Day 15, or Day 22
5. 29-day, 60-day, and 90-day all-cause mortality
6. Total number of ventilator days from randomization through 29 days
7. Total intensive care length of stay (LOS)
8. Total hospital LOS
9. Severity and duration of subject reported signs and symptoms of COVID-19 related illness using the Flu-PRO patient-reported outcome measurement instrument
10. Occurrence of cardiac, thromboembolic, renal, neurologic events
11. Detection of SARS-CoV-2 in nasal secretions by PCR at baseline and during follow-up period through Day 29
12. Sotrovimab pharmacokinetics (PK) in serum
13. Incidence and titers (if applicable) of serum ADA to Sotrovimab Exploratory endpoints of this study are as follows:
1. Proportion of subjects at Day 8, Day 15, Day 22, and Day 29 in each category of the ordinal scale for clinical improvement (see below)
2. Emergence of viral resistance mutants to mAb by SARS-CoV-2
3. Viral load in nasal secretions and blood by qRT-PCR
4. Host transcriptome and immunophenotyping analysis
5. FcγR polymorphisms as determined by genotyping
6. IgG1 allotypes as determined by genotyping
7. Change from baseline in Work Productivity and Activity Impairment (WPAI)
8. Change from baseline in health-related quality of life according to EQ-5D-5L

### The Ordinal Scale for Use in Categorizing Clinical Improvement is as Follows:

1. Uninfected: No clinical or virologic evidence of infection
2. Non-hospitalized, no limitation of activity (if using oxygen at home, report as category 4)
3. Non-hospitalized, limitation of activity
4. Hospitalized, no oxygen therapy
5. Hospitalized, low-flow oxygen by mask or nasal prongs
6. Hospitalized, high-flow oxygen, Continuous positive airway pressure (CPAP), bilevel positive airway pressure (BiPAP), non-invasive ventilation
7. Hospitalized, intubation and mechanical ventilation
8. Hospitalized, mechanical ventilation plus organ support (vasopressors, RRT, ECMO)
9. Death

### Number of Subjects

This study is comprised of two parts. The lead-in phase enrolls approximately 20 subjects with early, mild to moderate COVID-19 who are at high risk for progression of disease. The expansion phase enrolls approximately 850 subjects with early, mild to moderate COVID-19 who are at high risk for progression of disease.

### Diagnosis and Main Criteria for Inclusion

### Inclusion Criteria Include:

### Age:

1. Subjects aged 18 years and older at high risk of complications from COVID-19 disease including diabetes, obesity (BMI>30), chronic kidney disease, congestive heart failure, chronic obstructive pulmonary disease, and moderate to severe asthma
AND
2. Subjects ≥55 years old, irrespective of co-morbidities

### Disease Characteristics:

1. Subjects who have a positive SARS-CoV-2 test result (by any validated test e.g. RT-PCR on any specimen type)
AND
2. Have an oxygen saturation ≥94% on room air
AND
1. Have mild to moderate or moderate uncomplicated COVID-19 illness defined by one or more of the following symptoms:
fever, chills, cough, sore throat, malaise, headache, joint or muscle pain, change in smell or taste, vomiting, diarrhea, shortness of breath on exertion
AND
3. Are less than or equal to 4 days from onset of symptoms

### Sex:

1. No gender restriction
2. Female subjects must meet and agree to abide by the following contraceptive criteria. Contraception use by women should be consistent with local regulations regarding the methods of contraception for those participating in clinical studies.
3 A female subject is eligible to participate if she is not pregnant or breastfeeding, and one of the following conditions applies:
Is a woman of non-childbearing potential (WONCBP)
OR
Is a woman of child-bearing potential (WOCBP) and using a contraceptive method that is highly effective, with a failure rate of <1% during the study intervention period and for up to one year after the last dose of study intervention. The investigator should evaluate potential for contraceptive method failure (e.g., noncompliance, recently initiated) in relationship to the first dose of study intervention.
4. A WOCBP must have a negative highly sensitive pregnancy test (urine or serum as required by local regulations) at hospital admission or before the first dose of study intervention. If a urine test cannot be confirmed as negative (e.g., an ambiguous result), a serum pregnancy test is required. In such cases, the subject must be excluded from participation if the serum pregnancy result is positive.

### Informed Consent:

5 Willing to comply with the study requirements and capable of giving signed informed consent or, if not able to give signed informed consent, if alternative consent procedures are able to be followed.

### Exclusion Criteria:

### Medical Conditions

Currently hospitalized or judged by the investigator as likely to require hospitalization in the next 24 hours
Symptoms consistent with severe Covid-19 as defined by shortness of breath at rest or respiratory distress or requiring supplemental oxygen
Patients who, in the judgment of the investigator, are likely to die in the next 7 days
Severely immunocompromised patients, including but not limited to cancer patients actively receiving immunosuppressive chemotherapy or immunotherapy, those with a solid organ transplant or allogeneic stem cell transplant within the last 3 months, or those having conditions requiring the use of systemic corticosteroids equivalent to ≥0.5 mg/kg of body weight per day of prednisone within 6 weeks of randomization
Known hypersensitivity to any constituent present in the investigational product
Previous anaphylaxis or hypersensitivity to a monoclonal antibody

### Prior/Concurrent Clinical Study Experience

Enrollment in any investigational vaccine study within the last 180 days or any other investigational drug study within 30 days prior to day 1 or within five half-lives of the investigational compound, whichever is longer
Enrollment in any trial of an investigational vaccine for SARS-CoV-2

### Other Exclusions

Receipt of any vaccine within 48 hours prior to enrollment. Receipt of a SARS-CoV-2 vaccine prior to randomization at any time point. Vaccination (including vaccination for SARS-CoV-2) will not be allowed for 4 weeks after dosing
Receipt of convalescent plasma from a recovered Covid-19 patient or anti-SARS-CoV-2 monoclonal antibody within the last 3 months
Patients who, in the judgment of the investigator, will be unlikely or unable to comply with the requirements of the protocol through day 29

### Duration of Study Participation and Follow Up

The duration of study drug treatment is a single dose. The estimated total time on study, inclusive of screening and follow-up, for each subject is approximately nine months. All subjects are monitored through at least two hours post-dose prior to discharge from the study unit. Subjects enrolled in the lead-in portion of the study are monitored in an in-patient setting for a minimum of seven days. Subjects are subsequently actively monitored on an outpatient basis with in-person study visits at Weeks 1, 2, 3, and 4 and daily telephone calls on non-study visit days through Day 14. After Day 29, patients are monitored monthly via remote telehealth or phone call for a total of nine months from dosing.

### Study Design

An Independent Data Monitoring Committee (IDMC) actively monitors interim unblinded safety data (Lead-in Phase) and interim unblinded safety and efficacy data (Expansion Phase) to make recommendations regarding ongoing study conduct. The IDMC members include physicians with relevant medical specialist training and one statistician. The IDMC reviews unblinded safety data from the Lead-in Phase of the study prior to initiation of the Expansion Phase. In addition, the IDMC performs regular safety reviews during the Expansion Phase. The first safety review includes available safety and tolerability data though Day 14 from a total of 60 patients (30 per arm). If there are no safety or tolerability concerns according to pre-specified criteria, the IDMC recommends initiation of a separate study in hospitalized patients with severe to critical COVID-19. If necessary, additional safety reviews are conducted after each additional approximately 100 patients are enrolled (50 per arm). Finally, two interim analyses are conducted at approximately 33% of subjects enrolled (145 per arm) and approximately 67% of subjects enrolled (290 per arm) to evaluate safety, futility, and efficacy. A Joint Safety Review Team (JSRT) determines if a safety concern identified during the instream blinded data review needs to be escalated to the IDMC.

This study is a randomized, double-blind, multi-center, placebo-controlled trial of sotrovimab, a monoclonal antibody (mAb) against SARS-CoV-2 for the prevention of progression of mild to moderate COVID-19 disease in high-risk subjects, with interim monitoring to allow early stopping for futility, efficacy, or safety. Subjects with early, mild to moderate COVID-19 who are at high-risk for progression of disease are randomized 1:1 to receive a single, intravenous infusion of either sotrovimab or equal volume saline placebo. Comparisons of safety and efficacy are based on data from concurrently randomized participants.

The study is comprised of 2 parts. The lead-in phase enrolls 20 subjects who have early, mild to moderate COVID-19 and are at high risk of disease progression. Following a safety assessment of unblinded data by an independent data monitoring committee (IDMC), the expansion phase progresses, where additional subjects with early, mild to moderate COVID-19 and who are at high risk of disease progression are enrolled.

### Lead-In Phase:

The Lead-In Phase of the study evaluates the safety and tolerability of sotrovimab in subjects with early, mild to moderate COVID-19 who are at high-risk of progression to severe disease. Subjects are monitored in an in-patient setting for seven days including assessments of respiratory status, oxygenation and other vital signs and laboratory evaluations. A single dose level is studied and is delivered via intravenous infusion. Subjects are admitted to a study unit and monitored closely for adverse events, changes in laboratory parameters and progression or improvement in disease signs and symptoms.

The first two eligible subjects enrolled are randomized 1:1 to sotrovimab or placebo. These sentinel subjects are dosed and monitored for at least 48 hours in an in-patient setting. During dose administration, vital signs are monitored every 15 minutes over the one hour IV infusion. Vital signs are also monitored every one hour after infusion for two hours. Vital signs, ECG, symptom-directed physical examinations(s), and adverse events (AEs) are reviewed by the investigator. If the investigator has no immediate safety concerns, the remainder of the subjects in the Lead-In phase is dosed (total of ten per arm inclusive of the sentinel subjects).

All 20 subjects enrolled in the Lead-In (N=10 per arm; sotrovimab or placebo) are assigned to an intensive PK and immunogenicity substudy. Sparse samples are collected from all participants not enrolled in the substudy. Serum PK and anti-drug antibodies (ADA) samples are collected.

### Expansion Phase:

The Expansion Phase of the study progress following assessment of available unblinded safety data from the Lead-in Phase (N=10 per arm through 14 days of follow-up) by an IDMC. The purpose of the Expansion Phase is to evaluate the safety and efficacy of sotrovimab in comparison to the placebo control arm. Subjects with early mild to moderate COVID-19 who are at risk for progression to severe disease are randomized in a 1:1 ratio (435 per arm) to receive a single, IV infusion of sotrovimab or placebo.

Subjects are monitored through at least 2 hours post-dose prior to discharge from the study unit. Subjects are subsequently actively monitored on an outpatient basis with in-person study visits at Weeks 1, 2, 3, and 4 and daily telephone calls on non-study visit days through Day 14 for AEs and worsening of illness. In addition, the subjects are provided a device to monitor for hypoxemia.

After Day 29, patients are monitored monthly via remote telehealth or phone call to assess for the incidence and of severity of subsequent COVID-19 illness, if any, for a total of 9 months from dosing.

Two interim analyses at approximately 33% of subjects enrolled (145 per arm) and approximately 67% of subjects enrolled (290 per arm) are conducted to evaluate futility, efficacy, and safety, as well as allow for sample size reestimation if the overall disease progression rate is lower than expected.

A placebo control distinguishes safety and tolerability of sotrovimab from the background signs and symptoms of COVID-19 and evaluates its potential benefit on clinical outcomes. The use of a placebo arm allows for a valid evaluation of any changes in efficacy and safety attributable to sotrovimab versus those attributable to background supportive care given during the study.

A primary endpoint to assess efficacy of treatment is progression to severe disease defined as hypoxia requiring oxygen supplementation, or the development of critical disease (respiratory failure requiring at least one of the following: invasive mechanical ventilation, ECMO; OR shock; OR multi-organ dysfunction/failure), or death within 29 days of randomization. The transition from outpatient status without hypoxia or oxygen requirement to hypoxia with a need for oxygen therapy or higher levels of respiratory/organ support is a clinically significant milestone.

A significant secondary endpoints is all-cause mortality at Day 29. Other secondary efficacy endpoints related to hospital stay-length of hospital stay, ICU stay, time ventilated and the severity and duration of subject-reported signs and symptoms of COVID-19 are also clinically relevant.

### Immunology Assessment Substudy:

To explore the host immune response and potential biomarkers of infection, subjects may consent to an optional sub-study in which peripheral blood mononuclear cells (PBMCs) are collected at specified timepoints. This optional substudy is performed at selected sites. Approximately 20-50 participants per arm are enrolled in the optional sub-study. Subjects from both the Lead-In and Expansion Phases of the study may be included in the Immunology Assessment Substudy.

### Study Intervention Groups and Duration

Screening assessments are performed within 24 hours before the first dose. Eligible subjects are treated in a blinded manner with a single IV dose on Day 1 and followed up to 9 months (36 weeks).

In the Lead-In Phase, 20 subjects with mild to moderate COVID-19 are randomized 1:1 to receive a single IV dose of sotrovimab or placebo. Subjects enrolled in the Lead-in Phase of the study are closely monitored in an in-patient setting for a minimum of 7 days. At the end of 7 days they are either discharged to home, remain in the inpatient unit or are formally hospitalized based on investigator assessment of clinical status.

In the Expansion Phase, subjects with mild to moderate COVID-19 are randomized 1:1 to receive a single IV dose of sotrovimab or placebo.

Participants in the Expansion phase are stratified by the following criteria:
1. Duration of symptoms: ≤2 days vs. 3-4 days
2. Age: ≤70 vs. >70 years old

### Study Interventions

Study interventions and dosing instructions are as follows in Table 7:

TABLE 7

| | | |
|---|---|---|
| Dose Formulation | Solution in single-use vial | Sterile 0.9% (w/v) |
| | (25 mg/mL) | sodium chloride solution |
| Unit Dose Strength(s) | 250 mg/vial (250 mg/10 mL) | Not applicable |
| Dosage Level(s) | nn mg once | Once |
| Route of | IV infusion | IV infusion |
| Administration | | |
| Dosing instructions | Withdraw 10.0 mL from the | Dose per IV infusion |
| | vial into a 10-20 mL small | instructions |
| | syringe and transfer to a 150 | |
| | mL or 250 mL IV empty saline | |
| | bag by an unblinded | |
| | pharmacist. Repeat this | |
| | procedure until the required | |
| | dose is reached and administer | |
| | over 1 hour. | |
| Special instructions | Gently mix sotrovimab and | None |
| | saline prior to IV infusion. | |
| Packaging and | Study intervention is provided | Labelled as required per |
| Labelling | in a single-use vial in an | country requirement |
| | individual carton and labelled | |
| | as required per country | |
| | requirement. | |

Dose selection is guided by live virus neutralization data, resistance data, human PK projections informed by non-human primate PK and the no observed adverse effect level (NOAEL) in a cynomolgus monkey.

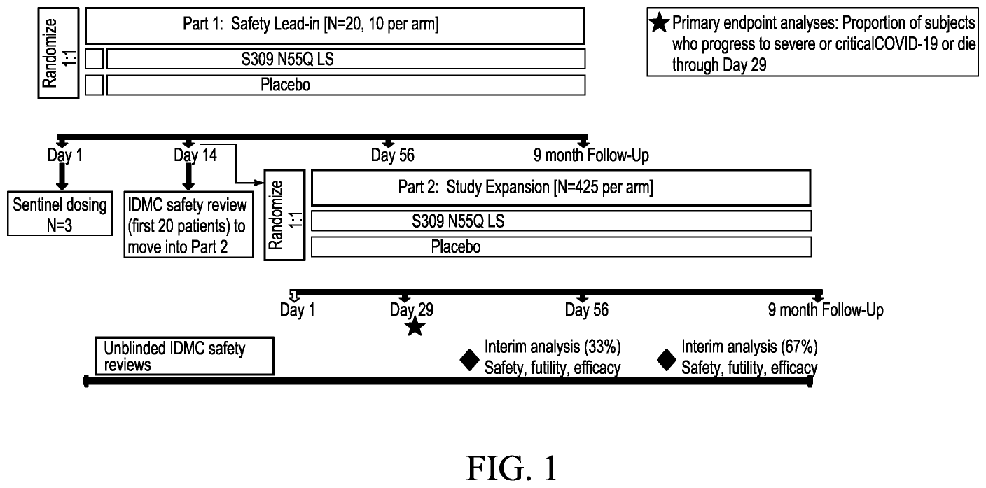

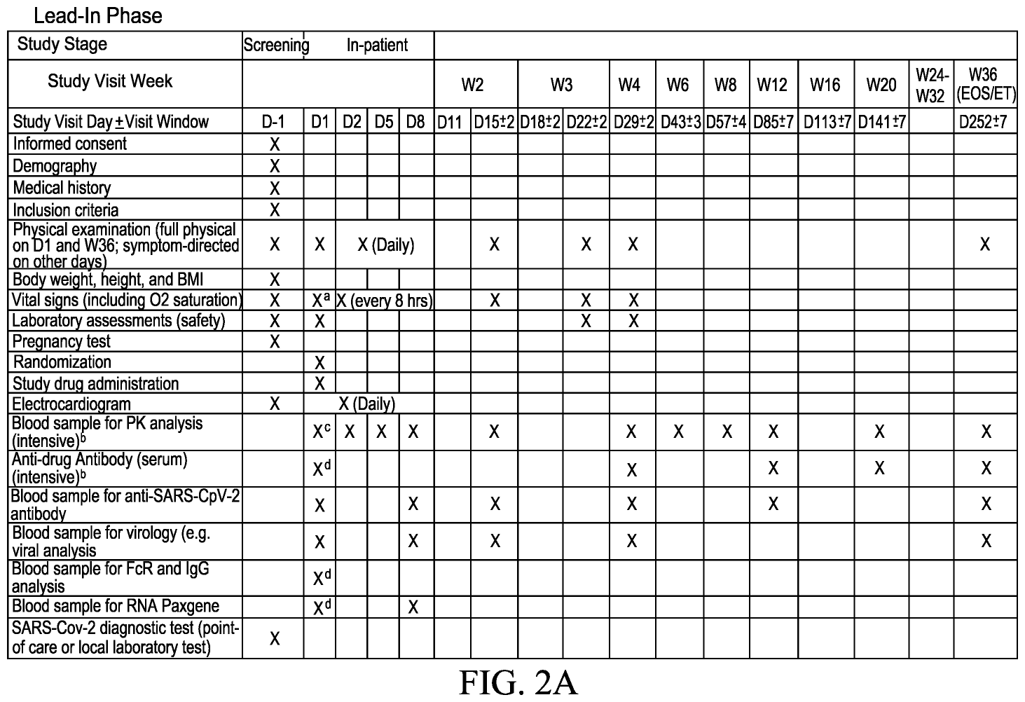

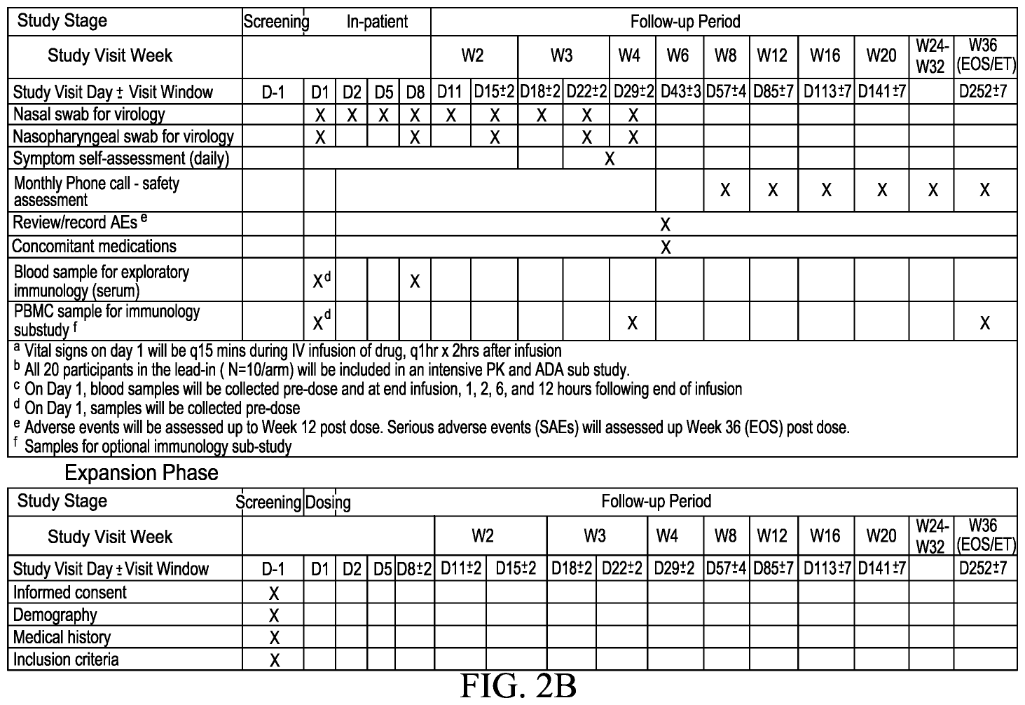

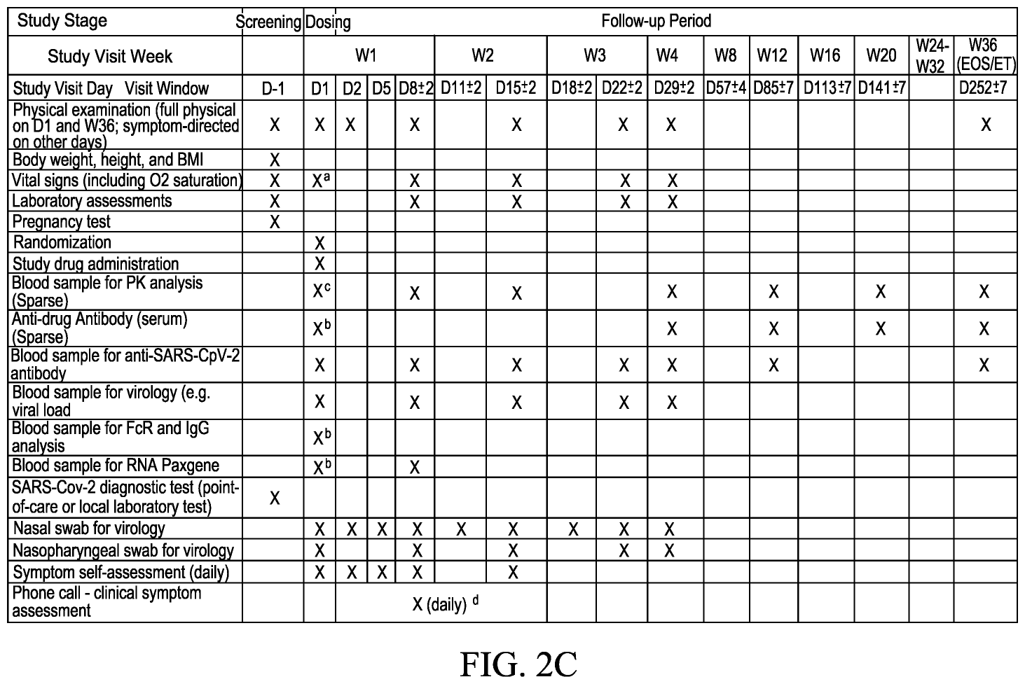

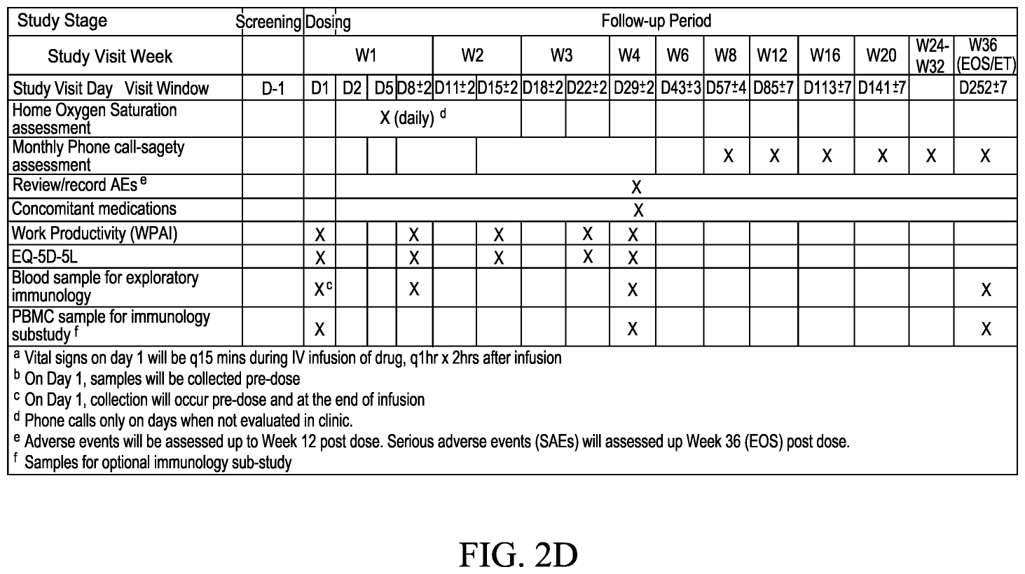

Further study details are shown in FIGS. 1-2D.

### Example 40

### Sotrovimab Monotherapy Reduces Hospitalization and Risk of Death in Early Treatment of Adults with COVID-19

In the Phase 3 portion of the clinical trial described in Example 39, safety and efficacy of a single intravenous infusion of sotrovimab (500 mg) or placebo was assessed in 583 non-hospitalized participants globally (291 patients in the treatment arm and 292 patients in the placebo arm). The primary efficacy endpoint is the proportion of patients who have progression of COVID-19 as defined by the need for hospitalization for at least 24 hours or death within 29 days of randomization. Among those studied, 63% were Hispanic or Latinx and 7% were Black or African-American. According to the Centers for Disease Control and Prevention, these populations are approximately three times more likely to be hospitalized and approximately two times more likely to die of COVID-19 (Data source: COVID-NET (www.cdc.gov/coronavirus/2019-ncov/covid-data/covid-net/purpose-methods.html, accessed Mar. 1, 2020, through Jan. 30, 2021); Numbers are ratios of age-adjusted rates standardized to the 2019 US standard COVID-NET catchment population; Data source: NCHS provisional death counts (data.cdc.gov/NCHS/Deaths-involving-coronavirus-disease-2019-COVID-19/ks3g-spdg, data through Jan. 30, 2021). Numbers are ratios of age-adjusted rates standardized to the 2019 US intercensal population estimate).

Interim analysis of data from the 583 patients enrolled in the trial demonstrated an 85% (p=0.002) reduction in hospitalization or death in patients sotrovimab as monotherapy compared to placebo, the primary endpoint of the trial. Sotrovimab was well tolerated. The trial remains ongoing and blinded with patients continuing to be followed for 24 weeks; additional results, including epidemiology and virology data, are collected.

This study and results are described in further detail:
Methods: In a multicenter, double-blind, phase 3 trial, nonhospitalized patients with symptomatic Covid-19 and at least one risk factor for disease progression were randomized (1:1) to an intravenous infusion of sotrovimab 500 mg or placebo. The primary efficacy endpoint was the proportion of patients with Covid-19 progression, defined as hospitalization longer than 24 hours or death, through day 29.
Results: In this preplanned interim analysis, which included an intent-to-treat population of 583 patients (sotrovimab, 291; placebo, 292), the primary efficacy endpoint was met. The risk of Covid-19 progression was significantly reduced by 85% (97.24% confidence interval, 44% to 96%; P=0.002) with a total of three (1%) patients progressing to the primary endpoint in the sotrovimab group versus 21 (7%) patients in the placebo group. All five patients admitted to intensive care, including one who died by day 29, received placebo. Safety was assessed in 868 patients (sotrovimab, 430; placebo, 438). Adverse events were reported by 17% and 19% of patients receiving sotrovimab and placebo, respectively; serious adverse events were less common with sotrovimab (2%) versus placebo (6%).
   In the intention-to-treat analysis (N=1057, full dataset), the adjusted relative risk reduction of hospitalization (all causes) was 79% (95% CI, 9% to
   50%; p<0.001) through Day 29 in recipients of sotrovimab (n=523) vs. placebo (n=526). Treatment with sotrovimab (ITT) resulted in a numerical reduction in the need for ER visits for illness management, hospitalisation for acute illness management (any duration) or death (any cause) compared to placebo. No participants on sotrovimab required ICU admission, compared to 9 participants on placebo, of whom 4 participants required mechanical ventilation. No participants who received sotrovimab died, compared to 4 participants on placebo. The incidence of adverse events was similar between treatment arms and SAEs were numerically more common in the placebo arm.
   Conclusion: Sotrovimab reduced progression of Covid-19 in patients with mild/moderate disease, was well tolerated, and no safety signals were identified.

### Methods

### Trial Objectives and Oversight

This phase 3, randomized, double-blind, multicenter, placebo-controlled study evaluates a single intravenous infusion of sotrovimab 500 mg for the prevention of progression of mild/moderate Covid-19 in high-risk, nonhospitalized patients. For this preplanned interim analysis, patients were recruited beginning on Aug. 27, 2020 and followed through Mar. 4, 2021 at 37 study sites in four countries (United States, Canada, Brazil, and Spain). Any changes made to the protocol and statistical analysis plan after the trial started are summarized in the Supplementary Appendix of this Example.

This study is conducted in accordance with the principles of the Declaration of Helsinki and Council for International Organizations of Medical Sciences International Ethical Guidelines, applicable International Council for Harmonisation Good Clinical Practice guidelines, and applicable laws and regulations. All patients provided written informed consent.

### Patients and Procedures

Adult patients 18 years of age or older with a positive reverse-transcriptase-polymerase-chain-reaction or antigen SARS-CoV-2 test result and onset of symptoms within the prior 5 days were screened for eligibility; screening was performed within 24 hours before study drug administration. Patients were required to be at high risk for Covid-19 progression, defined as older adults (age ≥55 years) or adults with at least one of the following risk factors: diabetes requiring medication, obesity (body-mass index >30 kg/m²), chronic kidney disease (estimated glomerular filtration rate <60 mL/min/1.73 m²),²³ congestive heart failure (New York Heart Association class II or higher), chronic obstructive pulmonary disease, and moderate to severe asthma.²⁴

Patients with already severe Covid-19, defined by shortness of breath at rest, respiratory distress, or requiring supplemental oxygen, were excluded. Inclusion criteria are described in the Supplementary Appendix of this Example.

Eligible patients were randomized 1:1 using an interactive web response system to receive either a single 500-mg, 1-hour infusion of sotrovimab or equal volume saline placebo on day 1 (FIG. 69).

### Efficacy Assessments

The primary endpoint was the proportion of patients with hospitalization for more than 24 hours or death, due to any cause, through day 29. Secondary efficacy endpoints included the proportion of patients with an emergency room visit, hospitalization, or death; mortality; patient-reported outcomes; changes in viral load; and the proportion of patients who progressed to require supplemental oxygen.

### Safety Assessments

Safety endpoints included adverse events, serious adverse events, and adverse events of special interest, defined as infusion-related reactions (including hypersensitivity reactions), immunogenicity testing for anti-drug antibodies, and evaluation of antibody-dependent enhancement. All hospitalizations, including those due to Covid-19, were counted as serious adverse events.

### Statistical Analyses

A preplanned interim analysis for safety, futility, and profound efficacy was triggered when approximately 41% of the required number of study patients reached day 29. Sample size calculations were based on a group sequential design with two interim analyses to assess both futility due to lack of efficacy or profound efficacy. A Lan-DeMets alpha spending function to control type I error was used, employing a Pocock analog rule for futility and a Hwang-Shih-DeCani (with parameter γ=1) analog for efficacy.²⁵ The overall sample size of 1360 would have provided approximately 90% power to detect a 37.5% relative efficacy in reducing progression of Covid-19 through day 29 at the overall two-sided 5% significance level, with an assumed progression rate of 16% in the placebo group.

The interim analysis intent-to-treat (ITT) population included all randomized patients through the prespecified interim analysis cutoff date of Jan. 19, 2021, irrespective of whether they received study drug. The interim analysis safety analysis population included all patients who received study medication and were randomized through Feb. 17, 2021; patients were grouped according to the actual treatment received. The primary endpoint was analyzed in the ITT population using a Poisson regression model with robust sandwich estimators adjusting for treatment, duration of symptoms, age, and gender. Missing progression status was imputed under a missing at random assumption, using multiple imputation. Based on this analysis model, the statistical significance testing, the relative risk of progression, and its appropriate confidence interval (CI) are provided using the adjusted significance level for this interim analysis.

As a result of the observed efficacy, an independent data monitoring committee recommended that enrollment in the study be stopped on Mar. 10, 2021, at which time 1057 patients had been randomized. Analyses of all secondary and exploratory endpoints is conducted when all patients have completed day 29.

### Results

### Patients

Of 795 patients screened, 583 were randomized through Jan. 19, 2021 to sotrovimab (291 patients) or placebo (292 patients); these patients comprise the interim analysis ITT population (Scheme 1, in the Supplementary Appendix of this Example). In this ITT population, similar disposition was observed across treatment groups. Overall, four patients each in the sotrovimab and placebo groups withdrew from the study. Three patients in the sotrovimab group withdrew prior to dosing, with a fourth patient withdrawing consent on day 5 for personal reasons. One patient in the placebo group withdrew consent prior to dosing, and three patients withdrew after dosing for personal reasons (days 16, 25, and 85). The median duration of follow-up in the ITT population was 72 days (range, 5 to 190) for the sotrovimab group and 72 days (range, 16 to 190) for the placebo group.

Overall, 868 patients (sotrovimab, 430 patients; placebo, 438 patients) were randomized and received study drug through Feb. 17, 2021; these patients comprise the interim analysis safety analysis population. The median duration of follow-up in this population was 56 days (range, 5 to 190) for the sotrovimab group and 55 days (range, 2 to 190) for the placebo group.

Treatment groups in the ITT population were well balanced for baseline demographic and disease characteristics (Table 8). Overall, 22% of patients were greater than 65 years of age, 7% were Black or African American, 63% were Hispanic or Latino, and 42% had two or more conditions considered to be risk factors for Covid-19 progression. The most common risk factors were obesity, age 55 years or older, and diabetes requiring medication. The most common presenting symptoms (>60% of all patients) were cough, muscle aches/myalgia, headache, and fatigue (Table 11, in the Supplementary Appendix of this Example). Baseline demographic and disease characteristics in the safety analysis population were similar across treatment groups and are reported in Table 12, in the Supplementary Appendix of this Example.

### Efficacy Outcomes

Treatment with sotrovimab resulted in an 85% reduction in the need for hospitalization over 24 hours or death, due to any cause, compared with placebo (relative risk, 0.15 [97.24% CI, 0.04 to 0.56]). Three (1%) patients progressed to the primary endpoint in the sotrovimab group versus 21 (7%) patients in the placebo group (P=0.002) (Table 9). The primary reasons for the 24 hospitalizations of more than 24 hours were consistent with progressive Covid-19 (Table 13, in the Supplementary Appendix of this Example), with one likely exception: a patient in the sotrovimab group, with a notable past medical history of small intestinal obstruction, presented 22 days after infusion with a small intestinal obstruction. Regarding the severity of these hospitalizations, all five patients who required admission to intensive care were in the placebo group; two of these five patients required invasive mechanical ventilation and a third declined intubation and subsequently died by day 29. Emergency room visits or hospitalizations for less than 24 hours were observed in fewer patients in the sotrovimab group compared with the placebo group (Table 9).

### Safety

The proportion of patients in the safety analysis population who reported an adverse event was 17% (73 of 430 patients) in the sotrovimab group and 19% (85 of 438 patients) in the placebo group (Table 10). A lower proportion of patients reported grade 3 or 4 adverse events in the sotrovimab group (2%) compared with the placebo group (6%). Overall, the only adverse event occurring in at least 1% of patients receiving sotrovimab was diarrhea, which occurred infrequently—six (1%) patients in the sotrovimab group versus three (<1%) patients in the placebo group. Among patients in the sotrovimab group, all cases of diarrhea were mild (five patients) or moderate (one patient) in severity.

Infusion-related reactions were observed in a similar proportion of patients receiving sotrovimab (1%) compared with placebo (1%). One patient receiving sotrovimab had an infusion-related reaction that was considered related to study treatment: moderate (grade 2) dyspnea.

Serious adverse events occurred in 2% of patients receiving sotrovimab and 6% of patients receiving placebo. Most of these events were hospitalizations due to Covid-19-related causes. No serious adverse events were considered related to sotrovimab.

One patient in the placebo group died after day 29; this patient died due to Covid-19 pneumonia on day 37.

No trends were observed in hematologic, liver, or chemistry laboratory data. Overall, laboratory results were similar in the sotrovimab and placebo groups.

### DISCUSSION

In this preplanned interim analysis of the COMET-ICE study, a single 500-mg dose of sotrovimab reduced the risk of hospitalization (>24 hours) or death in high-risk adults with symptomatic Covid-19 by 85% compared with placebo (P=0.002). For every 17 high-risk patients with symptomatic Covid-19, sotrovimab prevented one hospitalization. Among those who were hospitalized, no patient who received sotrovimab required admission to intensive care compared with five patients who received placebo, suggesting that sotrovimab may also prevent more severe complications of Covid-19 in addition to preventing the need for hospitalization itself. The full analysis provided similar results. Furthermore, as a result of investigator site selection, over 60% of the study population consisted of patients self-identifying as Hispanic or Latino; thus, this trial is one of the first to demonstrate efficacy in a population that has been largely underrepresented in Covid-19 clinical trials, despite the disproportionately negative impact the pandemic has had in this ethnic group. Overall, sotrovimab was well tolerated, and no safety signals were identified in this study. There was also no evidence of antibody-dependent enhancement with sotrovimab, which could have manifested as worsening of disease compared with placebo.²⁶

Treatments for Covid-19 will need to retain activity in the face of an evolving virus. To that end, sotrovimab may have an intrinsically high barrier to resistance as a result of targeting a pan-sarbecovirus epitope.¹⁴ In one analysis, among more than 584,000 sequences in the Global Influenza Surveillance and Response System database (Global Initiative on Sharing All Influenza Data), amino acid positions comprising the sotrovimab epitope were at least 99.96% conserved in naturally occurring viruses.¹⁴ Moreover, when necessary to further enhance breadth and barrier to resistance, sotrovimab can likely be combined with currently authorized receptor-binding motif-targeted antibodies due to its nonoverlapping resistance profile.

These results also indicate that a non-receptor-binding motif binding antibody, which does not directly block the ACE2 receptor interaction, can be clinically therapeutic, and suggests a role for other receptors.²⁷ Moreover, as sotrovimab has potent effector function, the absence of safety signals and observed efficacy strongly suggest that effector function is neither detrimental nor associated with antibody-dependent enhancement.²⁶ In fact, preclinical models of Covid-19 suggest that its potent effector function may be beneficial.^{13,14}

Results from this interim analysis (as well as from the full intent-to-treat analysis) of the COMET-ICE trial support that sotrovimab can be an important therapeutic for the outpatient treatment of Covid-19. Notably, a 500-mg dose may be administered intramuscularly, increasing the convenience of and access to antibody therapeutics for patients with Covid-19. Studies are currently underway to evaluate this route of administration.

### REFERENCES

1. Coronavirus Resource Center. (https://coronavirus.jhu.edu.)
2. Angulo F J, Finelli L, Swerdlow D L. Estimation of US SARS-CoV-2 infections, symptomatic infections, hospitalizations, and deaths using seroprevalence surveys. JAMA Network Open 2021; 4:e2033706-e.
3. Reese H, Iuliano A D, Patel N N, et al. Estimated incidence of COVID-19 illness and hospitalization-United States, February-September 2020. Clin Infect Dis 2020. doi: 10.1093/cid/ciaa1780. Epub ahead of print.
4. Petrilli C M, Jones S A, Yang J, et al. Factors associated with hospital admission and critical illness among 5279 people with coronavirus disease 2019 in New York City: prospective cohort study. BMJ 2020; 369: m1966.
5. Liang W, Liang H, Ou L, et al. Development and validation of a clinical risk score to predict the occurrence of critical illness in hospitalized patients with COVID-19. JAMA Intern Med 2020; 180:1081-9.
6. Cariou B, Hadjadj S, Wargny M, et al. Phenotypic characteristics and prognosis of inpatients with COVID-19 and diabetes: the CORONADO study. Diabetologia 2020; 63:1500-15.
7. Huang C, Wang Y, Li X, et al. Clinical features of patients infected with 2019 novel coronavirus in Wuhan, China. Lancet 2020; 395:497-506.
8 CDC COVID-19 Response Team. Severe outcomes among patients with coronavirus disease 2019 (COVID-19)—United States, Feb. 12-Mar. 16, 2020. MMWR Morb Mortal Wkly Rep 2020; 69:343-6.
9. Chen P, Nirula A, Heller B, et al. SARS-CoV-2 neutralizing antibody LY-CoV555 in outpatients with Covid-19. N Engl J Med 2021; 384:229-37.
10. Weinreich D M, Sivapalasingam S, Norton T, et al. REGN-COV2, a neutralizing antibody cocktail, in outpatients with Covid-19. N Engl J Med 2021; 384:238-51.
11. Wang P, Nair M S, Liu L, et al. Antibody resistance of SARS-CoV-2 variants B.1.351 and B.1.1.7. Nature 2021. doi: 10.1038/s41586-021-03398-2. Epub ahead of print.
12. Chen R E, Zhang X, Case J B, et al. Resistance of SARS-CoV-2 variants to neutralization by monoclonal and serum-derived polyclonal antibodies. Nat Med 2021; 27 (4): 717-26.
13. Pinto D, Park Y J, Beltramello M, et al. Cross-neutralization of SARS-CoV-2 by a human monoclonal SARS-CoV antibody. Nature 2020; 583:290-5.
14. Cathcart A L, Havenar-Daughton C, Lempp F A, et al. The dual function monoclonal antibodies VIR-7831 and VIR-7832 demonstrate potent in vitro and in vivo activity against SARS-CoV-2. bioRxiv 2021. doi: 10.1101/2021.03.09.434607. Epub ahead of print.
15. McCallum M, Bassi J, Marco A, et al. SARS-CoV-2 immune evasion by variant B.1.427/B.1.429. bioRxiv 2021. doi: 10.1101/2021.03.31.437925. Epub ahead of print.
16. Focosi D, Maggi F. Neutralising antibody escape of SARS-CoV-2 spike protein: risk assessment for antibody-based Covid-19 therapeutics and vaccines. Rev Med Virol 2021. doi: 10.1002/rmv.2231. Epub ahead of print.
17. Tada T, Dcosta B M, Zhou H, Vaill A, Kazmierski W, Landau N R. Decreased neutralization of SARS-CoV-2 global variants by therapeutic anti-spike protein monoclonal antibodies. bioRxiv 2021. doi: 10.1101/2021.02.18.431897. Epub ahead of print.
18. Starr T N, Greaney A J, Dingens A S, Bloom J D. Complete map of SARS-CoV-2 RBD mutations that escape the monoclonal antibody LY-CoV555 and its cocktail with LY-CoV016. Cell Rep Med 2021; 2:100255.
19. Liu H, Wei P, Zhang Q, et al. 501Y.V2 and 501Y.V3 variants of SARS-CoV-2 lose binding to Bamlanivimab in vitro. bioRxiv 2021. doi: 10.1101/2021.02.16.431305. Epub ahead of print.
20. Ko S Y, Pegu A, Rudicell R S, et al. Enhanced neonatal Fc receptor function improves protection against primate SHIV infection. Nature 2014; 514:642-5.
21. Zalevsky J, Chamberlain A K, Horton H M, et al. Enhanced antibody half-life improves in vivo activity. Nat Biotechnol 2010; 28:157-9.
22. Gaudinski M R, Coates E E, Houser K V, et al. Safety and pharmacokinetics of the Fc-modified HIV-1 human monoclonal antibody VRC01LS: a phase 1 open-label clinical trial in healthy adults. PLoS Med 2018; 15:e1002493.
23. Levey A S, Bosch J P, Lewis J B, Greene T, Rogers N, Roth D. A more accurate method to estimate glomerular filtration rate from serum creatinine: a new prediction equation. Modification of Diet in Renal Disease Study Group. Ann Intern Med 1999; 130:461-70.
24. COVID-19: developing drugs and biological products for treatment or prevention. May 2020. (Accessed Feb. 19, 2021, at www.fda.gov/meida/137926/download.)
25. Hwang I K, Shih W J, De Cani J S. Group sequential designs using a family of type I error probability spending functions. Stat Med 1990; 9:1439-45.
26. Arvin A M, Fink K, Schmid M A, et al. A perspective on potential antibody-dependent enhancement of SARS-CoV-2. Nature 2020; 584:353-63.
27. Lempp F A, Soriaga L, Montiel-Ruiz M, et al. Membrane lectins enhance SARS-CoV-2 infection and influence the neutralizing activity of different classes of antibodies. bioRxiv 2021. doi: 10.1101/2021.04.03.438258. Epub ahead of print.

TABLE 8
Baseline Demographic and Disease Characteristics (ITT Population)

| | Sotrovimab | Placebo | Total |
|---|---|---|---|
| | (N = 291) | (N = 292) | (N = 583) |
| Characteristic | | | |
| Age - yr, median (range) | 53.0 (18-96) | 52.5 (18-88) | 53.0 (18-96) |
| ≥65 yr - no. (%) | 63 (22) | 65 (22) | 128 (22) |
| >70 yr - no. (%) | 33 (11) | 32 (11) | 65 (11) |
| Male gender - no. (%) | 135 (46) | 131 (45) | 266 (46) |
| Race* - no. (%) | | | |
| White | 254 (88) | 252 (87) | 506 (87) |
| Black or African | 16 (6) | 22 (8) | 38 (7) |
| American | | | |
| Asian | 17 (6) | 17 (6) | 34 (6) |
| Mixed race | 2 (<1) | 0 | 2 (<1) |
| American Indian or | 1 (<1) | 0 | 1 (<1) |
| Alaska Native | | | |
| Hispanic or Latino ethnic | 190 (65) | 178 (61) | 368 (63) |
| group - no. (%) | | | |
| Body-mass index† - mean | 32.0 (6.4) | 32.1 (6.3) | 32.1 (6.3) |
| (SD) | | | |
| Duration of symptoms‡ - | | | |
| no. (%) | | | |
| ≤3 days | 167 (57) | 171 (59) | 338 (58) |
| 4-5 days | 123 (42) | 121 (41) | 244 (42) |
| Any risk factor for Covid- | 291 (100) | 290 (>99) | 581 (>99) |
| 19 progression - no. (%) | | | |
| Age ≥55 yr | 135 (46) | 141 (48) | 276 (47) |
| Diabetes requiring | 66 (23) | 66 (23) | 132 (23) |
| medication | | | |
| Obesity (body-mass | 182 (63) | 187 (64) | 369 (63) |
| index >30†) | | | |
| Chronic kidney disease | 1 (<1) | 4 (1) | 5 (<1) |
| (eGFR <60 by MDRD) | | | |
| Congestive heart failure | 1 (<1) | 3 (1) | 4 (<1) |
| (NYHA class II or more) | | | |
| Chronic obstructive | 14 (5) | 10 (3) | 24 (4) |
| pulmonary disease | | | |
| Moderate to severe | 46 (16) | 46 (16) | 92 (16) |
| asthma | | | |
| Number of concurrent risk | | | |
| factors for Covid-19 | | | |
| progression - no. (%) | | | |
| 0 | 0 | 2 (<1) | 2 (<1) |
| 1 | 170 (58) | 168 (58) | 338 (58) |
| 2 | 91 (31) | 86 (29) | 177 (30) |
| ≥3 | 30 (10) | 36 (12) | 66 (11) |

ITT denotes intent-to-treat, SD standard deviation, eGFR estimated glomerular filtration rate, MDRD Modification of Diet in Renal Disease, NYHA New York Heart Association.
*Race data were not available for one patient in the sotrovimab group and one patient in the placebo group.
†Body-mass index is the weight in kilograms divided by the square of the height in meters.
‡One patient in the sotrovimab group had a symptom duration of 6 days.

TABLE 9
Summary of Efficacy Outcomes Through Day 29 (ITT Population)

| | |
|---|---|
| Sotrovimab | Placebo |
| (N = 291) | (N = 292) |

Primary outcome

| | | |
|---|---|---|
| Hospitalized >24 hours or death for any cause - no. | 3 (1) | 21 (7) |
| (%) | | |
| Hospitalized >24 hours for any cause | 3 (1) | 21 (7) |
| Death by any cause | 0 | 1 (<1) |
| Alive and not hospitalized - no. (%) | 284 (98) | 270 (92) |
| Missing - no. (%) | 4 (1) | 1 (<1) |
| Withdrew consent prior to dosing - no. (%) | 3 (1) | 1 (<1) |

| | |
|---|---|
| Percent reduction (97.24% CI), P value | 85% (44% to 96%); |
| | P = 0.002 |

Other clinical outcomes*

| | | |
|---|---|---|
| Emergency room visit, hospitalization, or death for | 6 (2) | 28 (10) |
| any cause - no. (%) | | |
| Emergency room visit for any cause | 2 (<1) | 8 (3) |
| Hospitalized for any cause | 4 (1)† | 21 (7) |
| Death by any cause | 0 | 1 (<1) |
| Emergency room visit without hospitalization or | 3 (1) | 7 (2) |
| hospitalized for <24 hours for any cause - no. (%) | | |
| Severe or critical progression‡ - no. (%) | 2 (<1) | 19 (7) |
| Low-flow nasal cannula or face mask | 2 (<1) | 11 (4) |
| Non-rebreather mask, high-flow nasal cannula, or | 0 | 5 (2) |
| noninvasive ventilation | | |
| Invasive mechanical ventilation | 0 | 2 (<1) |
| Death by any cause | 0 | 1 (<1) |
| Admission to intensive care for any cause - no. (%) | 0 | 5 (2) |

ITT denotes intent-to-treat, CI confidence interval.
*Inferential testing of secondary endpoints has not been performed at this interim analysis.
†One patient was hospitalized for less than 24 hours for diabetes management.
‡Severe or critical progression as manifested by supplemental oxygen use.

TABLE 10
Summary of Adverse Events (Safety Analysis Population)

| | Sotrovimab | Placebo |
|---|---|---|
| | (N = 430) | (N = 438) |
| Any adverse event - no. (%) | 73 (17) | 85 (19) |
| Related to study treatment* | 8 (2) | 8 (2) |
| Leading to permanent discontinuation of | 0 | 0 |
| study treatment† | | |
| Leading to dose interruption/delay | 2 (<1)* | 0 |
| Occurring in ≥1% of patients receiving | | |
| sotrovimab by preferred term | | |
| Diarrhea | 6 (1) | 3 (<1) |
| Any infusion-related reaction§ - no. (%) | 6 (1) | 5 (1) |
| Related to study treatment* | 1 (<1) | 2 (<1) |
| Leading to permanent discontinuation of | 0 | 0 |
| study treatment† | | |
| Leading to dose interruption/delay | 0 | 0 |
| Any grade 3 or 4 adverse event - no. (%) | 7 (2) | 27 (6) |
| Any serious adverse event - no. (%) | 7 (2) | 26 (6) |
| Related to study treatment* | 0 | 1 (<1) |
| Fatal | 0 | 2 (<1)¶ |
| Related to study treatment* | 0 | 0 |

*Relatedness was determined by individual study investigators while blinded to study treatment.
†A patient was permanently discontinued from the completion of drug infusion if they experienced life-threatening infusion-related reactions, including severe allergic or hypersensitivity reactions during the intravenous infusion.
‡For both patients, the adverse event was infusion extravasation; both infusions were completed.
§Infusion-related reactions were defined as adverse events with preferred terms of pyrexia, chills, dizziness, dyspnea, pruritus, rash, and infusion-related reaction within 24 hours of study drug administration.
¶In addition to the patient in the placebo group who died by day 29, another patient in the placebo group died due to Covid-19 pneumonia on day 37; this patient was admitted to the hospital prior to day 29 and was thus considered to have met the primary endpoint definition of Covid-19 progression.

### SUPPLEMENTARY APPENDIX

### Table of Contents

Supplementary methods
Changes to protocol/SAP
Table 11. Presenting symptoms (ITT population)
Table 12. Baseline demographic and disease characteristics (safety analysis population)
Table 13. Primary reasons for hospitalizations of more than 24 hours (ITT population)
Scheme 1. Patient disposition (ITT population)

### Supplementary Methods

### Inclusion Criteria

Patients are eligible to be included in the study only if all of the following criteria apply:

### Age and Risk Factors

6. Patient must be 18 years of age or older AND at high risk of progression of Covid-19 based on presence of one or more of the following risk factors:
   1. Diabetes (requiring medication)
   2. Obesity (body-mass index >35 kg/m²)
1. Change: body-mass index threshold was >30 kg/m² in the original protocol. See “Changes to Protocol/SAP” section for more information
   3. Chronic kidney disease (i.e., estimated glomerular filtration rate <60 mL/min/1.73 m² according to the Modification of Diet in Renal Disease study equation)
   4. Congestive heart failure (New York Heart Association class II or more)
   5. Chronic obstructive pulmonary disease (history of chronic bronchitis, chronic obstructive lung disease, or emphysema with dyspnea on physical exertion) and moderate to severe asthma (patient requires an inhaled steroid to control symptoms or has been prescribed a course of oral steroids in the past year)
      or
      7. Patient 55 years of age or older, irrespective of comorbidities
      Note: target enrollment of ˜15% of patients over 70 years of age
      Change: target enrollment criterion was not in the original protocol. See “Changes to Protocol/SAP” section for more information

### Type of Patient and Disease Characteristics

8. Patients who have a positive SARS-CoV-2 test result (by any validated diagnostic test [e.g. RT-PCR, antigen-based testing on any specimen type])
and
9. Oxygen saturation ≥94% on room air
and
10. Have Covid-19 defined by one or more of the following symptoms: fever, chills, cough, sore throat, malaise, headache, joint or muscle pain, change in smell or taste, vomiting, diarrhea, shortness of breath on exertion
and
11. Less than or equal to 5 days from onset of symptoms

### Sex and Contraceptive/Barrier Requirements

12. No gender restrictions
13. Female patients must meet and agree to abide by the following contraceptive criteria:
   1. Contraception use by women should be consistent with local regulations regarding the methods of contraception for those participating in clinical studies
   2. A female patient is eligible to participate if she is not pregnant or breastfeeding and one of the following conditions applies:
14. Is a woman of nonchildbearing potential
15. Is a woman of childbearing potential and is using a contraceptive method that is highly effective, with a failure rate of <1%, during the study intervention period and for up to 24 weeks after the last dose of study intervention. The investigator should evaluate potential for contraceptive method failure (e.g., noncompliance, recently initiated) in relationship to the first dose of study intervention
16. A woman of childbearing potential must have a negative highly sensitive pregnancy test (urine or serum as required by local regulations) before the first dose of study intervention. If a urine test cannot be confirmed as negative (e.g., an ambiguous result), a serum pregnancy test is required. In such cases, the patient must be excluded from participation if the serum pregnancy result is positive
17. The investigator is responsible for the review of medical history, menstrual history, and recent sexual activity to decrease the risk for inclusion of a woman with an early undetected pregnancy

### Informed Consent

18. Capable of giving signed informed consent, which includes compliance with the requirements and restrictions listed in the informed consent form and in this protocol
or
19. If patients are not capable of giving written informed consent, alternative consent procedures will be followed

### Prior/Concurrent Clinical Study Experience

20. Enrollment in any investigational vaccine study within the last 180 days or any other investigational drug study within 30 days prior to day 1 or within five half-lives of the investigational compound, whichever is longer
21. Enrollment in any trial of an investigational vaccine for SARS-CoV-2

### Procedures

In-person study visits occurred on days 5, 8, 11, 15, 22, and 29, as well as daily telephone calls through day 15 (except on in-person visit days) to assess for adverse events and worsening of Covid-19. Starting at week 8, patients were monitored monthly via alternating telephone calls (weeks 8 and 16) and in-person visits (weeks 12, 20, and 24) for Covid-19 illness for a total of 24 weeks from dosing. Blood samples were collected for laboratory assessments at days 1, 15, 22, and 29 and for anti-drug antibodies on days 1 and 29. Patient-reported outcome assessments are administered through week 24.

### Changes to Protocol/SAP

22. Local safety laboratory assessments at screening were modified to specify that only ABO typing was required per protocol
23. Rationale:
24. While certain screening assessments to confirm study patient eligibility must be performed or documented locally (e.g., pregnancy test for women of childbearing potential and SARS-CoV-2 infection via a validated diagnostic test), ABO typing is the only required local laboratory assessment at screening for safety purposes
25. All other local safety laboratory assessments during the screening visit (hematology, clinical chemistry, and coagulation parameters) may or may not be performed according to the clinical discretion of the study investigator or as required by local regulations
26 Considering the time to randomization and requirement for central laboratory testing on day 1 (randomization), the amount of sample collection was overly cumbersome for sites
27 Study objectives and endpoints (secondary and exploratory endpoints) were modified, including the introduction of an additional secondary endpoint (i.e., evaluation of the proportion of patients who have progression of Covid-19 through day 29 as defined by a visit to a hospital emergency room for management of illness, hospitalization for acute management of illness, or death), amendment to existing patient-reported outcome and virology endpoints, and introduction of additional exploratory endpoints
1. Rationale:
   2. Preliminary clinical trial data generated for other monoclonal antibodies targeting Covid-19 suggest that the rate of progression of Covid-19 leading to the requirement for hospitalization may be lower than originally estimated and the efficacy of antispike protein monoclonal antibodies in the treatment of Covid-19 in the outpatient population is greater than a 50% decrease in the proportion of medically attended visits through day 29
   3. In light of new data from recent clinical trials of anti-SARS-CoV-2 mAbs, the secondary and exploratory endpoints were modified to reflect the clinical outcomes that are most relevant in the outpatient populations who receive Covid-19 monoclonal antibodies
28 Modified the “Age and Risk Factors” eligibility criteria (inclusion criteria #3 and #4) to enrich high-risk populations with the highest unmet medical need
1. Rationale:
   2. The “Age and Risk Factors” eligibility criteria has been modified to increase the body-mass index requirement defining obesity and introduce a targeted minimum number of patients (approximately 15%) >70 years of age
   3. In light of emerging clinical data on the progression of Covid-19 in the outpatient setting, eligibility criteria were amended to enrich for populations at high risk for progression to severe Covid-19
4. Modified the “Medical Conditions” eligibility criteria (exclusion criteria #10) to enrich high-risk populations with the highest unmet medical need
5. Rationale:
   6. The “Medical Conditions” eligibility criteria have been modified to clarify the definition of “severely immunocompromised”
   7. In light of emerging clinical data on the progression of Covid-19 in the outpatient setting, eligibility criteria were modified to enrich for immunosuppressed populations at high risk for progression to severe Covid-19
29 Modified the “Other Exclusions” eligibility criteria and “Medication Not Permitted During the Study” to clarify restrictions around dosing with a SARS-CoV-2 vaccine
1. Rationale:
   1. Considering the recent data published on SARS-CoV-2 vaccines, the protocol was amended to clarify restrictions surrounding administration of an experimental or approved SARS-CoV-2 vaccine
2. Clarified guidance for infusion-related reactions
1. Rationale:
   1. Protocol language was amended to clarify that sites should follow local or institutional guidelines for the treatment of infusion-related reactions
2. Statistical analysis plan (interim analysis #1, interim analysis #2, and statistical approach) were modified
1. Rationale:
   1. Preliminary clinical trial data generated for other monoclonal antibodies targeting Covid-19 suggest that the rate of progression of Covid-19 to requirement for hospitalization may be lower than originally estimated and the efficacy of antispike protein monoclonal antibodies in the treatment of Covid-19 in the outpatient population is greater than a 50% decrease in the proportion of medically attended visits through day 29
   2. In light of this emerging data, the interim analyses were modified to more accurately reflect the most recent data on progression of Covid-19 and the potential efficacy of monoclonal antibodies against SARS-CoV-2
3. Specified an intention to conduct a “non-Covid-19” safety analysis
1. Rationale:
   1. Since it is not be possible to delineate in a single patient whether the hospitalization is directly related to Covid-19 complications or could be related to sotrovimab causing more severe disease due to antibody-dependent enhancement, all hospitalizations regardless of cause are included in the primary endpoint and are counted as serious adverse events
   2. To inform on the number and nature of non-Covid-19 adverse events and serious adverse events, additional safety analyses are performed in which select, prespecified terms consistent with known progression of Covid-19 disease are excluded.

TABLE 11
Presenting Symptoms (ITT Population)

| | Sotrovimab | Placebo | Total |
|---|---|---|---|
| Symptom - no. (%) | (N = 291) | (N = 292) | (N = 538) |
| Cough | 240 (82) | 247 (85) | 487 (84) |
| Muscle | 215 (74) | 215 (74) | 430 (74) |
| aches/myalgia | | | |
| Headache | 202 (69) | 216 (74) | 418 (72) |
| Fatigue | 180 (62) | 183 (63) | 363 (62) |
| Malaise | 172 (59) | 172 (59) | 344 (59) |
| Sore throat | 171 (59) | 172 (59) | 343 (59) |
| Fever | 164 (56) | 168 (58) | 332 (57) |
| Loss of taste | 171 (59) | 159 (54) | 330 (57) |
| Loss of smell | 175 (60) | 152 (52) | 327 (56) |
| Chills | 164 (56) | 158 (54) | 322 (55) |
| Joint pain/arthralgia | 153 (53) | 153 (52) | 306 (52) |
| Shortness of breath | 131 (45) | 131 (45) | 262 (45) |
| Diarrhea | 87 (30) | 101 (35) | 188 (32) |
| Nausea | 85 (29) | 91 (31) | 176 (30) |
| Vomiting | 34 (12) | 37 (13) | 71 (12) |

ITT denotes intent-to-treat.

TABLE 12
Baseline Demographic and Disease Characteristics (Safety Analysis Population)

| | Sotrovimab | Placebo | Total |
|---|---|---|---|
| | (N = 430) | (N = 438) | (N = 868) |
| Characteristic | | | |
| Age - yr, median (range) | 53.0 (18-96) | 52.0 (17-88) | 53.0 (17-96) |
| ≥65 yr - no. (%) | 84 (20) | 88 (20) | 172 (20) |
| >70 yr - no. (%) | 42 (10) | 42 (10) | 84 (10) |
| Male gender - no. (%) | 194 (45) | 212 (48) | 406 (47) |
| Race* - no. (%) | | | |
| White | 374 (87) | 384 (88) | 758 (88) |
| Black or African American | 27 (6) | 33 (8) | 60 (7) |
| Asian | 21 (5) | 19 (4) | 40 (5) |
| Mixed race | 6 (1) | 0 | 6 (<1) |
| American Indian or Alaska Native | 1 (<1) | 1 (<1) | 2 (<1) |
| Hispanic or Latino ethnic group - no. | 280 (65) | 280 (64) | 560 (65) |
| (%) | | | |
| Body-mass index^{†} - mean (SD) | 32.1 (6.4) | 32.5 (6.7) | 32.3 (6.5) |
| Duration of symptoms‡ - no. (%) | | | |
| ≤3 days | 254 (59) | 260 (59) | 514 (59) |
| 4-5 days | 173 (40) | 178 (41) | 351 (40) |
| Any risk factor for Covid-19 | 427 (>99) | 434 (>99) | 861 (>99) |
| progression - no. (%) | | | |
| Age ≥55 yr | 195 (45) | 205 (47) | 400 (46) |
| Diabetes requiring medication | 93 (22) | 88 (20) | 181 (21) |
| Obesity (body-mass index >30^{†}) | 267 (62) | 292 (67) | 559 (64) |
| Chronic kidney disease (eGFR <60 by | 2 (<1) | 5 (1) | 7 (<1) |
| MDRD) | | | |
| Congestive heart failure (NYHA class II | 4 (<1) | 3 (<1) | 7 (<1) |
| or more) | | | |
| Chronic obstructive pulmonary disease | 24 (6) | 18 (4) | 42 (5) |
| Moderate to severe asthma | 69 (16) | 72 (16) | 141 (16) |
| Number of concurrent risk factors for | | | |
| Covid-19 progression - no. (%) | | | |
| 0 | 3 (<1) | 4 (<1) | 7 (<1) |
| 1 | 251 (58) | 250 (57) | 501 (58) |
| 2 | 132 (31) | 130 (30) | 262 (30) |
| ≥3 | 44 (10) | 54 (13) | 98 (11) |

SD denotes standard deviation, eGFR estimated glomerular filtration rate, MDRD Modification of Diet in Renal Disease, NYHA New York Heart Association.
*Race data were not available for one patient in the sotrovimab group and one patient in the placebo group.
^{†}Body-mass index is the weight in kilograms divided by the square of the height in meters.
‡One patient in the sotrovimab group had a symptom duration of 6 days. For two other patients in the sotrovimab group, symptom duration data were not available at the time of this interim analysis.

TABLE 13
Primary Reasons for Hospitalizations of More Than 24 Hours (ITT Population)

| | | | | | | | |
|---|---|---|---|---|---|---|---|
| | | | | | Intensive | Invasive | |
| | Age | | Hospitalization | Primary | care unit | mechanical | |
| Patient | (yr) | Sex | day | reason | admission | ventilation | Fatal |

Sotrovimab-treated patients

| | | | | | | | |
|---|---|---|---|---|---|---|---|
| A | 96 | M | 19 | Covid-19 | N | N | N |
| B | 65 | F | 22 | Small | N | N | N |
| | | | | intestinal | | | |
| | | | | obstruction | | | |
| C | 71 | F | 2 | Covid- | N | N | N |
| | | | | pneumonia | | | |

Placebo-treated patients

| | | | | | | | |
|---|---|---|---|---|---|---|---|
| D | 52 | F | 4 | Covid- | N | N | N |
| | | | | pneumonia | | | |
| E | 50 | F | 4* | Covid- | N | N | N |
| | | | | pneumonia | | | |
| F | 66 | F | 6 | Covid- | N | N | N |
| | | | | pneumonia | | | |
| G | 38 | M | 9* | Covid- | N | N | N |
| | | | | pneumonia | | | |
| H | 50 | F | 4* | Covid- | Y | N | N |
| | | | | pneumonia | | | |
| I | 82 | F | 7 | Acute | N | N | N |
| | | | | respiratory | | | |
| | | | | failure | | | |
| J | 70 | M | 12 | Respiratory | Y | Y | N |
| | | | | distress | | | |
| K | 70 | M | 5 | Pneumonia | Y | N | Y |
| L | 65 | M | 6 | Dehydration | N | N | N |
| M | 52 | F | 5 | Covid- | N | N | N |
| | | | | pneumonia | | | |
| N | 62 | M | 7 | Covid- | N | N | N |
| | | | | pneumonia | | | |
| O | 57 | M | 6 | Pneumonia | N | N | N |
| P | 65 | M | 12 | Pneumonia | N | N | N |
| Q | 68 | M | 7 | Covid- | N | N | N |
| | | | | pneumonia | | | |
| R | 55 | F | 7 | Pulmonary | N | N | N |
| | | | | embolism | | | |
| S | 60 | M | 10 | Covid- | N | N | N |
| | | | | pneumonia | | | |
| T | 71 | F | 10 | Covid- | Y | Y | Y |
| | | | | pneumonia | | | |
| U | 37 | F | 6* | Covid- | N | N | N |
| | | | | pneumonia | | | |
| V | 83 | M | 8 | Dyspnea | N | N | N |
| W | 56 | M | 2 | Covid- | Y | N | N |
| | | | | pneumonia | | | |
| X | 55 | M | 7 | Covid- | N | N | N |
| | | | | pneumonia | | | |

ITT denotes intent-to-treat, M male, F female, N no, Y yes.
*The adverse event associated with the hospitalization started the day before the patient was admitted to the hospital.

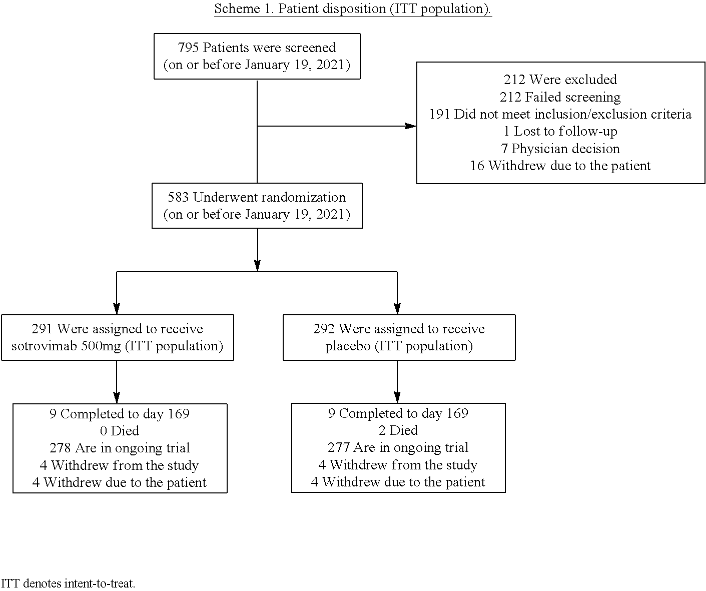

### Example 41

### Phase 2/3 Clinical Study of Sotrovimab for Treatment of Hospitalized Subjects with Severe to Critical COVID-19 Disease

A randomized, multi-center, double-blind, placebo-controlled study is performed to assess safety and efficacy of monoclonal antibody sotrovimab in patients with severe or critical COVID-19 disease.

A primary objective is to compare efficacy of sotrovimab versus placebo. Secondary objectives are to compare efficacy of sotrovimab versus placebo on viral shedding in upper and lower respiratory samples, to compare safety and tolerability of sotrovimab versus placebo, to determine efficacy of sotrovimab versus placebo for induction of an anti-viral response, to determine efficacy of sotrovimab versus placebo for increasing ICU- and hospital-free days, and to determine serum pharmacokinetics (PK) of sotrovimab.

Exploratory objectives are to compare efficacy of sotrovimab against versus placebo in preventing non-respiratory complications of COVID-19 disease, to compare effect of sotrovimab versus placebo on viral shedding in upper and lower respiratory samples and blood samples, to assess emergence of resistance to sotrovimab, to evaluate effect of sotrovimab on potential biomarkers of host response, and to assess correlations between efficacy, safety, PK, viral shedding, and immunogenicity.

### Details of Criteria for Evaluation

Primary endpoints of this study are as follows:
1. Proportion of subjects who progress to severe or critical COVID-19 or die through Day 29 as defined by:
   1. Severe disease: hypoxemia (O2 saturation ≤93% on room air or PaO2/FiO2<300) requiring oxygen supplementation >1 day OR subject requires ≥4 L/min oxygen supplementation or equivalent AND the investigator or designee determine that the measurement of O2 saturation on room air is medically unsafe
OR
   2. Critical disease: Respiratory failure requiring at least one of the following: invasive mechanical ventilation, ECMO; OR shock; OR multi-organ dysfunction/failure
OR
   3. Death
2 Time to clinical response, defined as being alive and independent of oxygen supplementation through Day 29, as compared to standard of care (SOC).

Secondary endpoints of this study are as follows:
3. Occurrence of adverse events (AEs)
4. Occurrence of serious adverse events (SAEs)
5. Occurrence of adverse events of special interest (AESI)
6. Proportion of subjects who progress to develop severe or critical COVID-19 at Day 8, Day 15, or Day 22
7. 29-day, 60-day, and 90-day all-cause mortality
8. Total number of ventilator days from randomization through 29 days
9. Total number of days requiring supplemental oxygen through 29 days
10. Total intensive care length of stay (LOS)
11 Total hospital LOS
12. Severity and duration of subject reported signs and symptoms of COVID-19 related illness using the Flu-PRO patient-reported outcome measurement instrument
13. Occurrence of cardiac, thromboembolic, renal, neurologic events
14. Detection of SARS-CoV-2 in nasal secretions by PCR at baseline and during follow-up period through Day 29
15. Time to no detectable viral RNA (<LLOQ) by quantitative RT-PCR from nasopharyngeal samples
16. Sotrovimab pharmacokinetics (PK) in serum
17. Incidence and titers (if applicable) of serum ADA to Sotrovimab

Exploratory endpoints of this study are as follows:
30. Proportion of subjects at Day 8, Day 15, Day 22, and Day 29 in each category of the ordinal scale for clinical improvement (see below)
31. Emergence of viral resistance mutants to mAb by SARS-CoV-2
32. Viral load in nasal secretions and blood by qRT-PCR
33. Host transcriptome and immunophenotyping analysis
34. FcγR polymorphisms as determined by genotyping
35. IgG1 allotypes as determined by genotyping
36. Change from baseline in Work Productivity and Activity Impairment (WPAI)
37. Change from baseline in health-related quality of life according to EQ-5D-5L

The ordinal scale to be used for categorization of clinical improvement is as follows:
1. Uninfected: No clinical or virologic evidence of infection
2. Non-hospitalized, no limitation of activity (if using oxygen at home, report as category 4)
3. Non-hospitalized, limitation of activity
4. Hospitalized, no oxygen therapy
5. Hospitalized, low-flow oxygen by mask or nasal prongs
6. Hospitalized, high-flow oxygen, Continuous positive airway pressure (CPAP), bilevel positive airway pressure (BiPAP), non-invasive ventilation
7. Hospitalized, intubation and mechanical ventilation
8 Hospitalized, mechanical ventilation plus organ support (vasopressors, RRT, ECMO)
9 Death

### Number of Subjects Planned

This study is comprised of two parts. Part 1 enrolls approximately 20 subjects with severe COVID-19 disease. Part 2 enrolls approximately 500 subjects with severe to critical COVID-19 disease; approximately 250 subjects per treatment arm.

### Randomization and Stratification

Subjects are randomized in a 1:1 ratio to receive a single IV dose of sotrovimab plus standard of care (SoC) or equal volume saline placebo plus SoC.

For Part 1, the first 20 subjects enrolled in the study (ten per arm) are in the severe COVID-19 disease population (Grade 4 or 5). These subjects are stratified by time from onset of symptoms, either 1) less than seven days or 2) seven days or more. All subjects enrolled in Part 1 are assigned to intensive PK sampling. Serum PK samples are collected from these subjects.

For Part 2, the clinical status of the subjects are stratified by disease status (severe or critical) and time from onset of symptoms, as follows:
1. Severe (Grade 4 or 5) and less than seven days since onset of symptoms
2. Severe (Grade 4 or 5) and seven days or more since onset of symptoms
3. Critical (Grade 6 or 7) and less than seven days since onset of symptoms
4. Critical (Grade 6 or 7) and seven days or more since onset of symptoms. Sparse PK samples are collected from subjects enrolled in Part 2.

### Diagnosis and Main Criteria for Inclusion

### Inclusion Criteria Include:

### Age:

10. Subjects aged 18 years and older

### Disease Characteristics:

11. Subjects who have a positive SARS-CoV-2 test result (by any validated test e.g. RT-PCR on any specimen type)
AND
12. Hospitalized with severe COVID-19 disease defined as requirement for supplemental oxygen or non-invasive ventilation consistent with Grade 4 or Grade 5 disease
OR
13. Hospitalized with critical COVID-19 disease defined as those on mechanical ventilation (Grade 6 or Grade 7 disease)
AND
14. Onset of COVID-19 symptoms within 14 days of Day 14
AND
15. Randomized within 48 hours of admission to hospital

### Sex:

16. No gender restriction
17. Female subjects must meet and agree to abide by the following contraceptive criteria. Contraception use by women should be consistent with local regulations regarding the methods of contraception for those participating in clinical studies.
18. A female subject is eligible to participate if she is not pregnant or breastfeeding, and one of the following conditions applies:
Is a woman of non-childbearing potential (WONCBP)
OR
Is a woman of child-bearing potential (WOCBP) and using a contraceptive method that is highly effective, with a failure rate of <1% during the study intervention period and for up to one year after the last dose of study intervention. The investigator should evaluate potential for contraceptive method failure (e.g., noncompliance, recently initiated) in relationship to the first dose of study intervention.
19. A WOCBP must have a negative highly sensitive pregnancy test (urine or serum as required by local regulations) at hospital admission or before the first dose of study intervention. If a urine test cannot be confirmed as negative (e.g., an ambiguous result), a serum pregnancy test is required. In such cases, the subject is excluded from participation if the serum pregnancy result is positive.

### Informed Consent:

20. Willing to comply with the study requirements and capable of giving signed informed consent or legally acceptable representative is willing and able to give written informed consent on behalf of the subject to participate in the study for unconscious adults and those incapable of consenting themselves due to their medical condition.

### Exclusion Criteria:

in the opinion of the responsible investigator, any condition for which participation would not be in the best interest of the participant, or that could limit protocol-specified assessments; expected inability to participate in study procedures; participants who, in the opinion of the investigator, are not likely to survive beyond 48 hours at the time of randomization; planned or anticipated discharge or transfer to another hospital within 72 hours at the time of randomization; history of hypersensitivity to other mAbs or any of the excipients present in the investigational product;
For the lead-in phase: participants with end organ failure or dysfunction are not eligible for Lead-in phase but are eligible for Expansion-Phase.
   End-organ dysfunction categories are: a. stroke b. meningitis c. encephalitis d. myelitis e. myocardial infarction f. myocarditis g. pericarditis h. symptomatic congestive heart failure (New York Heart Association [NYHA] class III-IV) i. arterial or deep venous thrombosis or pulmonary embolism
   End-organ failure categories are: a. requirement for high-flow oxygen, non-invasive ventilation, or invasive mechanical ventilation b. extracorporeal membrane oxygenation (ECMO) c. mechanical circulatory support (e.g., intra-aortic balloon pump, ventricular assist device) d. vasopressor therapy e. commencement of renal replacement therapy (RRT) during this admission (i.e. not patients on chronic RRT).
Prior receipt of
   any SARS-CoV-2 hyperimmune intravenous immunoglobulin (hIVIG) from COVID-19 survivors
   convalescent plasma from a person who recovered from COVID-19 or
   SARS-CoV-2 neutralizing mAbs at any time.
Participating in other clinical trials involving an investigational study intervention, including for COVID-19
Pregnant or breast-feeding females

### Duration of Study Participation and Follow Up

The duration of study drug treatment is a single dose. The estimated total time of study, inclusive of screening and follow-up, for each subject is approximately 36 weeks. Screening assessments are performed up to 48 hours before the first dose. After completion of screening assessments, eligible subjects are treated with a single IC dose of sotrovimab or placebo. Subjects are assessed daily up to the day of hospital discharge. Following discharge from hospital, assessments occur on Day 15 and Day 29 and follow up continues to Week 36 after randomization.

### Study Design

An Independent Data Monitoring Committee (IDMC) actively monitors interim unblinded safety data (Part 1) and interim unblinded safety and efficacy data (Part 2) to make recommendations regarding ongoing study conduct. The IDMC members include 3-4 physicians with relevant medical specialist training and one statistician. A Joint Safety Review Team (JSRT) determines if a safety concern based on instream blinded data review needs to be escalated to the IDMC.

This study is a randomized, double-blind, multi-center, placebo-controlled trial of sotrovimab, a monoclonal antibody (mAb) against SARS-CoV-2, to assess efficacy and safety of a single IV dose of sotrovimab versus placebo on top of investigator-defined standard of care (SoC) as treatment in adult participants hospitalized with COVID-19 disease. The study population consists of hospitalized subjects with COVID-19 disease with a positive SARS-CoV-2 test result. These participants can have either critical (Grade 6 or 7) or severe (Grade 4 or 5) disease. All subjects receive SoC as per institutional protocols, in addition to study treatment.

The study comprises 2 parts. Part 1 enrolls 20 participants who have severe COVID-19 disease. Following an initial safety assessment of unblinded data at Day 14 by the IDMC, Part 2 (the main cohort) progresses, where participants with both severe and critical COVID-19 disease are enrolled. Only severe participants are enrolled in Part 2 without pausing the study until the IDMC recommend recruiting critical participants as well.

To explore the host immune response and potential biomarkers of infection, subjects may consent to an optional sub-study in which peripheral blood mononuclear cells (PBMCs) are collected at specified timepoints. This optional substudy is performed at selected sites. Approximately 50-100 participants per arm are enrolled into the optional sub-study.

During this study, an unblinded safety data review conducted by an IDMC determines whether it is safe to expand from Part 1, which includes subjects with severe (Grade 4 or 5) COVID-19 disease to Part 2, which additionally includes subjects with critical (Grade 6 or 7) COVID-19 disease.

A placebo control distinguishes the safety and tolerability of sotrovimab from the background signs and symptoms of COVID-19 and evaluates its potential benefit on clinical outcomes. The use of a placebo arm allows for a valid evaluation of any changes in efficacy and safety attributable to sotrovimab versus those attributable to SoC and other treatments given during the study.

The primary endpoint to assess efficacy of treatment is time to clinical response defined as subjects being alive and independent of supplementary oxygen or return to pre-morbid oxygen requirement (for subjects with chronic oxygen use) for at least 24 hours within 29 days of randomization. The transition from requiring oxygen therapy or higher levels of respiratory/organ support is a clinically significant milestone.

A significant secondary endpoints is all-cause mortality at Day 29. Other secondary efficacy endpoints related to hospital stay-length of hospital stay, ICU stay, time ventilated and the severity and duration of subject-reported signs and symptoms of COVID-19 are also clinically relevant.

### Study Intervention Groups and Duration

Screening assessments are performed up to 48 hours before the first dose. Screening procedures include taking a medical and disease history; testing for SARS-CoV-2 infection by nasal, nasopharyngeal, or endotracheal swab for subjects without a prior confirmed positive test; a full physical examination including measurement of height and weight; measurement of vital signs including blood pressure, pulse, respiratory rate, and temperature; oxygen saturation (SpO2) for subjects not on invasive mechanical ventilation; oxygen delivery and ventilation status; 12-lead ECG (triplicate); urine or serum pregnancy test for all female subjects of childbearing potential; laboratory assessments including hematology, clinical chemistry profile, coagulation parameters, CRP, D-dimer, urinalysis, and cardiac troponin; chest radiography (chest x-ray or computed tomography scan taken after hospitalization per SoC); and review of prior or concomitant medication.

Eligible subjects are treated with a single IV dose (sotrovimab or placebo) plus SoC on Day 1. They are assessed daily up to the day of hospital discharge. Following discharge from hospital, assessments occur on Day 15 and Day 29 and follow up continues to Week 36 after randomization.

In Part 1, subjects with severe COVID-19 disease (Grade 4 or 5) are randomized 1:1 by interactive response technology (IRT) in a blinded manner to receive a single IV dose of sotrovimab or placebo. In Part 2 (the main cohort), subjects with severe (Grade 4 or 5) or critical (Grade 6 or 7) COVID-19 disease are randomized 1:1 to receive a single IV dose of sotrovimab or placebo. All participants receive SoC as per institutional protocols, in addition to study treatment.

The randomization is stratified based on the disease severity (severe or critical) and time from onset of symptoms (less than seven days or seven days or more) at randomization.

### Study Interventions

Study interventions and dosing instructions are as follows:

TABLE 14

| | | |
|---|---|---|
| Dose Formulation | Solution in single-use vial | Sterile 0.9% (w/v) sodium |
| | (25 mg/mL) | chloride solution |
| Unit Dose Strength(s) | 250 mg/vial (250 mg/10 | Not applicable |
| | mL) | |
| Dosage Level(s) | (tbc) mg once | Once |
| Route of Administration | IV infusion | IV infusion |
| Dosing instructions | Withdraw 10.0 mL from | Dose per IV infusion |
| | the vial into a 10-20 mL | instructions |
| | small syringe and transfer | |
| | to a 150 mL or 250 mL IV | |
| | empty saline bag by an | |
| | unblinded pharmacist. | |
| | Repeat this procedure until | |
| | the required dose is | |
| | reached and administer | |
| | over 1 hour. | |
| Special instructions | Gently mix sotrovimab | None |
| | and saline prior to IV | |
| | infusion. | |
| Packaging and Labelling | Study intervention are | Labelled as required per |
| | provided in a single-use | country requirement |
| | vial in an individual carton | |
| | and labelled as required | |
| | per country requirement. | |

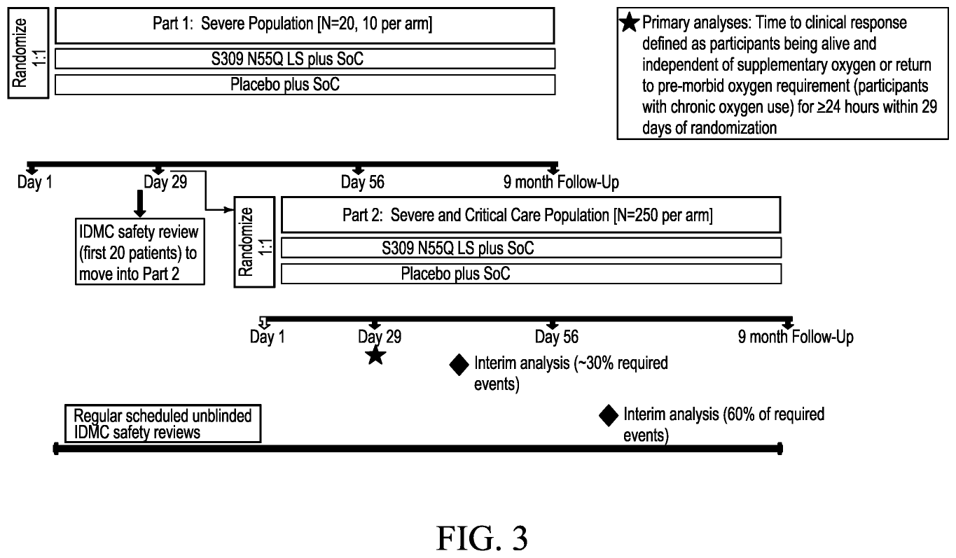

Further study details are shown in FIG. 3.

### Example 42

### Phase 3 Clinical Study of Sotrovimab in Patients Who have been Hospitalized with COVID-19

A Phase 3 clinical study is performed evaluate safety and effectiveness of different drugs in treating COVID-19 in people who have been hospitalized with the infection. Participants in the study are treated with either a study drug (e.g., sotrovimab) plus current standard of care (SOC), or with placebo plus current SOC.

The protocol is for a randomized, blinded, controlled platform study that allows investigational drugs to be added and dropped during the course of the study. This allows for efficient testing of drugs against placebo and standard of care (SOC) treatment within the same study. When more than one drug is tested at the same time, participants are randomly allocated to treatments or placebo.

Randomization are stratified by study site pharmacy and disease severity. There are 2 disease severity strata: Participants without organ failure (severity stratum 1); and participants with organ failure (severity stratum 2).

An independent Data and Safety Monitoring Board (DSMB) regularly reviews interim analyses and summarizes safety and efficacy outcomes. For investigational drugs with minimal pre-existing safety knowledge, the pace of enrollment is initially restricted, and there is an early review of safety data by the DSMB. For the study of each agent, at the outset of the trial, only participants in disease severity stratum 1 are enrolled. This continues until approximately 300 participants are enrolled and followed for 5 days. The exact number can vary according to the speed of enrollment and the timing of DSMB meetings. Prior to expanding enrollment to also include patients in disease severity stratum 2, safety is evaluated and a pre-specified futility assessment by the DSMB is carried out using 2 ordinal outcomes assessed at Day 5.

Both ordinal outcomes are used to assess futility because it is unclear whether the investigational agents under study will primarily influence non-pulmonary outcomes, for which risk is increased with SARS-CoV-2 infection, in part, through mechanisms that may be different from those that influence pulmonary outcomes.

For investigational agents passing this futility assessment, enrollment of participants is expanded, seamlessly and without any data unblinding, to include participants in disease severity stratum 2 as well as those in disease severity stratum 1. Future interim analyses are based on the primary endpoint of sustained recovery and use pre-specified guidelines to determine early evidence of benefit, harm or futility for the investigational agent. Participants are followed for 18 months following randomization.

The international trials within this protocol are conducted in several hundred clinical sites. Participating sites are affiliated with networks funded by the United States National Institutes of Health (NIH) and the US Department of Veterans Affairs.

Approximately 10,000 participants are enrolled. The allocation is randomized. The intervention model is parallel assignment. Masking is triple (Participant, Care Provider, Investigator). The primary purpose is treatment. In one arm, participants are randomized to receive drug (e.g. sotrovimab) plus SOC or placebo plus SOC. Subsequently, participants are no longer randomized to sotrovimab. In a placebo arm, placebo (commercially available 0.9% sodium chloride solution administered by IV infusion) plus remdesivir is provided absent contraindication.

### Primary Outcome Measures

1. Time from randomization to sustained recovery [Time Frame: Up to Day 90]. Sustained recovery defined as being discharged from the index hospitalization, followed by being alive and home for 14 consecutive days prior to Day 90.

### Secondary Outcome Measures

1. All-cause mortality [Time Frame: Thru Day 90]
2. Composite of time to sustained recovery and mortality [Time Frame: Thru Day 90]
3. Days alive outside short-term acute care hospital [Time Frame: Up to Day 90]
4. Pulmonary ordinal outcome [Time Frame: Days 1-7, 14 and 28]
   Oxygen requirements measured by 7 categories (1=least severe, 7=most severe). The participant's highest (i.e. most severe) observed score is used.
5. Pulmonary+ ordinal outcome [Time Frame: Days 1-7]
   Extrapulmonary complications and respiratory dysfunction measured by 7 categories (1=least severe, 7=most severe). The participant's highest (i.e. most severe) observed score is used.
6. Incidence of clinical organ failure [Time Frame: Thru Day 28]
7. Composite of death or serious clinical COVID-19 related events
   [Time Frame: Thru Day 90]
8. Composite of cardiovascular events and thromboembolic events
   [Time Frame: Thru Day 90]
9. Composite of grade 3 and 4 clinical adverse events, serious adverse events (SAEs) or death [Time Frame: Thru Days 5 and 28]
10. Incidence of infusion reactions [Time Frame: Thru Day 0]
11. Composite of SAEs or death [Time Frame: Thru 18 months]
12. Change in SARS-CoV-2 neutralizing antibody levels [Time Frame: Baseline to Days 1, 3, 5, 28 and 90]
13. Change in overall titers of antibodies [Time Frame: Baseline to Days 1, 3, 5, 28 and 90]
14. Change in neutralizing antibody levels [Time Frame: Baseline to Days 1, 3, 5, 28 and 90]
15. Incidence of home use of supplemental oxygen above pre-morbid oxygen use [Time Frame: 18 months]

Measured as: Alive at home and no use of continuous supplemental oxygen for an uninterrupted 14 day period
16. Incidence of no home use of supplemental oxygen above pre-morbid oxygen use [Time Frame: 14 days]

Measured as: alive at home for an uninterrupted 14 day period and no use of continuous supplemental oxygen at end of 14 day time period.

Adults aged 18 years and older (adults and older adults) of all sexes are eligible. Inclusion criteria are: signing of informed consent; positive test for COVID-19 and progressive disease suggestive of ongoing COVID-19 infection; symptoms of COVID-19 for ≤12 days; and require admission to hospital for acute medical care (not for purely public health or quarantine purposes). Exclusion criteria are:
Patients who have received plasma from a person who recovered from COVID-19 or who have received neutralizing monoclonal antibodies at any time prior to hospitalization.
Patients not willing to abstain from participation in other COVID-19 treatment trials until after Day 5 of the study. Co-enrollment in certain trials that compare recommended Standard of Care treatments may be allowed, based on the opinion of the study leadership team.
Any condition which, in the opinion of the responsible investigator, participation would not be in the best interest of the participant or that could prevent, limit, or confound the protocol-specified assessments.
Patients considered unable to participate in study procedures.
Women of child-bearing potential who are not already pregnant at study entry and who are unwilling to acknowledge strong advice to abstain from sexual intercourse with men or practice appropriate contraception through 18 months of the study.
Men who are unwilling to acknowledge the strong advice to abstain from sexual intercourse with women of child-bearing potential or to use barrier contraception through 18 months of the study.
Pregnant women
Nursing mothers
Presence at study enrollment of any of the following:
   1. stroke
   2. meningitis
   3. encephalitis
   4. myelitis
   5. myocardial ischemia
   6. myocarditis
   7. pericarditis
   8. symptomatic congestive heart failure
   9. arterial or deep venous thrombosis or pulmonary embolism
Current or imminent requirement for any of the following:
   1. invasive mechanical ventilation
   2. ECMO (extracorporeal membrane oxygenation)
   3. Mechanical circulatory support
   4. vasopressor therapy
   5. commencement of renal replacement therapy at this admission (i.e. not patients on chronic renal replacement therapy).

In addition, prior to the initial futility assessment which is performed when approximately 150 participants have been enrolled on sotrovimab and 150 on placebo, patients on high-flow oxygen or non-invasive ventilation (category 5 of the pulmonary ordinal outcome) will be excluded. These patients may be eligible for the trial if the initial futility assessment is passed by this agent.

A single dose of 500 mg sotrovimab is administered. The dose is selected based on in vitro neutralization data, in vitro resistance data, expected human PK extrapolated from a study in cynomolgus monkeys, and results of a GLP monkey toxicology study. Sotrovimab neutralized SARS-CoV-2 live virus with an average EC90 value of 186.3 ng/ml (range: 125.8-329.5 ng/mL). In resistance analyses, no viral breakthrough was observed through 10 passages at fixed concentrations of antibody even at the lowest dose tested (˜10× EC50), indicating the potential for sotrovimab to have a high barrier to resistance.

Using an increasing concentration selection method to force resistance emergence, modest fold changes in EC50 were observed during viral selection (5- to 6-fold change in EC50) for some passages. Sequencing and testing of spike variants from these passages using a pseudotyped virus system did not identify causal variants for this modest shift in potency. One passage of virus did demonstrate a >10-fold shift in EC50 which correlated with an E340A mutation. Further assessment has identified E340A to be a monoclonal antibody-resistant mutant (MARM) that confers a >100-fold reduction in susceptibility to sotrovimab. Notably, E340 is 100% conserved among available SARS-CoV-2 sequences. Due to the binary nature of the resistance selection results, a specific inhibitory quotient (IQ) was not informed by the resistance profiling. However, as very few strains are available to directly assess breadth of coverage, a conservative IQ (>10) is appropriate in this case. The cynomolgus monkey PK study (single IV dose, 5 mg/kg) was fit to a 2 compartment PK model. Human PK parameters were scaled from the cynomolgus monkey using an allometric scaling approach for fully human IgGs (allometric coefficient of 0.85 and 1 for CL and V, respectively; 10). The predicted serum clearance of sotrovimab in humans is estimated to be 141 mL/day and estimated volume of distribution is 6500 mL (˜93 mL/kg) assuming human weight of 70 kg. The projected human terminal elimination half-life is approximately 32 days.

In order to reduce risk to patients (treatment failure, emergence of viral resistance), a single therapeutic dose was selected that ensures sotrovimab concentrations in the lung are maintained far above levels anticipated to be protective for SARS-CoV-2 infection for the duration of the 28-day treatment window and beyond. A dose of 500 mg is expected to maintain serum levels at or above 38.5 μg/mL for the duration of the 28-day treatment period. Based on a conservative EC90 (0.33 μg/mL) from the highest end of the EC90 range, and accounting for the lung: serum ratio for IgG (assumed conservative value of 0.25; reported range 0.25-0.68 for whole lung and interstitial fluid, respectively; the serum trough concentration following a 500 mg dose is expected to result in lung concentrations associated with maximal (>99%) antiviral activity; >29× tissue-adjusted EC90 for the duration of the 28 day treatment period. This conservative inhibitory quotient (29-fold) in lung is believed to be appropriate to increase potential for treatment success and reduce risk for resistance.

Additionally, a 500 mg dose is anticipated to result in protective levels of sotrovimab in nasopharyngeal secretions (>5× tissue adjusted EC90 assuming NPS: serum ratio of 0.05, 12) which could potentially reduce transmission.

The NOAEL for sotromivab was 500 mg/kg, the highest dose tested, when sotrovimab was administered via IV infusion once a week for 2 weeks in cynomolgus monkeys. At this NOAEL, preliminary Cmax and area-under-the-curve (AUC)0-t (AUC from time 0 to 168 hr post-end of infusion following the 2nd dose) were 13500 μg/mL and 48200 μg·day/mL, respectively. The human equivalent dose (HED) (HED calculated via direct mg/kg conversion according to FDA guidance on proteins administered intravascularly with Mr >100,000 daltons; is 500 mg/kg or a 30,000 mg fixed dose (using human body weight of 60 kg). Using a safety factor of 10, the maximum recommended starting dose in humans is approximately 50 mg/kg or a 3,000 mg fixed dose. Based on the proposed 500 mg human dose, the margins based on the HED, Cmax, and AUC (conservative AUC margin based on partial AUC0-t from TX-7831-0102 and expected AUCinf in humans) are 60-, 87-, and 13.6-fold, respectively, supporting the proposed clinical dose of 500 mg.

In addition to the inclusion and exclusion criteria outlined in the master protocol, the following patients will be excluded: 1) pregnant women; and 2) nursing mothers. In addition, prior to the initial futility assessment which is performed when approximately 150 participants have been enrolled on sotrovimab and 150 on placebo, patients on high-flow oxygen or non-invasive ventilation (category 5 of the pulmonary ordinal outcome) are excluded. These patients may be eligible for the trial if the initial futility assessment is passed by this agent.

A single infusion is administered over a one-hour period. Study participants are monitored closely, and, per discretion of the physician supervising the infusion, adjustments in the infusion rate is made and/or the infusion paused or stopped.

Physician supervising the infusion may use supportive measures per local practice, if indicated. Sotrovimab is provided in vials of 10 ml solution containing 250 mg antibody each. Sotrovimab must be stored between 2° C. and 8° C. A total of 2 vials are required for dosing of the agent at 500 mg. The agent is administered at a concentration of 1 mg/ml to 10 mg/ml. Placebo is normal saline from local supply. If an infusion reaction occurs during administration or if the participant has a medical history suggesting a potential benefit from premedication, the study investigator(s) should determine the appropriate premedication. If the frequency of infusion reactions among participants warrants it, premedication may be recommended. If minor infusion reactions are observed, administration of acetaminophen, 500 mg to 1000 mg, antihistamines and/or other appropriately indicated medications may be given prior to the start of infusions for subsequent participants. The decision to implement premedication for infusions in subsequent participants is made by the investigator and sponsor and recorded in the study documentation. Any pre-medications given are documented as a concomitant therapy.

At Days 0, 28, and 90, venous blood samples are collected to determine antibody production against sotrovimab. Immunogenicity may be assessed by a validated assay designed to detect ADAs in the presence of sotrovimab. Antibodies may be further characterized for their ability to neutralize the activity of sotrovimab. Remaining volume from the PK sample may also be used for immunogenicity assessments as needed.

At Days 0, 1, 5, 28, and 90, venous blood samples are collected to determine sotrovimab serum concentration for pharmacokinetic assessment. The PK/Immunogenicity assessment requires 2 mL of the serum collected. PK samples may be assessed by a validated assay at a bioanalytical lab. The PK assessment uses the same 2 ml serum. Analysis of samples from placebo-treated subjects is not planned. Remaining sample used for PK may be pooled and used for exploratory metabolism or bioanalytical method experiments as deemed appropriate. Samples may be shipped to the lab for analyses in batches as the substudy is unfolding. Results from such analyses are returned to the trial central database. This data and all other data on the participants remain in the trial central database until the trial is unblinded. All participants should be monitored closely for 2 hours after the infusion, as there is a risk of infusion reaction and hypersensitivity (including anaphylaxis) with any biological agent.

Symptoms and signs that may occur as part of an infusion reaction, include, but are not limited to, fever, chills, nausea, headache, bronchospasm, hypotension, angioedema, throat irritation, rash including urticaria, pruritus, myalgia, and dizziness.

The clinical site should have necessary equipment, medications, adequately qualified and experienced staff with appropriate medical cover for the management of any infusion reaction, which may include, but is not limited to, oxygen, IV fluid, epinephrine (/adrenaline), acetaminophen (/paracetamol) and antihistamine.

If the participant experiences an infusion reaction, then supportive care should be used in accordance with the signs and symptoms. If a severe and potentially life-threatening infusion reaction occurs with sotrovimab/placebo, its use should be permanently discontinued. The following are AESIs for the agent sotrovimab or placebo for sotrovimab: infusion-related reactions; allergic/hypersensitivity reactions.

### REFERENCES

1. Pinto et al. Cross-neutralization of SARS-CoV-2 by a human monoclonal SARS-CoV antibody. Nature 2020; 583(7815): 290-295
2. https://clinicaltrials.gov/ct2/show/NCT04545060
3. Hope. Multiscale imaging of therapeutic antibody distribution and localisation. Presented at 2019 HIV & HBV Cure Forum; July 20-21; Mexico City, Mexico
4. Ko et al. Enhanced Fc receptor function improves protection against primate SHIV infection. Nature 2014; 514 (7524): 642-5.
5. VIR-7831 Investigator's Brochure, Edition 1, 28 Jul. 2020.
6. Arvin A M, Fink K, Schmid M A, Cathcart A, Spreadfico R, Havenar-Daughton C, Lanzavecchia A, Corti D, Virgin H W. A perspective on potential antibody-dependent enhancement of SARS-CoV-2. Nature. https://doi.org/10.1038/s41586-020-2538-8 (2020).
7. Duan K, Liu B, Li C, Zhang H, Yu T, Qu J, et al. Effectiveness of convalescent plasma therapy in severe COVID-19 patients. Proc Natl Acad Sci USA. 2020; 117 (17): 9490-6.
8. https://clinicaltrials.gov/ct2/show/NCT04033406?term=VIR-2482&draw=2&rank=1 (VIR-2482 against influenza)
9. Safety and pharmacokinetics of the Fc-modified HIV-1 human monoclonal antibody VRC01LS: A Phase 1 open-label clinical trial in healthy adults. Gaudinski M et al. Plos Medicine; 2018 https://doi.org/10.1371/journal.pmed.1002493
10. Deng R, Iyer S, Theil F P, Mortensen D L, Fielder P J, Prabhu S. Projecting human pharmacokinetics of therapeutic antibodies from nonclinical data: what have we
   learned? MAbs. 2011 January-February; 3 (1): 61-6. doi: 10.4161/mabs.3.1.13799. Epub 2011 Jan. 1. PMID: 20962582; PMCID: PMC3038012.
11. Baxter L T, Zhu H, Mackensen D G, Jain R K. Physiologically based pharmacokinetic model for specific and nonspecific monoclonal antibodies and fragments in normal tissues and human tumour xenografts in nude mice. Cancer Res. 1994 Mar. 15; 54(6): 1517-1528.
12. Covell D G, Barbet J, Holton O D, Black C D, Parker R J, Weinstein J N.
   Pharmacokinetics of monoclonal immunoglobulin G1, F(ab′)2, and Fab′ in mice. Cancer Res. 1986 August; 46 (8): 3969-78.
13. Datta-Mannan A. Mechanisms Influencing the Pharmacokinetics and Disposition of Monoclonal Antibodies and Peptides. Drug Metab Dispos. 2019 October; 47 (10): 1100-1110.
14. Lobo E D, Hansen R J, Balthasar J P. Antibody pharmacokinetics and pharmacodynamics. J Pharm Sci. 2004 November; 93 (11): 2645-68.
15. Lim J J, Derby M A, Zhang Y, Deng R, Larouche R, Anderson M, Maia M, Carrier S, Pelletier I, Girard J, Kulkarni P, Newton E, Tavel J A. A Phase 1, Randomized, Double-Blind, Placebo-Controlled, Single-Ascending-Dose Study To Investigate the Safety, Tolerability, and Pharmacokinetics of an Anti-Influenza B Virus Monoclonal Antibody, MHAB5553A, in Healthy Volunteers. Antimicrob Agents Chemother. 2017 Jul.
   25; 61 (8): e00279-17. doi: 10.1128/AAC.00279-17. PMID: 28559255; PMCID: PMC5527589.

### Example 43

### Phase 3 Clinical Study of Sotrovimab for Prevention of COVID-19 Among Adult and Adolescent Subjects Exposed to SARS-CoV-2

A randomized, multi-center, double-blind, placebo-controlled Phase 3 study is performed to assess safety and efficacy of monoclonal antibody sotrovimab in preventing COVID-19 in adult and adolescent subject exposed to SARS-CoV-2.

A primary objective is to evaluate efficacy of sotrovimab versus placebo for prevention of symptomatic COVID-19 among close contacts of persons with SARS-CoV-2 infection and to determine safety and tolerability of sotrovimab compared to placebo.

Secondary objectives are:
Evaluate efficacy of sotrovimab in the prevention of asymptomatic SARS-CoV-2 infection compared to placebo among close contacts of persons with SARS-CoV-2 infection
Evaluate efficacy of sotrovimab in reducing duration of viral shedding among those with SARS-CoV-2 infection
Evaluate efficacy of sotrovimab in reducing the incidence, severity, and duration of clinical signs and symptoms of COVID-19 among those with SARS-CoV-2 infection
Evaluate efficacy of sotrovimab on the time to alleviation of clinical symptoms among those with SARS-CoV-2 infection
Evaluate efficacy of sotrovimab in reducing progression to severe disease among those with SARS-CoV-2 infection as assessed by rate of hospitalization, ICU admission, or death due to COVID-19.
Evaluate the serum pharmacokinetics (PK) of sotrovimab
Evaluate potential immunogenicity (induction of ADA) response to sotrovimab

The exploratory objectives include:
Evaluate efficacy of sotrovimab in reducing viral load among those with SARS-CoV-2 infection
Evaluate association of SARS-CoV-2 viral load with treatment and with severity of disease
Monitor emergence of viral resistance to sotrovimab in subjects with confirmed SARS-CoV-2.
Evaluate potential relationships between subject genetic polymorphisms and sotrovimab mechanisms of action and/or PK
Measure impact of sotrovimab on time away from work and work productivity due to COVID-19
Evaluate effects of sotrovimab on potential biomarkers of host response

### Criteria for Evaluation

A primary efficacy endpoint of this study is as follows:
Proportion of subjects with laboratory confirmed (RT-PCR) COVID-19 defined as one of or more the following through Week 2 (Day 15±1):
   1 Acute respiratory symptoms (cough, sputum production, sore throat or shortness of breath);
   OR
   2. Fever >38° C.;
   OR
   3. 2 or more of the following symptoms (fatigue, myalgias/arthralgias, chills, nausea/vomiting, diarrhea, anosmia/dysgeusia)
   The primary safety endpoints of this study are as follows:
      The proportion of subjects with treatment emergent adverse events (AEs) and serious AEs (SAEs) following administration of sotrovimab
      Clinical assessments including but not limited to laboratory test results

Secondary endpoints of this study are as follows:
Incidence of laboratory confirmed (RT-PCR) asymptomatic SARS-CoV-2 infection through Week 2 (Day 15±1) in subjects with a negative baseline test
The difference in duration of viral shedding between sotrovimab and placebo treatment groups as assessed by RT-PCR from nasal swabs
The incidence, severity, and duration of clinical signs and symptoms of COVID-19.
Percentage of participants requiring hospitalization due COVID-19.
Percentage of participants requiring ICU care.
Percentage of participants requiring mechanical ventilation or ECMO.
Percentage of participants with COVID-19 related death.
Concentrations of sotrovimab in serum.
Incidence and titers (if applicable) of serum ADA to sotrovimab

The exploratory endpoints of this study include:
The difference in viral load at the time of illness onset between sotrovimab and placebo treatment groups as assessed by qRT-PCR from nasal swabs
Relationship between viral load (qRT-PCR) and severity of COVID-19
Incidence of laboratory confirmed SARS-CoV-2 infection by serology at Week 4 in subjects with a negative baseline test
FcR polymorphisms as determined by genotyping
Immunoglobulin G1 (IgG1) allotypes as determined by genotyping
Change from baseline in Work Productivity and Activity Impairment (WPAI)
Change from baseline in health-related quality of life according to EQ-5D-5L
Impact of sotrovimab administration on biomarkers of host response as assessed by host transcriptome and immunophenotyping analysis

### Number of Subjects

Approximately 1350 subjects (450 per treatment arm) are enrolled.

### Diagnosis and Main Eligibility Criteria

1. Male and female subjects aged 12 years or older
2. Close contact of a person (index) with known PCR-confirmed SARS-CoV-2 infection
   Close contact defined as:
a. Residing with the index case in the 7 days prior to index diagnosis, including residence or staff in a congregate setting such as long-term care facility or nursing home
b. Medical staff, first responders, or other care persons
c. Less than 3 days since last exposure (close contact with a person with SARS-CoV-2 infection) to the index case
4 Female subjects must have a negative pregnancy test or confirmation of post-menopausal status
5. Negative rapid test for SARS-CoV-2 infection within 4 hours prior to dosing

### Duration of Study Participation

The estimated total time on study for each subject, inclusive of screening, is up to 36 weeks. Subjects are screened within 24 hours prior to randomization. Following study drug administration, subjects are evaluated for up to Week 6 (D43±3) for endpoint assessments and up to 36 weeks for safety follow-up.

### Study Design

This is a randomized, double-blind, placebo-controlled study of 2 dose levels of sotrovimab for the prevention of symptomatic SARS-CoV-2 infection in adults exposed to the virus. The study is designed to evaluate efficacy of a low and a high dose sotrovimab compared to placebo in prevention of symptomatic SARS-CoV-2 infection.

This study enrolls approximately 1350 asymptomatic male and female subjects 12 years of age or older, including those with comorbidities, who are close contacts (i.e. household members or healthcare providers) of persons with reverse transcriptase-polymerase chain reaction (RT-PCR)-confirmed SARS-CoV-2 infection. A rapid diagnostic test is used at screening to enrich for subjects without established infection. Eligible subjects are randomized to receive either sotrovimab low dose, sotrovimab high dose, or placebo in a 1:1:1 ratio as a single administration on Day 1.

An independent data monitoring committee (DMC) consisting of members with relevant expertise convenes to review study data. The purpose of the DMC is to monitor safety, efficacy, and futility and determine the need for sample size readjustment based on observed infection rate in the placebo arm. Two interim analyses are conducted. The first interim analysis is conducted after approximately 20% of subjects have been enrolled (˜90 per arm) to assess safety and tolerability of each dose level of S309 N55Q LS.

The second interim analysis is conducted after approximately 50% of subjects have been enrolled (˜225 per arm) to assess safety, futility, and efficacy. A dose level may be discontinued due to safety concerns or lack of efficacy.

If data emerges on effective agents for SARS-CoV-2 prophylaxis, the protocol may be modified to include new standard of care agents in the control arm.

The study includes a PK sub-study in approximately 75 subjects (25 per arm) for intensive PK and ADA sample collections. All other subjects have sparse PK and ADA sample collections. If a new lot of sotrovimab is introduced into the study, additional subjects may be included into an intensive PK and immunogenicity sub-study.

### Study Procedures

### Screening:

Screening is performed within 24 hours prior to randomization and includes written informed consent, determination of eligibility, collection of demographics, past and current medical conditions (including known pregnancy and/or lactation status), concomitant medications and information regarding exposure to the Index Case.

### Dosing (Day 1):

Following collection of a baseline nasal swab for RT-PCR on Day 1, eligible subjects are randomized to receive either sotrovimab or placebo. Subjects remain in the clinic for a minimum of 4 hours post-dose to assess safety and local tolerability of sotrovimab and complete assessments.

### Follow-Up Period:

Subjects complete a daily diary from Day 1 through Week 2 visit. This survey includes documentation of clinical signs and symptoms of infection and concomitant medication review. In addition, all subjects collect a nasal swab at home on Days 4, 7, and 10 to monitor for asymptomatic SARS-CoV-2 infection. A study team member contacts subjects via telephone to evaluate adverse events and provide support for completion of study procedures. Subjects have an in-clinic visit at Week 2 (Day 15±1) and Week 6 (Day 42±3) and complete assessments.

Subjects are followed per the COVID-19 Monitoring Schedule if they experience symptom onset defined as acute onset of respiratory symptoms (dry cough, shortness of breath, sore throat, or sputum production); OR fever of >38.0° C.; OR ≥2 of the following symptoms: myalgias/arthralgias, chills, nausea/vomiting, diarrhea, anosmia/dysgeusia.

Subjects meeting criteria for potential symptomatic COVID-19 complete an in-clinic evaluation for SARS-CoV-2 infection, including nasal swab for confirmation of infection. Subjects continue to complete a daily diary for 21 days following symptom onset. Additional nasal swabs are collected to assess viral load and duration of viral shedding. In these subjects, nasal swabs are collected every other day for the first week following a positive test and then every 3 days during the second- and third-week post infection.

Subjects are followed for up to 36 weeks following dosing for assessment of safety, PK and immunogenicity. The duration of follow up is informed by the predicted human t1/2 of sotrovimab based on allometric scaling of NHP PK. The final follow up period is specified in the protocol. Between Week 6 visit and the last follow-up visit, subjects are contacted by phone for adverse event monitoring.

### Product, Dosage, and Mode of Administration

Sotrovimab is an engineered human IgG monoclonal antibody directed against SARS-CoV-2 spike protein with modifications to increase half-life. Sotrovimab is administered in a single dose IV or IM at either a low or high dose level. Subjects randomized to placebo are administered sterile, preservative-free normal saline 0.9% solution by IM injection.

### Statistical Methods

Not all individuals exposed to SARS-CoV-2 will become infected or develop symptoms, and early estimates place family members at a 3% to 15% risk of infection after likely exposure. For the purpose of sample size calculation an estimated 10% attack rate was used. Sample size was chosen to achieve 80% power with 0.05 two-sided Type 1 error for the endpoint of laboratory confirmed symptomatic COVID-19, an attack rate of 10% and 50% efficacy of sotrovimab in reducing symptomatic COVID-19. Based on these assumptions, approximately 450 subjects are enrolled per treatment arm (total of 1350).

Two interim analysis are conducted for this study. The first interim analysis is conducted after approximately 20% of subjects have been enrolled (90 per arm) to assess safety and tolerability of each dose level of sotrovimab. A dose level may be discontinued due to safety concerns. The second interim analysis is conducted after approximately 50% of subjects have been enrolled (225 per arm) to assess safety, futility, and efficacy. A dose level may be discontinued due to safety concerns or lack of efficacy. Based on the attack rate observed at the interim analysis, sample size may be adjusted in order to increase the likelihood of achieving the overall expected number of cases to meet the primary endpoint. In order to properly control the overall type 1 error rate (the ‘alpha’) at the 0.05 level, O'Brien-Fleming method is used to allocate alpha between the second interim analysis (0.0052) and the final analysis (0.048).

All efficacy analyses are performed using the ITT population (all randomized subjects). Incidence of symptomatic and asymptomatic SARS-CoV-2 infection is also evaluated in the mITT population (subjects who are SARS-CoV-2 negative at the baseline visit). Safety is assessed in all subjects who received any amount of study drug.

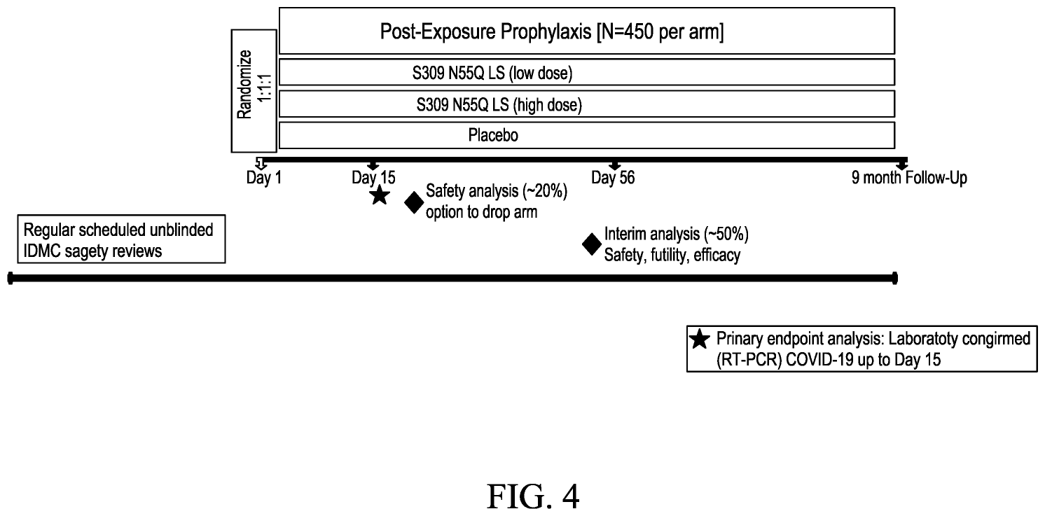

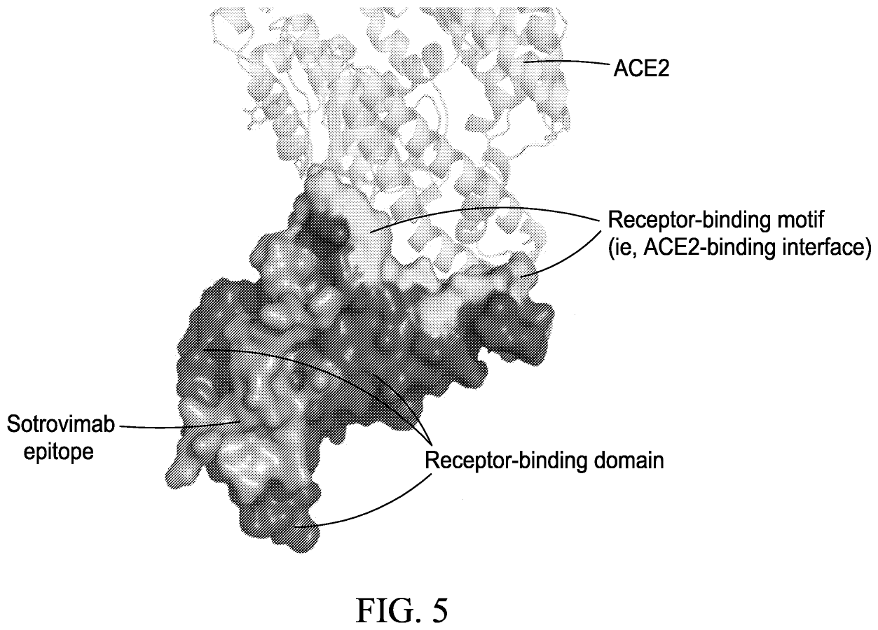

Further details of the study are shown in FIG. 4.

### Example 44

### Phase 3 Clinical Study of Sotrovimab for Prevention of COVID-19 Among Adult and Adolescent Subjects Exposed to SARS-CoV-2

A randomized, double-blind, placebo-controlled Phase 3 study is conducted to evaluate safety and efficacy of sotrovimab for prophylaxis of COVID-19. Approximately 3150 participants are randomized in a 2:1 ratio to receive either sotrovimab or placebo respectively to evaluate the safety and efficacy of sotrovimab for the prevention of COVID-19.

The primary objective is to evaluate safety and efficacy versus placebo in preventing COVID-19. The primary endpoint is incidence rate of protocol-defined COVID-19 through Week 13 (3 months) as defined by:
A positive SARS-CoV-2 reverse transcriptase polymerase chain reaction (RT-PCR) from a nasopharyngeal swab
AND
New onset (or exacerbation) of at least one or more of the following symptoms that persist or reoccur after a period of at least 24 hours:
   Fever >38° C., chills, cough, sore throat, malaise, headache, myalgia, change in smell or taste, nasal congestion/rhinorrhea, vomiting, diarrhea, shortness of breath on exertion

This is a clinically meaningful endpoint to both clinicians and participants as prevention of symptomatic COVID-19 may lead to decreased morbidity and mortality from this disease as well as potentially lead to decreased transmission. In addition, a statistical testing hierarchy approach is employed to control the overall type I error to determine the efficacy of sotrovimab in the prevention of protocol-defined symptomatic COVID-19 at additional timepoints (up to Weeks 17, 21 and 26).

Other secondary efficacy endpoints including prevention of CDC-defined symptomatic COVID-19, and prevention of asymptomatic SARS-CoV-2 infection (as assessed by seroconversion at Weeks 13 and 26) are also clinically relevant.

A participant is considered to have completed the study if he/she has completed the study through to Week 26.

Participants 18 years or older at the time of giving informed consent (except for in South Korea, where participants are 19 years or older) and of any gender are eligible. Female participants must meet and agree to abide by the contraceptive criteria listed below. Contraception use by women should be consistent with local regulations regarding the methods of contraception for those participating in clinical studies.

A female participant is eligible to participate if she is not pregnant or breastfeeding, and one of the following conditions applies:
a. Is a woman of non-childbearing potential (WONCBP), or
OR
b. Is a woman of childbearing potential (WOCBP) and using a contraceptive method that is highly effective, with a failure rate of <1%, during the study intervention
   period and for up to 26 weeks after the last dose of study intervention. The investigator should evaluate potential for contraceptive method failure (e.g., noncompliance, recently initiated) in relationship to the first dose of study intervention.

A WOCBP must have a negative highly sensitive pregnancy test (urine or serum as required by local regulations) before the first dose of study intervention.
a. If a urine test cannot be confirmed as negative (e.g., an ambiguous result), a serum pregnancy test is required. In such cases, the participant must be excluded from participation if the serum pregnancy result is positive.
b. Additional requirements for pregnancy testing during and after the study intervention may be in-place.

The investigator reviews medical history, menstrual history, and recent sexual activity to decrease the risk for inclusion of a woman with an early undetected pregnancy.

Participants are capable of giving informed consent. Participants agree to study procedures including the collection of nasopharyngeal swabs, self-collected nasal mid-turbinate swabs and venous blood as specified in the schedule of activities. Participants are able and willing to complete study surveillance questionnaires and activities.

Participants have an anti-spike SARS-CoV-2 antibody test during screening for enrollment. Participants with a positive anti-spike SARS-CoV-2 antibody test are not enrolled. Participants have a nasopharyngeal SARS-CoV-2 RT-PCR and an anti-N SARS-CoV-2 antibody test at Day 1 prior to dosing. Participants found to have a positive SARS-CoV-2 RT-PCR test or a positive anti-N SARS-CoV-2 antibody test at Day 1 are excluded from the primary analysis (mITT) population.

### Exclusion Criteria

Participants are excluded from the study if any of the following criteria apply:

### Medical Conditions

1. History of prior positive SARS-CoV-2 RT-PCR or antigen test or history of positive SARS-CoV-2 serology test including during screening.
2. Febrile illness with or without respiratory symptoms (e.g., cough, nasal congestion) within 7 days prior to randomization
3. Unstable medical condition and not expected to survive for the duration of study participation as judged by the investigator
4. Participant has any condition that would prohibit receipt of intramuscular injections in the investigator's opinion such as coagulation disorder, bleeding diathesis, or thrombocytopenia
5. Known hypersensitivity to any constituent present in the investigational product
6. Previous anaphylaxis or hypersensitivity to a monoclonal antibody
7. Participants who, in the judgment of the investigator, will be unlikely or unable to comply with the requirements of the protocol through Week 26

### Prior Medications

8. Receipt of a COVID-19 vaccine at any time prior to enrollment, or planned receipt of a COVID-19 vaccine during the first 13 weeks of the study
9. Receipt of any vaccine within 48 hours prior to enrollment. Vaccination will not be allowed for 2 weeks after dosing
10. Receipt of any SARS-CoV-2 hyperimmune intravenous immunoglobulin (hIVIG) from COVID-19 survivors
11. Receipt of convalescent plasma from a recovered COVID-19 patient or an anti-SARS-CoV-2 mAb within 3 months or 5 half-lives, whichever is longer, prior to screening and/or during the study

### Prior/Concurrent Clinical Study Experience

12. Enrolment in any investigational drug or device study for SARS-CoV-2/COVID-19 within 90 days prior to Day 1 or within 5 half-lives of the investigational compound, whichever is longer.

### Other Exclusions

13. Pregnant or breast-feeding females.
(all female participants regardless of childbearing potential status must have a negative pregnancy test at screening/pre-dosing)

Sotrovimab is formulated in solution in a single use vial (62.5 mg/mL). the unit dose strength is 500 mg/vial (500 mg/8 mL). Sotrovimab is administered in a single dose (500 mg, once) by intramuscular injection. Placebo is locally sourced sterile 0.9% (w/v) sodium chloride solution, administered in a single dose by intramuscular injection.

### BRIEF SUMMARY

### Lead-In Phase

The Lead-in phase of the study is intended to evaluate the safety, tolerability, and pharmacokinetics (PK) of sotrovimab with frequent assessments of injection site reactions (ISRs) and intensive blood sampling for PK analysis. These participants are pooled with the Expansion phase participants for efficacy evaluations. Approximately 21 participants are enrolled and randomized 2:1 to receive VIR-7831 (N=14) or equal volume saline placebo (N=7). Participants are monitored for 6 hours after dosing for safety monitoring and local injection site tolerability assessments. At the end of 6 hours, they are discharged. Randomization is paused upon enrollment of the Lead-in phase, until Day 8 safety assessment by the Joint Safety Review Team (JSRT) recommends continuing. If the JSRT requires escalation to the IDMC, then randomization pauses pending a recommendation to proceed by the IDMC.

### Intensive PK Sampling and Immunogenicity

All participants enrolled in the Lead-In phase have additional timepoints for PK blood sampling. Serum PK and anti-drug antibodies (ADA) samples are collected.

The lead-in phase of the study is summarized in Scheme 2, wherein sotrovimab is identified as VIR-7831.

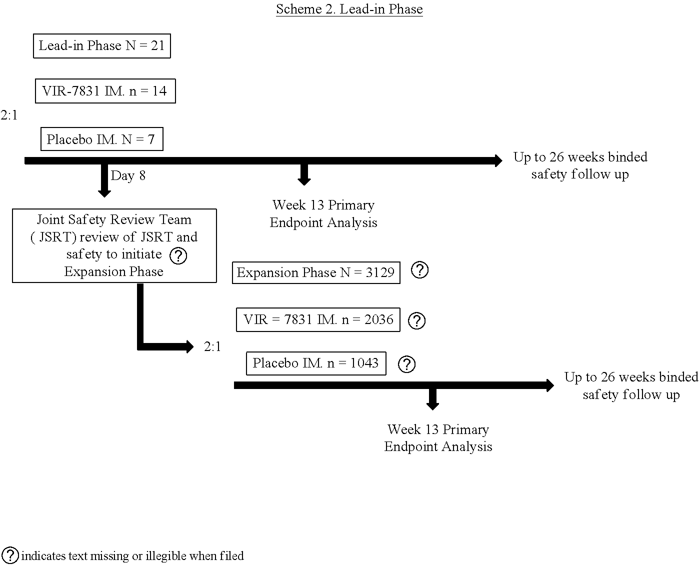

### Expansion Phase

The Expansion phase of the study is gated to a safe to proceed recommendation from the JSRT. Specifically, randomization into the Expansion phase starts following blinded assessment of local injection site tolerability assessment data (ISRs) and other safety data from the Lead-in phase through Day 8 of follow-up by the JSRT.

Approximately 3129 additional participants are enrolled and randomized in a 2:1 ratio to receive sotrovimab or equal volume saline placebo via IM administration respectively.

Participants in the Expansion phase are monitored through 1 or 2 hours post-dose prior to discharge from the clinic/study unit. The duration of monitoring (1 or 2 hours) is based on assessment of ISRs from the Lead-in phase. The decision regarding the duration of monitoring is communicated as part of the JSRT recommendations to sites, and as a protocol file note.

Participants from both the Lead-in and Expansion phases are monitored for COVID-19 throughout the study via two mechanisms, as follows:
1. Participants are provided with a printed guide of COVID-19 signs and symptoms to look out for, and a study site phone number to call within 24 hours of experiencing any of those symptoms, or if they have any other reason to suspect they may be infected with SARS-CoV-2.
2. In addition, the site makes a regular once-weekly phone call to the participant, from Day 1 through Week 13 (every two weeks from Week 13 to Week 26), to capture adverse events (AEs), serious adverse events (SAEs) and to review the COVID-19 symptoms checklist with the participant.

All participants are provided with a nasal (mid-turbinate) swab self-test kit and return envelope for central lab testing, at the start of the study. Any participant who experiences COVID-19 symptoms during the 26-weeks study follow-up period, are instructed to use the self-test kit immediately, and to follow any additional local guidance for managing suspected COVID-19. Those that test positive complete weekly self-reported COVID-19 symptom surveillance and weekly Work Productivity and Activity Impairment (WPAI) questionnaires and are followed per a COVID-19 Illness Monitoring Schedule for 4 weeks after symptom onset. If a participant tests negative and is still experiencing symptoms, a second self-collection kit is sent to the participant for repeat testing.

All participants have regular follow-up phone calls and attend specific follow-up visits for assessments of efficacy, safety, and PK (sparse).

### Number of Participants:

Approximately 3150 participants are randomly assigned to the study intervention in a 2:1 ratio to sotrovimab or placebo. Any participant who receives the study intervention are considered evaluable.

### Study Screening, Randomization, and Duration

Screening assessments are performed during the 14 days prior to Day 1. Eligible participants are treated in a blinded manner with an IM dose on Day 1 and followed up to End of Study (EOS)-26 weeks (6 months). Participants remain blinded through EOS. Participants who develop COVID-19 (hospitalized or not hospitalized) are followed through 4 weeks post-symptomatic infection or EOS, whichever is earlier.

Randomization of participants in both the Lead-in and Expansion phases are stratified based on the following:
Age strata (<65 years of age, ≥65 years of age) Increased risk exposure group
Presence of an immunocompromising condition

Block randomization occurs by country.

### Independent Data Monitoring Committee

An unblinded Independent Data Monitoring Committee (IDMC) evaluates the overall safety profile of sotrovimab including evaluation of the incidence and severity of COVID-19 cases in participants randomized to sotrovimab compared to those randomized to placebo throughout the conduct of the study. The roles and responsibilities of the IDMC, including membership, scope, frequency of meetings and communication plan are defined in the IDMC charter. IDMC meetings occur regularly throughout the study as outlined by the IDMC charter.

### Joint Safety Review Team

A Joint Safety Review Team (JSRT) comprised of team members from clinical research, pharmacovigilance, and statistics from study sponsors determines if a safety concern identified during instream blinded data review needs to be escalated to the IDMC. The JSRT is responsible for blinded safety review of the Lead-in phase as described above, with escalation to the IDMC if needed. The IDMC acts in accordance with the process defined in the IDMC Charter. Initiation of the Expansion phase is contingent on a safe to proceed recommendation from the JSRT. The responsibilities of the JSRT and frequency of assessments are available in relevant safety review team (SRT) documents.

The incidence rate of protocol-defined COVID-19 through Week 13 (3 months) and is analyzed using a Poisson regression model to estimate the relative risk. The model includes terms for treatment group, age strata, exposure risk group, immunocompromised group and country. However, for countries with no events, countries may be collapsed into regions for the model. The relative risk, 95% confidence interval and corresponding p-value are presented for the comparison of sotrovimab with placebo.

### Example 45

### Intramuscular Sotrovimab for Mild/Moderate COVID-19

A Phase 3 randomized, multi-center, open label study is conducted to assess the efficacy, safety, and tolerability of sotrovimab given intramuscularly versus intravenously for the treatment of mild/moderate COVID-19 in high-risk non-hospitalized patients. Arms and interventions are as in Table 15.

TABLE 15
Arms and Interventions

| | Assigned |
|---|---|
| Arms | Interventions |
| Active Comparator: Sotrovimab 500 mg IV | Sotrovimab |
| Sotrovimab given by intravenous infusion | 500 mg IV |
| Experimental: Sotrovimab 500 mg IM Sotrovimab | Sotrovimab |
| given by intramuscular injection | 500 mg IM |
| Experimental: Sotrovimab 250 mg IM Sotrovimab | Sotrovimab |
| given by intramuscular injection | 250 mg IM |

### Outcome Measures

### Primary Outcome Measure:

1. Progression of COVID-19
[Time Frame: Up to Day 29]

### Secondary Outcome Measure:

2. Occurrence of adverse events (AEs)
[Time Frame: Up to 24 weeks]
3. Occurrence of serious adverse events (SAEs)
[Time Frame: Up to 24 weeks]
4. Occurrence of adverse events of special interest (AESI)
[Time Frame: Up to 24 weeks]
5. Incidence and titers (if applicable) of serum anti-drug antibody (ADA) to sotrovimab
[Time Frame: Up to 24 weeks]
6. Mean area under the curve of SARS-CoV-2 viral load in nasal secretions as measured by qRT-PCR
[Time Frame: Up to Day 8]
7. Change from baseline in viral load by qRT-PCR
[Time Frame: Up to Day 8]
8. Proportion of participants with a persistently high SARS-CoV-2 viral load at Day 8 by qRT-PCR
[Time Frame: Up to Day 8]
9. Development of severe and/or critical respiratory COVID-19
[Time Frame: Up to Day 29]
10. IV and IM sotrovimab pharmacokinetics (PK) in serum
[Time Frame: Up to Week 24]

### Eligibility

Minimum Age: 12 Years
Maximum Age:
Sex: All
Gender Based: No
Accepts Healthy Volunteers: No
Criteria: Inclusion Criteria:
   Participant must be aged 12 years or older at time of consent AND at high risk of progression of COVID-19 or ≥65 years old
   Participants must have a positive SARS-CoV-2 test result and oxygen saturation ≥94% on room air and have COVID-19 symptoms and be less
      than or equal to 7 days from onset of symptoms

### Exclusion Criteria:

Currently hospitalized or judged by the investigator as likely to require hospitalization in the next 24 hours
Symptoms consistent with severe COVID-19
Participants who, in the judgement of the investigator are likely to die in the next 7 days
Known hypersensitivity to any constituent present in the investigational product

### Example 46

### Phase II Study of a Second Generation Sotrovmab Material in Non-Hospitalized Participants with Mild-to-Moderate Coronavirus Disease 2019 (COVID-19)

Sotrovimab drug material used in the studies described in Examples 39-40 was produced using a non-clonal pool of stably transfected cells. This is referred to as “Gen1” drug material. “Gen2” drug material is made using a clonal master cell bank derived from the original pool of transfectants. A multicenter, randomized, double-blind, parallel group phase II study is performed to evaluate safety, tolerability and pharmacokinetics of Gen2 drug materials in non-hospitalized participants with mild to moderate COVID-19.

The primary objective is to evaluate safety and tolerability of Gen2 and Gen1 sotrovimab drug material. Endpoints are: occurrence of adverse events (AEs) through Day 29; occurrence of serious adverse events (SAEs) through Day 29; occurrence of adverse events of special interest (AESIs) through Day 29; occurrence of clinically significant abnormalities on 12-lead electrocardiogram (ECG) readings through Day 29; and occurrence of disease progression events (not classified as AEs) through Day 29.

Secondary objectives are:
(1) To assess the pharmacokinetics (PK) of sotrovimab Gen2 and Gen1 in serum
   Endpoints: Cₘₐₓ, Cₗₐₛₜ, Tₘₐₓ, Tₗₐₛₜ, AUC_{D0-28}, AUC_{inf}, AUCₗₐₛₜ, % AUCₑₓₚ, t_{1/2}, λ_{z}, V_{z}, Vₛₛ, CL;
(2) To evaluate safety and tolerability profile of sotrovimab Gen2 and Gen1
   Endpoints: occurrence of SAEs through Week 24; occurrence of AESIs through Week 24; occurrence of clinically significant abnormalities on 12-lead ECG readings through Week 24; occurrence of disease progression events (not classified as AEs) through Week 24.

Exploratory endpoints are:
(1) To assess potential immunogenicity of sotrovimab Gen2 and Gen1
   Endpoint: Incidence and titers (if applicable) of serum antidrug antibodies (ADA) to sotrovimab
(2) To characterize the effect of sotrovimab Gen2 and Gen1 on the viral shedding profile in upper respiratory samples.
   Endpoint: Change from baseline in viral load at all visits through Day 29 as measured by quantitative reverse transcriptase polymerase chain reaction (qRT-PCR) from saliva samples.

This study is a randomized, double-blind, multi-center, parallel group phase II trial of sotrovimab, a monoclonal antibody (mAb) against SARS-CoV-2 for the prevention of progression of mild to moderate COVID-19 in non-hospitalized patients. Participants with early mild to moderate COVID-19 are randomized 3:1 to receive a single, 500 mg intravenous infusion of either Gen2 or Gen1 study material. Safety, tolerability and PK are evaluated.

Screening assessments are performed within 24 hours before the start of infusion. Eligible participants are treated in a blinded manner with a single IV dose on Day 1 and followed up to 24 weeks. Participants re monitored for approximately 9 hours on Day 1 for safety assessments and intensive PK sampling.

The study includes nonhospitalized patients with mild to moderate SARS-CoV-2 infection as confirmed by local laboratory tests and/or point of care tests. A single dose level of Gen1 or equal volume Gen2 material is studied and delivered via IV infusion. Participants receive the infusion in a clinic/study unit where they are monitored closely for adverse events in the post-infusion period. Subsequent visits for study activities and clinical monitoring are conducted via clinic or home nursing visits (except for Week 16). A double-blind design is a standard methodology for randomized, controlled studies to minimize bias. A Gen1 study arm is included to maintain blinding of Gen1 versus Gen2 sotrovimab study material receipt during the conduct of the study. This blinding is critical for minimizing bias that may occur during study assessments, including during evaluation of AEs and disease-related safety outcomes.

Blinded safety data are reviewed regularly by the JSRT through Week 24. There is no placebo in this study because the primary aim is to evaluate safety and tolerability of Gen2 material. All participants receive standard of care for COVID-19 disease during this study, including admission to a hospital if deemed necessary by the responsible investigator.

Analytical comparability studies support the use of Gen2 in clinical studies.

### Example 47

### ACE2-Independent Mechanism of SARS-CoV2 Neutralization by S309 Antibody

In the following experiments, S309 antibody (VH of SEQ ID NO.: 105, VL of SEQ ID NO.: 168) was expressed as recombinant IgG1 with M428L and N434S Fc mutations. The effect of ACE2 overexpression on S309 antibody neutralization of infection was investigated. Vero E6 or Vero E6-TMPRSS2 cells were infected with SARS-CoV-2 (isolate USA-WA1/2020) at MOI 0.01 in the presence of S309 (10 μg/ml). Cells were fixed 24 h post infection, viral nucleocapsid protein was immunostained and quantified. Nucleocapsid staining was effectively absent in antibody-treated cells. S309 had an IC50 (ng/ml) in Vero E6 cells of 65 and in Vero E6-TMPRSS2 of 91 (data not shown).

A panel of 7 cell lines (HeLa, 293T (wt), Vero E6, Huh7, 293T ACE2, MRC 5-ACE2-TMPRSS2, A549-ACE2-TMPRSS2 clone 5, A549-ACE2-TMPRSS2 clone 10) were infected with SARS-CoV-2-Nluc or VSV pseudotyped with the SARS-CoV-2 spike protein in the presence of S309. Luciferase signal was quantified 24 h post infection. S309 maximum neutralization values were as shown in Table 16.

TABLE 16
Maximum Neutralization Values of S309

Virus/Pseudotype

| | SARS-CoV- | VSV |
|---|---|---|
| Cell Type | 2-Nluc | Pseudotype |
| Vero E6 | >99% | >99% |
| Vero E6-TMPRSS2 | >99% | 96% |
| Huh7 | 98% | 78% |
| 293T ACE2 | 26% | 34% |
| MRC5-ACE2-TMPRSS2 | 87% | 45% |
| A549-ACE2-TMPRSS2 clone 5 | 89% | 65% |
| A549-ACE2-TMPRSS2 clone 10 | 81% | 42% |

Binding of purified, fluorescently-labeled SARS-CoV-2 spike protein binding to these cell lines was quantified by flow cytometry. HeLa and 239T WT cells had he lowest MFIs, followed by Huh7 and VeroE6 cells. 293T ACE2 cells (highest), MRC 5-ACE2-TMPRSS2 (third-highest), A549-ACE2-TMPRSS2 clone 5 (fourth-highest), and A549-ACE2-TMPRSS2 clone 10 (second-highest) had higher MFIs. Correlation analysis between spike binding maximum neutralization potential of S309 was determined; S309 Spearman correlation values were: r=−0.94 for both viral models. p=0.017.

To further characterize SARS-CoV-2-susceptible cell lines, the seven cell lines described above were incubated with purified, fluorescently-labeled SARS-CoV-2 spike protein or RBD protein and protein binding was quantified by flow cytometry. In descending order of MFI, the cell lines were: A549-ACE2-TMPRSS2 clone 10; 293T ACE2; MRC 5-ACE2-TMPRSS2; A549-ACE2-TMPRSS2 clone 5; Vero E6; Huh7; 293T (wt); and HeLa.

Selected lectins and published receptor candidates were screened using HEK293T cells infected with SARS-CoV-2 VSV pseudoviruses. ACE2, DC-SIGN, L-SIGN, and SIGLEC-1 gave the highest signals. ACE2 provided a signal of approximately 10⁵ relative luminescence units (RLUs), and DC-SIGN, SIGLEC-1, and L-SIGN had signals of approximately 10⁴ RLUs. All other lectins/candidates tested gave signals of approximately 10²-10³ RLUs.

HEK 293T, HeLa and MRC5 cells were transiently transduced to overexpress DC-SIGN, L-SIGN, SIGLEC1 or ACE2 and infected with SARS-CoV-2 VSV pseudoviruses. Uninfected cells and untransduced cells were included as controls. In HEK293T cells, ACE2, DC-SIGN, SIGLEC-1, and L-SIGN all provided substantial increases in infection. In HeLa and MRC5 cells, only ACE2 increased infection.

Stable HEK293T cell lines overexpressing DC-SIGN, L-SIGN, SIGLEC-1 or ACE2 were infected with authentic SARS-CoV-2 (MOI 0.1), fixed and immunostained at 24 hours for the SARS-CoV-2 nucleoprotein. Wild-type cells (infected and uninfected) were used as controls. Increased staining was observed in cells overexpressing DC-SIGN, L-SIGN, or SIGLEC-1, and staining was significantly increased in cells overexpressing ACE2.

Stable cell lines were infected with SARS-CoV-2-Nluc and luciferase levels were quantified at 24 hours. In ascending order of RLUs: uninfected (approx. 10²-10³ RLUs); parental 293T (approx. 10⁴ RLUs); DC-SIGN (approx. 10⁵ RLUs); L-SIGN (approx. 10⁵ RLUs); SIGLEC-1 (approx. 10⁵-10⁶ RLUs); ACE2 (>10⁷ RLUs).

Stable cell lines were incubated with different concentration of anti-SIGLEC1 mAb (clone 7-239) and infected with SARS-CoV-2-Nluc. Infection as a percentage of untreated cells remained near to exceeded 100% in 293T cells expressing DC-SIGN, L-SIGN, or ACE2, but dropped to below 50% (0.2 μg/mL anti-SIGLEC) to close to 0 (1 μg/mL or 5 μg/mL anti-SIGLEC) in 293T cells expressing SIGLEC-1.

Single cell expression levels of selected potential SARS-CoV-2 (co) receptor candidates were determined in different lung cell types derived from the Human Lung Cell Atlas (nature.com/articles/s41586-020-2922-4). DC-SIGN, L-SIGN and SIGLEC-1 are expressed in a variety of cell types in the lung at levels similar to or even higher than ACE2.

Binding of antibodies targeting DC-/L-SIGN, DC-SIGN, SIGLEC1 or ACE2 on HEK293T cells stably over-expressing the respective attachment receptor was analyzed by flow cytometry and immunofluorescence analysis. HEK 293T cells over-expressing the respective attachment receptors were infected with VSV pseudotyped with SARS-COV-2 wildtype spike or spike bearing mutations of the B1.1.7 lineage. Luminescence was analyzed one day post infection. Infection was increased in cells expressing the attachment receptors. Infection by VSV pseudotyped with either spike was similar for each test group. Cells expressing ACE2 gave the highest luminescence signal.

Vero E6 cells, in vitro differentiated moDCs or PBMCs were infected with SARS-CoV-2 at MOI 0.01. At 24 h post infection, cells were fixed, immunostained for viral nucleocapsid protein and infected cells were quantified. Only VeroE6 cells showed infection (approximately 7% of cells). Supernatant of the infected cells was taken at 24, 48 and 72 h and infectious viral titer was quantified by FFU assay on Vero E6 cells.

Major cell types with detectable SARS-CoV-2 genome in bronchoalveolar lavage fluid (BALF) and sputum of severe COVID-19 patients were assessed. A t-SNE plot was generated, and the count of each SARS-CoV-2+ cell type was determined (total n=3,085 cells from 8 subjects in Ren et al. Cell 2021). Cell types were T, NK, plasma, neutrophil, macrophage, ciliated, squamous, and secretory. Expression of ACE2, DC-SIGN, L-SIGN, SIGLEC-1, and combinations of these was assessed for each cell type.

ACE2, DC-SIGN (CD209), L-SIGN (CLEC4M), SIGLEC1 transcript counts were correlated with SARS-CoV-2 RNA counts in macrophages and in secretory cells. Correlation was based on counts (before log transformation), from Ren et al. Cell 2021.

Cell lines were generated to overexpress DC-SIGN, L-SIGN or ACE2 by transducing HEK293T cells with lentivirus encoding the transgene, and immunofluorescence assays were performed to assess transgene expression.

Expression of DC-SIGN or L-SIGN increased pseudovirus infection levels by over 10-fold compared to infection of WT HEK293T cells, and expression of ACE2 increased pseudovirus infection levels by over 100-fold compared to infection of WT HEK293T cells.

Neutralizing activity of exemplary mAb S309 against the VSV pseudovirus was assessed in the engineered HEK293T cells. S309 fully neutralized infection via DC-SIGN and L-SIGN, and to a lesser extent, ACE2.

The ability of live SARS-CoV-2 with luciferase reporter to infect the HEK293T cells was examined using a luminescence assay. Expression of DC-SIGN or L-SIGN increased live virus infection levels by over 3-fold compared to infection of WT HEK293T cells, and expression of ACE2 increased live virus infection levels by over 100-fold compared to infection of WT HEK293T cells.

Neutralizing activity of mAb S309 against the VSV pseudovirus was assessed in the engineered HEK293T cells. S309 fully neutralized infection via DC-SIGN and L-SIGN, and neutralized infection via ACE2 to a lesser extent.

Experiments were performed to investigate whether S309 antibody can neutralize entry of SARS-CoV-2 via SIGLEC-1. Briefly, stable cell HEK293T lines were generated as described above to overexpress DC-SIGN/L-SIGN, DC-SIGN, SIGLEC-1, or ACE2. Expression of DC-SIGN, L-SIGN, or SIGLEC increased live virus infection levels by over 10-fold compared to infection of WT HEK293T cells, and expression of ACE2 increased pseudovirus infection levels by over 100-fold compared to infection of WT HEK293T cells. S309 fully neutralized infection via DC-SIGN, L-SIGN, and SIGLEC-1.

Expression of DC-SIGN (CD209) and other cell surface receptor proteins including SIGLEC-1 and other SIGLECs was determined on a variety of cell types.

Further experiments were performed to investigate the function(s) of DC-SIGN, L-SIGN, and SIGLEC-1 in SARS-CoV-2 infection. In one set of experiments, HEK293T cells stably expressing DC-SIGN, L-SIGN, SIGLEC-1 or ACE2 were infected with live SARS-CoV-2 Nluc at three different multiplicities of infection (MOI): 0.01, 0.1, and 1). Infection was determined using relative luminescence units and compared to infection in HEK293T cells (parental). At the lowest MOI tested, an increase of infection in cells expressing DC-SIGN, L-SIGN, or SIGLEC was observed. At the highest MOI tested, infection was not further increased versus parental by expression of DC-SIGN, L-SIGN, or SIGLEC. These data indicate that the parental 293T cells are susceptible to infection by SARS-CoV-2 and L-SIGN, DC-SIGN, and SIGLEC-1 enhance infection levels but do not function as primary receptors for infection.

In another set of experiments, 293T cells, HeLa cells, and MRC5 cells were transiently transduced with lentivirus encoding DC-SIGN, L-SIGN, SIGLEC-1 or ACE2 and infected with VSV pseudovirus three days after transduction. While the 293T cells showed a low level of susceptibility (compare uninfected with untransduced), HeLa and MRC5 cells were completely refractory to the virus. The low level of infection in 293T cells can be increased by expression of L-SIGN, DC-SIGN, or SIGLEC-1, consistent with a role for these proteins as attachment factors. The HeLa and MRC5 cells remained refractory to infection even after expression of L-SIGN, DC-SIGN, or SIGLEC-1, and only become susceptible after expression of ACE2. These data indicate that L-SIGN, DC-SIGN, and SIGLEC-1 are not primary receptors for SARS-CoV-2.

### Example 48

### In Vivo Efficacy of S309 Antibody in an Animal Model of Infection

The efficacy of S309 was investigated in Syrian hamsters. This animal model represents to-date the most relevant model of SARS-CoV-2 infection that did not require in vivo over-expression of ACE2 to support productive infection and disease. Prophylactic administration of S309 induced dose-dependent protection against SARS-CoV-2 infection and tissue damage in hamsters, as demonstrated by the viral RNA levels, the viral load as well as the histopathological score in the lungs. These data indicate that poor and incomplete neutralization of entry by S309 in vitro when using ACE2 over-expressing cells did not compromise in vivo efficacy of non-RBM mAbs.

S309 carrying the N297A mutation has a reduced capacity to trigger effector functions as a consequence of diminished engagement to Fcγ receptors. This was further confirmed by the reduced binding of S309-N297A variant to hamster monocytes in the spleen. The in vivo efficacy measured with the N297A mAb is similar or just slightly inferior to the wt S309, suggesting that neutralizing capacity of the mAb is prevailing upon its effector function capacity in these conditions. The serum concentration of S309 required to reduce the viral RNA in the lung by 90% was 9 μg/ml.

### Example 49

### Antibody Activity Against SARS-CoV-2 Variants

A number of SARS-CoV-2 variants have emerged, with increasing numbers of infection by variants reported in late 2020. The Receptor Binding Motif (RBM) appears to be particularly variable to mutation. Notable emerging variants have been observed in Scotland, the UK, South Africa, California, Columbus, and in minks in Denmark, and some mutations have been reported to confer escape from antibodies or serum neutralization. Experiments were performed to assess the ability of S309 antibodies to neutralize variants. S309 N55Q MLNS (VH: SEQ ID NO.: 113; VL: SEQ ID NO.: 168; with M428L and N434S Fc mutations) was tested against SARS-CoV-2 bearing a panel of the 20 most-frequent SARS-CoV-2 RBD variant mutations, as determined by sequence reads. Antibodies REGN10933 and REGN10987 (Hansen et al., Science 369 (6506): 1010-1014; eabd0827-0810 (2020) and PDB 6XDG (rcsb.org/structure/6XDG)) were included for comparison. Results are summarized in Table 17.

TABLE 17
Summary of Neutralization by Antibodies Against SARS CoV-2 Variants

| Variant Mutation | S309 N55Q MLNS | REGN10933 | REGN10987 |
|---|---|---|---|
| N501Y (UK, South African, | Y | Y | Y |
| and Brazilian mutant) | | | |
| S477N | Y | Y | Y |
| N439K (Scottish mutant) | Y | Y | N |
| L452R (Californian mutant) | Y | P | P |
| E484K (South African and | Y | N | Y |
| Brazilian mutant) | | | |
| Y453F (mink mutant) | Y | N | N |
| | | | (4x decrease) |
| A520S | Y | Y | Y |
| K417N (South African | Y | N | Y |
| mutant) | (K417N/V) | | (K417N/E/V) |
| S494P | Y | N | P |
| S477R | P | ? | P |
| V367F | Y | Y | Y |
| P384L | Y | P | P |
| A522S | Y | P | P |
| A522V | Y | P | P |
| V382L | Y | P | P |
| N501T | Y | P | P |
| P330S | Y | P | P |
| T478I | Y | ? | P |
| S477I | Y | ? | P |
| P479S | Y | P | P |

Y = less than three-fold decrease in neutralizing of live virus or pseudovirus
N = greater than three-fold decrease in neutralizing of live virus or pseudovirus
P = neutralization by antibody is predicted due to variant amino acid being outside of epitope
? = unknown.

Total counts of SARS-CoV-2 sequenced mutants known to escape the antibodies (as of Jan. 29, 2021) were: S309 N55Q MLNS=29; REGN10987=10,425; REGN10933=3,621.

Binding of S309 antibodies to SARS-CoV-2 variant RBDs was assessed by BLI. S309 (VH: SEQ ID NO.: 105; VL: SEQ ID NO.: 168) with wild-type Fc and S309 N55Q (VH: SEQ ID NO.: 113; VL: SEQ ID NO.: 168) bearing MLNS or MLNS+GAALIE Fc mutations were assessed. REGN10987 and REGN10933 were included as comparators. Briefly, antibodies were diluted in kinetics buffer at 3 ug/ml and loaded on Protein-A sensors for 75 seconds. After a short equilibration step in kinetics buffer, loaded sensors were moved in wells containing the RBD variants at 5 ug/ml in kinetics buffer and association was recorded during 3 minutes. Dissociation of the complex was performed in kinetics buffer for 3 minutes. Data are shown in FIGS. 71A-71B; “WT”=Wuhan-Hu-1 with D614G; “Triple Mutant” in lower row=Wuhan-Hu-1 with D614G and added South Africa variant B.1.351 RBD mutations K417N, E484K, and N501Y. Other mutations present in the South Africa variant B.1.351 were not present in the “SA” RBD tested.

Neutralization of S309 antibodies against SARS-CoV-2 variants was assessed using MLV pseudovirus and Vero-E6 target cells expressing TMPRSS2. S309 (VH: SEQ ID NO.: 105; VL: SEQ ID NO.: 168) with wild-type Fc and S309 N55Q (VH: SEQ ID NO.: 113; VL: SEQ ID NO.: 168) bearing MLNS or MLNS+GAALIE Fc mutations were assessed. REGN10987, REGN10933, and the combination of REGN10987+REGN10933, were also assessed. S309 antibodies neutralized the SARS-Cov-2 variants.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/038,738, filed Jun. 12, 2020, U.S. Patent Application No. 63/066,966, filed Aug. 18, 2020, U.S. Patent Application No. 63/141,430, filed Jan. 25, 2021, U.S. Patent Application No. 63/159,404, filed Mar. 10, 2021, U.S. Patent Application No. 63/186,055, filed May 7, 2021, and U.S. Patent Application No. 63/196,089, filed Jun. 2, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of treating a SARS-CoV-2 infection in a subject, the method comprising administering to the subject a single dose of a composition comprising an antibody or antigen-binding fragment that is capable of binding to a surface glycoprotein of a SARS-CoV-2 expressed on a cell surface of a host cell and/or on a virion,
wherein the antibody comprises a heavy chain variable domain (VH) comprising a CDRH1, a CDRH2, and a CDRH3, and a light chain variable domain (VL) comprising a CDRL1, a CDRL2, and a CDRL3,
wherein CDRH1, CDRH2, CDRH3, CDRL1, CDRL2, and CDRL3 comprise the amino acid sequences set forth in:
(1) SEQ ID NOS.: 106, 121, 108, 169, 170, and 171, respectively; or
(2) SEQ ID NOS.: 106, 107, 108, 169, 170, and 171, respectively.

2. A method of treating a SARS-CoV-2 infection in a subject, the method comprising administering to the subject an effective amount of (i) an antibody or antigen-binding fragment that is capable of binding to a surface glycoprotein of a SARS-CoV-2 expressed on a cell surface of a host cell and/or on a virion, or (ii) a composition comprising (ii) (a) the antibody or antigen-binding fragment and (ii) (b) a pharmaceutically acceptable excipient, carrier, or diluent,
wherein the antibody comprises a heavy chain variable domain (VH) comprising a CDRH1, a CDRH2, and a CDRH3, and a light chain variable domain (VL) comprising a CDRL1, a CDRL2, and a CDRL3,
wherein CDRH1, CDRH2, CDRH3, CDRL1, CDRL2, and CDRL3 comprise the amino acid sequences set forth in:
(1) SEQ ID NOS.: 106, 121, 108, 169, 170, and 171, respectively; or
(2) SEQ ID NOS.: 106, 107, 108, 169, 170, and 171, respectively.

3. The method of claim 2, wherein the method comprises administering to the subject a single dose of the antibody or antigen-binding fragment of (i) or the composition of (ii).

4. The method of any one of claims 1-3, wherein the VH comprises the amino acid sequence set forth in SEQ ID NO.: 113 and the VL comprises the amino acid sequence set forth in SEQ ID NO.: 168.

5. The method of any one of claims 1-3, wherein the VH comprises the amino acid sequence set forth in SEQ ID NO.: 105 and the VL comprises the amino acid sequence set forth in SEQ ID NO.: 168.

6. The method of any one of claims 1-5, wherein the antibody or antigen-binding fragment further comprises an Fc polypeptide or a fragment thereof.

7. The method of any one of claims 1-6, which is an IgG, IgA, IgM, IgE, or IgD isotype.

8. The method of any one of claims 1-7, which is an IgG isotype selected from IgG1, IgG2, IgG3, and IgG4.

9. The method of any one of claims 1-8, which is an IgG1 isotype.

10. The method of any one of claims 6-9, wherein the Fc polypeptide or fragment thereof comprises:
(i) a mutation that enhances binding to a FcRn as compared to a reference Fc polypeptide that does not comprise the mutation; and/or
(ii) a mutation that enhances binding to a FcγR as compared to a reference Fc polypeptide that does not comprise the mutation.

11. The method of claim 10, wherein the mutation that enhances binding to a FcRn comprises: M428L; N434S; N434H; N434A; N434S; M252Y; S254T; T256E; T250Q; P257I; Q311I; D376V; T307A; E380A; or any combination thereof.

12. The method of claim 10 or 11, wherein the mutation that enhances binding to FcRn comprises:
(i) M428L/N434S;
(ii) M252Y/S254T/T256E;
(iii) T250Q/M428L;
(iv) P257I/Q311I;
(v) P257I/N434H;
(vi) D376V/N434H;
(vii) T307A/E380A/N434A; or
(viii) any combination of (i)-(vii).

13. The method of any one of claims 10-12, wherein the mutation that enhances binding to FcRn comprises M428L/N434S.

14. The method of any one of claims 10-13, wherein the mutation that enhances binding to a FcγR comprises S239D; I332E; A330L; G236A; or any combination thereof, and optionally does not comprise S239D.

15. The method of any one of claims 10-14, wherein the mutation that enhances binding to a FcγR comprises:
(i) S239D/I332E;
(ii) S239D/A330L/I332E;
(iii) G236A/S239D/I332E; or
(iv) G236A/A330L/I332E.

16. The method of any one of claims 1-15, wherein the antibody or antigen-binding fragment comprises the CH1-CH3 amino acid sequence of SEQ ID NO.: 173 or 265 and the CL amino acid sequence of SEQ ID NO.: 174.

17. The method of any one of claims 1-15, wherein the antibody or antigen-binding fragment comprises the CH1-CH3 amino acid sequence of SEQ ID NO.: 175 or 266 and the CL amino acid sequence of SEQ ID NO.: 174.

18. The method of any one of claims 1-17, wherein the antibody or antigen-binding fragment comprises a heavy chain polypeptide and a light chain polypeptide, wherein:
(i) the heavy chain polypeptide comprises the VH amino acid sequence set forth in SEQ ID NO.: 113 and the CH1-CH3 amino acid sequence set forth in SEQ ID NO.: 173 or 265; and
(ii) the light chain comprises the VL amino acid sequence set forth in SEQ ID NO.: 168 and the CL amino acid sequence set forth in SEQ ID NO.: 174, and wherein, optionally, the method comprises administering a single dose of the antibody or antigen-binding fragment the subject.

19. The method of any one of claims 1-17, wherein the antibody or antigen-binding fragment comprises a heavy chain polypeptide and a light chain polypeptide, wherein:
(i) the heavy chain polypeptide comprises the VH amino acid sequence set forth in SEQ ID NO.: 113 and the CH1-CH3 amino acid sequence set forth in SEQ ID NO.: 175 or 266; and
(ii) the light chain comprises the VL amino acid sequence set forth in SEQ ID NO.: 168 and the CL amino acid sequence set forth in SEQ ID NO.: 174, and wherein, optionally, the method comprises administering a single dose of the antibody or antigen-binding fragment the subject.

20. The method of any one of claims 1-19, wherein the subject:
(i) is aged 18 to 49 years;
(ii) is 18 years old or older;
(iii) has mild to moderate COVID-19;
(iv) has severe COVID-19;
(v) has severe to critical COVID-19;
(vi) has had fewer than seven days or 5 or fewer days since onset of symptoms;
(vii) has had seven days or more since onset of symptoms;
(viii) has had a positive reverse-transcriptase-polymerase-chain-reaction or antigen SARS-CoV-2 test result;
(ix) is 55 years of age or older;
(x) has one or more of: diabetes requiring medication, obesity (body-mass index >30 kg/m²), chronic kidney disease (estimated glomerular filtration rate <60 mL/min/1.73 m²), congestive heart failure (New York Heart Association class II or higher), chronic obstructive pulmonary disease (history of chronic bronchitis, chronic obstructive lung disease, or emphysema with dyspnea on physical exertion), and moderate to severe asthma (subject requires an inhaled steroid to control symptoms or has been prescribed a course of oral steroids in the past year); or
(xi) any combination of (i)-(x).

21. The method of any one of claims 1-20, comprising administering the antibody, antigen-binding fragment, or composition to the subject intravenously.

22. The method of claim 21, comprising administering the antibody, antigen-binding fragment, or composition to the subject intravenously over the course of 30 minutes, 60 minutes, or 90 minutes.

23. The method of any one of claims 1-22, comprising administering the antibody, antigen-binding fragment, or composition to the subject intramuscularly.

24. The method of any one of claims 1-23, wherein the method comprises administering the antibody or antigen-binding fragment to the subject at a dose of up to 100 mg, up to 150 mg, up to 200 mg, up to 250 mg, up to 300 mg, up to 350 mg, up to 400 mg, up to 450 mg, or up to 500 mg.

25. The method of any one of claims 1-24, wherein the method comprises administering the antibody or antigen-binding fragment to the subject at a dose in a range from about 50 mg to about 500 mg, or in a range from about 50 mg to about 250 mg, or in a range from about 50 mg to 100 mg, or in a range from about 100 mg to about 500 mg, or in a range from about 250 mg to about 500 mg.

26. The method of any one of claims 1-25, wherein the method comprises administering 50, 75, 100, 125, 150, 175, 200, 225, 250, 275, 300, 325, 350, 375, 400, 425, 450, 475, or 500 mg of the antibody or antigen-binding fragment to the subject.

27. The method of any one of claims 1-26, wherein the method comprises administering 50, 150, 250, or 500 mg of the antibody or antigen-binding fragment to the subject.

28. The method of any one of claims 1-27, wherein the method comprises administering 500 mg of the antibody or antigen-binding fragment to the subject.

29. The method of any one of claims 1-28, comprising administering the antibody, antigen-binding fragment, or composition to the subject 2, 3, 4, 5, 6, 7, 8, 9, or 10 times, or more.

30. The method of any one of claims 1-29, comprising administering the antibody, antigen-binding fragment, or composition to the subject a plurality of times, wherein a second or successive administration is performed at about 6, about 7, about 8, about 9, about 10, about 11, about 12, about 24, about 48, about 74, about 96 hours, or more, following the first or preceding administration.

31. The method of any one of claims 1-30, wherein the subject is 18 or more years of age with laboratory-confirmed (e.g., by PCR test) SARS-CoV-2 infection.

32. The method of any one of claims 1-31, wherein the subject has a clinical status of Grade 4 (hospitalized, oxygen by mask or nasal prongs), 5 (hospitalized, on non-invasive ventilation, or high flow oxygen), 6 (hospitalized, intubation and mechanical ventilation) or 7 (ventilation and additional organ support-pressors, renal replacement therapy (RRT), extracorporeal membrane oxygenation (ECMO)), as defined by the WHO clinical severity score, 9-point ordinal scale.

33. The method of any one of claims 1-31, wherein the subject has mild-to-moderate COVID-19.

34. The method of claim 33, wherein the subject is at-risk of progression to severe COVID-19.

35. The method of claim 34, wherein following administration of the antibody, antigen-binding fragment, or composition to the subject, the subject is at a reduced risk of hospitalization for COVID-19.

36. The method of claim 35, wherein following administration of the antibody, antigen-binding fragment, or composition to the subject, the risk of hospitalization for COVID-19 is reduced by 10% or more.

37. The method of claim 35, wherein following administration of the antibody, antigen-binding fragment, or composition to the subject, the risk of hospitalization for COVID-19 is reduced by 20% or more.

38. The method of claim 35, wherein following administration of the antibody, antigen-binding fragment, or composition to the subject, the risk of hospitalization for COVID-19 is reduced by 30% or more.

39. The method of claim 35, wherein following administration of the antibody, antigen-binding fragment, or composition to the subject, the risk of hospitalization for COVID-19 is reduced by 40% or more.

40. The method of claim 35, wherein following administration of the antibody, antigen-binding fragment, or composition to the subject, the risk of hospitalization for COVID-19 is reduced by 50% or more.

41. The method of claim 35, wherein following administration of the antibody, antigen-binding fragment, or composition to the subject, the risk of hospitalization for COVID-19 is reduced by 60% or more.

42. The method of claim 35, wherein following administration of the antibody, antigen-binding fragment, or composition to the subject, the risk of hospitalization for COVID-19 is reduced by 70% or more.

43. The method of claim 35, wherein following administration of the antibody, antigen-binding fragment, or composition to the subject, the risk of hospitalization for COVID-19 is reduced by 80% or more.

44. The method of claim 35, wherein following administration of the antibody, antigen-binding fragment, or composition to the subject, the risk of hospitalization for COVID-19 is reduced by 85% or more.

45. The method of any one of claims 1-44, wherein the subject has or is at risk for progressing to severe COVID-19, wherein, optionally, severe COVID-19 comprises (i) hypoxemia (O2 saturation ≤93% on room air or PaO2/FiO2<300) requiring oxygen supplementation for more than 1 day or (ii) the subject requiring ≥4 L/min oxygen supplementation or equivalent.

46. The method of any one of claims 1-45, wherein the subject has or is at risk for progressing to critical COVID-19, wherein, optionally, critical COVID-19 comprises respiratory failure requiring at least one of the following: invasive mechanical ventilation and ECMO; shock; and multi-organ dysfunction/failure.

47. The method of any one of claims 1-46, wherein the subject is less than seven days since onset of symptoms.

48. The method of any one of claims 1-46, wherein the subject is seven days or more since onset of symptoms.

49. The method of any one of claims 1-48, wherein the subject is any one or more of (i)-(iii):
(i) 18 or older and has a positive SARS-CoV-2 test result (by any validated test e.g. RT-PCR on any specimen type);
(ii) (1) hospitalized with severe COVID-19 disease defined as requirement for supplemental oxygen or non-invasive ventilation consistent with Grade 4 or Grade 5 disease or (2) hospitalized with critical COVID-19 disease defined as those on mechanical ventilation (Grade 6 or Grade 7 disease));
(iii) is male or female, wherein, optionally, (1) the woman is non-childbearing potential (WONCBP) or (2) is a woman of child-bearing potential (WOCBP) and uses a contraceptive method.

50. The method of any one of claims 1-49, wherein the method comprises administering 500 mg of the antibody or antigen-binding fragment to the subject.

51. The method of any one of claims 1-50, wherein the subject had or has close contacts to a person with a confirmed SARS-CoV-2 infection.

52. The method of any one of claims 1-51, wherein treating comprises preventing infection by SARS-CoV-2 and/or preventing COVID-19.

53. The method of any one of claims 1-52, wherein treating comprises preventing progression of COVID-19 in the subject.

54. The method of any one of claims 1-53, wherein treating comprises preventing contraction and/or transmission of symptomatic COVID-19.

55. The method of any one of claims 1-53, wherein treating comprises preventing contraction and/or transmission of asymptomatic COVID-19.

56. The method of any one of claims 1-55, wherein the subject is at-risk for contracting or progressing on COVID-19.

57. The method of any one of claims 1-56, wherein treating comprises preventing or reducing:
(1) one or more acute respiratory symptom selected from: cough; sputum production; sore throat; and shortness of breath; or
(2) fever of greater than 38° C.;
(3) two or more of the following symptoms: fatigue; myalgias/arthralgias; chills; nausea/vomiting; diarrhea; and anosmia/dysgeusia.

58. The method of any one of claims 1-57, wherein treating comprises preventing or reducing one or more of the following symptoms: fever of greater than 38° C.; chills; cough; sore throat; malaise; headache; myalgia; a change in smell or taste; nasal congestion/rhinorrhea; vomiting; diarrhea; shortness of breath on exertion.

59. The method of any one of claims 1-58, wherein the subject is an adult.

60. The method of any one of claims 1-59, wherein the subject is 18 or more years of age, or is 19 or more years of age.

61. The method of any one of claims 1-60, wherein the subject is 55 years of age or is, or is 65 years of age or is older

62. The method of any one of claims 1-61, wherein the administering the antibody, antigen-binding fragment, or composition comprises intravenous infusion.

63. The method of any one of claims 1-62, wherein administering the antibody, antigen-binding fragment, or composition comprises intramuscular injection.

64. The method of any one of claims 1-63, wherein the method comprises administering 250 mg of the antibody or antigen-binding fragment to the subject.

65. The method of any one of claims 1-63, wherein the method comprises administering 500 mg of the antibody or antigen-binding fragment to the subject.

66. The method of any one of claims 1-65, wherein the subject has a mild-to-moderate SARS-2-CoV infection (e.g., has mild-to-moderate COVID-19) and, optionally, is at risk for progression to severe disease.

67. The method of any one of claims 1-66, wherein the subject:
(i) is 12 years old or older; and
(ii) last had contact with a person with a confirmed SARS-CoV-2 infection less than three days prior to administration of the composition.

68. The method of any one of claims 1-67, wherein the subject has mild-to-moderate COVID-19 and the method comprises administering a single dose of the antibody, antigen-binding fragment, or composition to the subject intramuscularly.

69. The method of claim 68, wherein the single dose comprises 250 mg of the antibody or antigen-binding fragment.

70. The method of claim 68 or 69, wherein the single dose of comprises 500 mg of the antibody or antigen-binding fragment.

71. The method of any one of claims 68-70, wherein:
(i) (i)(a) the subject is 12 years of age or older and is at high risk of progression of COVID-19 or (i)(b) the subject is 65 years of age or older; and/or
(ii) the subject has a positive SARS-CoV-2 test result (e.g., by PCR test), has oxygen saturation ≥94% on room air, has COVID-19 symptoms, and is less than or equal to 7 days from onset of symptoms.

72. The method of any one of claims 1-71, wherein:
(i) (i)(a) the subject is 12 years of age or older and is at high risk of progression of COVID-19 or (i)(b) the subject is 65 years of age or older; and
(ii) the subject has a positive SARS-CoV-2 test result (e.g., by PCR test), has oxygen saturation ≥94% on room air, has COVID-19 symptoms, and is less than or equal to 7 days from onset of symptoms.

73. The method of any one of claims 1-72, wherein the subject is not hospitalized and is at high-risk for (i) hospitalization and/or (ii) progression of COVID-19.

74. The method of any one of claims 1-73, wherein the subject is:
(1) 12 or more years of age and, optionally, is at high risk of progression of COVID-19; and/or
(2) is 65 or more years of age.

75. The method of any one of claims 1-74, wherein the subject has had a positive SARS-CoV-2 test result, has oxygen saturation ≥94% on room air, has COVID-19 symptoms, and is less than or equal to 7 days from onset of symptoms.

76. The method of any one of claims 1-75, wherein the antibody or antigen-binding fragment was obtained from a non-clonal pool of cells stably transfected with a polynucleotide encoding the antibody or antigen-binding fragment.

77. The method of any one of claims 1-75, wherein the antibody or antigen-binding fragment was obtained from a clonal master cell bank.

78. The method of any one of claims 1-77, wherein the subject: is a resident of a nursing home or a long-term care facility; is a hospice care worker; is a healthcare provider or healthcare worker; is a first responder; is a family member or other close contact of a subject diagnosed with or suspected of having a SARS-CoV-2 infection, is overweight or clinically obese; is or has been a smoker; has or had chronic obstructive pulmonary disease (COPD); is asthmatic (e.g., having moderate to severe asthma); has an autoimmune disease or condition (e.g., diabetes); has a compromised or depleted immune system (e.g., due to AIDS/HIV infection, a cancer such as a blood cancer, a lymphodepleting therapy such as a chemotherapy, a bone marrow or organ transplantation, or a genetic immune condition); has chronic liver disease; has cardiovascular disease; and/or has a pulmonary or heart defect; and/or works or otherwise spends time in close proximity with others, such as in a factory, shipping center, hospital setting, or the like.

79. The method of any one of claims 1-78, wherein the subject has received a vaccine for SARS-CoV-2 and the vaccine is determined to be ineffective, e.g., by post-vaccine infection or symptoms in the subject, by clinical diagnosis or scientific or regulatory criteria.

80. The method of any one of claims 1-78, wherein the subject has not received a vaccine for SARS-CoV-2.

81. The method of any one of claims 1-80, wherein the subject has received convalescent plasma therapy, remdesivir, or both, for SARS-CoV-2.

82. The method of any one of claims 1-81, wherein treatment comprises pre-exposure or peri-exposure prophylaxis.

83. The method of any one of claims 1-82, wherein treatment is administered to the subject having mild-to-moderate disease, optionally in an outpatient setting.

84. The method of any one of claims 1-83, wherein treatment is administered to a subject with moderate-to-severe disease, such as requiring hospitalization.

85. The method of any one of claims 1-84, wherein the subject is hospitalized with COVID-19.

86. The method of any one of claims 1-85, wherein the subject having a SARS-CoV-2 infection: has mild-to-moderate COVID-19; is experiencing any one or more of: fever; cough; fatigue; shortness of breath or difficulty breathing; muscle aches; chills; sore throat; runny nose; headache; chest pain; loss of taste and/or smell; and pink eye (conjunctivitis); malaise; and abnormal imaging; has evidence of lower respiratory disease by clinical assessment or imaging and a saturation of oxygen (SaO2) greater than (>) 93 percent (%) on room air at sea level, has a positive SARS-CoV-2 viral testing result, and/or is at high risk for progressing to severe COVID-19 and/or hospitalization, e.g., the human subject (1) is 65 years of age or older (≥65); has a body mass index (BMI) of 35 or greater (≥35); has chronic kidney disease; has diabetes; (5) has immunosuppressive disease, is receiving immunosuppressive treatment; is 55 years of age or older (≥55) and has cardiovascular disease, hypertension, chronic obstructive pulmonary disease, or other chronic respiratory disease; and/or is 12-17 years of age and has a BMI ≥85% for their age and gender, or sickle cell disease, congenital or acquired heart disease, neurodevelopmental disorders (e.g., cerebral palsy), a medical-related technological dependence (e.g., tracheostomy, gastrostomy, or positive pressure ventilation not related to COVID-19), or asthma, reactive airway or other chronic respiratory disease that requires daily medication for control; has recently been diagnosed with COVID-19 (e.g., within 1 day, 2 days, 3 days, 4 days, 5 days, 6 days, 7 days, 8 days, 9 days, 10 days, 11 days, 12 days, 13 days, or 14 days) and/or is within 10 days of symptom onset; or has or is experiencing any combination of the foregoing.

87. The method of any one of claims 1-85, wherein the subject is (a) 18 years old or older, or (b) 55 years old or younger, provided that the subject is 18 years or older.

88. The method of any one of claims 1-87, wherein the subject has a laboratory confirmed COVID-19 infection by positive polymerase chain reaction (PCR; e.g., RT-PCR) test; e.g., on any type of respiratory tract sample).

89. The method of any one of claims 1-88, the subject has peripheral capillary oxygen saturation (SpO₂) >94% room air (RA), and has experienced one or more symptoms of COVID-19 for ≤120 h (5 days).

90. The method of any one of claims 1-89, wherein the subject is further receiving or has received remdesivir, supplemental oxygen, ventilation therapy, respiration therapy, dexamethasone, tocilizumab, or any combination thereof.
